(19) United States
(12) Patent Application Publication
(10) Pub. No.: US 2025/0134021 A1
(43) Pub. Date: May 1, 2025

(54) MONOCOT LEAF EXPLANT PREPARATION

(71) Applicant: PIONEER HI-BRED INTERNATIONAL, INC., JOHNSTON, IA (US)

(72) Inventors: WILLIAM JAMES GORDON-KAMM, URBANDALE, IA (US); THOMAS RICHARD HAYES, FOLSOM, CA (US); BRANDON MAURER, DES MOINES, IA (US); LARISA A. RYAN, ANKENY, IA (US); NAGESH SARDESAI, JOHNSTON, IA (US); NING WANG, JOHNSTON, IA (US); XINLI EMILY WU, JOHNSTON, IA (US)

(73) Assignee: PIONEER HI-BRED INTERNATIONAL, INC., JOHNSTON, IA (US)

(21) Appl. No.: 18/835,293

(22) PCT Filed: Feb. 3, 2023
(86) PCT No.: PCT/US2023/061901

§ 371 Date: Aug. 1, 2024

Related U.S. Application Data
(60) Provisional application No. 63/306,338, filed on Feb. 3, 2022.

(51) Int. Cl. A01H 4/00; A01C 1/02; A01G 22/00; C12N 15/82

(52) U.S. Cl. CPC A01H 4/008; A01G 22/00; C12N 15/8205; A01C 1/02

(57) ABSTRACT

Methods, including high-throughput, automated methods for preparing, by mechanical and/or robotic means, monocot leaf explants for transformation, are provided.

### PRIORITY CLAIM

This application claims priority to PCT Application Serial No. PCT/US2023/61901, filed Feb. 3, 2023, which claims priority to U.S. Provisional Application Ser. No. 63/306,338, filed Feb. 3, 2022, each of which is expressly incorporated by reference herein.

### FIELD OF THE DISCLOSURE

The present disclosure relates to the field of plant molecular biology, including genetic manipulation of plants. More particularly, the present disclosure pertains to systems and methods for high-throughput automated preparation and transformation of monocot leaf explants.

### REFERENCE TO SEQUENCE LISTING SUBMITTED ELECTRONICALLY

The official copy of the sequence listing is being electronically submitted herewith, as an xml formatted sequence listing named “108004-US-PCT-1_Sequence Listing” created on Jul. 29, 2024 and having a size of 283,665 bytes. The submitted sequence listing is expressly incorporated herein by reference in its entirety.

### BACKGROUND OF THE DISCLOSURE

In recent years, there has been a tremendous expansion of the capabilities for the genetic engineering of plants. Current transformation technology provides an opportunity to produce commercially viable transgenic plants, enabling the creation of new plant varieties containing desirable traits. One limitation of the genetic engineering of plants is the availability of plant tissue explants that are amenable to transformation since many plant tissue explants are recalcitrant to transformation and regeneration. Thus, there is a need for plant transformation systems and methods for efficient, high-throughput, non-destructive, automated approaches to produce large-scale preparation and transformation of monocot leaf explants with minimal manual involvement.

### SUMMARY OF THE DISCLOSURE

In an embodiment, the present disclosure comprises high-throughput automated methods and compositions for the preparation and transformation of monocot leaf explants for producing transgenic plants that contain a heterologous polynucleotide and high-throughput automated methods and compositions using monocot leaf explants for producing gene edited plants. In a further aspect, the present disclosure provides a seed from the plant produced by the methods disclosed herein.

In an aspect, a high-throughput, automated method of producing a population of leaf explants comprising providing a population of seed; sterilizing the population of seed to provide a sterilized seed population; transferring the sterilized seed population by mechanical and/or robotic means to a germination container, wherein the germination container comprises a germination medium; providing a distribution of the sterilized seed population by mechanical and/or robotic means in the germination container; germinating the sterilized seed population to provide a population of germinated seedlings; growing the germinated seedlings to a sufficient height for harvesting seedling leaves by mechanical and/or robotic means; harvesting the seedling leaves by mechanical and/or robotic means to provide harvested seedling leaves; transferring the harvested seedling leaves to a holding container by mechanical and/or robotic means, wherein the holding container comprises a chopping means, a cutting means, and/or a blending means and chopping, cutting, and/or blending the harvested seedling leaves to provide leaf segments of an appropriate size for a bacterial infection; providing to the holding container a bacterial infection solution; infecting the leaf segments in the holding container for a sufficient amount of time to provide bacterially infected leaf segments; removing by mechanical and/or robotic means the bacterial solution from the bacterially infected leaf segments; and providing further sub-culturing, resting, selection, heat-treatment, regeneration, maturation, or rooting processes by automated, mechanical, and/or robotic means is provided. In an aspect, the sterilizing comprises an ethanol treatment, a chlorine gas treatment, a dilute bleach solution treatment, and combinations of the foregoing. In an aspect, the germination medium is a solid, a semi-solid, or a hydroponic medium. In an aspect, harvesting comprises cutting and/or chopping the harvested seedling leaves with scissors, blades, wires, lasers, high-pressure water jet streams, high-pressure air, and combinations of the foregoing. In an aspect, the holding container further comprises a bacterial infection solution. In an aspect, the bacterial infection solution comprises Agrobacterium. In an aspect, the chopping, cutting and or blending is performed in a blender or a food processor. In an aspect, the leaf segments of an appropriate size comprise leaf segments of from about 0.1 mm to about 1 mm, from about 1 mm to about 2 mm, from about 2 mm to about 3 mm, and from about 3 mm to about 1 cm in size.

In an aspect, a high-throughput, automated method of producing a population of leaf explants comprising providing a population of seedlings; transporting each seedling to a first cutting device; removing a top portion of the seedlings using the first cutting device; transporting the seedlings to a second cutting device after removing the top portion of the seedlings; removing a second portion of the seedlings using the second cutting device to provide leaf segments; loading the leaf segments into a loading device configured to dispense a predetermined amount of leaf segments into a container each comprising means for chopping, a cutting, and/or a blending the leaf segments; dispensing the predetermined amount of leaf segments in the container; injecting a predetermined amount of bacterial infection solution into the container; attaching a cap to the container to close the container and prevent the leaf segments and bacterial infection solution from moving outside the container; and chopping, a cutting, and/or a blending the leaf segments to provide leaf fragments of a predetermined size. In an aspect, the transporting steps includes using a conveyor. In an aspect, the method further comprising measuring a height of each seedling included in the population of seedlings using an optical sensor; determining if the measured height of the seedlings is within a predetermined height threshold; and transporting each seedling to the first cutting device if the measured height of the seedlings is within the predetermined height threshold. In an aspect, the method further comprising measuring a length of each leaf segment using an optical sensor after removing the second portion of the seedlings; determining if the measured length of each leaf segment is within a predetermined length threshold; and loading the leaf segments into the loading device if the measured length of the leaf segment is within the predetermined length threshold. In an aspect, the first cutting device includes one of scissors, blades, wires, lasers, high-pressure water jet streams, and high-pressure air. In an aspect, the second cutting device includes one of scissors, blades, wires, lasers, high-pressure water jet streams, and high-pressure air.

### BRIEF DESCRIPTION OF THE DRAWINGS

FIG. 1 is a schematic flow-chart illustrating steps of leaf explant preparation and transformation.

FIG. 2 is a perspective view of a blade assembly for automated cutting that includes a rotor configured to rotate about an axis and a plurality of rotor blades coupled to the rotor for rotation therewith. The plurality of blades have a tilt angle relative to a radial axis of the blade assembly.

FIG. 3 is a perspective view of the blade assembly of FIG. 2. Each of the plurality of blades included in the blade assembly have a pitch angle.

FIG. 4 is a schematic flow-chart illustrating high throughput automated steps of harvesting leaf explants as described in Example 16.

FIG. 5 is a schematic flow-chart illustrating high throughput automated steps of explant segmentation and transformation as described in Example 17.

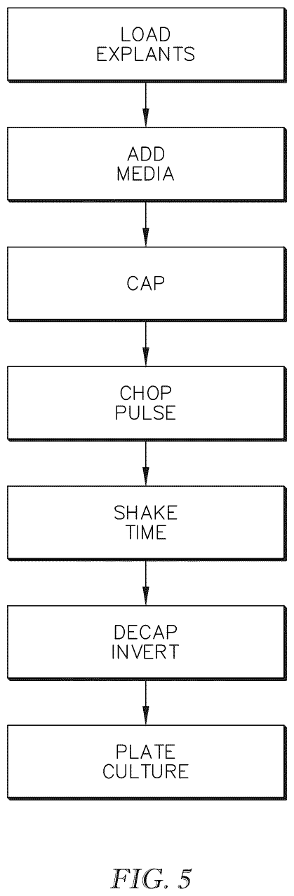

FIG. 6 is a schematic flow-chart illustrating high throughput automated explant preparation and processing as described in Example 18.

FIG. 7 is a schematic flow-chart illustrating high throughput automated explant preparation and processing as described in Example 19.

FIG. 8 is a schematic flow-chart illustrating high throughput automated explant preparation and processing as described in Example 21.

FIG. 9 is a diagrammatic view of an automated leaf explant preparation system as described in Example 22 illustrating the automated leaf explant preparation system includes a seed receiving station, a seed sterilization station, a seedling germination station, a leaf harvesting station, a leaf preparation station, a leaf cutting station, and a transformation station.

FIG. 10 is a diagrammatic view of the seed sterilization station included in the automated leaf explant preparation system of FIG. 9 illustrating the seed sterilization station include means for providing and decanting/removing a series of solutions to the container of seed to provide sterilized seed.

FIG. 11 is a diagrammatic view of the seedling germination station included in the automated leaf explant preparation system of FIG. 9 illustrating the seedling germination station includes growth containers containing germination media which receive the sterilized seed from the sterilization station. The sterilized seed are germinated until the seedlings reach a predetermined height.

FIG. 12 is a diagrammatic view of the leaf harvesting station included in the automated leaf explant preparation system of FIG. 9 illustrating the leaf harvesting station includes cutting devices configured to remove the leaf canopy for disposal and leaf segments for further processing.

FIG. 13 is a diagrammatic view of the leaf preparation station included in the automated leaf explant preparation system of FIG. 9 illustrating the leaf preparation station includes a conveyor system having a conveyor and bladed containers that are transported by the conveyor through the different loading steps to deposit the leaf segments into the containers.

FIG. 14 is a diagrammatic view of the leaf cutting station included in the automated leaf explant preparation system of FIG. 9 illustrating the leaf cutting station includes chopping, cutting, and/or blending means of the harvested seedling leaves to provide leaf segments of an appropriate size for further processing.

FIG. 15 is a diagrammatic view of the transformation station included in the automated leaf explant preparation system of FIG. 9 illustrating the transformation station includes moving the leaf segments between different media after the leaf segments have been infected with the bacterial infection solution.

FIG. 16 is a perspective view of an embodiment of the chopping, cutting, and/or blending means of FIG. 14 showing the chopping, cutting, and/or blending means is a blade assembly that includes a rotor configured to rotate about an axis and a plurality of rotor blades coupled to the rotor for rotation therewith. The plurality of blades includes two curved blades that extend circumferentially at least partway about the axis.

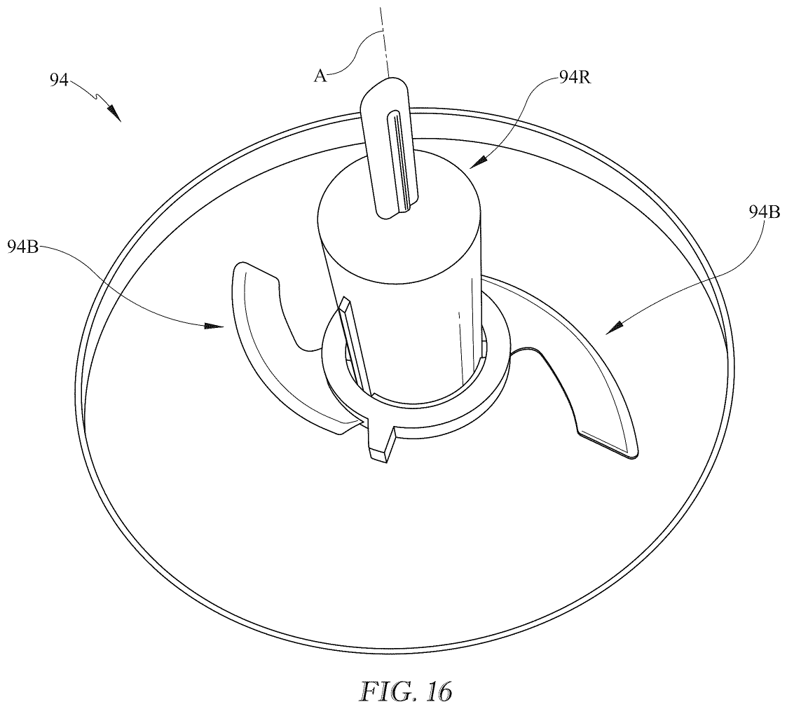

FIG. 17 is an elevation view of the leaf segments after being chopped, cut, or blended using the blade assembly of FIG. 16.

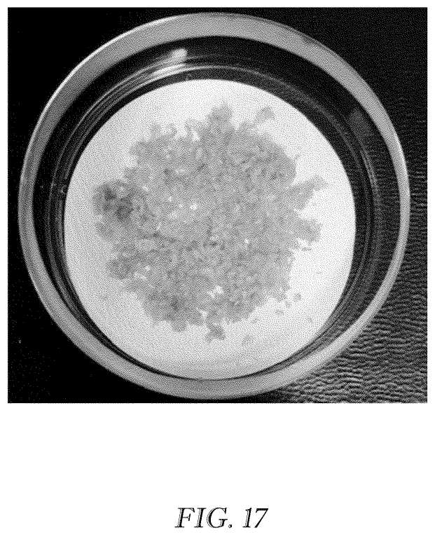

FIG. 18 is a perspective view of another embodiment of the chopping, cutting, and/or blending means of FIG. 14 showing the chopping, cutting, and/or blending means is a blade assembly that includes a rotor configured to rotate about an axis and a plurality of rotor blades coupled to the rotor for rotation therewith. The plurality of blades includes two #60 surgical scalpel blades.

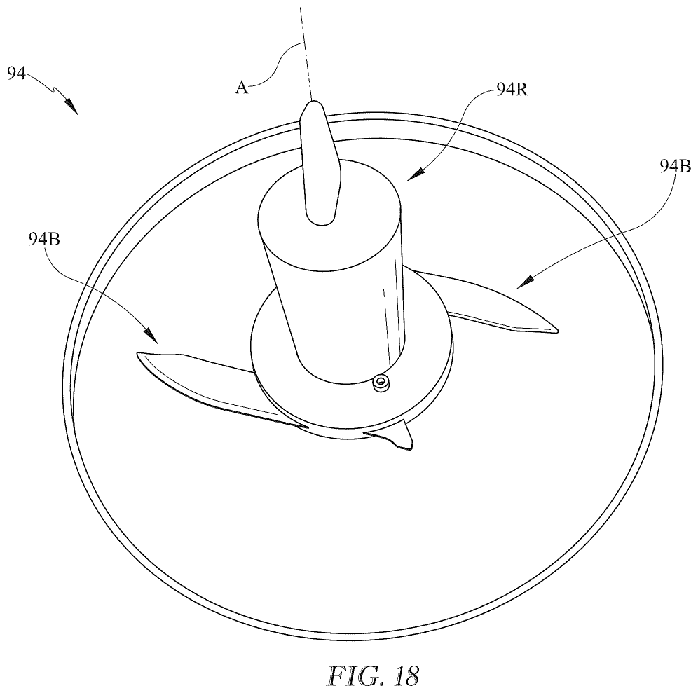

FIG. 19 is an elevation view of the leaf segments after being chopped, cut, or blended using the blade assembly of FIG. 18.

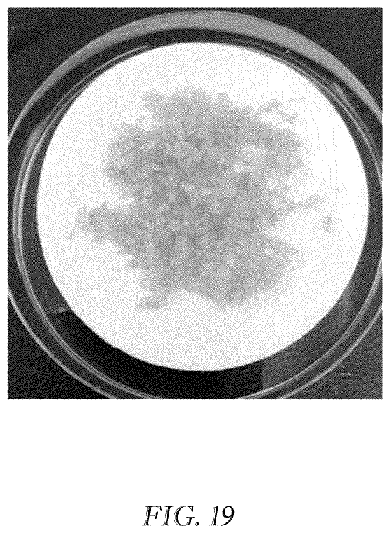

FIG. 20 is a perspective view of another embodiment of the chopping, cutting, and/or blending means of FIG. 14 showing the chopping, cutting, and/or blending means is a blade assembly that includes a rotor configured to rotate about an axis and a plurality of rotor blades coupled to the rotor for rotation therewith. The plurality of blades includes two curved blades.

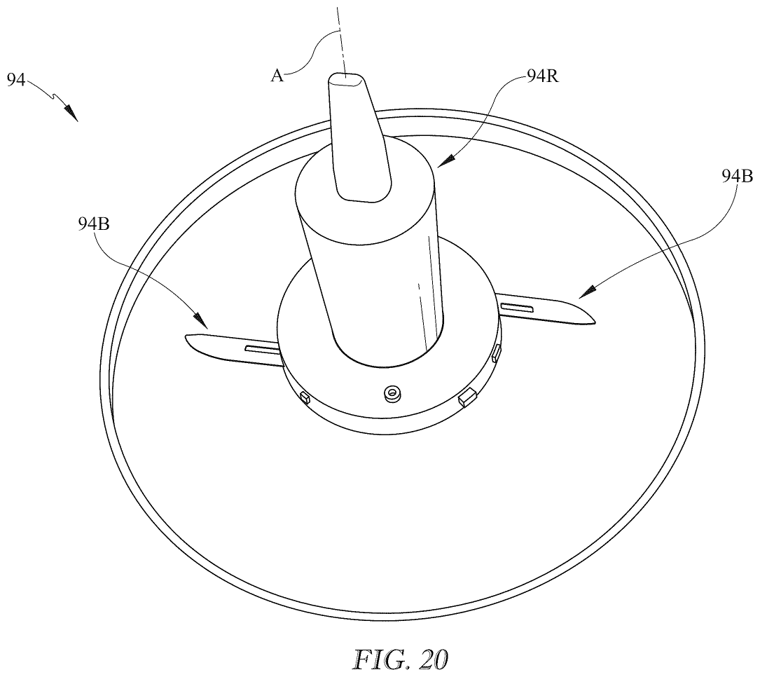

FIG. 21 is an elevation view of the leaf segments after being chopped, cut, or blended using the blade assembly of FIG. 20.

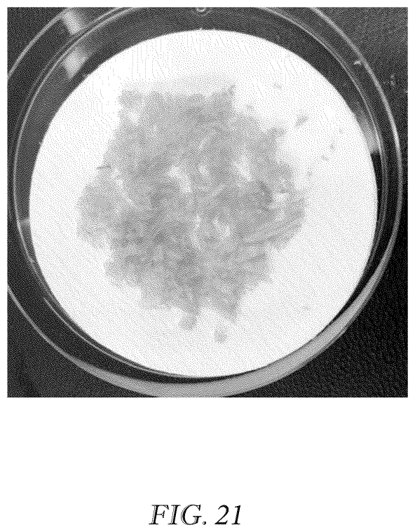

FIG. 22 is an elevation view of the leaf segments after being chopped, cut, or blended using the blade assembly of FIG. 20 with the blades of the blade assembly adjusted to be at a pitch angle of about 2 degrees.

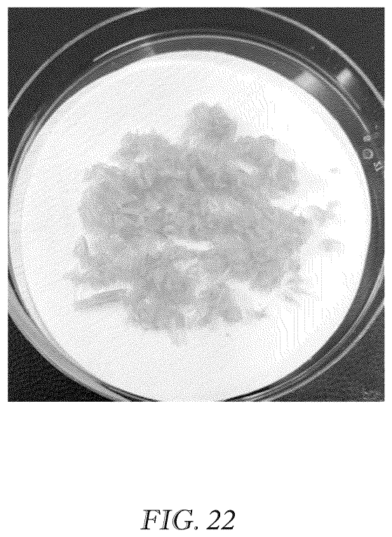

FIG. 23 is an elevation view of the leaf segments after being chopped, cut, or blended using the blade assembly of FIG. 20 with the blades of the blade assembly adjusted to be at a pitch angle of about 4 degrees.

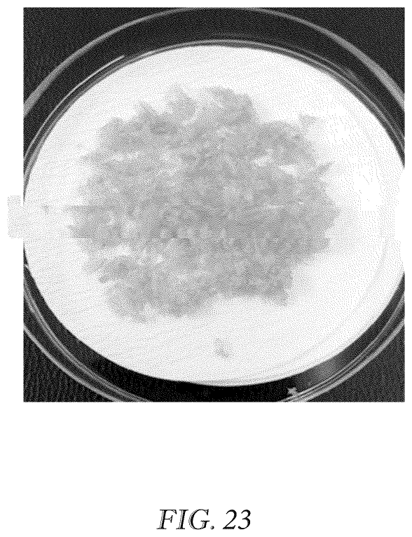

FIG. 24 is an elevation view of the leaf segments after being chopped, cut, or blended using the blade assembly of FIG. 20 with the blades of the blade assembly adjusted to be at a pitch angle of about 4 degrees.

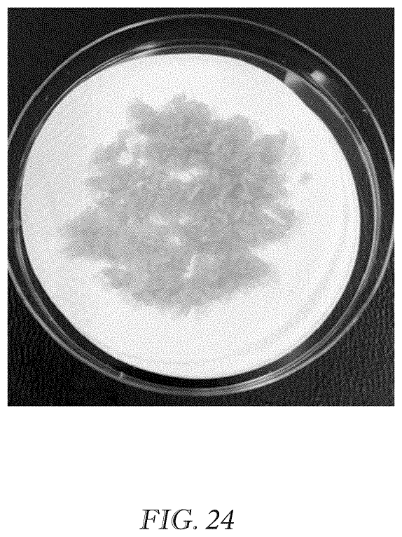

### DETAILED DESCRIPTION

The disclosures herein will be described more fully hereinafter with reference to the accompanying figures, in which some, but not all possible aspects are shown. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed methods pertain having the benefit of the teachings presented in the following descriptions. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of” Unless defined otherwise, all technical and scientific terms used herein have the same meaning as commonly understood by one of ordinary skill in the art to which the disclosed compositions and methods belong. In this specification and in the claims which follow, reference will be made to a number of terms which shall be defined herein.

The methods of the present disclosure improve productivity when used in plant transformation efforts by permitting efficiencies in plant explant preparation providing savings in greenhouse space and providing ergonomic safety in the workplace, while also being amenable to scale-up and further automation.

As used herein, “contacting”, “contact”, “contacted”, “comes in contact with” or “in contact with” means “direct contact” or “indirect contact”. For example, cells are placed in a condition where the cells can come into contact with an expression cassette, a nucleotide, a peptide, a RNP (ribonucleoprotein), or other substance disclosed herein. Such expression cassette, nucleotide, peptide, or other substance is allowed to be present in an environment where the cells survive (for example, medium or expressed in the cell or expressed in an adjacent cell) and can act on the cells. For example, medium comprising a selection agent may have direct contact with a cell or the medium comprising the selection agent may be separated from the cell by filter paper, plant tissues, or other cells thus, the selection agent is transferred through the filter paper, plant tissues, or other cells to the cell. The expression cassettes, nucleotides, peptides, and other substances disclosed herein may be contacted with a cell by T-DNA transfer, particle bombardment, electroporation, PEG transfection, or RNP (ribonucleoprotein) delivery.

As used herein, a “somatic embryo” is a multicellular structure that progresses through developmental stages that are similar to the development of a zygotic embryo, including formation of globular and transition-stage embryos, formation of an embryo axis and a scutellum, and accumulation of lipids and starch. Single somatic embryos derived from a zygotic embryo germinate to produce single non-chimeric plants, which may originally derive from a single-cell.

As used herein, an “embryogenic callus” or “callus” is a friable or non-friable mixture of undifferentiated or partially undifferentiated cells which subtend proliferating primary and secondary somatic embryos capable of regenerating into mature fertile plants.

As used herein, “germination” is the growth of a regenerable structure to form a plantlet which continues growing to produce a plant.

As used herein, a “transgenic plant” is a mature, fertile plant that contains a transgene.

The methods of the disclosure can be used to transform leaf explants. As used herein, “leaf explants” include but are not limited to radical leaves, cauline leaves, alternate leaves, opposite leaves, decussate leaves, opposite superposed leaves, whorled leaves, petiolate leaves, sessile leaves, subsessile leaves, stipulate leaves, exstipulate leaves, simple leaves, or compound leaves. Leaf explants include buds, including but not limited to lateral buds, leaf primordia, the leaf sheath, leaf base or the portion of the leaf immediately proximal to its attachment point to the petiole or stem. Such vegetative organs and their composite tissues can be used for transformation with nucleotide sequences encoding agronomically important traits.

As used herein, a “leaf” is a flat lateral structure that protrudes from a plant's stem, including the supporting stalk between the flattened leaf and the plant stem, but not including the axillary meristem located at the junction of the petiole and stem, including but not limited to a radical leaf, a cauline leaf, an alternate leaf, and opposite leaf, a decussate leaf, an opposite superposed leaf, a whorled leaf, a petiolate leaf, a sessile leaf, a subsessile leaf, a stipulate leaf, an exstipulate leaf, a simple leaf, or a compound leaf.

As used herein, the term “morphogenic gene” means a gene that when ectopically expressed stimulates formation of a somatically-derived structure that can produce a plant. More precisely, ectopic expression, or mutation, or silencing, or decreased expression of the morphogenic gene stimulates the de novo formation of a somatic embryo or an organogenic structure, such as a shoot meristem or an axillary meristem, that can produce a plant or stimulates regeneration of a plant. This stimulated de novo formation occurs either in the cell in which the morphogenic gene is expressed, or silenced, or repressed, or in a neighboring cell. A morphogenic gene can be a transcription factor that regulates expression of other genes, or a gene that influences hormone levels in a plant tissue, both of which can stimulate morphogenic changes. A morphogenic gene may be stably incorporated into the genome of a plant or it may be transiently expressed. In an aspect, expression of the morphogenic gene is controlled. The expression can be controlled transcriptionally or post-transcriptionally. The controlled expression may also be a pulsed expression of the morphogenic gene for a particular period of time. Alternatively, the morphogenic gene may be expressed in only some transformed cells and not expressed in others. The control of expression of the morphogenic gene can be achieved by a variety of methods as disclosed herein below. The morphogenic genes useful in the methods of the present disclosure may be obtained from or derived from any plant species.

As used herein, the term “morphogenic factor” means a morphogenic gene and/or the protein expressed by a morphogenic gene.

A morphogenic gene is involved in plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem or axillary meristem, initiation and/or development of shoots, or a combination thereof, such as WUS/WOX genes (WUS, WUS1, WUS2, WUS3, WOX2A, WOX4, WOX5, or WOX9) see U.S. Pat. Nos. 7,348,468 and 7,256,322 and United States Patent Application publications 20170121722 and 20070271628; Laux et al. (1996) Development 122:87-96; and Mayer et al. (1998) Cell 95:805-815; van der Graaff et al., 2009, Genome Biology 10:248; Dolzblasz et al., 2016, Mol. Plant 19:1028-39 are useful in the methods of the disclosure. Modulation of WUS/WOX is expected to modulate plant and/or plant tissue phenotype including plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof. Expression of Arabidopsis WUS can induce stem cells in vegetative tissues, which can differentiate into somatic embryos (Zuo, et al. (2002) Plant J 30:349-359). Also of interest in this regard would be a MYB118 gene (see U.S. Pat. No. 7,148,402), MYB115 gene (see Wang et al. (2008) Cell Research 224-235), a BABYBOOM gene (BBM; see Boutilier et al. (2002) Plant Cell 14:1737-1749), a CLAVATA gene (see, for example, U.S. Pat. No. 7,179,963), an Enhancer of Shoot Regeneration 1 (ESR1) gene (see Banno et al. (2001), The Plant Cell, Vol. 13:2609-2618), a Corngrass1 (Cg1) gene (see Chuck et al. (2007) Nature Genetics, Vol. 39(4):544-549), a Cup-Shaped Cotyledon (CUC) gene (see Hibara et al. (2006) The Plant Cell, Vol. 18:2946-2957), a REVOLUTA (REV) gene (see Otsuga et al. (2001) The Plant Journal 25(2):223-236), a More Axillary Growth1 (MAX1) gene (see Stirnberg et al. (2002) Development 129:1131-1141), a SUPERSHOOT (SPS) gene (see Tanikanjana, et al. (2001) Genes & Development 15:1577-1588), a Lateral Suppressor (LAS) gene (see Greb et al. (2003) Genes & Development 17:1175-1187), a More Axillary Growth4 (MAX4) gene (see Sorefan et al. (2003) Genes & Development 17:1469-1474), a Stem Cell-Inducing Factor 1 (STEMIN1) gene (see Ishikawa et al. (2019) Nature Plants 5:681-690), a Growth-Regulating Factor 4 (GRF4) gene and/or a GRF-Interacting Factor 1 (GIF1) gene (see Debernardi et al. bioRxiv 2020.08.23.263905; doi:https://doi.org/10.1101/2020.08.23.263905), and a Growth-Regulating Factor 5 (GRF5) gene (see Kong et al. bioRxiv 2020.08.23.263947; doi:https://doi.org/10.1101/2020.08.23.263947).

Morphogenic polynucleotide sequences and amino acid sequences of functional WUS/WOX polypeptides are useful in the disclosed methods. As defined herein, a “functional WUS/WOX nucleotide” is any polynucleotide encoding a protein that contains a homeobox DNA binding domain, a WUS box, and an EAR repressor domain (Ikeda et al., 2009 Plant Cell 21:3493-3505). As demonstrated by Rodriguez et al., 2016 PNAS www.pnas.org/cgi/doi/10.1073/pnas.1607673113 removal of the dimerization sequence which leaves behind the homeobox DNA binding domain, a WUS box, and an EAR repressor domain results in a functional WUS/WOX polypeptide. The Wuschel protein, designated hereafter as WUS, plays a key role in the initiation and maintenance of the apical meristem, which contains a pool of pluripotent stem cells (Endrizzi, et al., (1996) Plant Journal 10:967-979; Laux, et al., (1996) Development 122:87-96; and Mayer, et al., (1998) Cell 95:805-815). Arabidopsis plants mutant for the WUS gene contain stem cells that are misspecified and that appear to undergo differentiation. WUS encodes a novel homeodomain protein which presumably functions as a transcriptional regulator (Mayer, et al., (1998) Cell 95:805-815). The stem cell population of Arabidopsis shoot meristems is believed to be maintained by a regulatory loop between the CLAVATA (CLV) genes which promote organ initiation and the WUS gene which is required for stem cell identity, with the CLV genes repressing WUS at the transcript level, and WUS expression being sufficient to induce meristem cell identity and the expression of the stem cell marker CLV3 (Brand, et al., (2000) Science 289:617-619; Schoof, et al., (2000) Cell 100:635-644). Constitutive expression of WUS in Arabidopsis has been shown to lead to adventitious shoot proliferation from leaves (in planta) (Laux, T., Talk Presented at the XVI International Botanical Congress Meeting, Aug. 1-7, 1999, St. Louis, Mo.).

In an aspect, the functional WUS/WOX polypeptides useful in the methods of the present disclosure is a WUS, WUS1, WUS2, WUS3, WOX2A, WOX4, WOX5, WOX5A, or WOX9 polypeptide (see, U.S. Pat. Nos. 7,348,468 and 7,256,322 and US Patent Application Publication Numbers 2017/0121722 and 2007/0271628, herein incorporated by reference in their entirety and van der Graaff et al., 2009, Genome Biology 10:248). The functional WUS/WOX polypeptides useful in the methods of the present disclosure can be obtained from or derived from any plant including but not limited to monocots, dicots, Angiospermae, and Gymnospermae.

Other morphogenic genes useful in the present disclosure include, but are not limited to, LEC1 (U.S. Pat. No. 6,825,397 incorporated herein by reference in its entirety, Lotan et al., 1998, Cell 93:1195-1205), LEC2 (Stone et al., 2008, PNAS 105:3151-3156; Belide et al., 2013, Plant Cell Tiss. Organ Cult 113:543-553), KN1/STM (Sinha et al., 1993. Genes Dev 7:787-795), the IPT gene from Agrobacterium (Ebinuma and Komamine, 2001, In vitro Cell. Dev Biol—Plant 37:103-113), MONOPTEROS-DELTA (Ckurshumova et al., 2014, New Phytol. 204:556-566), the Agrobacterium AV-6b gene (Wabiko and Minemura 1996, Plant Physiol. 112:939-951), the combination of the Agrobacterium IAA-h and IAA-m genes (Endo et al., 2002, Plant Cell Rep., 20:923-928), the Arabidopsis SERK gene (Hecht et al., 2001, Plant Physiol. 127:803-816), the Arabidopsis AGL15 gene (Harding et al., 2003, Plant Physiol. 133:653-663), the FUSCA gene (Castle and Meinke, Plant Cell 6:25-41), and the PICKLE gene (Ogas et al., 1999, PNAS 96:13839-13844).

As used herein, the term “transcription factor” means a protein that controls the rate of transcription of specific genes by binding to the DNA sequence of the promoter and either up-regulating or down-regulating expression. Examples of transcription factors that are also morphogenic genes, include members of the AP2/EREBP family (including BBM (ODP2)), plethora and aintegumenta sub-families, CAAT-box binding proteins such as LEC1 and HAP3, and members of the MYB, bHLH, NAC, MADS, bZIP and WRKY families.

Morphogenic polynucleotide sequences and amino acid sequences of Ovule Development Protein 2 (ODP2) polypeptides, and related polypeptides, e.g., Babyboom (BBM) protein family proteins are useful in the methods of the disclosure. In an aspect, a polypeptide comprising two AP2-DNA binding domains is an ODP2, BBM2, BMN2, or BMN3 polypeptide see, US Patent Application Publication Number 2017/0121722, herein incorporated by reference in its entirety. ODP2 polypeptides useful in the methods of the disclosure contain two predicted APETALA2 (AP2) domains and are members of the AP2 protein family (PFAM Accession PF00847). The AP2 family of putative transcription factors has been shown to regulate a wide range of developmental processes, and the family members are characterized by the presence of an AP2 DNA binding domain. This conserved core is predicted to form an amphipathic alpha helix that binds DNA. The AP2 domain was first identified in APETALA2, an Arabidopsis protein that regulates meristem identity, floral organ specification, seed coat development, and floral homeotic gene expression. The AP2 domain has now been found in a variety of proteins.

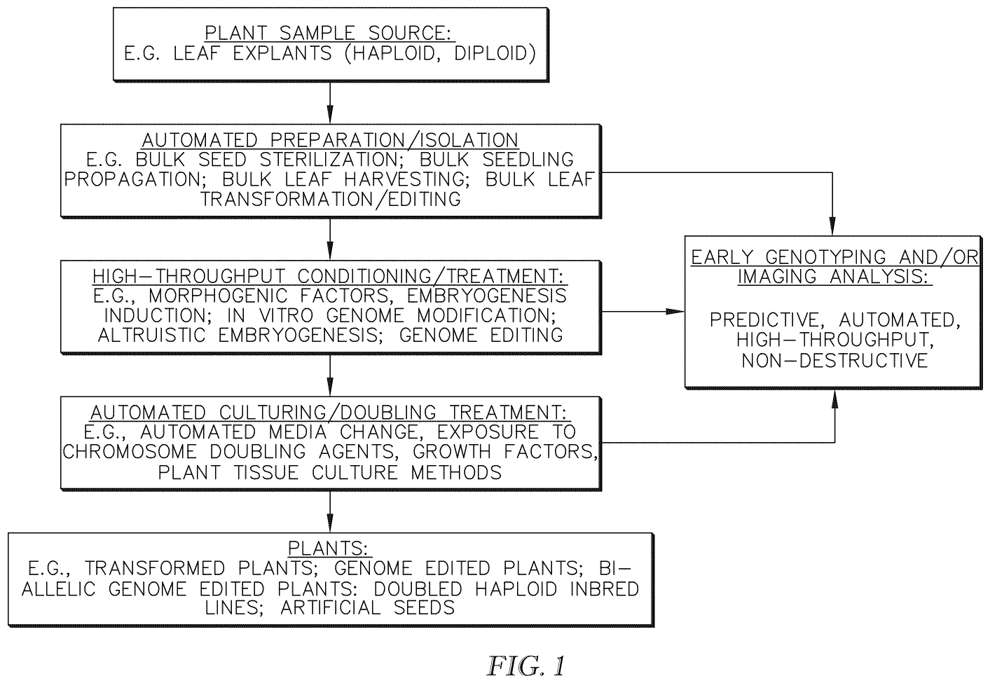

ODP2 polypeptides useful in the methods of the disclosure share homology with several polypeptides within the AP2 family, e.g., see FIG. 1 of U.S. Pat. No. 8,420,893, which is incorporated herein by reference in its entirety, and provides an alignment of the maize and rice ODP2 polypeptides with eight other proteins having two AP2 domains. A consensus sequence of all proteins appearing in the alignment of U.S. Pat. No. 8,420,893 is also provided in FIG. 1 therein. The polypeptide comprising the two AP2-DNA binding domains useful in the methods of the disclosure can be obtained from or derived from any of the plants described herein. In an aspect, the polypeptide comprising the two AP2-DNA binding domains useful in the methods of the disclosure is an ODP2 polypeptide. In an aspect, the polypeptide comprising the two AP2-DNA binding domains useful in the methods of the disclosure is a BBM2 polypeptide. The ODP2 polypeptide and the BBM2 polypeptide useful in the methods of the disclosure can be obtained from or derived from any plant including but not limited to monocots, dicots, Angiospermae, and Gymnospermae.

As used herein, the term “expression cassette” means a distinct component of vector DNA consisting of coding and non-coding sequences including 5′ and 3′ regulatory sequences that control expression in a transformed/transfected cell.

As used herein, the term “coding sequence” means the portion of DNA sequence bounded by a start and a stop codon that encodes the amino acids of a protein.

As used herein, the term “non-coding sequence” means the portions of a DNA sequence that are transcribed to produce a messenger RNA, but that do not encode the amino acids of a protein, such as 5′ untranslated regions, introns and 3′ untranslated regions. Non-coding sequence can also refer to RNA molecules such as micro-RNAs, interfering RNA or RNA hairpins, that when expressed can down-regulate expression of an endogenous gene or another transgene.

As used herein, the term “regulatory sequence” means a segment of a nucleic acid molecule which is capable of increasing or decreasing the expression of a gene. Regulatory sequences include promoters, terminators, enhancer elements, silencing elements, 5′ UTR and 3′ UTR (untranslated regions).

As used herein, the term “UBI” or “UBI1” or “UBI PRO” or “UBI1 PRO” or “ZM-UBI PRO” or “ZM-UBI1 PRO” or “ZM-UBI1 PRO Complete” (SEQ ID NO. 20) is made up of the UBI1ZM PRO sequence (SEQ ID NO. 14) and the UBI1ZM 5UTR (SEQ ID NO. 15) and the UBI1ZM INTRON1 (SEQ ID NO. 16).

As used herein, the term “3×ENH” (SEQ ID NO. 21) is made up of the FMV ENH (SEQ ID NO. 17) and the PCSV ENH (SEQ ID NO. 18) and the MMV ENH (SEQ ID NO. 19).

As used herein, the term “transfer cassette” means a T-DNA comprising an expression cassette or expression cassettes flanked by the right border and the left border.

As used herein, “T-DNA” means a portion of a Ti plasmid that is inserted into the genome of a host plant cell.

As used herein, the term “selectable marker” means a transgene that when expressed in a transformed/transfected cell confers resistance to selective agents such as antibiotics, herbicides and other compounds toxic to an untransformed/untransfected cell or provides a selective growth advantage to the transformed cells (eg. phosphomannose isomerase, PMI).

As used herein, the term “EAR” means an Ethylene-responsive element binding factor-associated Amphiphilic Repression motif having general consensus sequences that act as transcriptional repression signals within transcription factors. Addition of an EAR-type repressor element to a DNA-binding protein such as a transcription factor, dCAS9, or LEXA (as examples) confers transcriptional repression function to the fusion protein (Kagale, S., and Rozwadowski, K. 2010. Plant Signaling and Behavior 5:691-694).

In an aspect, the methods of the disclosure comprise contacting a monocot leaf explant with a recombinant expression cassette or construct comprising a nucleotide sequence encoding a functional WUS/WOX polypeptide, or a nucleotide sequence encoding a Babyboom (BBM) polypeptide or an Ovule Development Protein 2 (ODP2) polypeptide, or a combination of a nucleotide sequence encoding a functional WUS/WOX polypeptide and a nucleotide sequence encoding a Babyboom (BBM) polypeptide or an Ovule Development Protein 2 (ODP2) polypeptide to produce a transgenic monocot plant comprising a heterologous polynucleotide.

In an aspect, a nucleotide sequence encoding a functional WUS/WOX polypeptide, or a nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be targeted for excision by a site-specific recombinase. Thus, the expression of the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be controlled by excision at a desired time post-transformation. It is understood that when a site-specific recombinase is used to control the expression of the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide, the expression construct comprises appropriate site-specific excision sites flanking the polynucleotide sequences to be excised, e.g., Cre lox sites if Cre recombinase is utilized. It is not necessary that the site-specific recombinase be co-located on the expression construct comprising the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide. However, in an aspect, the morphogenic gene expression cassette further comprises a nucleotide sequence encoding a site-specific recombinase.

The site-specific recombinase used to control expression of the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be chosen from a variety of suitable site-specific recombinases. For example, in various aspects, the site-specific recombinase is FLP, FLPe, KD, Cre, SSV1, lambda Int, phi C31 Int, HK022, R, B2 (Nern et al., (2011) PNAS Vol. 108, No. 34 pp 14198-14203), B3 (Nern et al., (2011) PNAS Vol. 108, No. 34 pp 14198-14203), Gin, Tn1721, CinH, ParA, Tn5053, Bxb1, TP907-1, or U153. The site-specific recombinase can be a destabilized fusion polypeptide. The destabilized fusion polypeptide can be TETR(G17A)˜CRE or ESR(G17A)˜CRE.

In an aspect, the nucleotide sequence encoding a site-specific recombinase is operably linked to a constitutive promoter, an inducible promoter, a tissue-specific promoter, or a developmentally-regulated promoter. Suitable constitutive promoters, inducible promoters, tissue-specific promoters, and developmentally-regulated promoters include UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1, DR5, XVE, GLB1, OLE, LTP2 (Kalla et al., 1994. Plant J. 6:849-860 and U.S. Pat. No. 5,525,716 incorporated herein by reference in its entirety), HSP17.7, HSP26, HSP18A, AT-HSP811, AT-HSP811L, GM-HSP173B, promoters activated by tetracycline, ethametsulfuron or chlorsulfuron, PLTP, PLTP1, PLTP2, PLTP3, SDR, LGL, LEA-14A, or LEA-D34 (United States Patent Application publications 20170121722 and 20180371480 incorporated herein by reference in their entireties).

In an aspect, the chemically inducible promoter operably linked to the site-specific recombinase is XVE (Zuo et al. (2002) The Plant Journal 30(3):349-359). The chemically-inducible promoter can be repressed by the tetracycline repressor (TETR), the ethametsulfuron repressor (ESR), or the chlorsulfuron repressor (CR), and de-repression occurs upon addition of tetracycline-related or sulfonylurea ligands. The repressor can be TETR and the tetracycline-related ligand is doxycycline or anhydrotetracycline. (Gatz, C., Frohberg, C. and Wendenburg, R. (1992) Stringent repression and homogeneous de-repression by tetracycline of a modified CaMV 35S promoter in intact transgenic tobacco plants, Plant J. 2, 397-404). Alternatively, the repressor can be ESR and the sulfonylurea ligand is ethametsulfuron, chlorsulfuron, metsulfuron-methyl, sulfometuron methyl, chlorimuron ethyl, nicosulfuron, primisulfuron, tribenuron, sulfosulfuron, trifloxysulfuron, foramsulfuron, iodosulfuron, prosulfuron, thifensulfuron, rimsulfuron, mesosulfuron, or halosulfuron (US20110287936 incorporated herein by reference in its entirety). An alternative method for inducible expression is use of the glucocorticoid system in which an encoded glucocorticoid repressor (Ouwerkerk et al. (2001) Planta 213:370-378) is fused to an encoded gene of interest (e.g., a morphogenic protein such as WUS2 or ODP2 protein).

In an aspect, when the morphogenic gene expression cassette or construct comprises site-specific recombinase excision sites, the nucleotide sequence encoding the functional WUS/WOX polypeptide, or the nucleotide sequence encoding a Babyboom (BBM) polypeptide or an Ovule Development Protein 2 (ODP2) polypeptide, or the combination of a nucleotide sequence encoding a functional WUS/WOX polypeptide and a nucleotide sequence encoding a Babyboom (BBM) polypeptide or an Ovule Development Protein 2 (ODP2) polypeptide can be operably linked to an auxin inducible promoter, a developmentally regulated promoter, a tissue-specific promoter, or a constitutive promoter. Exemplary auxin inducible promoters, developmentally regulated promoters, tissue-specific promoters, and constitutive promoters useful in this context include UBI, LLDAV, EVCV, DMMV, BSV(AY) PRO, CYMV PRO FL, UBIZM PRO, SI-UB3 PRO, SB-UBI PRO (ALT1), USB1ZM PRO, ZM-GOS2 PRO, ZM-H1B PRO (1.2 KB), IN2-2, NOS, the −135 version of 35S, ZM-ADF PRO (ALT2), AXIG1 (U.S. Pat. No. 6,838,593 incorporated herein by reference in its entirety), DR5, XVE, GLB1, OLE, LTP2, HSP17.7, HSP26, HSP18A, AT-HSP811 (Takahashi, T, et al., (1992) Plant Physiol. 99 (2): 383-390), AT-HSP811L (Takahashi, T, et al., (1992) Plant Physiol. 99 (2): 383-390), GM-HSP173B (Schöffl, F., et al. (1984) EMBO J. 3(11): 2491-2497), promoters activated by tetracycline, ethamethsulfuron or chlorsulfuron, PLTP, PLTP1, PLTP2, PLTP3, SDR, LGL, LEA-14A, LEA-D34 (United States Patent Application publications 20170121722 and 20180371480 incorporated herein by reference in their entireties), and any of the promoters disclosed herein.

When using a morphogenic gene cassette and a trait gene cassette (heterologous polynucleotide) to produce transgenic plants it is desirable to have the ability to segregate the morphogenic gene locus away from the trait gene (heterologous polynucleotide) locus in co-transformed plants to provide transgenic plants containing only the trait gene (heterologous polynucleotide). This can be accomplished using an Agrobacterium tumefaciens two T-DNA binary system, with two variations on this general theme (see Miller et al., 2002). For example, in the first, a two T-DNA vector, where expression cassettes for morphogenic genes and herbicide selection (i.e. HRA) are contained within a first T-DNA and the trait gene cassette (heterologous polynucleotide) is contained within a second T-DNA, where both T-DNA's reside on a single binary vector. When a plant cell is transformed by an Agrobacterium containing the two T-DNA plasmid a high percentage of transformed cells contain both T-DNA's that have integrated into different genomic locations (for example, onto different chromosomes). In the second method, for example, two Agrobacterium strains, each containing one of the two T-DNA's (either the morphogenic gene T-DNA or the trait gene (heterologous polynucleotide) T-DNA), are mixed together in a ratio, and the mixture is used for transformation. After transformation using this mixed Agrobacterium method, it is observed at a high frequency that recovered transgenic events contain both T-DNA's, often at separate genomic locations. For both co-transformation methods, it is observed that in a large proportion of the produced transgenic events, the two T-DNA loci segregate independently and progeny T1 plants can be readily identified in which the T-DNA loci have segregated away from each other, resulting in the recovery of progeny seed that contain the trait genes (heterologous polynucleotides) with no morphogenic genes/herbicide genes. See, Miller et al. Transgenic Res 11(4):381-96.

The methods provided herein rely upon the use of bacteria-mediated and/or biolistic-mediated gene transfer, in addition to eletroporation, PEG transfection, or RNP (ribonucleoprotein) delivery to produce regenerable plant cells having an incorporated nucleotide sequence of interest. Bacterial strains useful in the methods of the disclosure include, but are not limited to, a disarmed Agrobacterium, an Ochrobactrum bacteria or a Rhizobiaceae bacteria. Disarmed Agrobacteria useful in the present methods include, but are not limited to, AGL-1, EHA105, GV3101, LBA4404, LBA4404 THY- (see U.S. Pat. No. 8,334,429 incorporated herein by reference in its entirety), and LBA4404 TD THY- in which both copies of the Tn904 transposon removed have been removed from LBA4404 THY- (see PCT/US20/24993 filed Mar. 26, 2020 which claims the benefit of U.S. Provisional Patent Application No. 62/825,054 filed on Mar. 28, 2019, all of which is hereby incorporated herein in its entirety by reference). Agrobacterium strain LBA4404 TD THY- is A. tumefaciens LBA4404 THY- strain deposited with the ATCC, assigned Accession Number PTA-10531 wherein a functional Tn904 transposon is not present or both copies of the Tn904 transposon have been deleted. Ochrobactrum bacterial strains useful in the present methods include, but are not limited to, those disclosed in U.S. Pat. Pub. No. US20180216123 incorporated herein by reference in its entirety. Rhizobiaceae bacterial strains useful in the present methods include, but are not limited to, those disclosed in U.S. Pat. No. 9,365,859 incorporated herein by reference in its entirety.

Also embodied is a plant with the described expression cassette stably incorporated into the genome of the plant, a seed of the plant, wherein the seed comprises the expression cassette. Further embodied is a plant wherein a gene or gene product of a heterologous polynucleotide or a polynucleotide of interest that confers a nutritional enhancement, increased yield, abiotic stress tolerance, drought tolerance, cold tolerance, herbicide tolerance, pest resistance, pathogen resistance, insect resistance, nitrogen use efficiency (NUE), disease resistance, or an ability to alter a metabolic pathway. A plant wherein expression of a heterologous polynucleotide or a polynucleotide of interest alters the phenotype of said plant is also embodied.

The disclosure encompasses isolated or substantially purified nucleic acid compositions. An “isolated” or “purified” nucleic acid molecule or biologically active portion thereof is substantially free of other cellular material or culture medium when produced by recombinant techniques or substantially free of chemical precursors or other chemicals when chemically synthesized. An “isolated” nucleic acid is substantially free of sequences (including protein encoding sequences) that naturally flank the nucleic acid (i.e., sequences located at the 5′ and 3′ ends of the nucleic acid) in the genomic DNA of the organism from which the nucleic acid is derived. For example, in various aspects, the isolated nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived.

As used herein, the term “fragment” refers to a portion of the nucleic acid sequence. Fragments of sequences useful in the methods of the present disclosure retain the biological activity of the nucleic acid sequence. Alternatively, fragments of a nucleotide sequence that are useful as hybridization probes may not necessarily retain biological activity. Fragments of a nucleotide sequence disclosed herein may range from at least about 20, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, or 1900 nucleotides, and up to the full length of the subject sequence. A biologically active portion of a nucleotide sequence can be prepared by isolating a portion of the sequence and assessing the activity of the portion.

Fragments and variants of nucleotide sequences and the proteins encoded thereby useful in the methods of the present disclosure are also encompassed. As used herein, the term “fragment” refers to a portion of a nucleotide sequence and hence the protein encoded thereby or a portion of an amino acid sequence. Fragments of a nucleotide sequence may encode protein fragments that retain the biological activity of the native protein. Alternatively, fragments of a nucleotide sequence useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins useful in the methods of the present disclosure.

As used herein, the term “variants” means sequences having substantial similarity with a promoter sequence disclosed herein. A variant comprises a deletion and/or addition of one or more nucleotides at one or more internal sites within the native polynucleotide and/or a substitution of one or more nucleotides at one or more sites in the native polynucleotide. As used herein, a “native” nucleotide sequence comprises a naturally occurring nucleotide sequence. For nucleotide sequences, naturally occurring variants can be identified with the use of well-known molecular biology techniques, such as, for example, with polymerase chain reaction (PCR) and hybridization techniques as outlined herein.

Variant nucleotide sequences also include synthetically derived nucleotide sequences, such as those generated, for example, by using site-directed mutagenesis. Generally, variants of a nucleotide sequence disclosed herein will have at least 40%, 50%, 60%, 65%, 70%, 75%, 80%, 85%, 90%, 91%, 92%, 93%, 94%, to 95%, 96%, 97%, 98%, 99% or more sequence identity to that nucleotide sequence as determined by sequence alignment programs described elsewhere herein using default parameters. Biologically active variants of a nucleotide sequence disclosed herein are also encompassed. Biological activity may be measured by using techniques such as Northern blot analysis, reporter activity measurements taken from transcriptional fusions, and the like. See, for example, Sambrook, et al., (1989) Molecular Cloning: A Laboratory Manual (2d ed., Cold Spring Harbor Laboratory Press, Cold Spring Harbor, N.Y.), hereinafter “Sambrook,” herein incorporated by reference in its entirety. Alternatively, levels of a reporter gene such as green fluorescent protein (GFP) or yellow fluorescent protein (YFP) or the like produced under the control of a promoter operably linked to a nucleotide fragment or variant can be measured. See, for example, Matz et al. (1999) Nature Biotechnology 17:969-973; U.S. Pat. No. 6,072,050, herein incorporated by reference in its entirety; Nagai, et al., (2002) Nature Biotechnology 20(1):87-90. Variant nucleotide sequences also encompass sequences derived from a mutagenic and recombinogenic procedure such as DNA shuffling. With such a procedure, one or more different nucleotide sequences can be manipulated to create a new nucleotide sequence. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer, (1994) Proc. Natl. Acad. Sci. USA 91:10747-10751; Stemmer, (1994) Nature 370:389 391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458, herein incorporated by reference in their entirety.

Methods for mutagenesis and nucleotide sequence alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein, herein incorporated by reference in their entirety. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

The nucleotide sequences of the present disclosure can be used to isolate corresponding sequences from other organisms, particularly other plants, more particularly other monocots or dicots. In this manner, methods such as PCR, hybridization and the like can be used to identify such sequences based on their sequence homology to the sequences set forth herein. Sequences isolated based on their sequence identity to the entire sequences set forth herein or to fragments thereof are encompassed by the present disclosure.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in, Sambrook, supra. See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York), herein incorporated by reference in their entirety. Known methods of PCR include, but are not limited to, methods using paired primers, nested primers, single specific primers, degenerate primers, gene-specific primers, vector-specific primers, partially-mismatched primers and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides and may be labeled with a detectable group such as 32P or any other detectable marker. Thus, for example, probes for hybridization can be made by labeling synthetic oligonucleotides based on the sequences of the present disclosure. Methods for preparation of probes for hybridization and for construction of genomic libraries are generally known in the art and are disclosed in Sambrook, supra.

In general, sequences that have activity and hybridize to the sequences disclosed herein will be at least 40% to 50% homologous, about 60%, 70%, 80%, 85%, 90%, 95% to 98% homologous or more with the disclosed sequences. That is, the sequence similarity of sequences may range, sharing at least about 40% to 50%, about 60% to 70%, and about 80%, 85%, 90%, 95% to 98% sequence similarity.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the algorithm of Smith, et al., (1981) Adv. Appl. Math. 2:482; the algorithm of Needleman and Wunsch, (1970) J. Mol. Biol. 48:443-453; the algorithm of Pearson and Lipman, (1988) Proc. Natl. Acad. Sci. 85:2444-2448; the algorithm of Karlin and Altschul, (1990) Proc. Natl. Acad. Sci. USA 872:264, modified as in Karlin and Altschul, (1993) Proc. Natl. Acad. Sci. USA 90:5873-5877, herein incorporated by reference in their entirety. Computer implementations of these mathematical algorithms are well known in the art and can be utilized for comparison of sequences to determine sequence identity.

As used herein, “sequence identity” or “identity” in the context of two nucleic acid or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of one and a non-conservative substitution is given a score of zero, a conservative substitution is given a score between zero and one. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, optimally at least 80%, more optimally at least 90% and most optimally at least 95%, compared to a reference sequence using an alignment program using standard parameters. One of skill in the art will recognize that these values can be appropriately adjusted to determine corresponding identity of proteins encoded by two nucleotide sequences by considering codon degeneracy, amino acid similarity, reading frame positioning and the like. Substantial identity of amino acid sequences for these purposes normally means sequence identity of at least 60%, 70%, 80%, 90% and at least 95%.

Another indication that nucleotide sequences are substantially identical is if two molecules hybridize to each other under stringent conditions. Generally, stringent conditions are selected to be about 5° C. lower than the Tm for the specific sequence at a defined ionic strength and pH. However, stringent conditions encompass temperatures in the range of about 1° C. to about 20° C. lower than the Tm, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The methods, sequences, and genes disclosed herein are useful for genetic engineering of plants, e.g. to produce a transformed or transgenic plant, to express a phenotype of interest. As used herein, the terms “transformed plant” and “transgenic plant” refer to a plant that comprises within its genome a heterologous polynucleotide. Generally, the heterologous polynucleotide is stably integrated within the genome of a transgenic or transformed plant such that the polynucleotide is passed on to successive generations. The heterologous polynucleotide may be integrated into the genome alone or as part of a recombinant DNA construct. It is to be understood that as used herein the term “transgenic” includes any cell, cell line, callus, tissue, plant part or plant the genotype of which has been altered by the presence of a heterologous nucleic acid including those transgenics initially so altered as well as those created by sexual crosses or asexual propagation from the initial transgenic.

A transgenic “event” is produced by transformation of plant cells with a heterologous DNA construct, including a nucleic acid expression cassette that comprises a gene of interest, the regeneration of a population of plants resulting from the insertion of the transferred gene into the genome of the plant and selection of a plant characterized by insertion into a particular genome location. An event is characterized phenotypically by the expression of the inserted gene. At the genetic level, an event is part of the genetic makeup of a plant. The term “event” also refers to progeny produced by a sexual cross between the transformant and another plant wherein the progeny include the heterologous DNA.

The term “plant” refers to whole plants, plant organs (e.g., leaves, stems, roots, etc.), plant tissues, plant cells, plant parts, seeds, propagules, embryos, and progeny of the same. Plant cells can be differentiated or undifferentiated (e.g. callus, undifferentiated callus, immature and mature embryos, immature zygotic embryo, immature cotyledon, embryonic axis, suspension culture cells, protoplasts, leaf, leaf cells, root cells, phloem cells and pollen). Plant cells include, without limitation, cells from seeds, suspension cultures, explants, immature embryos, embryos, zygotic embryos, somatic embryos, embryogenic callus, meristem, somatic meristems, meristematic regions, organogenic callus, callus tissue, protoplasts, embryos derived from mature ear-derived seed, leaves, leaf bases, leaves from mature plants, leaf tips, immature inflorescences, tassel, immature ear, silks, cotyledons, immature cotyledons, embryonic axes, cells from leaves, cells from stems, cells from roots, cells from shoots, roots, shoots, gametophytes, sporophytes, pollen, microspores, multicellular structures (MCS), regenerable plant structures (RPS), and embryo-like structures.

Plant parts include differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in a plant or in a plant organ, tissue or cell culture.

Grain is intended to mean the mature seed produced by commercial growers for purposes other than growing or reproducing the species. Progeny, variants and mutants of the regenerated plants are also included within the scope of the disclosure, provided these progeny, variants and mutants comprise the introduced polynucleotides.

The present disclosure also includes plants obtained by any of the methods disclosed herein. The present disclosure also includes seeds from a plant obtained by any of the methods disclosed herein.

In a further aspect, the disclosed methods provide automated preparation of leaf explants that can be derived from any plant, including higher plants of the Angiospermae class. Plants of the subclasses of the Monocotyledonae are suitable. Suitable species may come from the family Alliaceae, Alstroemeriaceae, Amaryllidaceae, Arecaceae, Bromeliaceae, Colchicaceae, Dioscoreaceae, Melanthiaceae, Musaceae, and Poaceae. Suitable species from which the leaf explant prepared by the disclosed methods can be derived include members of the genus, Allium, Alstroemeria, Ananas, Andropogon, Arundo, Colchicum, Cynodon, Dioscorea, Elaeis, Erianthus, Festuca, Galanthus, Hordeum, Lolium, Miscanthus, Musa, Oryza, Panicum, Pennisetum, Phalaris, Phleum, Poa, Saccharum, Secale, Sorghum, Spartina, Triticosecale, Triticum, Uniola, Veratrum, and Zea.

In a further aspect, the leaf explant prepared by the disclosed methods can be derived from a plant that is important or interesting for agriculture, horticulture, biomass for the production of liquid fuel molecules and other chemicals, and/or forestry. Non-limiting examples include, for instance, Panicum virgatum (switchgrass), Sorghum bicolor (sorghum, sudangrass), Miscanthus giganteus (miscanthus), Saccharum sp. (energycane), Zea mays (corn), Triticum aestivum (wheat), Oryza sativa (rice), Pennisetum glaucum (pearl millet), Panicum spp., Sorghum spp., Miscanthus spp., Saccharum spp., Erianthus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Triticosecale spp. (triticum-wheat X rye), Bamboo, Elaeis guineensis (palm), Musa paradisiaca (banana), Ananas comosus (pineapple), Allium cepa (onion), Colchicum autumnale, Veratrum californica., Dioscorea spp., Galanthus wornorii, Alstroemeria spp., Uniola paniculata (oats), bentgrass (Agrostis spp.), Hordeum vulgare (barley), Poa pratensis (bluegrass), Lolium spp. (ryegrass), and Phleum pratense (timothy). Of interest are plants grown for energy production, so called energy crops, such as cellulose-based energy crops like Panicum virgatum (switchgrass), Sorghum bicolor (sorghum, sudangrass), Miscanthus giganteus (miscanthus), Saccharum sp. (energycane), Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Triticosecale spp. (triticum—wheat X rye), and Bamboo; and starch-based energy crops like Zea mays (corn); and sucrose-based energy crops like Saccharum sp. (sugarcane); and biodiesel-producing energy crops like Elaeis guineensis (palm).

In a further aspect, the leaf explant prepared by the disclosed methods can be derived from any plant found within the monocot families listed in Table 1 along with representative genera and/or species.

TABLE 1

| Family Names | Order | Representative Genera/Species |
|---|---|---|
| Agavaceae | Asparagales | Agave: A. cantala (Maguey, fiber crop); Yucca |
| Alliaceae | Asparagales | Thirteen genera e.g. Allium: A. cepa (Onion, |
| | | spice crop); Ipheion, Leucocoryne, Tulbaghia |
| Amaryllidaceae | Asparagales | Fifty-nine genera e.g. Amaryllis: A. |
| | | belladona (Belladona lily, ornamental |
| | | crop); Crinum, Galanthus, Hippeastrum, |
| | | Leucojum, Lycoris, Narcissus |
| Araceae | Alismatales | Over one hundred genera e.g. Colocasia: |
| | | C. esculenta (Taro or Gabi), Alocasia, |
| | | Xanthosoma (food crops, root and tuber crops), |
| | | Aglaonema, Anthurium, Caladium, |
| | | Dieffenbachia, Monstera, Philodendron, |
| | | Spathiphylum, Syngonium, Syngonium, |
| | | Zantedeschia (ornamental crops), Lemna, |
| | | Pistia, Wolfia |
| Arecaceae or | Arecales | Areca: A. catechu (Betel |
| Palmae | | nut); Arenga, Cocos, Elaeis, Metroxylon, |
| | | Phoenix, Washingtonia, Lodoicea |
| | | maldivica (biggest seed), Rhapia spp. (largest |
| | | leaves), Calamus (rattan) |
| Asparagaceae | Asparagales | Asparagus: A. officinalis (Vegetable |
| | | Asparagus) |
| Asphodelaceae | Asparagales | Fifteen genera e.g. Aloe: A. vera (Aloe or |
| | | sabila); Asphodelus, Bulbine, Gasteria, |
| | | Haworthia, Kniphofia |
| Bromeliaceae | Poales | Ananas: A. comosus (Pineapple, fruit and fiber |
| | | crop); Aechmea, Neoregalia, Puya, Tillandsia |
| Cannaceae | Zingiberales | One genus i.e, Canna: Canna spp. |
| Colchicaceae | Liliales | Colchicum: C. autumnale (Autumn |
| | | Crocus); Burchardia |
| Commelinaceae | Commelinales | Commelina: C. diffusa (Climbing |
| | | dayflower); Rhoeo, Cyanotis, Tradescantia, |
| | | Zebrina |
| Costaceae | Zingiberales | Costus: C. speciosus (Common spiral ginger) |
| Cyclanthacea | Pandanales | Carludovica palmata (fiber) |
| Cyperaceae | Poales | One hundred four genera e.g. Cyperus: C. |
| | | alternifolius, Carex, Eleocharis, Scirpus |
| Dioscoreaceae | Dioscoreales | Four genera e.g. Dioscorea: D. alata (Yam, |
| | | tuber crop); Stenomeris, Tacca, Trichopus |
| Eriocaulaceae | Poales | Ten genera e.g. Eriocaulon (pipewort), |
| | | Leiothrix, Paepalanthus, Syngonanthus |
| Heliconiaceae | Zingiberales | Heliconia: H. humilis (ornamental crop) |
| Iridaceae | Asparagales | Seventy genera e.g. Iris: I. |
| | | reticulata (Reticulated Iris, ornamental |
| | | crop); Crocus, Dietes, Freesia, Gladiolus |
| Juncaceae | Poales | Seven genera e.g. Juncus: J. effusus (Soft |
| | | rush); Distichia |
| Liliaceae | Liliales | Sixteen generae.g. Lilium: L. |
| | | longiflorum (White Trumpet Lily, ornamental |
| | | crop); Tulipa, Calochortus, Erythronium, |
| | | Fritillaria, Medeola |
| Marantaceae | Zingiberales | Maranta: M. arundinacea (Arrow root, root |
| | | crop); Calathea, Thalia |
| Musaceae | Zingiberales | Three genera e.g. Musa: Musa spp. (Banana, |
| | | fruit crop; Abaca, fiber crop); Ensete, Musella |
| Orchidaceae | Asparagales | Seven to eight hundred genera |
| | | e.g. Cattleya, Cymbidium, Dendrobium, |
| | | Phalaenopsis, Vanda: V. sanderiana |
| | | (waling-waling orchid, ornamental crop), Vanilla |
| Pandanaceae | Pandanales | Three genera e.g. Pandanus: P. |
| | | tectorius (Screw Pine, fiber crop); Freycinetia |
| Poaceae or | Poales | Six hundred sixty-eight genera e.g. Avena, |
| Gramineae | | Hordeum, Sorghum, Oryza, Triticum, Zea |
| | | (cereals); Bambusa, Dendrocalamus (bamboos); |
| | | Saccharum: S. officinarum (sugarcane); |
| | | Cymbopogon (lemon grass, spice, essential-oil); |
| | | Brachiaria, Cynodon, Panicum, Pennisetum |
| | | (forage crops); Axonopus, Paspalum, Zoysia |
| | | (turfgrasses) |
| Pontederiaceae | Commelinales | Pontederia: P. cordata (Pickerel |
| | | weed); Eichhornia, Heteranthera, Monochoria |
| Smilacaceae | Liliales | Smilax: S. bracteata (Sarsaparilla) |
| Strelitziaceae | Zingiberales | Three genera e.g. Strelitzia: S. reginae (Bird of |
| | | Paradise, ornamental |
| | | crop); Phenakospermum, Ravanela |
| Typhaceae | Poales | One genus: Typha: T. angustifolia (Cattail, |
| | | aquatic ornamental crop and food crop) |
| Xyridaceae | Poales | Five genera e.g. Xyris: X. pauciflora (grass-like |
| | | weed), Abolboda, Achlyphila, Aratitiyopea, Orectanth |
| Zingiberaceae | Zingiberales | Zingiber: Z. officinalis (Ginger, spice |
| | | crop), Alpinia, Curcuma, Elettaria, Hedychium |
| Zosteraceae | Alismatales | Zostera marina (eelgrass), Phyllospadix |
| | | serrulatus (surfgrass) |

In yet a further aspect, leaf explants from the Poaceae family, including leaf explants from the sub-families Chloridoideae, Danthonioideae, Micrairoideae, Arundinoideae, Panicoideae, Aristidoideae. Oryzoideae, Bambusoideae, Pooideae, Puelioideae, Pharoideae, and Anomochlooideae can be prepared by the methods of the present disclosure. Poaceae (also refered to historically as the Gramineae) is a large family of monocotyledonous flowering plants known as grasses. It includes the cereal grasses, bamboos and the grasses of natural grassland and species cultivated in lawns and pasture. Examples of species within the Poaceae useful in the methods of the present disclosure include, but are not limited to bamboo (Phyllostachys edulis), barley (Hordeum vulgare), bentgrass (Agrostis sp.), creeping bent (Agrostis stolonifera), bluegrass (Poa sp.), fescue (Festuca sp.), green bristlegrass (Setaria viridis), reed canarygrass (Phalaris arundinacea), guinea grass (Megathyrsus maximus), golden bamboo (Phyllostachys aurea), elephant grass (Arundo donax), desert grass (Stipagrostis plumosa), inland sea oats (Chasmanthium latifolium), silver grass (Miscanthus sinensis), foxtail millet (Setaria italica), finger millet (Eleusine coracana), little millet (Panicum sumatrance), kodo millet (Paspalum scrobiculatum), barnyard millet (Echinochloa frumentacea) and proso millet (Panicum miliaceum), orchard grass (Dactylis glomerata), switchgrass (Panicum virgatum), pearl millet (Pennisetum glaucum), purple false brome (Brachypodium distachyon), rice (Oryza sativa; both Japonica and Indica varieties), rye (Secale cereale), ryegrass (Lolium perenne), sorghum (Sorghum bicolor), Saint Augustine grass (Stenotaphrum secundatum), sugarcane (Saccharum officinarum), teff (Eragrostis tef), fonio (Digitaria exilis), timothy (Phleum pratense), triticale (Triticosecale sp.), wheat (Triticum aestivum), durum wheat (Triticum durum), emmer wheat (Triticum dicoccum), einkorn wheat (Triticum monococcum), spelt wheat (Triticum spelta), goatgrass (Aegilops spp), wheatgrass (Agropyron cristatum), oats (Avena sativa), corn (Zea mays), teosinte (Zea mays spp. mexicana or spp. parviglumis), and perennial teosinte (Zea diploperennis).

In specific aspects, leaf explants prepared by the automated methods of the present disclosure include, but are not limited to leaf explants of bamboo (Phyllostachys edulis), barley (Hordeum vulgare), bentgrass (Agrostis sp.), creeping bent (Agrostis stolonifera), bluegrass (Poa sp.), fescue (Festuca sp.), green bristlegrass (Setaria viridis), reed canarygrass (Phalaris arundinacea), guinea grass (Megathyrsus maximus), golden bamboo (Phyllostachys aurea), elephant grass (Arundo donax), desert grass (Stipagrostis plumosa), inland sea oats (Chasmanthium latifolium), silver grass (Miscanthus sinensis), foxtail millet (Setaria italica), finger millet (Eleusine coracana), little millet (Panicum sumatrance), kodo millet (Paspalum scrobiculatum), barnyard millet (Echinochloa frumentacea) and proso millet (Panicum miliaceum), orchard grass (Dactylis glomerata), switchgrass (Panicum virgatum), pearl millet (Pennisetum glaucum), purple false brome (Brachypodium distachyon), rice (Oryza sativa; both Japonica and Indica varieties), rye (Secale cereale), ryegrass (Lolium perenne), sorghum (Sorghum bicolor), Saint Augustine grass (Stenotaphrum secundatum), sugarcane (Saccharum officinarum), teff (Eragrostis tef), fonio (Digitaria exilis), timothy (Phleum pratense), triticale (Triticosecale sp.), wheat (Triticum aestivum), durum wheat (Triticum durum), emmer wheat (Triticum dicoccum), einkorn wheat (Triticum monococcum), spelt wheat (Triticum spelta), goatgrass (Aegilops spp), wheatgrass (Agropyron cristatum), oats (Avena sativa), corn (Zea mays), teosinte (Zea mays spp. mexicana or spp. parviglumis), and perennial teosinte (Zea diploperennis).

The methods of the present disclosure may be used for the preparation of leaf explants for transformation of any plant species, including, but not limited to, monocots and dicots. Monocots include, but are not limited to, barley, maize (corn), millet (e.g., pearl millet (Pennisetum glaucum), proso millet (Panicum miliaceum), foxtail millet (Setaria italica), finger millet (Eleusine coracana), teff (Eragrostis tef), oats, rice, rye, Setaria sp., sorghum, triticale, or wheat, or leaf and stem crops, including, but not limited to, bamboo, marram grass, meadow-grass, reeds, ryegrass, sugarcane; lawn grasses, ornamental grasses, and other grasses such as switchgrass and turf grass. Alternatively, dicot plants used in the present disclosure, include, but are not limited to, kale, cauliflower, broccoli, mustard plant, cabbage, pea, clover, alfalfa, broad bean, tomato, peanut, cassava, soybean, canola, alfalfa, sunflower, safflower, tobacco, Arabidopsis, or cotton.

Heterologous coding sequences, heterologous polynucleotides, and polynucleotides of interest may be used in the methods of the disclosure for varying the phenotype of a plant. Various changes in phenotype are of interest including modifying expression of a gene in a plant, altering a plant's pathogen or insect defense mechanism, increasing a plant's tolerance to herbicides, altering plant development to respond to environmental stress, modulating the plant's response to salt, temperature (hot and cold), drought and the like. These results can be achieved by the expression of a heterologous nucleotide sequence of interest comprising an appropriate gene product. In specific aspects, the heterologous nucleotide sequence of interest is an endogenous plant sequence whose expression level is increased in the plant or plant part. Results can be achieved by providing for altered expression of one or more endogenous gene products, particularly hormones, receptors, signaling molecules, enzymes, transporters or cofactors or by affecting nutrient uptake in the plant. These changes result in a change in phenotype of the transformed plant.

General categories of heterologous polynucleotides or nucleotide sequences of interest for use in the methods of the present disclosure include, for example, those genes involved in information, such as zinc fingers, those involved in communication, such as kinases and those involved in housekeeping, such as heat shock proteins. More specific categories of transgenes (heterologous polynucleotides or nucleotide sequences of interest), for example, include genes encoding important traits for agronomics, insect resistance, disease resistance, herbicide resistance, environmental stress resistance (altered tolerance to cold, salt, drought, etc.) and grain characteristics. Still other categories of transgenes include genes for inducing expression of exogenous products such as enzymes, cofactors, and hormones from plants and other eukaryotes as well as prokaryotic organisms. It is recognized that any gene or polynucleotide of interest can be operably linked to a promoter and expressed in a plant using the methods disclosed herein.

Many agronomic traits can affect “yield”, including without limitation, plant height, pod number, pod position on the plant, number of internodes, incidence of pod shatter, grain size, efficiency of nodulation and nitrogen fixation, efficiency of nutrient assimilation, resistance to biotic and abiotic stress, carbon assimilation, plant architecture, resistance to lodging, percent seed germination, seedling vigor, and juvenile traits. Other traits that can affect yield include, efficiency of germination (including germination in stressed conditions), growth rate (including growth rate in stressed conditions), ear number, seed number per ear, seed size, composition of seed (starch, oil, protein) and characteristics of seed fill. Also of interest is the generation of transgenic plants that demonstrate desirable phenotypic properties that may or may not confer an increase in overall plant yield. Such properties include enhanced plant morphology, plant physiology or improved components of the mature seed harvested from the transgenic plant.

“Increased yield” of a transgenic plant of the present disclosure may be evidenced and measured in a number of ways, including test weight, seed number per plant, seed weight, seed number per unit area (i.e. seeds, or weight of seeds, per acre), bushels per acre, tons per acre, kilo per hectare. For example, maize yield may be measured as production of shelled corn kernels per unit of production area, e.g. in bushels per acre or metric tons per hectare, often reported on a moisture adjusted basis, e.g., at 15.5% moisture. Increased yield may result from improved utilization of key biochemical compounds, such as nitrogen, phosphorous and carbohydrate, or from improved tolerance to environmental stresses, such as cold, heat, drought, salt, and attack by pests or pathogens. Trait-enhancing recombinant DNA may also be used to provide transgenic plants having improved growth and development, and ultimately increased yield, as the result of modified expression of plant growth regulators or modification of cell cycle or photosynthesis pathways.

An “enhanced trait” as used herein describing the aspects of the present disclosure includes improved or enhanced water use efficiency or drought tolerance, osmotic stress tolerance, high salinity stress tolerance, heat stress tolerance, enhanced cold tolerance, including cold germination tolerance, increased yield, improved seed quality, enhanced nitrogen use efficiency, early plant growth and development, late plant growth and development, enhanced seed protein, and enhanced seed oil production.

Multiple genes of interest (heterologous polynucleotides or nucleotide sequences of interest) can be used in the methods of the disclosure and expressed in a plant, for example insect resistance traits herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, nutritional enhancement, and the like).

Such genes (heterologous polynucleotides or nucleotide sequences of interest) include, for example, Bacillus thuringiensis toxic protein genes, U.S. Pat. Nos. 5,366,892; 5,747,450; 5,736,514; 5,723,756; 5,593,881 and Geiser, et al., (1986) Gene 48:109, the disclosures of which are herein incorporated by reference in their entirety. Genes (heterologous polynucleotides or nucleotide sequences of interest) encoding disease resistance traits can also be used in the methods of the disclosure including, for example, detoxification genes, such as those which detoxify fumonisin (U.S. Pat. No. 5,792,931); avirulence (avr) and disease resistance (R) genes (Jones, et al., (1994) Science 266:789; Martin, et al., (1993) Science 262:1432; and Mindrinos, et al., (1994) Cell 78:1089), herein incorporated by reference in their entirety.

Herbicide resistance traits (heterologous polynucleotides or nucleotide sequences of interest) can be used in the methods of the disclosure including genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), genes coding for resistance to glyphosate (e.g., the EPSPS gene and the GAT gene; see, for example, US Patent Application Publication Number 2004/0082770 and WO 03/092360, herein incorporated by reference in their entirety) or other such genes known in the art. The bar gene encodes resistance to the herbicide basta, the nptII gene encodes resistance to the antibiotics kanamycin and geneticin and the ALS-gene mutants encode resistance to the herbicide chlorsulfuron any and all of which can be operably linked to a promoter and used in the methods of the disclosure.

Glyphosate resistance is imparted by mutant 5-enolpyruvl-3-phosphikimate synthase (EPSPS) and aroA genes which can be operably linked to a promoter and used in the methods of the disclosure. See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes which can be operably linked to a promoter and used in the methods of the disclosure. See also, U.S. Pat. Nos. 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E and 5,491,288 and international publications WO 97/04103; WO 97/04114; WO 00/66746; WO 01/66704; WO 00/66747 and WO 00/66748, which are incorporated herein by reference in their entirety. Glyphosate resistance is also imparted to plants that express a gene that encodes a glyphosate oxido-reductase enzyme as described more fully in U.S. Pat. Nos. 5,776,760 and 5,463,175, which are incorporated herein by reference in their entirety. Glyphosate resistance can also be imparted to plants by the over expression of genes encoding glyphosate N-acetyltransferase. See, for example, U.S. patent application Ser. Nos. 11/405,845 and 10/427,692, herein incorporated by reference in their entirety.

Sterility genes (heterologous polynucleotides or nucleotide sequences of interest) can be used in the methods of the disclosure to provide an alternative to physical detasseling. Examples of genes used in such ways include male tissue-preferred genes and genes with male sterility phenotypes such as QM, described in U.S. Pat. No. 5,583,210, herein incorporated by reference in its entirety. Other genes which can be operably linked to a promoter and used in the methods of the disclosure include kinases and those encoding compounds toxic to either male or female gametophytic development.

Commercial traits can also be produced using the methods of the disclosure that could increase for example, starch for ethanol production, or provide expression of proteins. Another important commercial use of transformed plants is the production of polymers and bioplastics such as described in U.S. Pat. No. 5,602,321, herein incorporated by reference in its entirety. Genes such as β-Ketothiolase, PHBase (polyhydroxybutyrate synthase), and acetoacetyl-CoA reductase which can be operably linked to a promoter and used in the methods of the disclosure (see, Schubert, et al., (1988) J. Bacteriol. 170:5837-5847, herein incorporated by reference in its entirety) facilitate expression of polyhydroxyalkanoates (PHAs).

Numerous trait genes (heterologous polynucleotides or nucleotide sequences of interest) are known in the art and can be used in the methods disclosed herein. By way of illustration, without intending to be limiting, trait genes (heterologous polynucleotides) that confer resistance to insects or diseases, trait genes (heterologous polynucleotides) that confer resistance to a herbicide, trait genes (heterologous polynucleotides) that confer or contribute to an altered grain characteristic, such as altered fatty acids, altered phosphorus content, altered carbohydrates or carbohydrate composition, altered antioxidant content or composition, or altered essential seed amino acids content or composition are examples of the types of trait genes (heterologous polynucleotides) which can be operably linked to a promoter for expression in plants transformed by the methods disclosed herein. Additional genes known in the art may be included in the expression cassettes useful in the methods disclosed herein. Non-limiting examples include genes that create a site for site specific DNA integration, genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress, or other genes and transcription factors that affect plant growth and agronomic traits such as yield, flowering, plant growth and/or plant structure.

The methods of the disclosure can be used to transform a plant with a heterologous nucleotide sequence that is an antisense sequence for a targeted gene. As used herein, “antisense orientation” includes reference to a polynucleotide sequence that is operably linked to a promoter in an orientation where the antisense strand is transcribed. The antisense strand is sufficiently complementary to an endogenous transcription product such that translation of the endogenous transcription product is often inhibited. “Operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terminology “antisense DNA nucleotide sequence” is intended to mean a sequence that is in inverse orientation to the 5′-to-3′ normal orientation of that nucleotide sequence. When delivered into a plant cell, expression of the antisense DNA sequence prevents normal expression of the DNA nucleotide sequence for the targeted gene. The antisense nucleotide sequence encodes an RNA transcript that is complementary to and capable of hybridizing to the endogenous messenger RNA (mRNA) produced by transcription of the DNA nucleotide sequence for the targeted gene. In this case, production of the native protein encoded by the targeted gene is inhibited to achieve a desired phenotypic response. Modifications of the antisense sequences may be made as long as the sequences hybridize to and interfere with expression of the corresponding mRNA. In this manner, antisense constructions having 70%, 80%, 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides or greater may be used. Thus, the promoter sequences disclosed herein may be operably linked to antisense DNA sequences to reduce or inhibit expression of a native protein in the plant. “RNAi” refers to a series of related techniques to reduce the expression of genes (see, for example, U.S. Pat. No. 6,506,559, herein incorporated by reference in its entirety).

Older techniques referred to by other names are now thought to rely on the same mechanism but are given different names in the literature. These include “antisense inhibition,” the production of antisense RNA transcripts capable of suppressing the expression of the target protein and “co-suppression” or “sense-suppression,” which refer to the production of sense RNA transcripts capable of suppressing the expression of identical or substantially similar foreign or endogenous genes (U.S. Pat. No. 5,231,020, incorporated herein by reference in its entirety). Such techniques rely on the use of constructs resulting in the accumulation of double stranded RNA with one strand complementary to the target gene to be silenced. The methods of the disclosure may be used to express constructs that will result in RNA interference including microRNAs and siRNAs.

As used herein, the terms “promoter” or “transcriptional initiation region” mean a regulatory region of DNA usually comprising a TATA box or a DNA sequence capable of directing RNA polymerase II to initiate RNA synthesis at the appropriate transcription initiation site for a particular coding sequence. A promoter may additionally comprise other recognition sequences generally positioned upstream or 5′ to the TATA box or the DNA sequence capable of directing RNA polymerase II to initiate RNA synthesis, referred to as upstream promoter elements, which influence the transcription initiation rate. It is recognized that having identified the nucleotide sequences for the promoter regions disclosed herein, it is within the state of the art to isolate and identify further promoters in the 5′ untranslated region upstream from the particular promoter regions identified herein. Additionally, chimeric promoters may be provided. Such chimeras include portions of the promoter sequence fused to fragments and/or variants of heterologous transcriptional regulatory regions. Thus, the promoter regions disclosed herein can comprise upstream promoters such as, those responsible for tissue and temporal expression of the coding sequence, enhancers and the like.

As used herein, the term “regulatory element” also refers to a sequence of DNA, usually, but not always, upstream (5′) to the coding sequence of a structural gene, which includes sequences which control the expression of the coding region by providing the recognition for RNA polymerase and/or other factors required for transcription to start at a particular site. An example of a regulatory element that provides for the recognition for RNA polymerase or other transcriptional factors to ensure initiation at a particular site is a promoter element. A promoter element comprises a core promoter element, responsible for the initiation of transcription, as well as other regulatory elements that modify gene expression. It is to be understood that nucleotide sequences, located within introns or 3′ of the coding region sequence may also contribute to the regulation of expression of a coding region of interest. Examples of suitable introns include, but are not limited to, the maize IVS6 intron, or the maize actin intron. A regulatory element may also include those elements located downstream (3′) to the site of transcription initiation, or within transcribed regions, or both. In the context of the present disclosure a post-transcriptional regulatory element may include elements that are active following transcription initiation, for example translational and transcriptional enhancers, translational and transcriptional repressors and mRNA stability determinants.

A “heterologous nucleotide sequence”, “heterologous polynucleotide of interest”, or “heterologous polynucleotide” as used throughout the disclosure, is a sequence that is not naturally occurring with or operably linked to a promoter sequence. While this nucleotide sequence is heterologous to the promoter sequence, it may be homologous or native or heterologous or foreign to the plant host. Likewise, the promoter sequence may be homologous or native or heterologous or foreign to the plant host and/or the polynucleotide of interest.

It is recognized that to increase transcription levels, enhancers may be. Enhancers are nucleotide sequences that act to increase the expression of a promoter region. Enhancers are known in the art and include the SV40 enhancer region, the 35S enhancer element and the like. Some enhancers are also known to alter normal promoter expression patterns, for example, by causing a promoter to be expressed constitutively when without the enhancer, the same promoter is expressed only in one specific tissue or a few specific tissues.

Modifications of promoter sequences can provide for a range of expression of a heterologous nucleotide sequence. Thus, they may be modified to be weak promoters or strong promoters. Generally, a “weak promoter” means a promoter that drives expression of a coding sequence at a low level. A “low level” of expression is intended to mean expression at levels of about 1/10,000 transcripts to about 1/100,000 transcripts to about 1/500,000 transcripts. Conversely, a strong promoter drives expression of a coding sequence at a high level, or at about 1/10 transcripts to about 1/100 transcripts to about 1/1,000 transcripts.

The transformation methods disclosed herein are useful in the genetic manipulation of any plant, thereby resulting in a change in phenotype of the transformed plant. Changes in phenotype can be accomplished by T-DNA transfer, particle bombardment, electroporation, PEG transfection, or RNP (ribonucleoprotein) delivery.

The term “operably linked” means that the transcription or translation of a heterologous nucleotide sequence is under the influence of a promoter sequence. In this manner, the nucleotide sequences for the promoters may be provided in expression cassettes along with heterologous nucleotide sequences of interest for expression in the plant of interest, more particularly for expression in the reproductive tissue of the plant.

In one aspect of the disclosure, expression cassettes comprise a transcriptional initiation region comprising a promoter nucleotide sequence or variants or fragments thereof, operably linked to a morphogenic gene and/or a heterologous nucleotide sequence. Such an expression cassette can be provided with a plurality of restriction sites for insertion of the nucleotide sequence to be under the transcriptional regulation of the regulatory regions. The expression cassette may additionally contain selectable marker genes as well as 3′ termination regions.

The expression cassette can include, in the 5′-3′ direction of transcription, a transcriptional initiation region (i.e., a promoter, or variant or fragment thereof), a translational initiation region, a heterologous nucleotide sequence of interest, a translational termination region and optionally, a transcriptional termination region functional in the host organism. The regulatory regions (i.e., promoters, transcriptional regulatory regions, and translational termination regions) and/or the polynucleotide of the aspects may be native/analogous to the host cell or to each other. Alternatively, the regulatory regions and/or the polynucleotide of the aspects may be heterologous to the host cell or to each other. As used herein, “heterologous” in reference to a sequence is a sequence that originates from a foreign species or, if from the same species, is substantially modified from its native form in composition and/or genomic locus by deliberate human intervention. For example, a promoter operably linked to a heterologous polynucleotide is from a species different from the species from which the polynucleotide was derived or, if from the same/analogous species, one or both are substantially modified from their original form and/or genomic locus or the promoter is not the native promoter for the operably linked polynucleotide.

The termination region may be native with the transcriptional initiation region, may be native with the operably linked DNA sequence of interest, may be native with the plant host, or may be derived from another source (i.e., foreign or heterologous to the promoter, the DNA sequence being expressed, the plant host, or any combination thereof). Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; and Joshi, et al., (1987) Nucleic Acid Res. 15:9627-9639, herein incorporated by reference in their entirety.

The expression cassette useful in the methods of the disclosure may also contain at least one additional nucleotide sequence for a gene, heterologous nucleotide sequence, heterologous polynucleotide of interest, or heterologous polynucleotide to be co-transformed into the organism. Alternatively, the additional nucleotide sequence(s) can be provided on another expression cassette.

Where appropriate, the nucleotide sequences may be optimized for increased expression in the transformed plant. That is, these nucleotide sequences can be synthesized using plant preferred codons for improved expression. See, for example, Campbell and Gowri, (1990) Plant Physiol. 92:1-11, herein incorporated by reference in its entirety, for a discussion of host-preferred codon usage. Methods are available in the art for synthesizing plant-preferred genes. See, for example, U.S. Pat. Nos. 5,380,831, 5,436,391 and Murray, et al., (1989) Nucleic Acids Res. 17:477-498, herein incorporated by reference in their entirety.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the heterologous nucleotide sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include, without limitation: picornavirus leaders, for example, EMCV leader (Encephalomyocarditis 5′ noncoding region) (Elroy-Stein, et al., (1989) Proc. Nat. Acad. Sci. USA 86:6126-6130); potyvirus leaders, for example, TEV leader (Tobacco Etch Virus) (Allison, et al., (1986) Virology 154:9-20); MDMV leader (Maize Dwarf Mosaic Virus); human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) Molecular Biology of RNA, pages 237-256) and maize chlorotic mottle virus leader (MCMV) (Lommel, et al., (1991) Virology 81:382-385), herein incorporated by reference in their entirety. See, also, Della-Cioppa, et al., (1987) Plant Physiology 84:965-968, herein incorporated by reference in its entirety. Methods known to enhance mRNA stability can also be utilized, for example, introns, such as the maize Ubiquitin intron (Christensen and Quail, (1996) Transgenic Res. 5:213-218; Christensen, et al., (1992) Plant Molecular Biology 18:675-689) or the maize AdhI intron (Kyozuka, et al., (1991) Mol. Gen. Genet. 228:40-48; Kyozuka, et al., (1990) Maydica 35:353-357) and the like, herein incorporated by reference in their entirety.

The DNA expression cassettes or constructs useful in the methods of the disclosure can also include further enhancers, either translation or transcription enhancers, as may be required. These enhancer regions are well known to persons skilled in the art and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene and can be specifically modified to increase translation of the mRNA. It is recognized that to increase transcription levels enhancers may be utilized in combination with the promoter regions of the aspects. Enhancers are known in the art and include the SV40 enhancer region, the 35S enhancer element, and the like.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, for example, transitions and transversions, may be involved.

Reporter genes or selectable marker genes may also be included in the expression cassettes useful in the methods of the present disclosure. Examples of suitable reporter genes known in the art can be found in, for example, Jefferson, et al., (1991) in Plant Molecular Biology Manual, ed. Gelvin, et al., (Kluwer Academic Publishers), pp. 1-33; DeWet, et al., (1987) Mol. Cell. Biol. 7:725-737; Goff, et al., (1990) EMBO J. 9:2517-2522; Kain, et al., (1995) Bio Techniques 19:650-655 and Chiu, et al., (1996) Current Biology 6:325-330, herein incorporated by reference in their entirety.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Other genes that could serve utility in the recovery of transgenic events would include, but are not limited to, examples such as GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414) and the maize genes encoding for anthocyanin production (Ludwig, et al., (1990) Science 247:449), herein incorporated by reference in their entirety.

As used herein, “vector” refers to a DNA molecule such as a plasmid, cosmid or bacterial phage for introducing a nucleotide construct, for example, an expression cassette or construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

The methods of the disclosure involve introducing a polypeptide or polynucleotide into a plant. As used herein, “introducing” means presenting to the plant the polynucleotide or polypeptide in such a manner that the sequence gains access to the interior of a cell of the plant. The methods of the disclosure do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods and virus-mediated methods.

A “stable transformation” is a transformation in which the nucleotide construct introduced into a plant integrates into the genome of the plant and is capable of being inherited by the progeny thereof. “Transient transformation” means that a polynucleotide is introduced into the plant and does not integrate into the genome of the plant or a polypeptide is introduced into a plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (Townsend, et al., U.S. Pat. No. 5,563,055 and Zhao, et al., U.S. Pat. No. 5,981,840), direct gene transfer (Paszkowski, et al., (1984) EMBO J. 3:2717-2722) and ballistic particle acceleration (see, for example, U.S. Pat. Nos. 4,945,050; 5,879,918; 5,886,244; 5,932,782; Tomes, et al., (1995) in Plant Cell, Tissue, and Organ Culture: Fundamental Methods, ed. Gamborg and Phillips (Springer-Verlag, Berlin); McCabe, et al., (1988) Biotechnology 6:923-926) and Lec1 transformation (WO 00/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens), all of which are herein incorporated by reference in their entirety. Methods and compositions for rapid plant transformation of immature embryos are also found in US 2017/0121722, herein incorporated in its entirety by reference. Vectors useful in plant transformation are found in US 2019/0078106, herein incorporated by reference in its entirety.

In specific aspects, the DNA expression cassettes or constructs can be provided to a plant using a variety of transient transformation methods. Such transient transformation methods include, but are not limited to, viral vector systems and the precipitation of the polynucleotide in a manner that precludes subsequent release of the DNA. Thus, transcription from the particle-bound DNA can occur, but the frequency with which it is released to become integrated into the genome is greatly reduced. Such methods include the use of particles coated with polyethylenimine (PEI; Sigma #P3143).

In other aspects, the polynucleotide may be introduced into plants by contacting plants with a virus or viral nucleic acids. Generally, such methods involve incorporating a nucleotide construct within a viral DNA or RNA molecule. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221, herein incorporated by reference in their entirety.

The cells that have been transformed may be grown into plants in accordance with conventional ways. See, for example, McCormick, et al., (1986) Plant Cell Reports 5:81-84, herein incorporated by reference in its entirety. These plants may then be grown, and either pollinated with the same transformed strain or different strains, and the resulting progeny having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a nucleotide construct, for example, an expression cassette, stably incorporated into its genome.

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In: Methods for Plant Molecular Biology, (Eds.), Academic Press, Inc., San Diego, Calif., herein incorporated by reference in its entirety). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the aspects containing a desired polynucleotide is cultivated using methods well known to one skilled in the art.

Methods are known in the art for the targeted insertion of a polynucleotide at a specific location in the plant genome. The insertion of the polynucleotide at a desired genomic location is achieved using a site-specific recombination system. See, for example, U.S. Pat. No. 9,222,098 B2, U.S. Pat. No. 7,223,601 B2, U.S. Pat. No. 7,179,599 B2, and U.S. Pat. No. 6,911,575 B1, all of which are herein incorporated by reference in their entirety. Briefly, a polynucleotide of interest, flanked by two non-identical recombination sites, can be contained in a T-DNA transfer cassette. The T-DNA transfer cassette is introduced into a plant having stably incorporated into its genome a target site which is flanked by two non-identical recombination sites that correspond to the sites of the transfer cassette. Alternatives to T-DNA transfer include but are not limited to, particle bombardment, electroporation, PEG transfection, or RNP (ribonucleoprotein) delivery. An appropriate recombinase is provided, and the transfer cassette is integrated at the target site. The polynucleotide of interest is thereby integrated at a specific chromosomal position in the plant genome.

In an aspect, the disclosed methods can be used to introduce into leaf explants with increased efficiency and speed polynucleotides useful to target a specific site for modification in the genome of a plant. Site specific modifications that can be introduced with the disclosed methods include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. For example, the disclosed methods can be used to introduce a CRISPR-Cas system into a plant cell or plant, for the purpose of genome modification of a target sequence in the genome of a plant or plant cell, for selecting plants, for deleting a base or a sequence, for gene editing, and for inserting a polynucleotide of interest into the genome of a plant or plant cell. Thus, the disclosed methods can be used together with a CRISPR-Cas system to provide for an effective system for modifying or altering target sites and nucleotides of interest within the genome of a plant, plant cell or seed. The Cas endonuclease gene is a plant optimized Cas9 endonuclease, wherein the plant optimized Cas9 endonuclease is capable of binding to and creating a double strand break in a genomic target sequence the plant genome.

The Cas endonuclease is guided by the guide nucleotide to recognize and optionally introduce a double strand break at a specific target site into the genome of a cell. The CRISPR-Cas system provides for an effective system for modifying target sites within the genome of a plant, plant cell or seed. Further provided are methods and compositions employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest. The disclosed compositions and methods can be used to introduce a CRISPR-Cas system for editing a nucleotide sequence in the genome of a cell. The nucleotide sequence to be edited (the nucleotide sequence of interest) can be located within or outside a target site that is recognized by a Cas endonuclease.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

Cas gene includes a gene that is generally coupled, associated or close to or in the vicinity of flanking CRISPR loci. The terms “Cas gene” and “CRISPR-associated (Cas) gene” are used interchangeably herein.

In another aspect, the Cas endonuclease gene is operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas codon region.

As related to the Cas endonuclease, the terms “functional fragment,” “fragment that is functionally equivalent,” and “functionally equivalent fragment” are used interchangeably herein. These terms refer to a portion or subsequence of the Cas endonuclease sequence in which the ability to create a double-strand break is retained.

As related to the Cas endonuclease, the terms “functional variant,” “variant that is functionally equivalent” and “functionally equivalent variant” are used interchangeably herein. These terms refer to a variant of the Cas endonuclease in which the ability to create a double-strand break is retained. Fragments and variants can be obtained via methods such as site-directed mutagenesis and synthetic construction.

In an aspect, the Cas endonuclease gene is a plant codon optimized Streptococcus pyogenes Cas9 gene that can recognize any genomic sequence of the form N(12-30)NGG which can in principle be targeted.

Endonucleases are enzymes that cleave the phosphodiester bond within a polynucleotide chain and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (Patent application PCT/US 12/30061 filed on Mar. 22, 2012). Meganucleases have been classified into four families based on conserved sequence motifs. These motifs participate in the coordination of metal ions and hydrolysis of phosphodiester bonds. Meganucleases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. This cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families. TAL effector nucleases are a new class of sequence-specific nucleases that can be used to make double-strand breaks at specific target sequences in the genome of a plant or other organism. (Miller, et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ms endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

A “Dead-CAS9” (dCAS9) as used herein, is used to supply a transcriptional repressor domain. The dCAS9 has been mutated so that can no longer cut DNA. The dCAS9 can still bind when guided to a sequence by the gRNA and can also be fused to repressor elements. The dCAS9 fused to the repressor element, as described herein, is abbreviated to dCAS9-REP, where the repressor element (REP) can be any of the known repressor motifs that have been characterized in plants. An expressed guide RNA (gRNA) binds to the dCAS9-REP protein and targets the binding of the dCAS9-REP fusion protein to a specific predetermined nucleotide sequence within a promoter (a promoter within the T-DNA). For example, if this is expressed beyond—the border using a ZM-UBI PRO::dCAS9-REP::PINII TERM cassette along with a U6-POL PRO::gRNA::U6 TERM cassette and the gRNA is designed to guide the dCAS9-REP protein to bind the SB-UBI promoter in the expression cassette SB-UBI PRO::moPAT::PINII TERM within the T-DNA, any event that has integrated the beyond-the-border sequence would be bialaphos sensitive. Transgenic events that integrate only the T-DNA would express moPAT and be bialaphos resistant. The advantage of using a dCAS9 protein fused to a repressor (as opposed to a TETR or ESR) is the ability to target these repressors to any promoter within the T-DNA. TETR and ESR are restricted to cognate operator binding sequences. Alternatively, a synthetic Zinc-Finger Nuclease fused to a repressor domain can be used in place of the gRNA and dCAS9-REP (Urritia et al., 2003, Genome Biol. 4:231) as described above.

The type II CRISPR/Cas system from bacteria employs a crRNA and tracrRNA to guide the Cas endonuclease to its DNA target. The crRNA (CRISPR RNA) contains the region complementary to one strand of the double strand DNA target and base pairs with the tracrRNA (trans-activating CRISPR RNA) forming a RNA duplex that directs the Cas endonuclease to cleave the DNA target. As used herein, the term “guide nucleotide” relates to a synthetic fusion of two RNA molecules, a crRNA (CRISPR RNA) comprising a variable targeting domain, and a tracrRNA. In an aspect, the guide nucleotide comprises a variable targeting domain of 12 to 30 nucleotide sequences and a RNA fragment that can interact with a Cas endonuclease.

As used herein, the term “guide polynucleotide” relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide nucleotide”.

Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In an aspect, the guide nucleotide and Cas endonuclease are capable of forming a complex that enables the Cas endonuclease to introduce a double strand break at a DNA target site.

In an aspect of the methods of the disclosure the variable target domain is 12, 13, 14, 15, 16, 17, 18, 19, 20, 21, 22, 23, 24, 25, 26, 27, 28, 29 or 30 nucleotides in length.

In an aspect of the methods of the disclosure, the guide nucleotide comprises a cRNA (or cRNA fragment) and a tracrRNA (or tracrRNA fragment) of the type II CRISPR/Cas system that can form a complex with a type II Cas endonuclease, wherein the guide nucleotide Cas endonuclease complex can direct the Cas endonuclease to a plant genomic target site, enabling the Cas endonuclease to introduce a double strand break into the genomic target site. The guide nucleotide can be introduced into a plant or plant cell directly using any method known in the art such as, but not limited to, particle bombardment or topical applications.

In an aspect, the guide nucleotide can be introduced indirectly by introducing a recombinant DNA molecule comprising the corresponding guide DNA sequence operably linked to a plant specific promoter that is capable of transcribing the guide nucleotide in the plant cell. The term “corresponding guide DNA” includes a DNA molecule that is identical to the RNA molecule but has a “T” substituted for each “U” of the RNA molecule.

In an aspect, the guide nucleotide is introduced via particle bombardment or using the disclosed methods and compositions for Agrobacterium transformation of a recombinant DNA construct comprising the corresponding guide DNA operably linked to a plant U6 polymerase III promoter.

In an aspect, the RNA that guides the RNA Cas9 endonuclease complex, is a duplexed RNA comprising a duplex crRNA-tracrRNA. One advantage of using a guide nucleotide versus a duplexed crRNA-tracrRNA is that only one expression cassette needs to be made to express the fused guide nucleotide.

The terms “target site,” “target sequence,” “target DNA,” “target locus,” “genomic target site,” “genomic target sequence,” and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature.

As used herein, terms “endogenous target sequence” and “native target sequence” are used interchangeably herein to refer to a target sequence that is endogenous or native to the genome of a plant and is at the endogenous or native position of that target sequence in the genome of the plant. In an aspect, the target site can be similar to a DNA recognition site or target site that is specifically recognized and/or bound by a double-strand break inducing agent such as a LIG3-4 endonuclease (US patent publication 2009/0133152 A1 (published May 21, 2009) or a MS26++ meganuclease (U.S. patent application Ser. No. 13/526,912 filed Jun. 19, 2012).

An “artificial target site” or “artificial target sequence” are used interchangeably herein and refer to a target sequence that has been introduced into the genome of a plant. Such an artificial target sequence can be identical in sequence to an endogenous or native target sequence in the genome of a plant but be located in a different position (i.e., a non-endogenous or non-native position) in the genome of a plant.

An “altered target site,” “altered target sequence” “modified target site,” and “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

In an aspect, the disclosed methods can be used to introduce into plants polynucleotides useful for gene suppression of a target gene in a plant. Reduction of the activity of specific genes (also known as gene silencing, or gene suppression) is desirable for several aspects of genetic engineering in plants. Many techniques for gene silencing are well known to one of skill in the art, including but not limited to antisense technology.

In an aspect, the disclosed methods can be used to introduce into plants polynucleotides useful for the targeted integration of nucleotide sequences into a plant. For example, the disclosed methods can be used to introduce T-DNA expression cassettes comprising nucleotide sequences of interest flanked by non-identical recombination sites are used to transform a plant comprising a target site. In an aspect, the target site contains at least a set of non-identical recombination sites corresponding to those on the T-DNA expression cassette. The exchange of the nucleotide sequences flanked by the recombination sites is affected by a recombinase. Thus, the disclosed methods can be used for the introduction of T-DNA expression cassettes for targeted integration of nucleotide sequences, wherein the T-DNA expression cassettes which are flanked by non-identical recombination sites recognized by a recombinase that recognizes and implements recombination at the nonidentical recombination sites. Accordingly, the disclosed methods and composition can be used to improve efficiency and speed of development of plants containing non-identical recombination sites.

Thus, the disclosed methods can further comprise methods for the directional, targeted integration of exogenous nucleotides into a transformed plant. In an aspect, the disclosed methods use novel recombination sites in a gene targeting system which facilitates directional targeting of desired genes and nucleotide sequences into corresponding recombination sites previously introduced into the target plant genome.

In an aspect, a nucleotide sequence flanked by two non-identical recombination sites is introduced into one or more cells of an explant derived from the target organism's genome establishing a target site for insertion of nucleotide sequences of interest. Once a stable plant or cultured tissue is established a second construct, or nucleotide sequence of interest, flanked by corresponding recombination sites as those flanking the target site, is introduced into the stably transformed plant or tissues in the presence of a recombinase protein. This process results in exchange of the nucleotide sequences between the non-identical recombination sites of the target site and the T-DNA expression cassette.

It is recognized that the transformed plant prepared in this manner may comprise multiple target sites; i. e., sets of non-identical recombination sites. In this manner, multiple manipulations of the target site in the transformed plant are available. By target site in the transformed plant is intended a DNA sequence that has been inserted into the transformed plant's genome and comprises non-identical recombination sites.

Examples of recombination sites for use in the disclosed method are known. The two-micron plasmid found in most naturally occurring strains of Saccharomyces cerevisiae, encodes a site-specific recombinase that promotes an inversion of the DNA between two inverted repeats. This inversion plays a central role in plasmid copy-number amplification.

The protein, designated FLP protein, catalyzes site-specific recombination events. The minimal recombination site (FRT) has been defined and contains two inverted 13-base pair (bp) repeats surrounding an asymmetric 8-bp spacer. The FLP protein cleaves the site at the junctions of the repeats and the spacer and is covalently linked to the DNA via a 3′phosphate. Site specific recombinases like FLP cleave and religate DNA at specific target sequences, resulting in a precisely defined recombination between two identical sites. To function, the system needs the recombination sites and the recombinase. No auxiliary factors are needed. Thus, the entire system can be inserted into and function in plant cells. The yeast FLP\FRT site specific recombination system has been shown to function in plants. To date, the system has been utilized for excision of unwanted DNA. See, Lyznik et at. (1993) Nucleic Acid Res. 21: 969-975. In contrast, the present disclosure utilizes non-identical FRTs for the exchange, targeting, arrangement, insertion and control of expression of nucleotide sequences in the plant genome.

In an aspect, a transformed organism of interest, such as an explant from a plant, containing a target site integrated into its genome is needed. The target site is characterized by being flanked by non-identical recombination sites. A targeting cassette is additionally required containing a nucleotide sequence flanked by corresponding non-identical recombination sites as those sites contained in the target site of the transformed organism. A recombinase which recognizes the non-identical recombination sites and catalyzes site-specific recombination is required.

It is recognized that the recombinase can be provided by any means known in the art. That is, it can be provided in the organism or plant cell by transforming the organism with an expression cassette capable of expressing the recombinase in the organism, by transient expression, or by providing messenger RNA (mRNA) for the recombinase or the recombinase protein.

By “non-identical recombination sites” it is intended that the flanking recombination sites are not identical in sequence and will not recombine or recombination between the sites will be minimal. That is, one flanking recombination site may be a FRT site where the second recombination site may be a mutated FRT site. The non-identical recombination sites used in the methods of the present disclosure prevent or greatly suppress recombination between the two flanking recombination sites and excision of the nucleotide sequence contained therein. Accordingly, it is recognized that any suitable non-identical recombination sites may be utilized in the present disclosure, including FRT and mutant FRT sites, FRT and lox sites, lox and mutant lox sites, as well as other recombination sites known in the art.

By suitable non-identical recombination site implies that in the presence of active recombinase, excision of sequences between two non-identical recombination sites occurs, if at all, with an efficiency considerably lower than the recombinationally-mediated exchange targeting arrangement of nucleotide sequences into the plant genome. Thus, suitable non-identical sites for use in the present disclosure include those sites where the efficiency of recombination between the sites is low; for example, where the efficiency is less than about 30 to about 50%, preferably less than about 10 to about 30%, more preferably less than about 5 to about 10%.

As noted above, the recombination sites in the targeting cassette correspond to those in the target site of the transformed plant. That is, if the target site of the transformed plant contains flanking non-identical recombination sites of FRT and a mutant FRT, the targeting cassette will contain the same FRT and mutant FRT non-identical recombination sites.

It is furthermore recognized that the recombinase, which is used in the disclosed methods, will depend upon the recombination sites in the target site of the transformed plant and the targeting cassette. That is, if FRT sites are utilized, the FLP recombinase will be needed. In the same manner, where lox sites are utilized, the Cre recombinase is required. If the non-identical recombination sites comprise both a FRT and a lox site, both the FLP and Cre recombinase will be required in the plant cell.

The FLP recombinase is a protein which catalyzes a site-specific reaction that is involved in amplifying the copy number of the two-micron plasmid of S. cerevisiae during DNA replication. FLP protein has been cloned and expressed. See, for example, Cox (1993) Proc. Natl. Acad. Sci. U.S.A 80: 4223-4227. The FLP recombinase for use in the present disclosure may be that derived from the genus Saccharomyces. It may be preferable to synthesize the recombinase using plant preferred codons for optimum expression in a plant of interest. See, for example, U.S. application Ser. No. 08/972,258 filed Nov. 18, 1997, entitled “Novel Nucleic Acid Sequence Encoding FLP Recombinase,” herein incorporated by reference.

The bacteriophage recombinase Cre catalyzes site-specific recombination between two lox sites. The Cre recombinase is known in the art. See, for example, Guo et al. (1997) Nature 389: 40-46; Abremski et al. (1984) J. Biol. Chem. 259: 1509-1514; Chen et al. (1996) Somat. Cell Mol. Genet. 22: 477-488; and Shaikh et al. (1977) J. Biol. Chem. 272: 5695-5702. All of which are herein incorporated by reference. Such Cre sequence may also be synthesized using plant preferred codons.

Where appropriate, the nucleotide sequences to be inserted in the plant genome may be optimized for increased expression in the transformed plant. Where mammalian, yeast, or bacterial genes are used in the present disclosure, they can be synthesized using plant preferred codons for improved expression. It is recognized that for expression in monocots, dicot genes can also be synthesized using monocot preferred codons. Methods are available in the art for synthesizing plant preferred genes. See, for example, U.S. Pat. Nos. 5,380,831, 5,436,391, and Murray et al. (1989) Nucleic Acids Res. 17: 477-498, herein incorporated by reference. The plant preferred codons may be determined from the codons utilized more frequently in the proteins expressed in the plant of interest. It is recognized that monocot or dicot preferred sequences may be constructed as well as plant preferred sequences for particular plant species. See, for example, EPA 0359472; EPA 0385962; WO 91/16432; Perlak et al. (1991) Proc. Natl. Acad. Sci. USA, 88: 3324-3328; and Murray et al. (1989) Nucleic Acids Research, 17: 477-498. U.S. Pat. Nos. 5,380,831; 5,436,391; and the like, herein incorporated by reference. It is further recognized that all or any part of the gene sequence may be optimized or synthetic. That is, fully optimized or partially optimized sequences may also be used.

Additional sequence modifications are known to enhance gene expression in a cellular host and can be used in the present disclosure. These include elimination of sequences encoding spurious polyadenylation signals, exon-intron splice site signals, transposon-like repeats, and other such well-characterized sequences, which may be deleterious to gene expression. The G-C content of the sequence may be adjusted to levels average for a given cellular host, as calculated by reference to known genes expressed in the host cell. When possible, the sequence is modified to avoid predicted hairpin secondary RNA structures.

The present disclosure also encompasses novel FLP recombination target sites (FRT). The FRT has been identified as a minimal sequence comprising two 13 base pair repeats, separated by an eight (8) base spacer. The nucleotides in the spacer region can be replaced with a combination of nucleotides, so long as the two 13-base repeats are separated by eight nucleotides. It appears that the actual nucleotide sequence of the spacer is not critical; however, for the practice of the present disclosure, some substitutions of nucleotides in the space region may work better than others. The eight-base pair spacer is involved in DNA-DNA pairing during strand exchange. The asymmetry of the region determines the direction of site alignment in the recombination event, which will subsequently lead to either inversion or excision. As indicated above, most of the spacer can be mutated without a loss of function. See, for example, Schlake and Bode (1994) Biochemistry 33: 12746-12751, herein incorporated by reference.

Novel FRT mutant sites can be used in the practice of the disclosed methods. Such mutant sites may be constructed by PCR-based mutagenesis. Although mutant FRT sites are known (see SEQ ID Nos 2, 3, 4 and 5 of WO1999/025821), it is recognized that other mutant FRT sites may be used in the practice of the present disclosure. The present disclosure is not restricted to the use of a particular FRT or recombination site, but rather that non-identical recombination sites or FRT sites can be utilized for targeted insertion and expression of nucleotide sequences in a plant genome. Thus, other mutant FRT sites can be constructed and utilized based upon the present disclosure.

As discussed above, bringing genomic DNA containing a target site with non-identical recombination sites together with a vector containing a T-DNA expression cassette with corresponding non-identical recombination sites, in the presence of the recombinase, results in recombination. The nucleotide sequence of the T-DNA expression cassette located between the flanking recombination sites is exchanged with the nucleotide sequence of the target site located between the flanking recombination sites. In this manner, nucleotide sequences of interest may be precisely incorporated into the genome of the host.

It is recognized that many variations of the present disclosure can be practiced. For example, target sites can be constructed having multiple non-identical recombination sites. Thus, multiple genes or nucleotide sequences can be stacked or ordered at precise locations in the plant genome. Likewise, once a target site has been established within the genome, additional recombination sites may be introduced by incorporating such sites within the nucleotide sequence of the T-DNA expression cassette and the transfer of the sites to the target sequence. Thus, once a target site has been established, it is possible to subsequently add sites, or alter sites through recombination.

Another variation includes providing a promoter or transcription initiation region operably linked with the target site in an organism. Preferably, the promoter will be 5′ to the first recombination site. By transforming the organism with a T-DNA expression cassette comprising a coding region, expression of the coding region will occur upon integration of the T-DNA expression cassette into the target site. This aspect provides for a method to select transformed cells, particularly plant cells, by providing a selectable marker sequence as the coding sequence.

Other advantages of the present system include the ability to reduce the complexity of integration of transgenes or transferred DNA in an organism by utilizing T-DNA expression cassettes as discussed above and selecting organisms with simple integration patterns. In the same manner, preferred sites within the genome can be identified by comparing several transformation events. A preferred site within the genome includes one that does not disrupt expression of essential sequences and provides for adequate expression of the transgene sequence.

The disclosed methods also provide for means to combine multiple expression cassettes at one location within the genome. Recombination sites may be added or deleted at target sites within the genome.

Any means known in the art for bringing the three components of the system together may be used in the present disclosure. For example, a plant can be stably transformed to harbor the target site in its genome. The recombinase may be transiently expressed or provided. Alternatively, a nucleotide sequence capable of expressing the recombinase may be stably integrated into the genome of the plant. In the presence of the corresponding target site and the recombinase, the T-DNA expression cassette, flanked by corresponding non-identical recombination sites, is inserted into the transformed plant's genome.

Alternatively, the components of the system may be brought together by sexually crossing transformed plants. In this aspect, a transformed plant, parent one, containing a target site integrated in its genome can be sexually crossed with a second plant, parent two, that has been genetically transformed with a T-DNA expression cassette containing flanking non-identical recombination sites, which correspond to those in plant one. Either plant one or plant two contains within its genome a nucleotide sequence expressing recombinase. The recombinase may be under the control of a constitutive or inducible promoter. In this manner, expression of recombinase and subsequent activity at the recombination sites can be controlled.

The disclosed methods are useful in targeting the integration of transferred nucleotide sequences to a specific chromosomal site. The nucleotide sequence may encode any nucleotide sequence of interest. Particular genes of interest include those which provide a readily analyzable functional feature to the host cell and/or organism, such as marker genes, as well as other genes that alter the phenotype of the recipient cells, and the like. Thus, genes effecting plant growth, height, susceptibility to disease, insects, nutritional value, and the like may be utilized in the present disclosure. The nucleotide sequence also may encode an ‘antisense’ sequence to turn off or modify gene expression.

It is recognized that the nucleotide sequences will be utilized in a functional expression unit or T-DNA expression cassette. By functional expression unit or T-DNA expression cassette is intended, the nucleotide sequence of interest with a functional promoter, and in most instances a termination region. There are various ways to achieve the functional expression unit within the practice of the present disclosure. In one aspect of the present disclosure, the nucleic acid of interest is transferred or inserted into the genome as a functional expression unit.

Alternatively, the nucleotide sequence may be inserted into a site within the genome which is 3′ to a promoter region. In this latter instance, the insertion of the coding sequence 3′ to the promoter region is such that a functional expression unit is achieved upon integration. The T-DNA expression cassette will comprise a transcriptional initiation region, or promoter, operably linked to the nucleic acid encoding the peptide of interest. Such an expression cassette is provided with a plurality of restriction sites for insertion of the gene or genes of interest to be under the transcriptional regulation of the regulatory regions.

A high-throughput, automated method of producing a transgenic monocot plant that contains a heterologous polynucleotide comprising contacting a monocot leaf explant with a heterologous polynucleotide expression cassette and a morphogenic gene expression cassette; selecting a monocot leaf explant containing the heterologous polynucleotide expression cassette, wherein the monocot leaf explant forms a regenerable plant structure containing the heterologous polynucleotide expression cassette within about eight weeks or less, or within about 6 weeks or less, or within about 4 weeks or less, or within about ten days to about fourteen days of the contacting; and regenerating a transgenic plant from the regenerable plant structure containing the heterologous polynucleotide expression cassette is provided. In a further aspect, the morphogenic gene expression cassette comprises (i) a nucleotide sequence encoding a functional WUS/WOX polypeptide; or (ii) a nucleotide sequence encoding a Babyboom (BBM) polypeptide or an Ovule Development Protein 2 (ODP2) polypeptide; or (iii) a combination of (i) and (ii). In an aspect, the morphogenic gene expression cassette comprises the nucleotide sequence encoding the functional WUS/WOX polypeptide. In a further aspect, wherein the nucleotide sequence encodes the functional WUS/WOX polypeptide, the functional WUS/WOX polypeptide is selected from WUS, WUS1, WUS2, WUS3, WOX2A, WOX4, WOX5A, and WOX9. In an aspect, the morphogenic gene expression cassette comprises the nucleotide sequence encoding the encoding the Babyboom (BBM) polypeptide or the Ovule Development Protein 2 (ODP2) polypeptide. In a further aspect, wherein the nucleotide sequence encodes the Babyboom (BBM) polypeptide or the Ovule Development Protein 2 (ODP2) polypeptide, the Babyboom (BBM) polypeptide is selected from BBM, BBM1, BBM2, BBM3, BMN2, and BMN3 or the Ovule Development Protein 2 (ODP2) polypeptide is ODP2. In an aspect, the morphogenic gene expression cassette comprises the nucleotide sequence encoding the functional WUS/WOX polypeptide and the nucleotide sequence encoding the Babyboom (BBM) polypeptide or the Ovule Development Protein 2 (ODP2) polypeptide. In a further aspect, wherein the nucleotide sequence encodes the functional WUS/WOX polypeptide and the nucleotide sequence encodes the Babyboom (BBM) polypeptide or the Ovule Development Protein 2 (ODP2) polypeptide, the nucleotide sequence encoding the functional WUS/WOX polypeptide is selected from WUS, WUS1, WUS2, WUS3, WOX2A, WOX4, WOX5A, and WOX9 and the nucleotide sequence encoding the the Babyboom (BBM) polypeptide is selected from BBM, BBM1, BBM2, BBM3, BMN2, and BMN3 or the Ovule Development Protein 2 (ODP2) polypeptide is ODP2. In an aspect, the contacting is selected from T-DNA transfer, particle bombardment, electroporation, PEG transfection, or RNP (ribonucleoprotein) delivery. In an aspect, the heterologous polynucleotide expression cassette comprises a heterologous polynucleotide selected from the group consisting of a heterologous polynucleotide conferring a nutritional enhancement, a heterologous polynucleotide conferring a modified oil content, a heterologous polynucleotide conferring a modified protein content, a heterologous polynucleotide conferring a modified metabolite content, a heterologous polynucleotide conferring increased yield, a heterologous polynucleotide conferring abiotic stress tolerance, a heterologous polynucleotide conferring drought tolerance, a heterologous polynucleotide conferring cold tolerance, a heterologous polynucleotide conferring herbicide tolerance, a heterologous polynucleotide conferring pest resistance, a heterologous polynucleotide conferring pathogen resistance, a heterologous polynucleotide conferring insect resistance, a heterologous polynucleotide conferring nitrogen use efficiency (NUE), a heterologous polynucleotide conferring disease resistance, a heterologous polynucleotide conferring increased biomass, a heterologous polynucleotide conferring an ability to alter a metabolic pathway, and a combination of the foregoing. In an aspect, the leaf explant useful in the methods of the disclosure is selected from the group consisting of a leaf, a radical leaf, a cauline leaf, an alternate leaf, an opposite leaf, a decussate leaf, an opposite superposed leaf, a whorled leaf, a petiolate leaf, a sessile leaf, a subsessile leaf, a stipulate leaf, an exstipulate leaf, a simple leaf, a compound leaf, leaf primordia, a leaf sheath, a leaf base, a portion of a leaf immediately proximal to its attachment point to a petiole or stem, a bud, including but not limited to a lateral bud, and a combination of the foregoing. In an aspect, monocots useful in the methods of the disclosure are selected from the group consisting of Panicum virgatum (switchgrass), Sorghum bicolor (sorghum, sudangrass), Miscanthus giganteus (miscanthus), Saccharum sp. (energycane), Zea mays (corn), Triticum aestivum (wheat), Oryza sativa (rice), Pennisetum glaucum (pearl millet), Panicum spp., Sorghum spp., Miscanthus spp., Saccharum spp., and Erianthus spp. In an aspect the monocot useful in the methods of the disclosure is selected from the Poaceae family. In an aspect, wherein the monocot is from the Poaceae family, the monocot is selected from a Poaceae sub-family selected from Chloridoideae, Panicoideae, Oryzoideae, and Pooideae. In an aspect, wherein the monocot is from the Poaceae sub-family Chloridoideae, the monocot is Eragrostis tef In an aspect, wherein the monocot is from the Poaceae sub-family Panicoideae the monocot is selected from Zea mays, Sorghum bicolor, Pennisitum glaucum, and Panicum virgatum. In an aspect, wherein the monocot is from the Poaceae sub-family Oryzoideae the monocot is Oryza sativa. In an aspect, wherein the monocot is from the Poaceae sub-family Pooideae the monocot is selected from Hordeum vulgare, Secale cereal, and Triticum aestivum. In an aspect, the morphogenic gene expression cassette comprises a polynucleotide selected from a ZM-MIR-Corngrass1 nucleotide, a ZM-GRF5 nucleotide, a ZM-GRF4 nucleotide, a ZM-GIF1 nucleotide, a ZM-GRF4-GIF1 nucleotide, a ZM-STEMIN1 nucleotide, a ZM-REV nucleotide, a ZM-ESR1 nucleotide, a ZM-LAS nucleotide, a ZM-CUC3 nucleotide, a ZM-MIR-SPS1 nucleotide, a ZM-MIR-MAX1 nucleotide, or a ZM-MIR-MAX4 nucleotide. In a further aspect, the morphogenic gene expression cassette further comprises a polynucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter. In an aspect, the morphogenic gene expression cassette is excised to provide a transgenic monocot plant that contains the heterologous polynucleotide. In an aspect, the morphogenic gene expression cassette is bred away from to provide the transgenic monocot plant that contains the heterologous polynucleotide. In an aspect, a transgenic plant produced by the methods disclosed herein is provided, wherein the plant comprises the heterologous polynucleotide. In an aspect, a seed of the transgenic plant produced by the methods disclosed herein is provided, wherein the seed comprises the heterologous polynucleotide.

A high-throughput, automated method of producing a genome-edited monocot plant comprising contacting a monocot leaf explant with a morphogenic gene expression cassette and providing a polynucleotide encoding a site-specific polypeptide or a site-specific nuclease; selecting a monocot leaf explant containing a genome edit, wherein the monocot leaf explant forms a regenerable plant structure containing the genome edit within about eight weeks or less, or within about 6 weeks or less, or within about 4 weeks or less, or within about ten days to about fourteen days of the contacting; and regenerating a genome-edited plant from the regenerable plant structure containing the genome edit is provided. In a further aspect, the morphogenic gene expression cassette comprises (i) a nucleotide sequence encoding a functional WUS/WOX polypeptide; or (ii) a nucleotide sequence encoding a Babyboom (BBM) polypeptide or an Ovule Development Protein 2 (ODP2) polypeptide; or (iii) a combination of (i) and (ii). In an aspect, the morphogenic gene expression cassette comprises the nucleotide sequence encoding the functional WUS/WOX polypeptide. In a further aspect, wherein the nucleotide sequence encodes the functional WUS/WOX polypeptide, the functional WUS/WOX polypeptide is selected from WUS, WUS1, WUS2, WUS3, WOX2A, WOX4, WOX5A, and WOX9. In an aspect, the morphogenic gene expression cassette comprises the nucleotide sequence encoding the encoding the Babyboom (BBM) polypeptide or the Ovule Development Protein 2 (ODP2) polypeptide. In a further aspect, wherein the nucleotide sequence encodes the Babyboom (BBM) polypeptide or the Ovule Development Protein 2 (ODP2) polypeptide, the Babyboom (BBM) polypeptide is selected from BBM, BBM1, BBM2, BBM3, BMN2, and BMN3 or the Ovule Development Protein 2 (ODP2) polypeptide is ODP2. In an aspect, the morphogenic gene expression cassette comprises the nucleotide sequence encoding the functional WUS/WOX polypeptide and the nucleotide sequence encoding the Babyboom (BBM) polypeptide or the Ovule Development Protein 2 (ODP2) polypeptide. In a further aspect, wherein the nucleotide sequence encodes the functional WUS/WOX polypeptide and the nucleotide sequence encodes the Babyboom (BBM) polypeptide or the Ovule Development Protein 2 (ODP2) polypeptide, the nucleotide sequence encoding the functional WUS/WOX polypeptide is selected from WUS, WUS1, WUS2, WUS3, WOX2A, WOX4, WOX5A, and WOX9 and the nucleotide sequence encoding the the Babyboom (BBM) polypeptide is selected from BBM, BBM1, BBM2, BBM3, BMN2, and BMN3 or the Ovule Development Protein 2 (ODP2) polypeptide is ODP2. In an aspect, the site-specific polypeptide or the site-specific nuclease is selected from the group consisting of a zinc finger nuclease, a meganuclease, TALEN, and a CRISPR-Cas nuclease. In a further aspect, the CRISPR-Cas nuclease is Cas9 or Cpf1 nuclease and further comprising providing a guide RNA. In an aspect, the site-specific polypeptide or the site-specific nuclease effects an insertion, a deletion, or a substitution mutation. In an aspect, the guide RNA and CRISPR-Cas nuclease is a ribonucleoprotein complex. In an aspect, the leaf explant useful in the methods of the disclosure is selected from the group consisting of a leaf, a radical leaf, a cauline leaf, an alternate leaf, an opposite leaf, a decussate leaf, an opposite superposed leaf, a whorled leaf, a petiolate leaf, a sessile leaf, a subsessile leaf, a stipulate leaf, an exstipulate leaf, a simple leaf, a compound leaf, leaf primordia, a leaf sheath, a leaf base, a portion of a leaf immediately proximal to its attachment point to a petiole or stem, a bud, including but not limited to a lateral bud, and a combination of the foregoing. In an aspect, monocots useful in the methods of the disclosure are selected from the group consisting of Panicum virgatum (switchgrass), Sorghum bicolor (sorghum, sudangrass), Miscanthus giganteus (miscanthus), Saccharum sp. (energycane), Zea mays (corn), Triticum aestivum (wheat), Oryza sativa (rice), Pennisetum glaucum (pearl millet), Panicum spp., Sorghum spp., Miscanthus spp., Saccharum spp., and Erianthus spp. In an aspect the monocot useful in the methods of the disclosure is selected from the Poaceae family. In an aspect, wherein the monocot is from the Poaceae family, the monocot is selected from a Poaceae sub-family selected from Chloridoideae, Panicoideae, Oryzoideae, and Pooideae. In an aspect, wherein the monocot is from the Poaceae sub-family Chloridoideae, the monocot is Eragrostis tef In an aspect, wherein the monocot is from the Poaceae sub-family Panicoideae the monocot is selected from Zea mays, Sorghum bicolor, Pennisitum glaucum, and Panicum virgatum. In an aspect, wherein the monocot is from the Poaceae sub-family Oryzoideae the monocot is Oryza sativa. In an aspect, wherein the monocot is from the Poaceae sub-family Pooideae the monocot is selected from Hordeum vulgare, Secale cereal, and Triticum aestivum. In an aspect, the morphogenic gene expression cassette comprises a polynucleotide selected from a ZM-MIR-Corngrass1 nucleotide, a ZM-GRF5 nucleotide, a ZM-GRF4 nucleotide, a ZM-GIF1 nucleotide, a ZM-GRF4-GIF1 nucleotide, a ZM-STEMIN1 nucleotide, a ZM-REV nucleotide, a ZM-ESR1 nucleotide, a ZM-LAS nucleotide, a ZM-CUC3 nucleotide, a ZM-MIR-SPS1 nucleotide, a ZM-MIR-MAX1 nucleotide, or a ZM-MIR-MAX4 nucleotide. In a further aspect, the morphogenic gene expression cassette further comprises a polynucleotide sequence encoding a site-specific recombinase selected from the group consisting of FLP, FLPe, KD, Cre, SSV1, lambda Int, phi C31 Int, HK022, R, B2, B3, Gin, Tn1721, CinH, ParA, Tn5053, Bxb1, TP907-1, or U153, wherein the site-specific recombinase is operably linked to a constitutive promoter, an inducible promoter, a tissue-specific promoter, or a developmentally regulated promoter. In an aspect, the morphogenic gene expression cassette is excised to provide a genome-edited plant. In an aspect, the morphogenic gene expression cassette is bred away from to provide the genome-edited plant that contains the genome edit. In an aspect, a genome-edited plant produced by the methods disclosed herein is provided, wherein the plant comprises genome edit. In an aspect, a seed of the genome-edited plant produced by the methods disclosed herein is provided, wherein the seed comprises the genome edit.

A method of high-throughput, automated seedling propagation to produce shoots and leaf tissue/segments for transformation is provided, the method includes germinating a sterilized bulk population of seed, growing the germinated seedlings to a sufficient height for harvesting seedling leaves by mechanical and/or robotic means; harvesting the seedling leaves by mechanical and/or robotic means including, but not limited to, cutting and/or chopping with scissors, blades, wires, lasers, high-pressure water jet streams, high-pressure air, and a combination of the foregoing to provide a population of leaf segments having exposed leaf primordia for use in further high-throughput, automated transformation methods and processes including sub-culturing, resting, selection, heat-treatment, regeneration, maturation, and rooting processes by automated, mechanical, and/or robotic means resulting in rapid somatic embryo formation on the transformed leaf segments.

The rapidly formed somatic embryos or T0, T1, or further progeny of the rapidly formed somatic embryos are characterized by high-throughput, automated analyses for use in automated, high-throughput breeding programs. In an aspect, a method of high-throughput, automated analysis of rapidly formed somatic embryos or T0, T1, or further progeny of the rapidly formed somatic embryos is provided, the method includes characterizing a large number of rapidly formed somatic embryos or T0, T1, or further progeny of the rapidly formed somatic embryos, wherein the characterization includes data obtained from one or more genotyping and/or phenotyping experiments; predicting phenotypic performance of the rapidly formed somatic embryos or T0, T1, or further progeny of the rapidly formed somatic embryos or a substantial portion thereof using a biological model based on the genotyping and/or phenotyping data of population of rapidly formed somatic embryos or T0, T1, or further progeny of the rapidly formed somatic embryos that are characterized; and selecting a rapidly formed somatic embryos or T0, T1, or further progeny of the rapidly formed somatic embryos from the population of rapidly formed somatic embryos or T0, T1, or further progeny of the rapidly formed somatic embryos based on the predicted phenotypic performance; and regenerating a plant derived from the selected rapidly formed somatic embryos or T0, T1, or further progeny of the rapidly formed somatic embryos.

In an embodiment, the characterizing step includes one or more processes selected from the group consisting of: high-throughput genotyping of DNA isolated from the rapidly formed somatic embryos or T0, T1, or further progeny of the rapidly formed somatic embryos or the cell derived from the rapidly formed somatic embryos or T0, T1, or further progeny of the rapidly formed somatic embryos; or high-throughput measurement or detection of RNA transcripts isolated from the rapidly formed somatic embryos or T0, T1, or further progeny of the rapidly formed somatic embryos or the cell derived from the rapidly formed somatic embryos or T0, T1, or further progeny of the rapidly formed somatic embryos; or high-throughput measurement or detection of nucleosome abundance or densities of chromatin isolated from the rapidly formed somatic embryos or T0, T1, or further progeny of the rapidly formed somatic embryos or the cell derived from the rapidly formed somatic embryos or T0, T1, or further progeny of the rapidly formed somatic embryos; or high-throughput measurement or detection of post-translational modifications of histone proteins of chromatin isolated from the rapidly formed somatic embryos or T0, T1, or further progeny of the rapidly formed somatic embryos or the cell derived from the rapidly formed somatic embryos or T0, T1, or further progeny of the rapidly formed somatic embryos; or high-throughput measurement or detection of epigenetic modifications of DNA or RNA isolated from the rapidly formed somatic embryos or T0, T1, or further progeny of the rapidly formed somatic embryos or the cell derived from the rapidly formed somatic embryos or T0, T1, or further progeny of the rapidly formed somatic embryos; or high-throughput measurement or detection of protein:DNA interactions of chromatin isolated from the rapidly formed somatic embryos or T0, T1, or further progeny of the rapidly formed somatic embryos or the cell derived from the rapidly formed somatic embryos or T0, T1, or further progeny of the rapidly formed somatic embryos; high-throughput measurement or detection of protein:RNA interactions or complexes isolated from the rapidly formed somatic embryos or T0, T1, or further progeny of the rapidly formed somatic embryos or the cell derived from the rapidly formed somatic embryos or T0, T1, or further progeny of the rapidly formed somatic embryos; and a combination of the foregoing.

In an embodiment, the predicting phenotypic performance is selected from the group consisting of: using large-scale genomic data based on genotyping by DNA sequencing of the rapidly formed somatic embryos or T0, T1, or further progeny of the rapidly formed somatic embryos or the cell derived from the rapidly formed somatic embryos or T0, T1, or further progeny of the rapidly formed somatic embryos; or using genomic data based on genotyping by assay of rapidly formed somatic embryos or T0, T1, or further progeny of the rapidly formed somatic embryos or the cell derived from the rapidly formed somatic embryos or T0, T1, or further progeny of the rapidly formed somatic embryos; or using large-scale genomic data based on a known or predicted expression state of the rapidly formed somatic embryos or T0, T1, or further progeny of the rapidly formed somatic embryos or the cell derived from the rapidly formed somatic embryos or T0, T1, or further progeny of the rapidly formed somatic embryos; or using large-scale genomic data based on a known or predicted chromatin state of the rapidly formed somatic embryos or T0, T1, or further progeny of the rapidly formed somatic embryos or the cell derived from the rapidly formed somatic embryos or T0, T1, or further progeny of the rapidly formed somatic embryos; or using large-scale genomic data based on a known or predicted epigenetic regulatory state of the rapidly formed somatic embryos or T0, T1, or further progeny of the rapidly formed somatic embryos or the cell derived from the rapidly formed somatic embryos or T0, T1, or further progeny of the rapidly formed somatic embryos; or using large-scale genotype imputation of shared haplotype genomic data of the rapidly formed somatic embryos or T0, T1, or further progeny of the rapidly formed somatic embryos or the cell derived from the rapidly formed somatic embryos or T0, T1, or further progeny of the rapidly formed somatic embryos; or using large-scale pedigree history data of the rapidly formed somatic embryos or T0, T1, or further progeny of the rapidly formed somatic embryos or the cell derived from the rapidly formed somatic embryos or T0, T1, or further progeny of the rapidly formed somatic embryos; and a combination of the foregoing.

Automated methods described herein are suitable to applications where a large sample number of tissues are provided, for example, in high-throughput processes, scaled screening methods, large-scale processing of transformation or tissue culture. Automation of the whole process or sub-steps thereof is accomplished, for example, by employing robotic or mechanical handling seeds, seedlings, leaf segments, or transformed leaf segments. Such automation methods may rely on one or more sensors, including optical, mechanical, electrical sensors to aid in identification, positioning, extracting, processing, sub-culturing, resting, selecting, heat-treating, regenerating, maturing, or rooting processes of seeds, seedlings, leaf segments, or transformed leaf segments either in a single step, or in multiple separate steps. In an embodiment, the method provides substantially isolated seeds, seedlings, leaf segments, or transformed leaf segments at a rate of between about 1000 to 100,000 or more seeds, seedlings, leaf segments, or transformed leaf segments per employee-day; or between about 1000 to about 100,000, or about 1000 to about 50,000, or about 1000 to about 20,000, or about 1000 to about 10,000, or about 1000 to about 5000, or about 1000 to about 3000, or about 1000 to about 3000 seeds, seedlings, leaf segments, or transformed leaf segments per employee-day; or between about 800 to about 100,000, or about 2000 to about 50,000, or about 2500 to about 20,000, or about 3000 to about 10,000, or about 5000 to about 15000, or about 800 to about 3000, or about 800 to about 1000 seeds, seedlings, leaf segments, or transformed leaf segments per employee-day; or between about 2500 and about 100,000, or about 2500 to about 50,000, or about 2500 to about 20,000, or about 2500 to about 10,000, or about 2500 to about 5000, or about 2500 to about 3000 seeds, seedlings, leaf segments, or transformed leaf segments per employee-day; or between about 5000 and about 100,000, or about 5000 to about 50,000, or about 5000 to about 20,000, or about 5000 to about 10,000 seeds, seedlings, leaf segments, or transformed leaf segments per full-time equivalent (FTE), or any fraction or whole number in between any of the aforementioned ranges. The aforementioned ranges are also suitable for transformed somatic embryos, T0, T1, or further progeny of the rapidly formed somatic embryos.

Methods described herein are suitable to applications where a large number of sample tissues are provided, for example, in high-throughput processes or screening, or in batch processing for genetic transformation, double haploid induction, tissue culture, seedling derived material, embryonic axes and their derived material for any type of genetic modulation. Automated methods include deploying robotic handling of the ears or seeds, pods, plants, silks, tassels, and any electro, electro-mechanical application of force to any plant part, wherein those operations are controlled by one or more algorithms operable on premises or delivered through a cloud-based server operation. Automation methods described herein include sensors of various types, including optical or mechanical sensors, imaging, hyperspectral, or other non-destructive analytics to aid in positioning of the tissue being used or processed.

Automated methods of characterizing plant cells including, but not limited to, genotyping DNA isolated from a plant cell, measuring RNA transcripts isolated from a plant cell, measuring nucleosome abundance or densities of chromatin isolated from a plant cell, measuring post-translational modifications of histone proteins of chromatin isolated from a plant cell, measuring or estimating methylation status of genomic DNA, measuring epigenetic modifications of DNA or RNA isolated from a plant cell, measuring protein:DNA interactions of chromatin isolated from a plant cell, and measuring protein:RNA interactions or complexes isolated from a plant cell are useful in the methods of the present disclosure. Imaging includes, for example, shape, size, thickness of leaf segments or somatic embryos obtained during the leaf transformation process.

In certain embodiments, a system is provided for preparing a population of leaf explants suitable for transformation including a seed receiving station for receiving a population of seed, a seed sterilization station, a seedling germination station, a leaf harvesting station, a leaf cutting station, a leaf segment infection station, a resting station, a selection station, a regeneration station, a maturation station, a rooting station, a potting station, and a container transport mechanism. The seed receiving station may be configured to receive a bulk population of seed into a container for sterilization and for further processing at additional stations. The sterilization station may comprise means for providing and decanting/removing a series of solutions to the container of seed to provide a bulk population of sterilized seed. The seedling gemination station may comprise growth containers or trays containing germination media for receiving the sterilized seed for germination of the seed. The leaf harvesting station may comprise means to measure and determine when the germinated seedlings leaves are of a sufficient height for harvesting in the leaf cutting station. The leaf cutting station may comprise a chopping means, a cutting means, and/or a blending means and chopping, cutting, and/or blending means of the harvested seedling leaves to provide leaf segments of an appropriate size for further processing in the leaf segment infection station. The leaf segment infection station may comprise means for providing and decanting/removing a bacterial infection solution. Each of the resting station, selection station, regeneration station, maturation station, and rooting station may comprise means for providing and decanting/removing appropriate media for the operations carried out at each station. The potting station may comprise means for potting plantlets from the rooting station. The container transport mechanism may be configured to automatically move the container from the seed receiving station to the seed sterilization station and from the seedling sterilization station to the seedling germination station and from the seedling germination station to the leaf harvesting station and from the leaf harvesting station to the leaf cutting station and from the leaf cutting station to the leaf segment infection station and from the leaf segment infection station to the resting station and from the resting station to the selection station and from the selection station to the regeneration station and from the regeneration station to the maturation station and from the maturation station to the rooting station and from the rooting station to the potting station and from the potting station to a greenhouse upon completion of a respective operation of the seedling receiving station, seedling germination station, leaf harvesting station, leaf cutting station, leaf segment infection station, resting station, selection station, regeneration station, maturation station, the rooting station, and the potting station.

In still other embodiments, a method of preparing a representative sample (e.g., leaf segments or somatic embryos) for analysis is provided. The method includes receiving a sample container having at least one isolated compartment, each isolated compartment having a sample therein. In some cases, a force applying member includes at least one protrusion, and applying a force to the sample comprises pressing each protrusion into direct contact with the sample in each isolated compartment to break the sample into smaller portions, suitable for sampling.

In some embodiments, the method may also include directing the sample particles into corresponding collection cavities. The sample particles may be directed into the corresponding collection cavity of a sample collector using a directing member that includes at least one channel configured to provide an isolated passageway between each isolated compartment of the sample container and the corresponding collection cavity of the sample collector.

The characterization information and data from leaf segments or somatic embryos generated in the methods of the present disclosure is used to predict phenotypic performance of the leaf segments or somatic embryos and facilitate breeding decisions earlier in the development process. Methods of predicting phenotypic performance of leaf segments or somatic embryos including, but not limited to, using genomic data based on genotyping by DNA sequencing of a leaf segment or somatic embryo, using genomic data based on genotyping by assay of a leaf segment or somatic embryo, using genomic data based on a known or predicted expression state of a leaf segment or somatic embryo, using genomic data based on a known or predicted chromatin state of a leaf segment or somatic embryo, using genomic data based on a known or predicted epigenetic regulatory state of a leaf segment or somatic embryo, and using genotype imputation of shared haplotype genomic data of a leaf segment or somatic embryo and/or pedigree history data of a leaf segment or somatic embryo are useful in the methods of the present disclosure and facilitate the plant breeding process.

The piece of leaf segment or somatic embryo may be analyzed for one or more characteristics selected from the group consisting of: a genetic marker, a single nucleotide polymorphism, a simple sequence repeat, a restriction fragment length polymorphism, a haplotype, a tag SNP, an allele of a genetic marker, a gene, a DNA-derived sequence, an RNA-derived sequence, a promoter, a 5′-untranslated region of a gene, a 3-untranslated region of a gene, microRNA, siRNA, a QTL, a satellite marker, a transgene, mRNA, ds mRNA, a transcriptional profile, a methylation pattern, and ploidy level.

Automated methods of analyzing a leaf segment or somatic embryo of a plant are provided herein. In such methods, a leaf segment or somatic embryo is isolated, wherein said isolation does not cause a significant reduction in the germination potential of the leaf segment or somatic embryo. A plurality of leaf segment or somatic embryo samples are then analyzed in a high through-put manner for the presence or absence of one or more characteristics indicative of at least one genetic trait, through automated or semi-automated methods.

Methods of characterizing plant cells and predicting phenotypic performance can be accomplished using one or more methods disclosed in U.S. Pat. Nos. 6,399,855, 8,039,686, 8,321,147, U.S. Ser. No. 10/031,116, U.S. Ser. No. 10/102,476, US20160321396, US20170245446, US20170359978, and US20180363069 all of which are incorporated herein by reference in their entireties.

### EXAMPLES

The following Examples are offered by way of illustration and not by way of limitation.

### EXAMPLES

The aspects of the disclosure are further defined in the following Examples, in which parts and percentages are by weight and degrees are Celsius, unless otherwise stated. These Examples, while indicating aspects of the disclosure, are given by way of illustration only. From the above discussion and these Examples, one skilled in the art can ascertain the essential characteristics of the aspects of the disclosure, and without departing from the spirit and scope thereof, can make various changes and modifications of them to adapt to various usages and conditions. Thus, various modifications in addition to those shown and described herein will be apparent to those skilled in the art from the foregoing description. Such modifications are also intended to fall within the scope of the appended claims.

### Example 1: Sequences

Sequences useful in the methods of the disclosure are presented in Table 2.

TABLE 2

| | Polynucleotide | | |
|---|---|---|---|
| | (DNA) or | | |
| SEQ ID | Polypeptide | | |
| NO: | (PRT) | NAME | DESCRIPTION |
| 1 | DNA | PHP5096 | UBI1ZM 5UTR::UBI1ZM |
| | | | INTRON1::LOXP::FLPM::PINII TERM |
| 2 | DNA | PHP71539 | VIRB1 + VIRB2 + VIRB3 + VIRB4 + |
| | | | VIRB5 + VIRB6 + VIRB7 + VIRB8 + |
| | | | VIRB9 + VIRB10 + VIRB11 + VIRG + |
| | | | VIRC2 + VIRC1 + VIRD1 + VIRD2 + |
| | | | VIRD3 + VIRD4 + VIRD5 + VIRE1 + |
| | | | VIRE2 + VIRE3 + GENT + COLE1 |
| | | | ORI + PVS1 ORI |
| 3 | DNA | PHP81858 | RB + LOXP + NOS PRO::ZM- |
| | | | WUS2::IN2-1 TERM + PLTP |
| | | | PRO::TOP::ZM-ODP2::OS-T28 TERM + |
| | | | RAB17 PRO::MO-CRE-EXON1::ST- |
| | | | LS1 INTRON1::MO-CRE |
| | | | EXON2::PINII + LOXP + UBI1ZM |
| | | | PRO::UBI1ZM 5UTR::UBI1ZM |
| | | | INTRON1::FRT1::NPTII::PINII TERM + |
| | | | UBI1ZM PRO::UBI1ZM |
| | | | 5UTR::UBI1ZM INTRON1::AM- |
| | | | CYAN1::PINII TERM + FRT6 + LB |
| 4 | DNA | PHP89030 | ZM-PLTP PRO::ZM-ODP2::OS-T28 |
| | | | TERM + FMV ENH + PCSV ENH |
| 5 | DNA | PHP89179 | ZM-PLTP PRO::ZM-WUS2::IN2-1 |
| | | | TERM + PSW1 + GZ-W64A TERM + |
| | | | FL2 TERM |
| 6 | DNA | PHP93739 | RB + LOXP + ZM-HSP17.7 PRO::MO- |
| | | | CRE-EXON1::ST-LS1 INTRON1::MO- |
| | | | CRE EXON2::PINII TERM + NOS |
| | | | PRO::ZM-WUS2::ZM-IN2-1 TERM + |
| | | | UBI1ZMPRO::UBI1ZM |
| | | | 5UTR::UBI1ZM INTRON1::ZM- |
| | | | ODP2::ZM-UBI TERM + LOXP + SB- |
| | | | UBI PRO::SB-UBI INTRON1::ZS- |
| | | | GREEN1::OS-UBI TERM + SI-ALS |
| | | | PRO::SI-ALS 5UTR::ZM-ALS |
| | | | (HRA)::SB-UBI TERM + LB |
| 7 | DNA | PHP96037 | RB + LOXP + NOS PRO::ZM- |
| | | | WUS2::IN2-1 TERM + FMV |
| | | | ENH::PCSV ENH::MMV |
| | | | ENH::UBI1ZM PRO::UBI1ZM |
| | | | 5UTR::UBI1ZM INTRON1::ZM- |
| | | | ODP2::OS-T28 TERM + AT-5-IV-2 |
| | | | INS + ZM-HSP17.7 PRO::MO-CRE- |
| | | | EXON1::ST-LS1 INTRON1::MO-CRE |
| | | | EXON2::PINII TERM + AT-5-IV-2 INS + |
| | | | LOXP + SB-UBI PRO::SB-UBI |
| | | | INTRON1::ZS-GREEN1::OS-UBI |
| | | | TERM + SB-ALS PRO::ZM-ALS |
| | | | (HRA)::SB-PEPC1 TERM + LB |
| 8 | DNA | PHP97334 | RB + LOXP + NOS PRO::ZM- |
| | | | WUS2::IN2-1 TERM + FMV |
| | | | ENH::PCSV ENH::MMV |
| | | | ENH::UBI1ZM PRO::UBI1ZM |
| | | | 5UTR::UBI1ZM INTRON1::ZM- |
| | | | ODP2::OS-T28 TERM + AT-5-IV-2 |
| | | | INS + ZM-HSP17.7 PRO::MO-CRE- |
| | | | EXON1::ST-LS1 INTRON1::MO-CRE |
| | | | EXON2::PINII TERM + AT-5-IV-2 INS + |
| | | | LOXP + SB-UBI PRO::SB-UBI |
| | | | INTRON1::ZS-GREEN1::OS-UBI |
| | | | TERM + SB-UBI1 PRO::SB-UBI1 |
| | | | INTRON1::NPTII::SB-UBI TERM + |
| | | | LB |
| 9 | DNA | PHP8418-0004 | FRT1::PMI::PINII TERM::CZ19B1 |
| | | | TERM + UBI1ZM PRO::UBI1ZM |
| | | | 5UTR::UBI1ZM INTRON1::DS- |
| | | | RED2::PINII TERM + FRT6 |
| 10 | DNA | PHP54733 | RB + LOXP + RAB17 PRO::MO-CRE- |
| | | | EXON1::ST-LS1 INTRON1::MO-CRE |
| | | | EXON2::PINII TERM + NOS |
| | | | PRO::ZM-WUS2::PINII TERM + |
| | | | UBI1ZM PRO PRO::UBI1ZM |
| | | | 5UTR::UBI1ZM INTRON1::ZM- |
| | | | ODP2::PINII TERM + LOXP + |
| | | | UBI1ZM PRO PRO::UBI1ZM |
| | | | 5UTR::UBI1ZM INTRON1::ZS- |
| | | | GREEN1::PINII TERM::SB-ACTIN |
| | | | TERM _ SB-UBI PRO::SB- |
| | | | INTRON1::PMI::SB-UBI TERM + |
| | | | FRT87 + LB |
| 11 | DNA | PHP70298 | VIRB1 + VIRB2 + VIRB3 + VIRB4 + |
| | | | VIRB5 + VIRB6 + VIRB7 + VIRB8 + |
| | | | VIRB9 + VIRB10 + VIRB11 + VIRG + |
| | | | VIRC2 + VIRC1 + VIRD1 + VIRD2 + |
| | | | GENT + COLE1 ORI + PVS1 ORI |
| 12 | DNA | RV005393 | VIRA + VIRJ + VIRB1 + VIRB2 + |
| | | | VIRB3 + VIRB4 + VIRB5 + VIRB6 + |
| | | | VIRB7 + VIRB8 + VIRB9 + VIRB10 + |
| | | | VIRB11 + VIRG-V1 + VIRC2 + |
| | | | VIRC1(FL) + VIRD1 + VIRD2 + |
| | | | VIRD3 + VIRD4 + VIRD5 + VIRE1 + |
| | | | VIRE2 + VIRE3 + GENT + ORI V + |
| | | | CTL + TRF A + PARDE |
| 13 | DNA | RV007497 | AR-VIRA-ALT1 + AR-VIRB1 + AR- |
| | | | VIRB2 + AR-VIRB3 + AR-VIRB4- |
| | | | ALT1 + AR-VIRB5-V1 + AR-VIRB6 + |
| | | | AR-VIRB7 + AR-VIRB8 + AR-VIRB9 + |
| | | | AR-VIRB10 + AR-VIRB11 + AR- |
| | | | VIRG + AR-VIRC2 + AR-VIRC1-V1 + |
| | | | AR-VIRD1 + AR-VIRD2 + AR-VIRD3- |
| | | | V1 + AR-VIRD4 + AR-VIRD5-ALT1 + |
| | | | AR-VIRF + AR-VIRE3 + AR-GALLS- |
| | | | V1 + GENT + COLE1 ORI + PVS1 ORI |
| 14 | DNA | ZM-UBI1 PRO | Zea mays ubiquitin1 promoter |
| | | (UBI1ZM PRO) | |
| 15 | DNA | ZM-UBI1 | Zea mays ubiquitin1 5′ untranslated |
| | | 5UTR | region |
| | | (UBI1ZM | |
| | | 5UTR) | |
| 16 | DNA | ZM-UBI1 | Zea mays ubiquitin1 intron1 |
| | | INTRON1 | |
| | | (UBI1ZM | |
| | | INTRON1) | |
| 17 | DNA | FMV ENH | Figwort Mosaic Virus enhancer |
| 18 | DNA | PCSV ENH | Peanut Chlorotic Streak Caulimovirus |
| | | | enhancer |
| 19 | DNA | MMV ENH | Mirabilis Mosaic Virus enhancer |
| 20 | DNA | ZM-UBI1 PRO | Zea mays ubiquitin1 promoter (SEQ ID |
| | | Complete | NO: 14) + Zea mays ubiquitin1 intron1 |
| | | | (SEQ ID NO: 15) + Zea mays ubiquitin1 |
| | | | intron1 (SEQ ID NO: 16) |
| 21 | DNA | 3 × ENH | Figwort Mosaic Virus enhancer (SEQ ID |
| | | | NO: 17) + Peanut Chlorotic Streak |
| | | | Caulimovirus enhancer (SEQ ID NO: 18) + |
| | | | Mirabilis Mosaic Virus enhancer (SEQ |
| | | | ID NO: 19) |

### Example 2: Media Compositions

Various media are referenced in the Examples for use in transformation and cell culture. The composition of these media are provided below in Tables 3-7.

TABLE 3

| | Units | | | |
|---|---|---|---|---|
| Medium Components | per liter | 12V | 810I | 700A |
| MS BASAL SALT MIXTURE | g | | | 4.3 |
| N6 MACRONUTRIENTS 10X | ml | | | |
| POTASSIUM NITRATE | g | | | |
| B5H MINOR SALTS 1000X | ml | | | |
| NaFe EDTA FOR B5H 100X | ml | | | |
| ERIKSSON'S VITAMINS 1000X | ml | | | |
| S&H VITAMIN STOCK 100X | ml | | | |
| THIAMINE•HCL | mg | | | 10.0 |
| L-PROLINE | g | | | |
| CASEIN HYDROLYSATE (ACID) | g | | | |
| SUCROSE | g | | | 68.5 |
| GLUCOSE | g | 5.0 | | 36.0 |
| MALTOSE | g | | | |
| 2,4-D | mg | | | 1.5 |
| AGAR | g | 15.0 | 15.0 | |
| PHYTAGEL | g | | | |
| DICAMBA | g | | | |
| SILVER NITRATE | mg | | | |
| AGRIBIO Carbenicillin | mg | | | |
| Timentin | mg | | | |
| Cefotaxime | mg | | | |
| MYO-INOSITOL | g | | | 0.1 |
| NICOTINIC ACID | mg | | | 0.5 |
| PYRIDOXINE•HCL | mg | | | 0.5 |
| VITAMIN ASSAY CASAMINO ACIDS | g | | | 1.0 |
| MES BUFFER | g | | | |
| ACETOSYRINGONE | uM | | | |
| ASCORBIC ACID 10 MG/ML (7S) | mg | | | |
| MS VITAMIN STOCK SOL. | ml | | | |
| ZEATIN | mg | | | |
| CUPRIC SULFATE | mg | | | |
| IAA 0.5 MG/ML (28A) | ml | | | |
| ABA 0.1 mm | ml | | | |
| THIDIAZURON | mg | | | |
| AGRIBIO Carbenicillin | mg | | | |
| PPT(GLUFOSINATE-NH4) | mg | | | |
| BAP | mg | | | |
| YEAST EXTRACT (BD Difco) | g | | 5.0 | |
| PEPTONE | g | | 10.0 | |
| SODIUM CHLORIDE | g | | 5.0 | |
| SPECTINOMYCIN | mg | 50.0 | 100.0 | |
| FERROUS SULFATE•7H20 | ml | 2.0 | | |
| AB BUFFER 20X (12D) | ml | 50.0 | | |
| AB SALTS 20X (12E) | ml | 50.0 | | |
| Benomyl | mg | | | |
| pH | | | | 5.2 |

TABLE 4

| | Units | | | | |
|---|---|---|---|---|---|
| | per | | | | |
| Medium Components | liter | 710I | 605J | 605T | 289Q |
| MS BASAL SALT MIXTURE | g | 4.3 | 4.3 | 4.3 | 4.3 |
| N6 MACRONUTRIENTS 10X | ml | | 60.0 | 60.0 | |
| POTASSIUM NITRATE | g | | 1.7 | 1.7 | |
| B5H MINOR SALTS 1000X | ml | | 0.6 | 0.6 | |
| NaFe EDTA FOR B5H 100X | ml | | 6.0 | 6.0 | |
| ERIKSSON'S VITAMINS 1000X | ml | | 0.4 | 0.4 | |
| S&H VITAMIN STOCK 100X | ml | | 6.0 | 6.0 | |
| THIAMINE•HCL | mg | 10.0 | 0.5 | 0.5 | |
| L-PROLINE | g | 0.7 | 2.0 | 2.0 | 0.7 |
| CASEIN HYDROLYSATE (ACID) | g | | 0.3 | 0.3 | |
| SUCROSE | g | 20.0 | 20.0 | 20.0 | 60.0 |
| GLUCOSE | g | 10.0 | 0.6 | 0.6 | |
| MALTOSE | g | | | | |
| 2,4-D | mg | 2.0 | 0.8 | 0.8 | |
| AGAR | g | 8.0 | 6.0 | 6.0 | 8.0 |
| PHYTAGEL | g | | | | |
| DICAMBA | g | | 1.2 | 1.2 | |
| SILVER NITRATE | mg | | 3.4 | 3.4 | |
| AGRIBIO Carbenicillin | mg | | 100.0 | | |
| Timentin | mg | | | 150.0 | 150.0 |
| Cefotaxime | mg | | | 100.0 | 100.0 |
| MYO-INOSITOL | g | 0.1 | | | 0.1 |
| NICOTINIC ACID | mg | 0.5 | | | |
| PYRIDOXINE•HCL | mg | 0.5 | | | |
| VITAMIN ASSAY CASAMINO | g | | | | |
| ACIDS | | | | | |
| MES BUFFER | g | 0.5 | | | |
| ACETOSYRINGONE | uM | 100.0 | | | |
| ASCORBIC ACID 10 MG/ML (7S) | mg | 10.0 | | | |
| MS VITAMIN STOCK SOL. | ml | | | | 5.0 |
| ZEATIN | mg | | | | 0.5 |
| CUPRIC SULFATE | mg | | | | 1.3 |
| IAA 0.5 MG/ML (28A) | ml | | | | 2.0 |
| ABA 0.1 mm | ml | | | | 1.0 |
| THIDIAZURON | mg | | | | 0.1 |
| AGRIBIO Carbenicillin | mg | | | | 100.0 |
| PPT(GLUFOSINATE-NH4) | mg | | | | |
| BAP | mg | | | | 1.0 |
| YEAST EXTRACT (BD Difco) | g | | | | |
| PEPTONE | g | | | | |
| SODIUM CHLORIDE | g | | | | |
| SPECTINOMYCIN | mg | | | | |
| FERROUS SULFATE•7H20 | ml | | | | |
| AB BUFFER 20X (12D) | ml | | | | |
| AB SALTS 20X (12E) | ml | | | | |
| Benomyl | mg | | | | |
| pH | | | | | 5.6 |

TABLE 5

| | Units | | | | |
|---|---|---|---|---|---|
| | per | | | | |
| Medium Components | liter | 13266K | 272X | 272V | 13158 |
| MS BASAL SALT MIXTURE | g | 4.3 | 4.3 | 4.3 | 4.3 |
| N6 MACRONUTRIENTS 10X | ml | 60.0 | | | |
| POTASSIUM NITRATE | g | 1.7 | | | |
| B5H MINOR SALTS 1000X | ml | 0.6 | | | |
| NaFe EDTA FOR B5H 100X | ml | 6.0 | | | |
| ERIKSSON'S VITAMINS | ml | 0.4 | | | |
| 1000X | | | | | |
| S&H VITAMIN STOCK 100X | ml | 6.0 | | | |
| THIAMINE•HCL | mg | 0.5 | | | |
| L-PROLINE | g | 2.0 | | | |
| CASEIN HYDROLYSATE | g | 0.3 | | | |
| (ACID) | | | | | |
| SUCROSE | g | 20.0 | 40.0 | 40.0 | 40.0 |
| GLUCOSE | g | 0.6 | | | |
| MALTOSE | g | | | | |
| 2,4-D | mg | 1.6 | | | |
| AGAR | g | 6.0 | 6.0 | 6.0 | 6.0 |
| PHYTAGEL | g | | | | |
| DICAMBA | g | 1.2 | | | |
| SILVER NITRATE | mg | 1.7 | | | |
| AGRIBIO Carbenicillin | mg | 2.0 | | | |
| Timentin | mg | | | | |
| Cefotaxime | mg | 25 | | | |
| MYO-INOSITOL | g | | 0.1 | 0.1 | 0.1 |
| NICOTINIC ACID | mg | | | | |
| PYRIDOXINE•HCL | mg | | | | |
| VITAMIN ASSAY | g | | | | |
| CASAMINO ACIDS | | | | | |
| MES BUFFER | g | | | | |
| ACETOSYRINGONE | uM | | | | |
| ASCORBIC ACID 10 MG/ML | mg | | | | |
| (7S) | | | | | |
| MS VITAMIN STOCK SOL. | ml | | 5.0 | 5.0 | 5.0 |
| ZEATIN | mg | | | | |
| CUPRIC SULFATE | mg | | | | |
| IAA 0.5 MG/ML (28A) | ml | | | | |
| ABA 0.1 mm | ml | | | | |
| THIDIAZURON | mg | | | | |
| AGRIBIO Carbenicillin | mg | | | | |
| PPT(GLUFOSINATE-NH4) | mg | | | | |
| BAP | mg | | | | |
| YEAST EXTRACT (BD Difco) | g | | | | |
| PEPTONE | g | | | | |
| SODIUM CHLORIDE | g | | | | |
| SPECTINOMYCIN | mg | | | | |
| FERROUS SULFATE•7H20 | ml | | | | |
| AB BUFFER 20X (12D) | ml | | | | |
| AB SALTS 20X (12E) | ml | | | | |
| Benomyl | mg | | | | 100.0 |
| pH | | | 0.5 | 5.6 | |

TABLE 6

| | Units | | | | |
|---|---|---|---|---|---|
| | per | | | | |
| Medium Components | liter | 12R | 810K | 700J | 710N |
| 404 Modified MS Basal Salts | | | | | |
| MS BASAL SALT MIXTURE | g | | | | 4.3 |
| N6 BASAL SALTS | g | | | | |
| N6 MACRONUTRIENTS 10X | ml | | | | |
| POTASSIUM NITRATE | g | | | | |
| B5H MINOR SALTS 1000X | ml | | | | |
| NaFe EDTA FOR B5H 100X | ml | | | | |
| ERIKSSON'S VITAMINS | ml | | | | |
| 1000X | | | | | |
| S&H VITAMIN STOCK | g | | | | |
| THIAMINE•HCL | mg | | | | 1.0 |
| L-PROLINE | g | | | | 0.7 |
| CASEIN HYDROLYSATE | g | | | | |
| (ACID) | | | | | |
| SUCROSE | g | | | | |
| GLUCOSE | g | 5.0 | | | 10.0 |
| MALTOSE | g | | | 5.0 | 20.0 |
| 2,4-D | mg | | | | 2.0 |
| AGAR | g | 15.0 | | | 8.0 |
| BACTO-AGAR | g | | 15.0 | | |
| PHYTAGEL | g | | | | |
| DICAMBA | g | | | | |
| SILVER NITRATE | mg | | | | |
| AGRIBIO Carbenicillin | mg | | | | |
| Timentin | mg | | | | |
| Cefotaxime | mg | | | | |
| MYO-INOSITOL | g | | | | 0.1 |
| NICOTINIC ACID | mg | | | | 0.5 |
| PYRIDOXINE•HCL | mg | | | | 0.5 |
| VITAMIN ASSAY CASAMINO | g | | | | |
| ACIDS | | | | | |
| MES BUFFER | g | | | | 0.5 |
| ACETOSYRINGONE | uM | | | | 100.0 |
| ASCORBIC ACID 10 MG/ML | mg | | | | 10.0 |
| (7S) | | | | | |
| MS VITAMIN STOCK SOL. | ml | | | | |
| ZEATIN | mg | | | | |
| CUPRIC SULFATE | uM | | | | 100.0 |
| IAA 0.5 MG/ML (28A) | ml | | | | |
| ABA 0.1 mm | ml | | | | |
| 12N-a-NAA 1 MG/ML | mg | | | | |
| THIDIAZURON | mg | | | | |
| PPT(GLUFOSINATE-NH4) | mg | | | | |
| BAP | mg | | | | 1.0 |
| YEAST EXTRACT (BD Difco) | g | | 5.0 | | |
| PEPTONE | g | | 10.0 | | |
| SODIUM CHLORIDE | g | | 5.0 | | |
| SPECTINOMYCIN | mg | 50.0 | 50.0 | | |
| FERROUS SULFATE•7H20 | ml | 2.0 | | | |
| AB BUFFER 20X (12D) | ml | 50.0 | | | |
| AB SALTS 20X (12E) | ml | 50.0 | | | |
| THYMIDINE | mg | 50.0 | 50.0 | 50.0 | 50.0 |
| GENTAMYCIN | mg | 50.0 | 50.0 | | |
| Benomyl | mg | | | | |
| Magnesium Sulfate, Anhydrous | g | | | 1.204 | |
| 17F-MEROPENEM | mg | | | | |
| 7V-Vitamin E in EtOH 1 mg/mL | mg | | | | |
| 28E-IBA 1 MG/ML | mg | | | | |
| pH | | | 6.8 | | 5.8 |

TABLE 7

| | Units | | | | |
|---|---|---|---|---|---|
| | per | | | | |
| Medium Components | liter | 605B | 13329B | 404J | 90O |
| 404 Modified MS Basal Salts | | | | 4.96 | |
| MS BASAL SALT MIXTURE | g | 4.3 | 4.3 | | 2.165 |
| N6 BASAL SALTS | g | | | | |
| N6 MACRONUTRIENTS 10X | ml | 60.0 | | | |
| POTASSIUM NITRATE | g | 1.7 | | | |
| B5H MINOR SALTS 1000X | ml | 0.6 | | | |
| NaFe EDTA FOR B5H 100X | ml | 6.0 | | | |
| ERIKSSON'S VITAMINS | ml | 0.4 | | | |
| 1000X | | | | | |
| S&H VITAMIN STOCK | g | 0.6 | | | |
| THIAMINE•HCL | mg | 0.2 | | 0.1 | |
| L-PROLINE | g | 2.0 | 0.7 | | |
| CASEIN HYDROLYSATE | g | 0.3 | | | |
| (ACID) | | | | | |
| SUCROSE | g | 20.0 | 60.0 | 65.0 | 20 |
| GLUCOSE | g | 0.6 | | | |
| MALTOSE | g | | | | |
| 2,4-D | mg | 0.8 | | | |
| AGAR | g | 6.0 | 8.0 | 6.0 | 5 |
| BACTO-AGAR | g | | | | |
| PHYTAGEL | g | | | | |
| DICAMBA | g | 1.2 | | | |
| SILVER NITRATE | mg | 3.4 | | | |
| AGRIBIO Carbenicillin | mg | | | | |
| Timentin | mg | | | | |
| Cefotaxime | mg | | | | |
| MYO-INOSITOL | g | | 0.1 | 0.1 | |
| NICOTINIC ACID | mg | | | | |
| PYRIDOXINE•HCL | mg | | | | |
| VITAMIN ASSAY | g | | | | |
| CASAMINO ACIDS | | | | | |
| MES BUFFER | g | | | | |
| ACETOSYRINGONE | uM | | | | |
| ASCORBIC ACID 10 MG/ML | mg | | | | |
| (7S) | | | | | |
| MS VITAMIN STOCK SOL. | ml | | 5 | 5 | |
| ZEATIN | mg | | 0.5 | | |
| CUPRIC SULFATE | uM | | 4.0 | | |
| IAA 0.5 MG/ML (28A) | ml | | 1 | | |
| ABA 0.1 mm | ml | | | | |
| 12N-a-NAA 1 MG/ML | mg | | | 0.5 | |
| THIDIAZURON | mg | | | | |
| PPT(GLUFOSINATE-NH4) | mg | | | | |
| BAP | mg | | 1.0 | | |
| YEAST EXTRACT (BD Difco) | g | | | | |
| PEPTONE | g | | | | |
| SODIUM CHLORIDE | g | | | | |
| SPECTINOMYCIN | mg | | | | |
| FERROUS SULFATE•7H20 | ml | | | | |
| AB BUFFER 20X (12D) | ml | | | | |
| AB SALTS 20X (12E) | ml | | | | |
| THYMIDINE | mg | | | | |
| GENTAMYCIN | mg | | | | |
| Benomyl | mg | | | | 50 |
| Magnesium Sulfate, Anhydrous | g | | | | |
| 17F-MEROPENEM | mg | 10.0 | 10.0 | 10.0 | 10 |
| 7V-Vitamin E in EtOH 1 mg/mL | mg | | | 1.0 | |
| 28E-IBA 1 MG/ML | mg | | | 1.0 | |
| pH | | 5.8 | | 5.8 | |

### Example 3: Particle Bombardment

Standard protocols for particle bombardment (Finer and McMullen, 1991, In Vitro Cell Dev. Biol.—Plant 27:175-182) can be used with the methods of the disclosure.

Pioneer inbred PH184C (disclosed in U.S. Pat. No. 8,445,763, incorporated herein by reference in its entirety) that contains in chromosome-1 a pre-integrated Site-Specific Integration (SSI) target site (Chrom-1 target site) composed of UBI PRO:FRT1:NPTII::PINII TERM+FRT87 is used. Prior to bombardment, 10-12 DAP (days after pollination) immature embryos are isolated from ears of Pioneer inbred PH184C and placed on 605J culture medium plus 16% sucrose for three hours to plasmolyze the scutellar cells.

Four plasmids are typically used for each particle bombardment:
1) a donor plasmid (100 ng/μl) containing a FRT-flanked donor cassette for Recombinase-Mediated Cassette Exchange, for example a plasmid containing FRT1:PMI::PINII TERM::CZ19B1 TERM+UBI1ZM PRO::UBI1ZM 5 UTR::UBI1ZM INTRON1::DS-RED2::PINII TERM+FRT6 (PHP8418-0004);
2) a plasmid (2.5 ng/μl) containing the expression cassette UBI1ZM PRO::UBI1ZM 5 UTR::UBI1ZM INTRON1::FLPm::PINII TERM (PHP5096);
3) a plasmid (10 ng/μl) containing the expression cassette ZM-PLTP PRO::ZM-ODP2::OS-T28 TERM+FMV & PCSV ENHANCERS (PHP89030); and
4) a plasmid (5 ng/ul) containing the expression cassette ZM-PLTP PRO::ZM-WUS2::IN2-1 TERM+PSW1+GZ-W64A TERM+FL2 TERM (PHP89179).

To attach the DNA to 0.6 μm gold particles, the four plasmids are mixed by adding 10 μl of each plasmid together in a low-binding microfuge tube (Sorenson Bioscience 39640T) for a total of 40 μl. To this suspension, 50 μl of 0.6 μm gold particles (30 μg/μl) and 1.0 μl of Transit 20/20 (Cat No MIR5404, Mirus Bio LLC) are added, and the suspension is placed on a rotary shaker for 10 minutes. The suspension is centrifuged at 10,000 RPM (˜9400×g) and the supernatant is discarded. The gold particles are re-suspended in 120 μl of 100% ethanol, briefly sonicated at low power and 10 μl is pipetted onto each carrier disc. The carrier discs are then air-dried to remove all remaining ethanol. Particle bombardment is performed using a Biolistics PDF-1000, at 28 inches of Mercury using a 200 PSI rupture disc. After particle bombardment, the immature embryos are selected on 605J medium modified to contain 12.5 g/l mannose and 5 g/l maltose and no sucrose. After 10-12 weeks on selection, plantlets are regenerated and analyzed using qPCR. It is expected that co-delivery of PLTP::ODP2 (PHP89030) and PLTP::WUS2 (PHP89179) along with the SSI components (Donor DNA (PHP8418-0004)+UBI::FLP (PHP5096)) will produce high frequencies of site-specific integration of the donor fragment into the Chrom-1 target site (i.e. at rates of 4-7% relative to the number of bombarded immature embryos).

### Example 4: Agrobacterium-Mediated Transformation of Maize

### A. Preparation of Agrobacterium Master Plate.

Agrobacterium tumefaciens harboring a binary donor vector was streaked out from a −80° C. frozen aliquot onto solid 12R medium and cultured at 28° C. in the dark for 2-3 days to make a master plate.

### B. Growing Agrobacterium on Solid Medium.

A single colony or multiple colonies of Agrobacterium were picked from the master plate and streaked onto a second plate containing 810K medium and incubated at 28° C. in the dark overnight.

Agrobacterium infection medium (700A; 5 ml) and 100 mM 3′-5′-Dimethoxy-4′-hydroxyacetophenone (acetosyringone; 5 μL) were added to a 14 mL conical tube in a hood. About 3 full loops of Agrobacterium from the second plate were suspended in the tube and the tube was then vortexed to make an even suspension. The suspension (1 ml) was transferred to a spectrophotometer tube and the optical density (550 nm) of the suspension was adjusted to a reading of about 0.35-1.0. The Agrobacterium concentration was approximately 0.5 to 2.0×10⁹ cfu/mL. The final Agrobacterium suspension was aliquoted into 2 mL microcentrifuge tubes, each containing about 1 mL of the suspension. The suspensions were then used as soon as possible.

### C. Growing Agrobacterium on Liquid Medium.

Alternatively, Agrobacterium can be prepared for transformation by growing in liquid medium. One day before infection, a 125 ml flask was prepared with 30 ml of 557A medium (10.5 g/l potassium phosphate dibasic, 4.5 g/l potassium phosphate monobasic anhydrous, 1 g/l ammonium sulfate, 0.5 g/l sodium citrate dehydrate, 10 g/l sucrose, 1 mM magnesium sulfate) and 30 μL spectinomycin (50 mg/mL) and 30 μL acetosyringone (20 mg/mL). A half loopful of Agrobacterium from a second plate was suspended into the flasks and placed on an orbital shaker set at 200 rpm and incubated at 28° C. overnight. The Agrobacterium culture was centrifuged at 5000 rpm for 10 min. The supernatant was removed and the Agrobacterium infection medium (700A) with acetosyringone solution was added. The bacteria were resuspended by vortex and the optical density (550 nm) of the Agrobacterium suspension was adjusted to a reading of about 0.35 to 2.0.

### D. Maize Transformation.

Ears of a maize (Zea mays L.) cultivar were surface-sterilized for 15-20 min in 20% (v/v) bleach (5.25% sodium hypochlorite) plus 1 drop of Tween 20 followed by 3 washes in sterile water. Immature embryos (IEs) were isolated from ears and were placed in 2 ml of the Agrobacterium infection medium (700A) with acetosyringone solution. The optimal size of the embryos varies based on the inbred, but for transformation with WUS2 and ODP2 a wide size range of immature embryo sizes could be used. The Agrobacterium infection medium (810K) was drawn off and 1 ml of the Agrobacterium suspension was added to the embryos and the tube was vortexed for 5-10 sec. The microfuge tube was allowed to stand for 5 min in the hood. The suspension of Agrobacterium and embryos were poured onto 710I co-cultivation medium. Any embryos left in the tube were transferred to the plate using a sterile spatula. The Agrobacterium suspension was drawn off and the embryos placed axis side down on the media. The plate was incubated in the dark at 21° C. for 1-3 days of co-cultivation.

Embryos were transferred to resting medium (605T medium) without selection. Three to 7 days later, the embryos were transferred to maturation medium (289Q medium) supplemented with a selective agent.

### Example 5: Transformation of Maize Leaf Segments

Constitutive expression of WUS2 and ODP2 after Agrobacterium-mediated transformation of maize leaf segments resulted in production of embryogenic callus and/or rapidly formed somatic embryos which regenerate into healthy, fertile T0 plants.

The general protocol for Agrobacterium-mediated maize transformation described in Example 4 was used, with the modifications described below for using leaf tissue as the target explant.

### In Vitro Seed Germination to Produce Seedling Target Tissue

Mature seeds were surface sterilized by immersion in a series of solutions under agitation using a magnetic stir bar; first in an 80% ethanol solution for 3 minutes, the ethanol solution was decanted and replaced with a 30% Clorox bleach solution containing 0.1% Tween-20 for 20 minutes, the Clorox bleach solution was decanted.

Alternatively, chlorine gas or oxidizing agents can be used for seed sterilization. Chlorine gas can be generated using a variety of compounds (or agents), including bleaching powders, calcium hypochlorite, sodium hypochlorite, industrial bleach, household bleach, chlorine dioxide monochloramine, dichloramine, and trichloramine. Oxidizing agents that can be used in the methods disclosed herein include but are not limited to, ozone, hydrogen peroxide, hypochlorous acid, hypobromous acid, chlorine dioxide, and ethylene dioxide.

After seed sterilization, the mature seeds were rinsed (three 5-minute rinses) in autoclaved sterile water. The sterilized seeds were transferred onto solid 90O medium after the final sterile water rinse. In vitro germination and seedling growth were carried out at 26° C. with a 16 h light/8 h dark photoperiod. The first 2.5 to 3 cm of leaf whorl above the mesocotyl was removed from each 12-18 day-old seedling for further processing for transformation.

### Agrobacterium Preparation

Agrobacterium tumefaciens strain LBA4404 TD THY- harboring helper plasmid PHP71539 (pVIR9, see US20190078106A1, herein incorporated by reference in its entirety) and a binary donor vector, PHP96037, containing a WUS2/ODP2 T-DNA with a selectable marker (ZM-ALS (HRA)) and a screenable marker (ZS-GREEN1) or a binary donor control vector containing a selectable marker and/or a screenable marker T-DNA (lacking WUS2/ODP2) was streaked out from a −80° C. frozen aliquot onto solid 12V medium and cultured at 28° C. in the dark for 2 days to make a master plate. A working plate was prepared by streaking 4-5 colonies from the 12V-grown master plate across fresh 810K media, incubating overnight in the dark at 28° C. prior to using for Agrobacterium infection. Additional helper plasmids, including but not limited to PHP70298 (containing vir genes from A. tumefaciens), RV005393 (containing vir genes from A. tumefaciens), and RV007497 (containing vir genes from A. rhizogenes), are useful in the methods of the disclosure and are listed in Table 2.

Agrobacterium infection medium (700J medium, 10 ml) with the addition of 20 μL of acetosyringone and 20 μL of a previously 10-fold-diluted surfactant (Break Thru S 233, Evonik Industries GmbH, Goldschmidtstraße 100, 45127 Essen, Germany, Product Code 99982498) was added to a 50 mL conical tube in a hood. About 5 full loops of Agrobacterium were collected from the working plate, transferred to the infection medium in the 50 ml tube, and then vortexed until uniformly suspended. The suspension (1 ml) was transferred to a spectrophotometer tube and the optical density (550 nm) of the suspension was adjusted to a reading of 0.6. The final Agrobacterium suspension was aliquoted into Corning six-well plates containing 0.4 μm permeable culture inserts (Falcon, Part Numbers 353046 and 353090, respectively) with each well containing about 8 mL of the Agrobacterium suspension.

Seed of maize inbred PH85E were surface sterilized as previously described, and then germinated at 28° C. under low light on solid 90B medium (1/2 strength MS salts plus 20 g/l sucrose and 50 mg/l benomyl). The leaf base segment (an approximate 2.5-3.0 cm section above the mesocotyl) was removed from each 12-18 day-old in vitro-germinated seedling with sterilized scissors. These leaf segments were placed into a 150 mm×15 mm Petri dish. Forceps were used to hold each leaf whorl section at the upper green end and the section was bisected longitudinally into 2 lengthwise halves using a sterile #10 scalpel blade. The outer leaf was removed and the inner leaves of the whorl were then cross-cut (diced) into smaller sections/segments (approximately 1 to 3 mm in size, preferably 2.5-3.0 mm in size). These smaller leaf sections/segments were collected and directly transferred into the permeable culture inserts containing the Agrobacterium suspension and incubated at room temperature (25° C.) for a 15-minute infection period.

After infection, the culture insert containing the Agrobacterium-infected leaf segments was removed from the 6-well plate and placed on an autoclaved dry filter paper to wick up and remove any residual Agrobacterium solution. The infected leaf segments were then transferred onto a fresh filter paper (VWR 7.5 CM) resting on 710N solid co-cultivation medium. Forceps were used to evenly disperse the leaf segments on the 710N plates and to ensure they have enough room to grow. The infected leaf tissue was incubated at 21° C. in the dark for 2-3 days.

After 2-3 days co-cultivation, the paper supporting the leaf tissue was removed from the 710N medium and transferred onto 605B medium for 4 week resting culture. Tissue was sub-cultured every 2 weeks. After the 4 weeks culture on resting medium (605B) the plates were placed into a controlled temperature/humidity incubator (45° C./70% RH) for a 2-hour heat treatment. The plates were removed from the incubator and kept at room temperature (25° C.) for 1-2 hours until the plates had cooled down. Depending on the maize inbred and expression cassettes used, a single two-hour heat treatment, or two 2-hour heat treatments on two consecutive days, were applied to stimulate the drought-inducible RAB17 promoter, or the heat-inducible HSP26 promoter, or the heat-inducible HSP17.7 promoter and induce CRE-mediated excision of WUS2, ODP2, and CRE recombinase.

After the heat treatment and temperature equilibration at room temperature, leaf segments with newly-developed somatic embryos were transferred onto 13329B maturation medium without filter papers, cultured in the dark at 28° C. for 2 weeks, and then moved into a 26° C. light room for an additional week. Leaf segments that now supported small shoots were transferred onto 404J rooting medium for an additional 2-3 weeks until well formed roots had developed, at which point the plantlets were ready for transfer to the greenhouse.

As an alternative to transferring the leaf tissue using forceps, the leaf segments were transferred onto filter paper, and in subsequent sub-culture steps the entire filter paper with the leaf segments it supported was moved to a new plate containing fresh medium. For example, after infection, the culture insert containing the Agrobacterium-infected leaf segments was removed from the 6-well plate and the leaf segments were transferred to 710N solid co-cultivation medium. This was accomplished by first inverting the culture insert (used for the infection step) and tapping on the bottom of the insert causing the leaf segments to fall onto an autoclaved dry filter paper and allowed to sit for 2-5 minutes to wick up and remove any residual Agrobacterium solution. The infected leaf segments were then transferred onto a fresh filter paper (VWR 7.5 CM) resting on 710N solid co-cultivation medium. Forceps were used to evenly disperse the leaf segments on 710N plates and make sure they have enough room to grow. The infected leaf segments were incubated at 21° C. in the dark for 2-3 days.

After 2-3 days co-cultivation, the paper supporting the leaf segments was removed from the 710N medium and transferred onto 605B medium for a 4-week resting period. The leaf segments were sub-cultured every 2-weeks. After the 4-weeks on resting medium (605B), the plates were placed into a controlled temperature/humidity incubator (45° C./70% RH) for a 2-hour heat treatment. The plates were removed from the incubator and kept at room temperature (25° C.) for 1-2 hours until the plates had cooled down. Depending on the maize inbred, a single two-hour heat treatment, or two 2-hour heat treatments on two consecutive days, were applied in order to stimulate the drought-inducible RAB17 promoter, or the heat-inducible HSP26 promoter, or the heat-inducible HSP17.7 promoter and induce CRE-mediated excision of WUS2, ODP2, and CRE recombinase.

After the heat treatment and temperature equilibration at room temperature, leaf segments with newly-developed somatic embryos were transferred onto 13329B maturation medium without filter papers, cultured in the dark at 28° C. for 2 weeks, and then moved into a 26° C. light room for an additional week. Leaf segments that now supported small shoots were transferred onto 404J rooting medium for an additional 2-3 weeks until well formed roots had developed, at which point the plantlets were ready for transfer to the greenhouse.

Results from five experiments using WUS2/ODP2 are shown in Table 8, in which 10 starting seedlings per experiment (50 total) were used to produce the starting leaf segments for Agrobacterium infection, the number of transgenic T0 plants recovered ranged from 18 (Exp. 1) to 51 (Exp. 4), resulting in a mean transformation frequency of 360%+/−112 (Standard Deviation (SD)). This is in contrast to experiments in which only a selectable marker gene and/or a screenable marker gene (fluorescent protein gene) were contained in the T-DNA, in which no culture response was observed and no T0 plants were produced.

In addition to a high transformation frequency, a high percentage of the recovered T0 plants were single-copy (SC) for the T-DNA (containing the selectable marker and/or the screenable marker) with no contaminating sequences from Agrobacterium being detected. Such SC/No-Agro events (T0 plants) ranged from 23% to 37% with a mean of 31.4% (+/−5.2% SD). By comparing the number of high-quality transgenic T0 plants (SC for the T-DNA with no contaminating Agrobacterium backbone sequences) to the number of starting seedlings used in these experiments provided a clear measure of overall efficiency, with a mean frequency of 114% (+/−44% SD). This method using WUS2/ODP2 obviated the need for growing mature maize plants for 90-120 days in the greenhouse to produce immature embryo explants for transformation and provided transgenic events from leaf explants generated from germinated seed in the lab.

TABLE 8

| | | # of | | # of | SC | SC | |
|---|---|---|---|---|---|---|---|
| | | T0 | | SC No- | No- | No- | |
| | # of | plants | | Agro | Agro | Agro | Escape |
| Exp | Seedlings | (events) | T0% | Seq | %* | %** | Freq. |
| 1 | 10 | 18 | 180% | 5 | 28% | 50% | 39% |
| 2 | 10 | 41 | 410% | 14 | 34% | 140% | 32% |
| 3 | 10 | 40 | 400% | 9 | 23% | 90% | 45% |
| 4 | 10 | 51 | 510% | 18 | 35% | 180% | 40% |
| 5 | 10 | 30 | 300% | 11 | 37% | 110% | 43% |
| | 50 | 180 | 360% | 57 | 29% | 114% | 39% |

*Frequency of T0 plants with single-copy T-DNA and no plasmid sequences, relative to the total number of T0 plants
**Frequency of T0 plants with single-copy T-DNA and no plasmid sequences, relative to the starting number of seedlings

### Example 6: Automated Leaf Explant Preparation

Use of a blender or food processor for automated, mechanized leaf explant preparation results in efficient Agrobacterium-mediated transformation and recovery of transgenic plants.

Agrobacterium strain LBA4404 TD THY- containing the helper virulence plasmid PHP71539 and the binary T-DNA-containing plasmid PHP97334 was cultured as described in Example 4 with modifications for leaf transformation as described in Example 5. Mature dry seed of Pioneer inbred PH85E were surface sterilized and germinated as described and eight seedlings were manually dissected and the leaves manually cut into segments to produce 2-3 mm leaf segments as described in Example 5. These manually cut leaf segments were then added to the Agrobacterium suspension and allowed to incubate at room temperature for 30 minutes.

For the remaining eight seedlings in each experiment, 2.5-3.0 cm of leaf tissue was dissected from each seedling and transferred directly into the Agrobacterium suspension in the bowl of a Cuisinart Mini-Blender (Model No. DLC-1SS) food processor. The 2.5-3 cm leaf sections were immersed in 100 ml of Agrobacterium suspension in the Cuisinart Mini-Blender bowl. The Cuisinart Mini-Blender food processor was then pulsed until the average leaf segment length was approximately 2-3 mm. The leaf segments were allowed to incubate in the Agrobacterium suspension for 30 minutes. Alternatively, the leaf tissue dissected from the seedlings is transferred to the bowl of a Cuisinart Mini-Blender food processor and the blender is then pulsed until the average leaf segment length is approximately 2-3 mm. After preparation of the leaf segments the Agrobacterium suspension is added to the bowl.

Four experiments were performed on different dates using seedling derived leaf segments from eight seedlings for the manual dissection/preparation method and eight seedlings for the blender preparation method as described above. Agrobacterium strain LBA4404 TD THY- containing PHP71539 (pVIR9) plus PHP97334 was used for transformation at a bacterial concentration with an OD of 0.6, mixed into 700J medium as indicated in Table 6 (acetosyringone (AS), Break Thru S 223 (BT233)).

After Agrobacterium infection for 30 minutes, the Agrobacterium suspension (either from the manual preparation or the blender preparation) was poured through a 0.4 μm nylon mesh, collecting the leaf segments on top. The nylon mesh was then inverted and tapped repeatedly to dislodge the leaf segments which fell onto a filter paper resting on solid co-cultivation medium. All subsequent sub-culturing, resting, selection, heat-treatment, regeneration, maturation, and rooting was the same for both treatments (the manually-prepared control leaf segments and the blender-prepared test leaf segments) as described above.

Transformation (TXN) results for the four experiments (eight seedlings used for the manually-prepared control leaf segments and eight seedlings used for the blender-prepared test leaf segments for each experiment) are summarized in Table 9. T-DNA delivery was visually determined by the relative area of leaf segments within a treatment exhibiting transient ZS-GREEN expression, with 0=none, 1=<10%, 2=10-20%, 3=20-30%, and 4=>30% of the leaf segments expressing ZS-GREEN and beginning to show signs of cell proliferation 3-days after Agrobacterium infection. This ZS-GREEN expression provided a relative measure of the fraction of leaf segments that received T-DNA. Based on this criterion, T-DNA delivery was assessed as being very good, and equivalent for both methods (experiments summarized in Table 9). Based on these experiments, for either manually-prepared or blender-prepared leaf segments, the mean transformation frequency (+/−standard deviation) was 175% (+/−20.4%) and 172% (+/−15.9%) respectively, showing no significant difference using a Student's Paired T-Test with p>0.05. This comparison demonstrated that preparing leaf tissue using the blender or manual cutting produced a population of leaf segments that were equivalent in terms of efficacy for Agrobacterium transformation and production of T0 plants.

TABLE 9

| Explant | | | | Agro | | | TXN |
|---|---|---|---|---|---|---|---|
| Preparation | Infection | # | | Infection | T-DNA | # T0 | Freq. |
| Method | medium | Seedlings | Plasmid | Duration | Delivery* | Plants | (%) |
| Manual | 40 ml | 8 | PHP97334 | 30 min | 3 | 14 | 175 |
| | 700 J + 80 μl | 8 | PHP97334 | 30 min | 3 | 12 | 150 |
| | AS + 40 μl | 8 | PHP97334 | 30 min | 3 | 14 | 175 |
| | diluted | 8 | PHP97334 | 30 min | 3 | 16 | 200 |
| | BT233 | | | | | | |
| Blender | 100 ml | 8 | PHP97334 | 30 min | 3 | 15 | 188 |
| | 700 J + 200 μl | 8 | PHP97334 | 30 min | 3 | 12 | 150 |
| | AS + 20 μl | 8 | PHP97334 | 30 min | 3 | 14 | 175 |
| | BT233 | 8 | PHP97334 | 30 min | 3 | 14 | 175 |

*T-DNA delivery inferred by relative area of leaf segments within a treatment exhibiting transient ZS-GREEN expression, with 0 = none, 1 = <10%, 2 = 10-20%, 3 = 20-30%, and 4 = >30% of the leaf segments expressing ZS-green and beginning to show signs of cell proliferation 3-days after Agrobacterium infection.

Other food processors and/or automated/semi-automated grinding devices may be used in the methods disclosed herein. In addition, other automated/semi-automated means may be used to prepare leaf explants for transformation including the use of automated or semi-automated chopping and/or milling devices.

### Example 7: Use of Movable Supports for Transfer of Leaf Tissue and Leaf-Derived Somatic Embryos

Using the methods described above seedlings of inbred PH85E were prepared by immersing the leaf segments (2-3 cm above the mesocotyl) in the Agrobacterium suspension in a Cuisinart Mini-Blender food processor. Agrobacterium strain LBA4404 TD THY-containing PHP71539 (pVIR9) plus PHP97334 was used for transformation at a bacterial concentration with an OD of 0.6, mixed into 100 ml of 700J medium+200 μl of acetosyringone+20 μl Break Thru S 223. The 2.5-3 cm leaf sections were immersed in 100 ml of bacterial suspension in the Cuisinart Mini-Blender food processor bowl, which was pulsed until the average leaf segment length was approximately 2-3 mm. Infection, co-culture, resting, selection, and regeneration processes were performed as described above.

After Agrobacterium infection, leaf segments from the Cuisinart Mini-Blender food processor were divided in half. One half of the leaf segments were transferred manually at each subculture step. The other half of the leaf segments were distributed evenly on filter papers which were then placed on co-cultivation medium. Subsequently, the filter papers supporting the leaf segments were picked up using forceps and moved to fresh medium at each sub-culture step. Manual transfer of individual leaf segments and bulk transfer on filter paper of leaf segments provided equivalent growth rates, rapid formation of somatic embryos, and rates of production of T0 plants.

### Example 8: Mechanized Leaf Explant Preparation in Species and Varieties Across the Poaceae

Seed from various species within the Poaceae were surface sterilized and germinated under sterile conditions. Using the protocol developed for maize, leaf tissue from the resulting various seedlings within the Poaceae were harvested and manually cut into 2-3 mm segments or were prepared in the Cuisinart Mini-Blender food processor as described above. Agrobacterium strain LBA4404 TD THY- containing both PHP71539 (pVIR9) and a plasmid with a T-DNA having the components NOS::WUS2+3×ENH::UBI PRO::ODP2+UBI::ZS-GREEN+HRA was used for transformation. All steps in the protocol and all media formulations used for these experiments were as described for maize, and the plasmids used (PHP54733, PHP81858, PHP93739, and PHP96037; SEQ ID NO: 10, 3, 6, and 7, respectively) contained maize promoters and maize WUS2/ODP2 genes.

For all species tested, seedling-derived leaf segments, whether manually-prepared or blender-prepared, were successfully used to recover somatic embryos and regenerate T0 plants that were confirmed to contain the respective T-DNA of the plasmid used for transformation. The species successfully transformed using these leaf transformation methods are indicated in bold in Table 10 below, and include corn, sorghum, pearl millet, rice, switchgrass, barley, rye, wheat, and teff. These species span four sub-families within the Poaceae (Chloridoideae, Panicoideae, Oryzoideae, and Pooideae). These sub-families span almost the entire phylogenetic breadth of the grass family (Poaceae). These various cereal crops, some of which are generally regarded as being recalcitrant or difficult to transform using conventional methods were readily transformed through leaf transformation. In addition, this method also produced somatic embryos and regenerated T0 plants in Zea mays ssp Mexicana and Zea mays ssp parviglumis, two varieties of teosinte that have historically been very difficult to transform. When leaf segments were subjected to Agrobacterium infection with PHP96037 and subcultured as described above as in previous Examples, multiple transgenic plants were produced for both Zea mays ssp Mexicana and Zea mays ssp parviglumis, with 47 and 8 (respectively) T0 plants being confirmed to contain the T-DNA with the components RB+LOXP+NOS::WUS2+3×ENH::UBIODP2+INS+HSP PRO::CRE+INS+LOXP+ZS-GREEN+HRA+LB.

TABLE 10

| Family | Sub-Family | Species | Common Name |
|---|---|---|---|
| Poaceae | Chloridoideae | Eragrostis tef | teff |
| | Danthonioideae | | |
| | Micrairoideae | | |
| | Arundinoideae | | |
| | Panicoideae | Zea mays | corn |
| | | Sorghum bicolor | sorghum (milo) |
| | | Pennisitum glaucum | pearl millet |
| | | Panicum virgatum | switchgrass |
| | Aristidoideae | | |
| | Oryzoideae | Oryza sativa cv | rice |
| | | Kataake | |
| | | Oryza sativa cv | indica rice |
| | | indica IRV94 | |
| | Bambusoideae | | |
| | Pooideae | Hordeum vulgare | barley |
| | | Secale cereale | rye |
| | | Triticum aestivum | wheat |
| | Puelioideae | | |
| | Pharoideae | | |
| | Anomochlooideae | | |

Automated leaf preparation and subsequent transformation of these ten species, which span four sub-families within the grass family and cover the breadth of phylogenetic diversity within the family, while using our unmodified maize protocol, was surprising and unexpected. Further, it is expected that i) screening members of other sub-families such as the bamboos (Bambusoideae) will meet with similar success, and ii) further optimization, for example using the cognate orthologs for promoters, WUS2 and ODP2 for a given species, and using species-optimized media formulations will provide further improvements in transformation efficiency and breadth of transformable species. It is expected that mechanical preparation of leaf explants of other sub-families will produce similar results.

### Example 9: Bulk Automated Seed Preparation

Seed can be sterilized in large bulk batches. Mature seeds are placed in vats and are surface sterilized by immersion in a series of solutions under agitation. For example, such immersions in a series of solutions under agitation include, but are not limited to a first immersion in an 80% ethanol solution for 3 minutes, the ethanol solution is decanted and replaced with a 30% Clorox bleach solution containing 0.1% Tween-20 for 20 minutes, the Clorox bleach solution is decanted, and the mature seeds are rinsed (three 5-minute rinses) in autoclaved sterile water. The sterilized seeds are transferred in bulk onto solid 90O medium after the final sterile water rinse for germination. Depending on the size of the vat sterilization and rinsing times will be adjusted accordingly.

Other means such as bulk sterilization by chlorine gas exposure may also be used. Chlorine gas can be generated using a variety of compounds (or agents), including bleaching powders, calcium hypochlorite, sodium hypochlorite, industrial bleach, household bleach, chlorine dioxide monochloramine, dichloramine, and trichloramine. In addition, oxidizing agents can be used for seed sterilization. Oxidizing agents that can be used in the methods disclosed herein include but are not limited to, ozone, hydrogen peroxide, hypochlorous acid, hypobromous acid, chlorine dioxide, and ethylene dioxide.

### Example 10: Bulk Automated Seed Germination

Sterilized seed are germinated on solid 90O medium or in liquid 90O medium. Seed are transferred by automated, mechanical, and or robotic means to large trays of solid 90O medium for germination. Alternatively, the seed is germinated and seedlings are grown in hydroponic trays which allows for dense planting and the ability to add and/or remove liquid media components after transfer by automated, mechanical, and or robotic means.

### Example 11: Bulk Automated Leaf Harvesting

After seedling germination, the seeds are grown to a height sufficient for leaf harvesting. Harvesting may be performed by any automated, mechanical, and/or robotic means that provides cutting the leaf portions from the seedlings for further processing.

### Example 12: Bulk Automated Leaf Preparation and Bacterial Infection

The harvested leaf portions are placed in vats containing any of the bacterial strains disclosed herein for further processing and infection by any automated, mechanical, and/or robotic means. The vats contain chopping means, cutting means, and/or blending means to provide leaf segments of an appropriate size (approximately 1 to 3 mm in size, preferably 2.5-3.0 mm in size) for bacterial infection. The bacterial infection proceeds for a sufficient amount of time, from about 15 minutes to about 30 minutes, or more, before the bacterial solution is automatically decanted/removed by any mechanical and/or robotic means. It is expected that other bacterial strains harboring other plasmids will be useful in the methods of bulk automated leaf preparation and bacterial infection disclosed herein.

### Example 13: Bulk Automated Processing of Bacterially Infected Leaf Explants

After decanting the bacterial solution, the bacterially infected leaf explants are transferred and uniformly spaced by automatic, mechanical, and/or robotic means to a support surface on large trays for further sub-culturing, resting, selection, heat-treatment, regeneration, maturation, and rooting. Each step of the sub-culturing, resting, selection, heat-treatment, regeneration, maturation, and rooting processes are performed by automated, mechanical, and/or robotic means. In addition, each step of the sub-culturing, resting, selection, regeneration, and maturation processes are performed hydroponicly wherein transfers and media additions and removals are performed by automated, mechanical, and/or robotic means.

### Example 14: Automated Preparation of Leaf Cell Suspensions and Infection with Agrobacterium Solution

After seedling germination, the seedlings are grown to a height sufficient for leaf harvesting. Harvested leaves and Agrobacterium containing (PHP97334) are added into the stainless-steel bowl of a laboratory blender (Waring Part No. TSI-0089), and the suspension is blended until no macroscopic leaf fragments are visible. Under the microscope, this mixture is observed to consist of single leaf cells and/or small clumps containing a few leaf cells with the expected cellular debris such as hairs, trichomes and vascular elements. The slurry is either poured or automatically pipetted onto filters or directly onto culture medium. After 2-6 weeks, macroscopic somatic embryos have formed and can be distinguished visually to be manually transferred or automatically transferred to fresh medium for continued culture, maturation, root formation, and regeneration to produce T0 plants.

### Example 15: Mincing Maize Leaf Tissue Using Modified Rotors to Reduce Leaf Fragment Injury and Improve Transformation Efficiency

The efficacy of a custom spinning scalpel blade assembly mounted within a Cuisinart Mini-Prep 2.6 cup food processor was evaluated by accessing the efficiency of mincing to produce leaf fragments/segments from 14-day old corn seedlings germinated under sterile conditions as a pre-treatment for Agrobacterium mediated transfection. In this context, the term “mincing” is used to describe the cutting of leaf explant tissue to produce tissue segments ranging ideally between 1-3 mm (but which can range from 0.2 to 5 mm in size) which produces cleanly-sliced edges on the leaf segments with a minimum of tissue bruising, tearing or shredding. Alternative tissue cutting/mincing devices include, but are not limited to, handheld blenders, food choppers, lab blenders, or other food processors. Successful outcomes were determined by visually comparing leaf segment/fragment size, the degree of leaf segment/fragment damage, and the absence of leaf segment/fragment ‘shredding’ along cut edges as metrics for evaluating the mincing process.

Components of the system that were varied included the number and type of blades, the geometry of the blade support, and the tilt/pitch of the attached blades. These variables are summarized below (Table 11) including the rating system used in evaluation. Rotation rpm (˜6000) and direction (counterclockwise from above), pulse time (˜100 msec) were held constant across all experiments. The number of pulses was variable, from 6 to 10 pulses. The blades were attached to the blade attachment with the sharp side as the leading edge.

TABLE 11

| | | No. of | Tilt of Bladeⁱⁱ | Pitch of | |
|---|---|---|---|---|---|
| Rotorⁱ | Blade Type | Blades | (deg) | Bladeⁱⁱⁱ (deg) | Rating^{iv} |
| A | No. 10 | 12 | 0 | 0 | 2 |
| A | No. 10 | 6 | 6 | 0 | 3 |
| A | No. 10 | 3 | 6 | 0 | 4 |
| A | No. 10 | 1 | 0 | 6 | 4 |
| B | No. 10 | 2 | 0 | 0 | 4 |
| B | No. 10 | 2 | 0 | 2 | 4 |
| B | No. 10 | 2 | 0 | 4 | 4 |
| C | No. 60 | 2 | 3 | 0 | 5 |

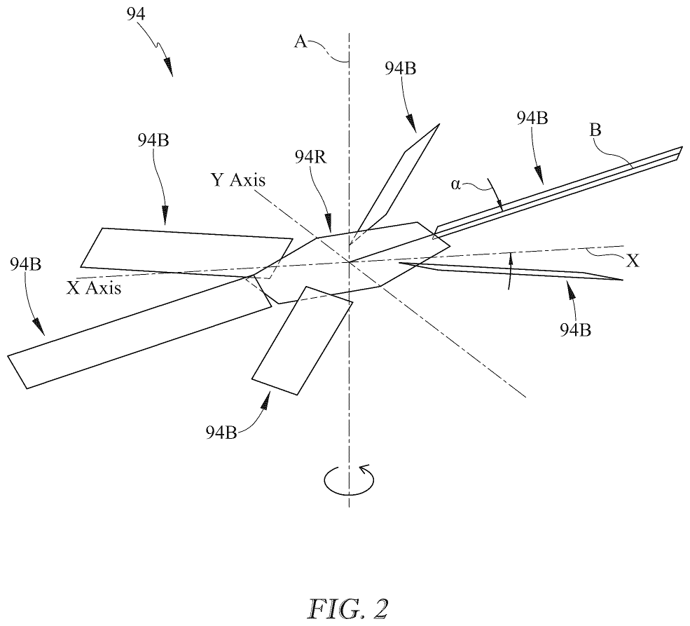

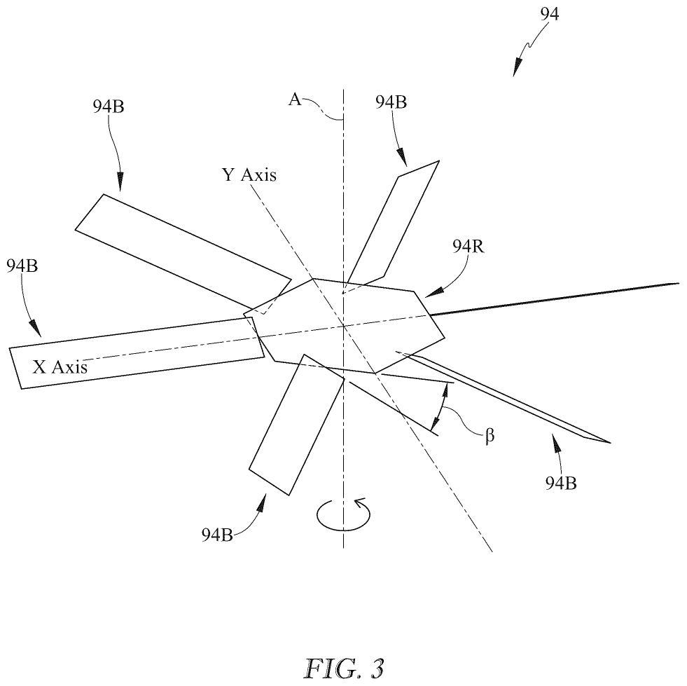

ⁱA: ‘fat’ conical assembly, 12 blade pos, no. 3 scalpel blade handle fitment
B: ‘slender’ cylindrical assembly, 4 blade pairs, no. 3 scalpel blade handle fitment
C: ‘slender’ cylindrical assembly, 1 blade pair, no. 4 scalpel blade handle fitment
ⁱⁱRotation of blade plane about the Y axis (Tilt = as shown in FIG. 2).
ⁱⁱⁱRotation of blade upward in X axis (Pitch − as shown in FIG. 3).
^{iv}Ratings [described in brackets below] designate a combined score summarized leaf fragment/segment size (leaf fragments/segments over 4-5 mm to [1] to uniformly 1-3 mm leaf fragment/segments [5]), and leaf fragment/segment quality (heavily torn and shredded [1] to smoothly cut edges [5]).

The results indicated a strong advantage in performance with optimized blade configurations. By using a lower number of blades and optimizing blade tilt and blade pitch, leaf tissue in liquid suspension was cut more efficiently with limited injury and splashing. Optimal results providing uniform leaf segments (1-3 mm) with minimal injury and efficient T-DNA delivery were produced using large #60 scalpel blades (with 3° tilt) to slice leaf tissue into a more consistent fragment/segment size.

Based on the results above, the thin rotor (B and C in Table 11 above) produced less splashing of the leaf/Agrobacterium solution, and was used for further evaluation of transformation frequencies. In those experiments, seed of maize inbred PHH5E were sown and germinated for 14 days on 90AE medium (ancymidol pretreatment (90O) media plus 2 mg/l ancymidol)), seedlings were incubated at 45° C. (70% RH) for 3 hours prior to transformation. For transformation, six seedlings were used per treatment, with the excised 3 cm of leaf whorl tissue being minced in the blender while suspended in the Agrobacterium solution (strain LBA4404 TD THY- containing PHP71539 plus PHP97334). After 15 minutes infection, the leaf segments were transferred onto filter papers lying on top of solid resting medium for one week, then transferred onto filter papers onto G418 selection medium, and then onto maturation medium. T-DNA delivery was scored 3 days after infection and the number of shoots after culture on maturation medium.

The standard Cuisinart food processor rotor and blade was compared to a thin rotor holding either two #10 blades at different tilts or pitch, or to a single #60 blade set at a 3° tilt. Using a single large blade (Large blade, 3° tilt) produced smaller leaf segments more rapidly, so the number of pulses for this treatment was reduced to 7 to reduce unnecessary injury. Results are shown in Table 12.

TABLE 12

| | No. Pulses | T-DNA | | |
|---|---|---|---|---|
| | on Low | Delivery | No. T0 | % T0 |
| Blade type | Setting | (1-5) | Shoots | Shoots |
| Large blade, 3° tilt | 7 | 5 | 42 | 700% |
| 2 blades no tilt | 8 | 4 | 34 | 567% |
| 2 blades, 2° tilt | 8 | 5 | 35 | 583% |
| 2 blades, 4° tilt | 8 | 4 | 29 | 483% |
| 2 blades, 2° pitch | 8 | 4 | 30 | 500% |
| Cuisinart rotor & blade | 8 | 5 | 41 | 683% |

Based on this preliminary experiment in which only one replicate was performed, it was clear that all the treatments were successful for producing transgenic T0 shoots (Table 12), with the single large blade (Large blade, 3° tilt) and the standard Cuisinart food processor rotor and blade producing similarly high results. Further replicates will permit statistical treatment of larger data sets and it is expected that the thin rotor with the single large, tilted blade (Large blade, 3° tilt) will significantly produce higher transformation frequencies in a production transformation setting.

A secondary experiment was performed with different blade settings. Specifically, the standard Cuisinart food processor rotor and blade was compared to a thin rotor holding either two #10 blades at different tilts, pitch, or offset, or two #60 blades at no tilt, pitch, or offset. Unlike the previous experiments, the blades were offset. In other words, the two blades were spaced apart axialy relative to the rotation axis A, i.e. one blade is vertically spaced apart from the other axially along the rotation axis A. Results are shown in Table 13.

TABLE 13

| | No. Pulses | | |
|---|---|---|---|
| | on Low | No. T0 | % T0 |
| Blade type | Setting | Shoots | Shoots |
| 2 large blades (No. 60), no offset, no | 7 | 40 | 667% |
| angle | | | |
| 2 blades (No. 10), no offset, no angle | 8 | 32 | 533% |
| 2 blades (No. 10), no offset, 2° pitch | 8 | 33 | 550% |
| 2 blades (No. 10), no offset, 4° pitch | 8 | 27 | 450% |
| 2 blades (No. 10), 2 mm offset, 4° pitch | 8 | 29 | 483% |
| Cuisinart rotor & blade | 8 | 40 | 667% |

Based on this secondary experiment, it was clear that all the treatments were successful for producing transgenic T0 shoots (Table 13), with the single large blade (2 large blades, no offset, no angle) and the standard Cuisinart food processor rotor and blade producing similarly high results.

In this experiment (Table 13), seed of maize inbred ED85E were sown and germinated for 14 days on 90AE medium (ancymidol pretreatment (90O) media plus 2 mg/l ancymidol)), seedlings were incubated at 45° C. (70% RH) for 2 hours prior to transformation. For transformation, six seedlings were used per treatment, with the excised 3 cm of leaf whorl tissue being minced in the blender while suspended in the Agrobacterium solution (strain LBA4404 TD THY- containing PHP71539 plus PHP97334). After 15 minutes infection, the leaf segments were transferred onto filter papers lying on top of solid resting medium for one week, then transferred onto filter papers onto G418 selection medium, and then onto maturation medium.

Manual control of the timing/number of pulses was compared to automatic control using an electronic controller (Table 14). For the manual control, the cutting included five pulses of blade rotation each lasting for one seconc each. For the electronic controller, the cutting included five pulses each lasting for 400 milliseconds. Results are shown in Table 14.

TABLE 14

| | | No. of | No. T0 | % T0 |
|---|---|---|---|---|
| Cutting Method | Timing/Pulse | Seedlings | Shoots | Shoots |
| With Control Box | 400 ms/5 pulses | 10 | 40 | 400% |
| Manual Control | 1 second/5 pulse | 10 | 25 | 250% |
| With Control Box | 400 ms/5 pulses | 7 | 29 | 414% |
| Manual Control | 1 second/5 pulse | 7 | 30 | 429% |

Based on this experiment, it was clear that using an electronic controller to control the timing of the pulses results in more consistent performance (Table 14). Again, in this experiment, seed of maize inbred ED85E were sown and germinated for 14 days on 90AE medium (ancymidol pretreatment (90O) media plus 2 mg/l ancymidol)), seedlings were incubated at 45° C. (70% RH) for 2 hours prior to transformation. For transformation, seven to ten seedlings were used per treatment, with the excised 3 cm of leaf whorl tissue being minced in the blender while suspended in the Agrobacterium solution (strain LBA4404 TD THY- containing PHP71539 plus PHP97334). After 15 minutes infection, the leaf segments were transferred onto filter papers lying on top of solid resting medium for one week, then transferred onto filter papers onto G418 selection medium, and then onto maturation medium.

### Example 16: Method to Harvest Whorl Explants from Maize Seedlings

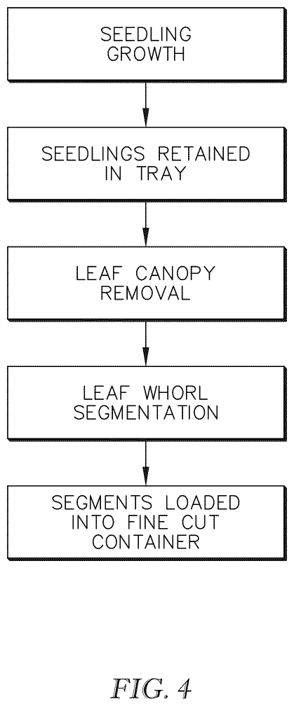

This Example 16 describes a method of preparing approximately 3 cm whorl leaf explant segments originating from multiple maize seedlings using a high throughput technique (see FIG. 4). Plant leaf whorls are extracted for downstream processes with high volumes and efficiency. Seedlings germinated under sterile conditions are provided in a tray/bed retained near the stem portion of the seedling by a mesh with adequate mechanical rigidity. The construction of the mesh is intended to hold the plant seedlings in the tray during processing and is made up of a material suitable for decontamination. Seedling production methods may also include, but are not limited to aeroponics or hydroponics under sterile conditions using similar methods for seedling retention during processing. Trays containing one to many plants are conveyed at an oblique angle to a cutting mechanism made up of sharp saw-like teeth on multilayered blades sliding in opposing directions. Conveyor speed, blade oscillation speed, and teeth geometry are optimized to reduce bending of the seedling during cutting. Additionally, the cutting mechanism may include, but is not limited to, rotating, banded, or scissor like blades at the cut location. Seedlings pass through the blade assembly and the leaf canopy is cut from the seedling immediately above the retaining mesh. Leaf material from the canopy is diverted perpendicularly into a chute for extraction. Pneumatic devices ensure elimination of loose material. The remaining plant stem and root is further conveyed to the second blade assembly for segmentation. Trays and the remaining contents are driven into the blade assembly with 3 cm segments exposed above the blades. Once cut, the segments are collected into a chute and measured and/or counted prior to reaching the container loading device. Optimal explant segments that measure within targeted cut dimensions with minimal contamination or injury are produced through this process.

### Example 17: Method of Using a Rotary Blender to Mince Maize Seedling Whorl Explants for Transformation

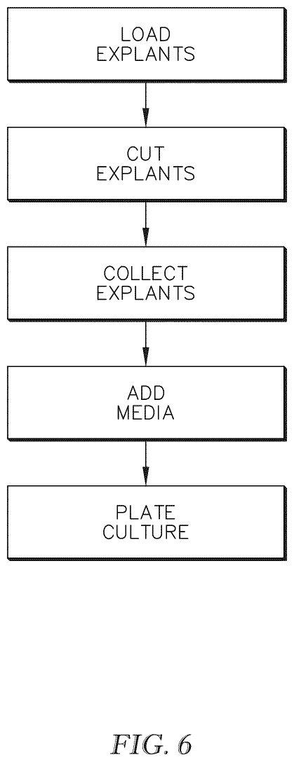

This Example describes an embodiment for mechanically pre-treating germinated maize seedlings for co-cultivation with Agrobacterium for genetic transformation. The high transformation efficiencies previously described are obtained by reducing ˜3 cm leaf whorl explants in a rotary kitchen processor with suitable media, using the staged process shown in the schematic in FIG. 6. This embodiment achieves higher efficiencies and unattended process flow by utilizing a scalpel blade rotor assembly in a presterilized container. One to many containers are automatically conveyed to an explant loading device, followed by sterile media addition and capping. The cap includes an internal and detachable mesh filter. The capped containers are engaged by a programmable spindle motor, the scalpel blade rotor assembly pulses in on-off cycles suitable for tissue mincing to achieve the desired range of leaf-fragment/segment size. The containers are then conveyed to an incubation/shaking device for a timed duration (Agrobacterium infection stage). The container is then conveyed to a decapper/inverter device where the external cap is removed and the inoculated tissue is collected onto the mesh. The mesh(s) is transferred to Petri plates and the plates capped and transferred to an incubator to complete the pretreatment process. The automated process described herein is intended to provide substantial control and repeatability of the process parameters enabling fine tuning for production operations and customization to enable higher transformation efficiencies of recalcitrant genotypes.

### Example 18: Other Methods to Mechanically Chop Whorl Explants for Transformation

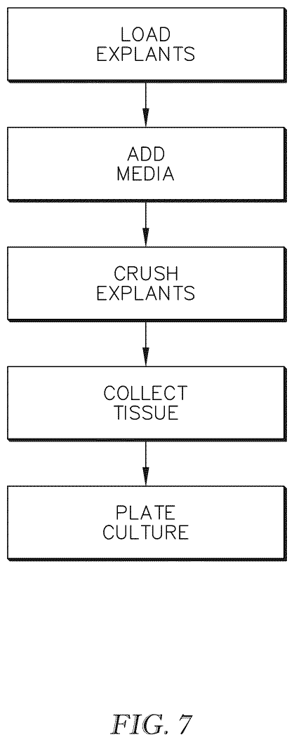

Methods to reduce explants by cutting other than by rotary spindle (spinning blades) are also envisioned. These embodiments include linear tracked blades (band saw), single pivot double blades (scissors), sharp wire (cheese slicer), ganged rolling blades (pasta roller), single and double blade choppers (guillotine), dual action reciprocating blades (hedge trimmer) in combination with sliding stages as needed to engage plant material with blades. The devices employed are sterilized prior to tissue reduction and the seedlings are germinated under sterile conditions and the explants acquired as described previously. The reduced leaf explants are collected and transferred to a container for media addition and transfection/transformation (FIG. 7).

### Example 19: Methods to Mechanically Homogenize Whorl Explants for Transformation

Tissue reduction of sterile maize seedling explants is also considered as a pretreatment for Agrobacterium transfection (FIG. 7). The use of pressure in the presence of media to render suspensions of viable tissue/cells is the objective. The tissue may be reduced with the use of rollers or mills or opposing plates or ball bearings. The reduced leaf tissue is collected and a liquid suspension containing the Agrobacterium cells and the leaf tissue is incubated prior to plating the transformed suspension cell tissue for resting tissue culture, selection, somatic embryo maturation, rooting, and transfer of T0 plantlets to soil.

### Example 20: Maize Seedling Transfection Using Spray-on Agrobacterium and Cellulolytic Enzymes

Maize seedlings grown under sterile conditions are sprayed with Agrobacterium suspensions in the presence of cellulolytic enzymes to transfect leaf cells. The leaves are removed, reduced to small segments as described in any of the modalities above and cultured as described above. The cultured cell lines are induced to form shoots in-vitro and selected in the presence of the vector specific selectable marker. The utilization of the cellulolytic enzyme(s) improves transformation efficiency of recalcitrant genotypes. Cellulolytic enzymes useful in the methods disclosed herein include, but are not limited to, cellobiohydrolases (CBHs), endoglucanases, and β-D-glucosidases.

### Example 21: Method to Harvest Whorl Explants from Maize Seedlings

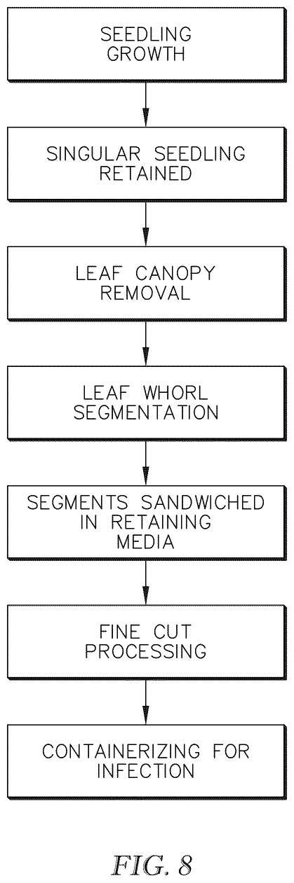

This Example 21 describes a method to cut whorl leaf segments using a laser, high-pressure water, or high-pressure air (FIG. 8). Seedlings germinated under sterile conditions in a tray with discrete root containers are conveyed to the first cut station. Seedlings are manipulated into a single-file line to minimize the depth of the cut. Individual plants pass through the cut mechanism described as a laser, high-pressure water jet stream, or high-pressure air removing the top canopy of the seedling. In the case of high-pressure water or air, the liquid or gas stream is provided from a sterile source and evacuated upon use through a collection point opposite the nozzle. The design of the nozzle is optimized with a high precision tip and ultra-high pressure to minimize injury at the cut and reduce reaction force of the plant. Similarly, the laser beam is optimized to reduce heat damage of the tissue by high frequency pulsing and operating within a near-infrared wavelength. Once the leaf canopy is removed, the material is diverted by a chute above the cut zone and disposed of. Leaf whorl segments are conveyed to the second cut station. Similarly, segments are cut using the methods describe herein; however, the whorl segments above the cut zone are harvested into a chute for extraction and further processing. Leaf whorl segments are individually sent into the chute with vibratory, mechanical, or pneumatic means.

Leaf whorl segments drop into a V-shaped conveyer and are passed through a laser, air, or water jet cut mechanism slicing each segment into many sections in the direction parallel to the plant stem. Directly after being cut, leaf segments are split and set cut-face-down onto a retaining material provided on the top side of the laser, waterjet, or air cut bed. The retaining material is also unrolled above the leaf segments to create a sandwich-like retainer for the fine cutting process. Retained material is continued through the grid conveyor with an appropriate bed material including, but not limited to, aluminum, stainless steel, corrugated plastic, or hardened steel depending on the cut process type. Leaf material is cut as it is conveyed through the laser, waterjet, or air cut bed in various cut patterns including, but not limited to, swiping back and forth or multiple cut heads set up as an array. Fluids for the waterjet are decontaminated or single use to maintain sterility. Additionally, cut beds are sent through decontamination chambers along the conveyor to preserve sterile conditions within the equipment. Once segments are cut to specified dimensions, leaf tissue with retaining media are sectioned and loaded into containers for infection and further processing.

### Example 22: Automated Leaf Explant Preparation System

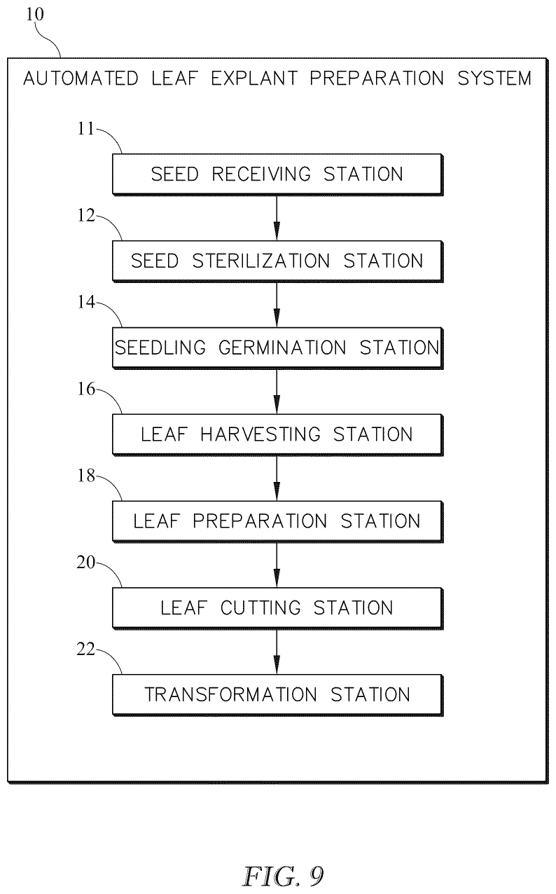

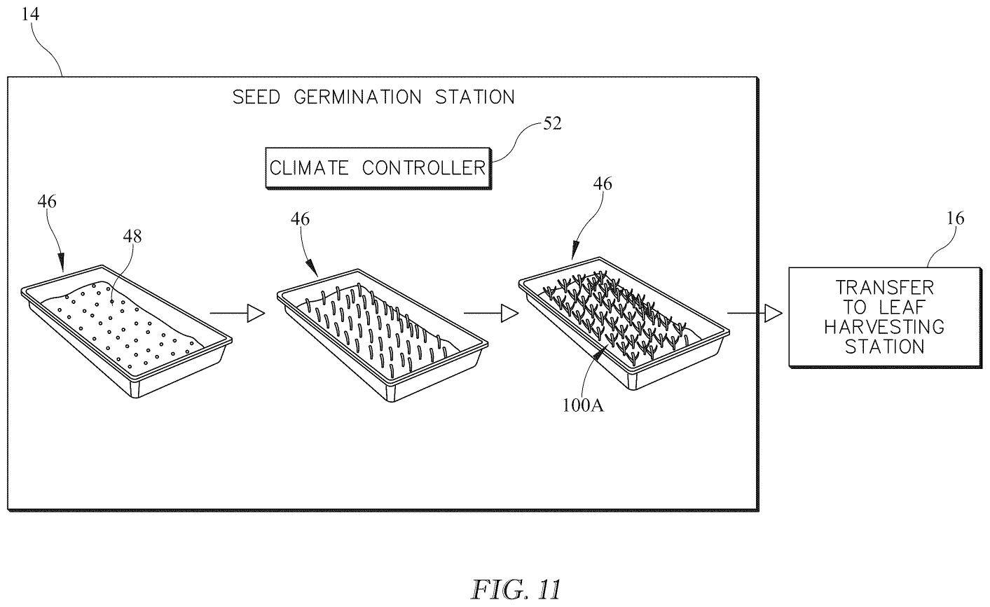

This Example 22 describes an automated leaf explant preparation system 10 as shown in FIG. 9. Automated, high-throughput, non-destruction processes may include bulk seed sterilization, bulk seed germination, bulk seedling propagation, bulk harvesting of seedling leaves, bulk transformation/genome editing, and plant tissue-culture methods. The automated leaf explant preparation system 10 includes a seed receiving station 11 for receiving a population of seed, a seed sterilization station 12, a seedling germination station 14, a leaf harvesting station 16, a leaf preparation station 18, a leaf cutting station 20, and a transformation station 22 as shown in FIGS. 9-13. The transformation station 22 includes a leaf segment infection station, a resting station, a selection station, a regeneration station, a maturation station, a rooting station, a potting station, and a container transport mechanism.

The seed receiving station 11 may be configured to receive a bulk population of seed and divide the seed into containers for sterilization. The seed receiving station 11 may include a dispensing device configured to dispense certain amounts of seed into containers 24 for seed sterilization. The seed receiving station 11 may also include a conveyor or other transportation means to transport the containers 24 to the seed sterilization station 12.

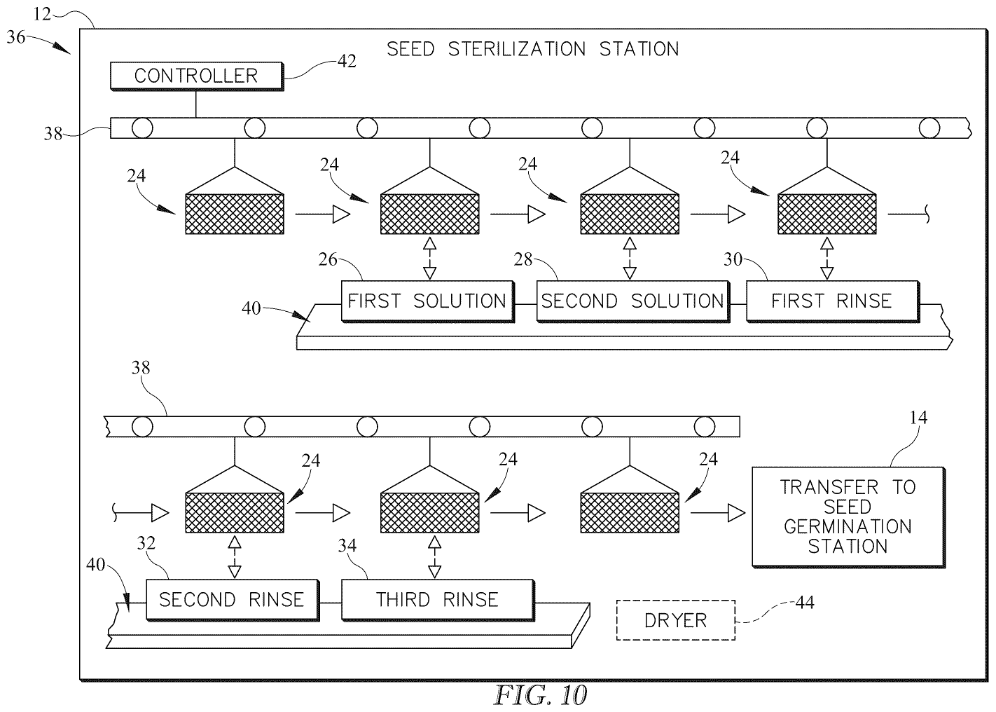

The seed sterilization station 12 may comprise means for providing and decanting/removing a series of solutions to the container of seed to provide a bulk population of sterilized seed as shown in FIG. 10. In the illustrative embodiment, the seed sterilization station 12 includes a conveyor system 36 configured to transfer the containers 24 to the vats 26, 28, 30, 32, 34 containing different solutions for sterilizing the seed. The conveyor system 36 includes a conveyor 38, a stand 40 for supporting the vats 26, 28, 30, 32, 34, and a controller 42 configured to control movement of the conveyor 38.

In the illustrative embodiment, the containers 24 are attached to a conveyor 38 configured to transport the containers 24 to the different vats 26, 28, 30, 32, 34 as shown in FIG. 10. The vats 26, 28, 30, 32, 34 are on the stand 40 that is configured to vibrate and cause the solution in the vats 26, 28, 30, 32, 34 to be under constant agitation. In other instances, the vats 26, 28, 30, 32, 34 themselves are configured to agitate the solution therein.

First, the conveyor 38 transports the container 24 with the seed to a first solution vat 26 as shown in FIG. 10. The first solution vat 26 contains a first solution, i.e. an 74% ethanol solution. The conveyor 38 is then configured to lower the container 24 into the vat 26 so that the seed is immersed in the first solution. In the illustrative embodiment, the container 24 is made of a mesh material so that when the container 24 is lowered into the series of solutions, the solution flows into the container 24 and immerses the seed.

The container 24 with the seed is immersed in the first solution for about 3 minutes. Then the conveyor 38 raises the container 24 containing the seed from the first solution vat 26. By raising the container 24, the solution drains from the container 24, while the seed remains inside.

In the illustrative embodiment, a controller 42 is connected to the conveyor 38 and is configured to control the raising and lowering of the container 24, as well as the duration at which the containers 24 are immersed in the different solutions. Depending on the size of the vat, the sterilization and rinsing times will be adjusted accordingly.

After the conveyor raises the container 24 from the first solution vat 26, the conveyor 38 transports the container 24 to the seconds solution vat 26 as shown in FIG. 10. The second solution vat 28 contains a second solution, i.e. a 30% Clorox bleach solution containing 0.1% Tween-18. The conveyor 38 is then configured to lower the container 24 into the vat 28 so that the seed is immersed in the second solution. The container 24 with the seed is immersed in the second solution for about 18 minutes. Then the conveyor 38 raises the container 24 containing the seed from the second solution vat 28. Then the conveyor 38 transports the seed to be rinsed.

The conveyor 38 transports the container 24 to a first rinse vat 30 as shown in FIG. 10. The conveyor 38 is configured to lower the container 24 containing the seed into the vat 30 so that the seed is immersed in the rinse solution, i.e. autoclaved sterile water. The container 24 with the seed is immersed in the rinse solution for about 5 minutes. Then the conveyor 38 raises the container 24 containing the seed from the first rinse vat 30.

The conveyor 38 then transports the container 24 to a second rinse vat 32 as shown in FIG. 10. The conveyor 38 is configured to lower the container 24 containing the seed into the vat 32 so that the seed is immersed in the rinse solution for a second time. The container 24 with the seed is immersed in the rinse solution for about another 5 minutes. Then the conveyor 38 raises the container 24 containing the seed from the second rinse vat 32.

The conveyor 38 then transports the container 24 to a third rinse vat 34 as shown in FIG. 10. The conveyor 38 is configured to lower the container 24 containing the seed into the vat 34 so that the seed is immersed in the rinse solution for a third time. The container 24 with the seed is immersed in the rinse solution for about another 5 minutes. Then the conveyor 38 raises the container 24 containing the seed from the third rinse vat 34.

In some embodiments, the seed sterilization station 12 may include additional rinse vats like the rinse vats 30, 32, 34 before the first solution vat 26 and/or between the first solution vat 26 and the second solution vat 28. The conveyor 38 is configured to raise and lower the container 24 into the additional rinse vats before the first solution vat 26 and after the first solution vat 26. In other embodiments, the seed sterilization station 12 may be configured to accommodate gas sterilization methods such as those described in Example 9.

The conveyor 38 is then configured to transport the container 24 to allow the seed to dry as shown in FIG. 10. The seed may be air dried or the sterilization station 12 may include an air dryer 44 to dry the seed. Once the seed is dried, the conveyor 38 is configured to transfer the seed in the containers 24 onto solid 90O medium in a tray 46 after the final sterile water rinse for germination. Then the conveyor 38 transports the seed to the seedling germination station 14.

In other embodiments, the conveyor 38 may be configured to transport the containers 24 to different solution dispensers that provide the solution to the container 24. The conveyor 38 may be configured to move the containers 24 with the seeds to the different dispensers. The dispenser is then configured to dispense the solution into the container to immerse the seed in the solution. The seed is allowed to be immersed for the set duration before the solution is removed from the container 24.

The seedling germination station 14 may comprise growth containers 46 or trays 46 containing germination media 48 for receiving the sterilized seed for germination of the seed as shown in FIG. 10. Sterilized seed are germinated on solid 90O medium or in liquid 90O medium. Seed are transferred by automated, mechanical, and or robotic means from the containers 24 to large trays 46 of solid 90O medium for germination.

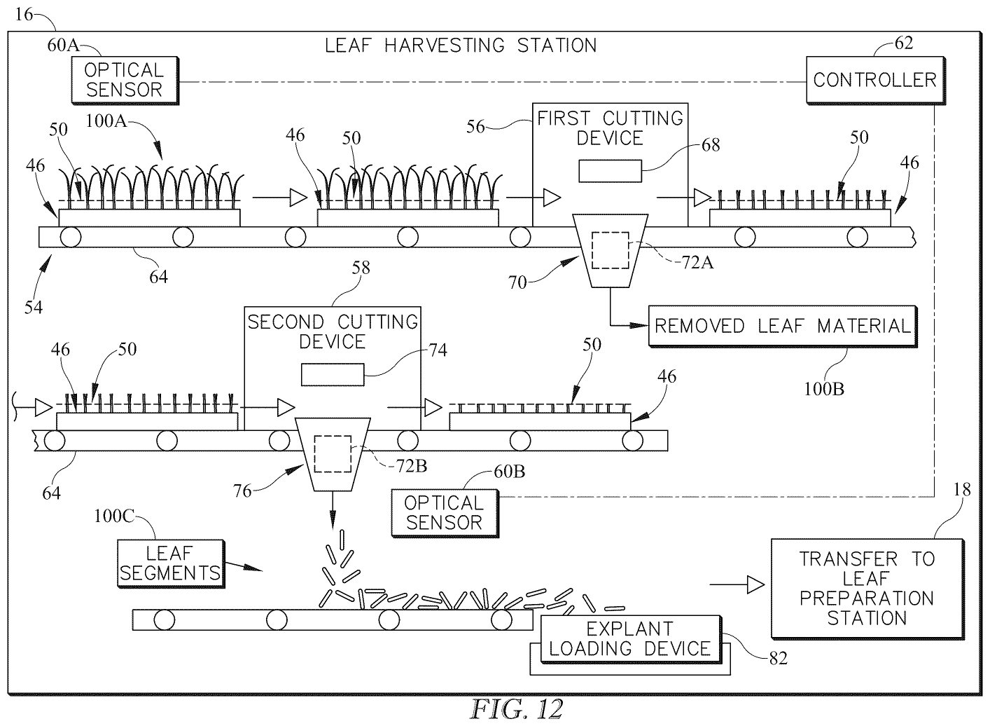

The tray 46 may include a mesh material 50 as shown in FIG. 12. The tray 46 includes the mesh 50 so that as the seed germinates, the mesh 50 retains the seedling growths near the stem portions of the seedlings. The mesh 50 has an adequate mechanical rigidity and the construction of the mesh 50 is intended to hold the plant seedlings in the tray during processing in the leaf harvesting station 16. The mesh 50 is made up of a material suitable for decontamination. In some embodiments, the trays 46 are hydroponic trays, which allow for dense planting and the ability to add and/or remove liquid media components after transfer by automated, mechanical, and or robotic means. In some embodiments, the seedling germination station 14 may comprise a climate controller 52 (light sensors, humidity sensors, temperature sensors, air circulation sensors, etc) for incubating the seedlings.

The leaf harvesting station 16 includes a conveyor system 54, a first cutting device 56, a second cutting device 58, optical sensors 60A, 60B, and a controller 62 as shown in FIG. 12. The conveyor system 54 includes a conveyor 64 configured to transport the trays 46 with seedlings 100A through the different cutting devices 56, 58. The first cutting device 56 is configured to remove the leaf canopy 100B of the seedlings 100A in the tray 46. The second cutting device 58 is configured to remove the leaf segments 100C for transformation after the leaf canopy 100B has been removed. The optical sensor 60A is configured to measure the height of the seedlings 100A before the leaf canopy 100B is removed. The optical sensor 60B is configured to measure the length of the leaf segments 100C removed for transformation. The controller 62 is connected to the conveyor system 54, cutting devices 56, 58, and optical sensors 60A, 60B to control the harvesting process.

The tray 46 containing the seedlings 100A are conveyed by the conveyor 64 at an oblique angle to the first cutting device 56. The first cutting device 56 includes cutting means 68 as shown in FIG. 12. The cutting means 68 may be made up a blade assembly 68 having sharp saw-like teeth on multilayered blades sliding in opposing directions.

Seedlings 100A in the tray 46 pass through the first cutting device 56 so that the blade assembly 68 cuts the leaf canopy 100B from the seedling 100A above the retaining mesh 50. Leaf material 100B from the canopy is diverted perpendicularly into a chute 70 included in the cutting device 56 for extraction. The cutting device 56 may also include a pneumatic device 72A configured to divert the removed leaf canopy 100B out of the cutting device 56.

The controller 62 is configured to control the conveyor speed and blade oscillation. The controller 62 may change the speed of the conveyor 64 and/or the oscillation speed of the blade assembly 68 so as to reduce bending of the seedlings 100A during cutting.

The remaining plant stem and root in the tray 46 is further conveyed by the conveyor 64 to the second cutting device 58 for segmentation. The second cutting device 58 includes another blade assembly 74. The cutting means 74 may be made up a blade assembly 74 having sharp saw-like teeth on multilayered blades sliding in opposing directions like the bladed assembly 64. The trays 46 with the remaining plant stem are driven into the second cutting device 58 with 3 cm segments exposed above the blade assembly 74. The blade assembly 74 removes the leaf segments 100C and the segments 100C are collected into a chute 76 as shown in FIG. 12. The cutting device 58 may also include a pneumatic device 72B configured to divert the removed leaf segments 100C out of the cutting device 58.

In some embodiments, the first cutting device 56 may be bypassed and a single cut is performed by the second cutting device 58. In this way, the entire leaf material above the retaining mesh 50 may be used as the explant for transformation.

Alternatively or additionally, the each of the cutting means 68, 74 may include, but is not limited to, rotating, banded, or scissor like blades at the cut location. These embodiments include linear tracked blades (band saw), single pivot double blades (scissors), sharp wire (cheese slicer), ganged rolling blades (pasta roller), single and double blade choppers (guillotine), dual action reciprocating blades (hedge trimmer) in combination with sliding stages as needed to engage plant material with blades.

In some embodiments, the each of the cutting means 68, 74 may include a laser, high-pressure water, or high-pressure air. Instead of the trays 46 containing several seedlings, the seedlings 100A are manipulated into a single-file line on the conveyor 64 to minimize the depth of the cut. Individual plants pass through the cutting means 68 of the device 56. The cutting means 68, in this case a laser, a high-pressure water jet stream, or a high-pressure air stream, removes the top canopy 100B of the seedling 100A. Alternatively, only one laser, high-pressure water, or high-pressure air cut is made and the entire seedling, including leaf canopy and leaf whorl, is used as the explant for transformation.

In the case of high-pressure water or air, the liquid or gas stream is provided from a sterile source and evacuated upon use through a collection point opposite the nozzle. The design of the nozzle is optimized with a high precision tip and ultra-high pressure to minimize injury at the cut and reduce reaction force of the plant. Similarly, the laser beam is optimized to reduce heat damage of the tissue by high frequency pulsing and operating within a near-infrared wavelength. The controller 62 may be configured to control the pressure of the water or air stream. The controller 62 may be configured to control the high frequency pulsing of the laser.

Like in the other embodiments, once the leaf canopy 100A is removed, the material is diverted by the chute 70 above the cut zone and disposed of. Leaf whorl segments are then conveyed by the conveyor 64 to the second cutting device 58. The individual plants pass through the cutting means 74 of the device 58. The cutting means 74, in this case a laser, a high-pressure water jet stream, or a high-pressure air stream, removes the leaf segments 100C. The whorl segments 100C are directed into the chute 76 above the cut zone for extraction and further processing. Leaf whorl segments are individually sent into the chute 76 with vibratory, mechanical, or pneumatic means 72B.

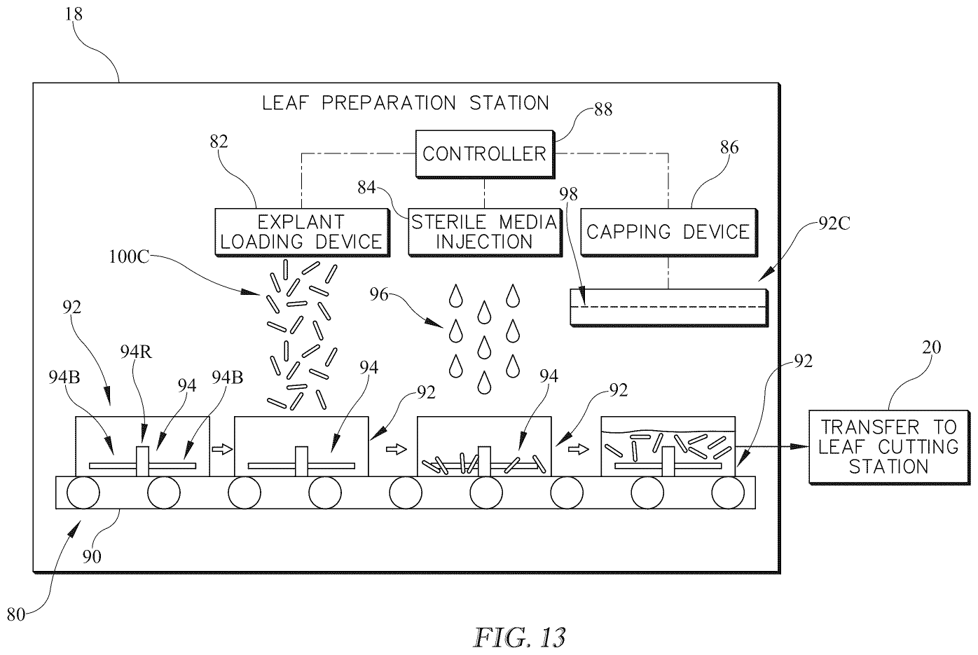

The chute 76 transfers the leaf segments 100C to a second conveyor 66 included in the conveyor system 54 as shown in FIG. 12. The second optical sensor 60B then measures and counts the leaf segments 100C before the leaf segments 100C are loaded into a loading device 82 included in the leaf preparation station 18 as shown in FIG. 12 and FIG. 13. The optical sensor 60B is configured to detect explant segments 100C that measure within targeted cut dimensions. The controller 62 may include additional sensors and/or controllers to provide additional functionality to the processes for quality control purposes.

The leaf preparation station 18 includes a conveyor system 80, the loading device 82, a sterile media injector 84, a capping device 86, and a controller 88 as shown in FIG. 13. The conveyor system 80 includes a conveyor 90 and bladed containers 92 that are transported by the conveyor 90 through the different loading steps. The controller 88 is connected to the conveyor system 80, the loading device 82, the sterile media injector 84, and the capping device 86 to control the preparation process.

The bladed containers 92 have a scalpel blade rotor assembly 94 as shown in FIG. 13. The bladed assembly 94 is coupled to the container 92 at the bottom of the container 92.

The blade assembly 94 has a rotor 94R and rotary blades 94B as shown in FIG. 2, FIG. 3, and FIG. 13. The rotor 94R is configured to rotate about an axis A. The blades 94B are coupled to the rotor 94R for rotation therewith.

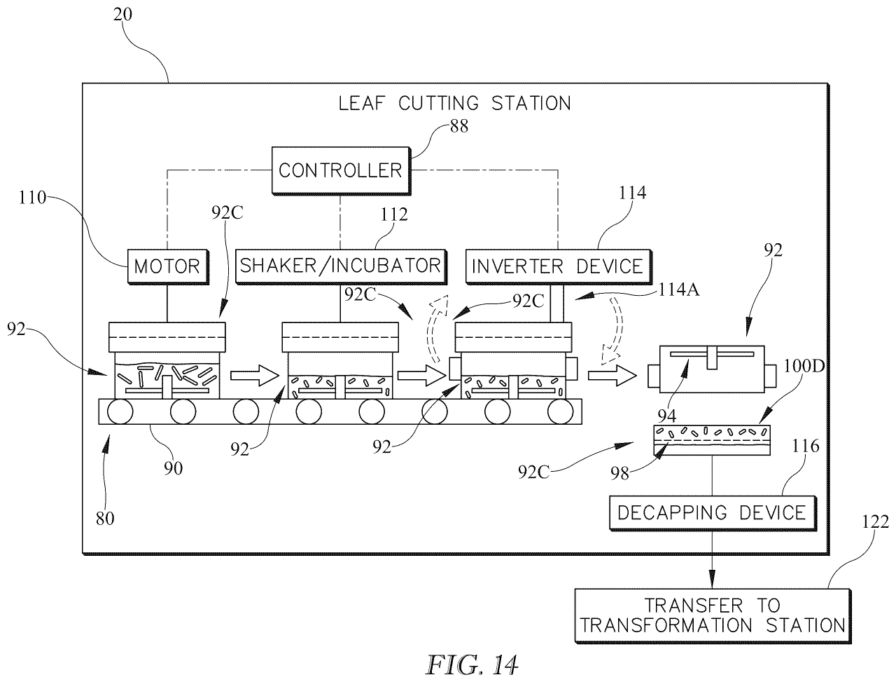

In the illustrative embodiment, the blade assembly 94 only has two blades 94B. In other embodiments, the blade assembly 94 may have a single blade, two blades, or more than two blades 94B. For example, the blade assembly 94 may have six blades 94B, like as shown in FIG. 2, FIG. 3, and FIG. 14. In some embodiments, different sized blades 94B may be used as suggested in Example 15. The leading edge 94E of the blades 94B has the sharp edge for cutting the leaf segments 100C. Alternatively, blades 94B may have more than one sharp edge for cutting the leaf segments 100C. Additionally, the rotor 94R may rotate clockwise or counter-clockwise.

The blades 94B of the blade assembly 94R have a tilt angle α and a pitch angle β as shown in FIG. 2 and FIG. 3. The tilt angle α is the angle between an axis X perpendicular to the rotation axis A and the radial axis B of the blade 94B. The pitch angle β is the angle between the horizontal plane of the rotor 94R and the chord line of the blade 94B. The number and type of blades 94B, the geometry of the rotor 94R, and the tilt angle α and the pitch angle β of the blades 94B may be optimized to achieve the best segmentation of the leaf tissue. This is discussed further in Example 15 above.

In some embodiments, the blades 94B may be axially offset. For example, one of the blades 94B may be spaced apart axially from the other blade(s) 94B relative to the rotation axis A so that the blades 94B are vertically spaced apart.

The conveyor 90 transports the container 92 to the explant loading device 82 as shown in FIG. 13. The explant loading device 82 dispenses the leaf segments 100C from the leaf harvesting station 16 into the container 92. The controller 88 is connected to the loading device 82 to control the amount of leaf segments 100C loaded into each container 92.

The conveyor 90 then transports the container 92 to the sterile media injector 84 as shown in FIG. 13. The injector 84 dispenses sterile media 96 into the container 90 with the leaf segments 100C. The controller 88 is connected to the injector 84 to control the amount of sterile media 96 loaded into each container 92. Sterile media may contain various components depending on the stage of the container in the conveyor process. For example, the media may contain various materials including, but not limited to, Agrobacterium, acetosyringone, plant growth hormones, and/or surfactants.

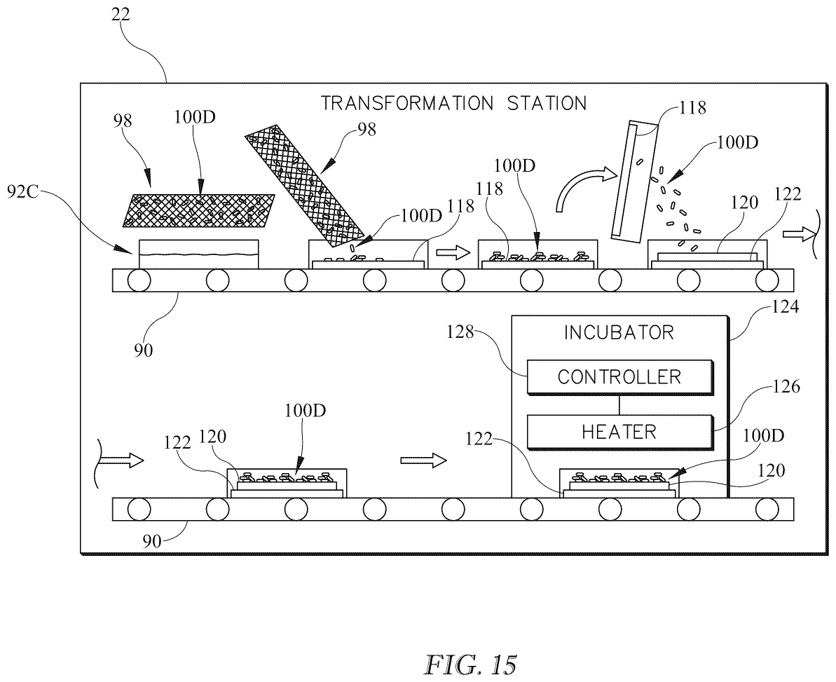

The conveyor 90 then transports the container 92 to the capping device 86 as shown in FIG. 13. The capping device 86 has a mechanical arm to attach a cap 92C to the container 92. The cap 92C has an internal and detachable mesh filter 98 as shown in FIG. 14 and FIG. 15. Once the container 92 is capped, the conveyor 90 transports the capped container 92 to the leaf cutting station 20.

The leaf cutting station 20 includes the controller 88, the conveyor 90, a motor 110, a shaker 112, an inverter device 114, and a decapping device 116 as shown in FIG. 14. The conveyor 90 transports the capped containers 92 to the motor 110. The controller 88 is configured to direct the motor 110 to couple to the blade rotor assembly 94 and to pulse in on-off cycles. The pulsing of the motor 110 causes the bladed rotor assembly 94 to rotate and mince the leaf segments 100C into smaller leaf-fragments.

The controller 88 is configured to direct the motor 110 to cycle according to a predetermined schedule in order to achieve the desired range of leaf-fragment/segment size. In some embodiments, the controller 88 is configured to direct the motor 110 to pulse a predetermined number of times and each pulse may be a predetermined amount of time as discussed further in Example 15.

After the leaf segments 100C are minced so that the average leaf segment length was approximately 2-3 mm. The minced leaf segments 100D are then transported by the conveyor 90 to the shaker 112 to incubate. The shaker 112 is configured rotate the contents of the container 92. The leaf segments 100D are allowed to incubate in the Agrobacterium suspension for a timed duration, i.e. about 30 minutes. Alternatively, prior to mincing the Agrobacterium is added to the sterile media 96.

The conveyer 90 then transports the container 92 to the inverter device 114 as shown in FIG. 14. The inverter device 114 is configured to invert the container 92 as suggested in FIG. 14. By inverting the container 92, the leaf segments 100D are collected onto the mesh filter 98.

Once the container 92 is inverted, the decapping device 116 removes the cap 92C. The inoculated tissue is collected onto the mesh filter 98 as shown in FIG. 15. The caps 92C with the leaf segments 100D on the mesh filter 98 are then transferred to the transformation station 22 as shown in FIG. 14.

In some embodiments, the mesh filter 98 is removed from the cap 92C and the leaf segments 110D are transferred to a Petri plate. The plates are capped with a lid so that they may be transferred to another incubator included in the station 20. The leaf segments 100D are allowed to incubate for a predetermined amount of time before being transferred to the transformation station 22.

In another embodiment, the leaf cutting station 20 may use a laser, an air stream, or a water jet to cut the leaf segments 100C into the leaf segments 100D. Instead of the cotainers 92, the leaf whorl segments 100C drop into a V-shaped conveyer 90 and are passed through a laser, air, or water jet cut mechanism slicing each segment into many sections in the direction parallel to the plant stem. Directly after being cut, leaf segments are split and set cut-face-down onto a retaining material provided on the top side of the laser, waterjet, or air cut bed.

The retaining material is also unrolled above the leaf segments 100D to create a sandwich-like retainer for the fine cutting process. Retained material is continued through the grid conveyor 90 with an appropriate bed material including, but not limited to, aluminum, stainless steel, corrugated plastic, or hardened steel depending on the cut process type. Leaf material is cut as it is conveyed through the laser, waterjet, or air cut bed in various cut patterns including, but not limited to, swiping back and forth or multiple cut heads set up as an array.

Fluids for the waterjet are decontaminated or single use to maintain sterility. Additionally, cut beds are sent through decontamination chambers along the conveyor to preserve sterile conditions within the equipment. Once segments are cut to specified dimensions, leaf tissue with retaining media are sectioned and loaded into containers for infection and further processing in the transformation station 22.

In another embodiment, pressure or force loads may be used to reduce the leaf segments 100C. The tissue may be reduced with the use of rollers or mills or opposing plates or ball bearings along the conveyor 90. The reduced leaf tissue is collected and a liquid suspension containing the Agrobacterium cells and the leaf tissue is incubated prior to moving to the transformation station 22.

In the transformation station 22, the mesh filter 98 is removed from the cap 92C as shown in FIG. 15. The leaf segments 100D on the mesh filter 98 are then transferred to on an autoclaved dry filter paper 118 to wick up and remove any residual Agrobacterium solution. Then the leaf segments 100D are transferred again onto fresh filter paper 120 that is resting on a co-cultivation medium 122 as suggested in FIG. 15. The infected leaf tissue 100D was then transferred to an incubator 124 to be incubated at 21° C. in the dark for 2-3 days.

The transformation process continues as described above in Example 5. The incubator 124 includes a heater 126 and a controller 128 in the illustrative embodiment. The heater 126 is configured to apply heat to the leaf tissue when needed during the transformation process.

In the illustrative embodiment, the automated system 10 has different controllers 42, 62, 88, 128 for each of the stations 12, 14, 16, 18, 20, 22. The different controllers 42, 62, 88, 128 are also connected to communicate operation of the different stations 12, 14, 16, 18, 20, 22 therebetween. In other embodiments, a single controller may be used to communicate the operation of the different stations 12, 14, 16, 18, 20, 22. The single controller may control operation of the different stations 12, 14, 16, 18, 20, 22 to streamline the process.

As used herein the singular forms “a”, “an”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

All patents, publications and patent applications mentioned in the specification are indicative of the level of those skilled in the art to which this disclosure pertains. All patents, publications and patent applications are herein incorporated by reference in the entirety to the same extent as if each individual patent, publication or patent application was specifically and individually indicated to be incorporated by reference in its entirety.

Although the foregoing disclosure has been described in some detail by way of illustration and example for purposes of clarity of understanding, certain changes and modifications may be practiced within the scope of the appended claims.

That which is claimed:

1. A high-throughput, automated method of producing a population of leaf explants comprising:
sterilizing a population of seed to provide a sterilized seed population;
transferring the sterilized seed population by mechanical and/or robotic means to a germination container, wherein the germination container comprises a germination medium;
providing a distribution of the sterilized seed population by mechanical and/or robotic means in the germination container;
germinating the sterilized seed population to provide a population of germinated seedlings;
growing the germinated seedlings to a sufficient height for harvesting seedling leaves by mechanical and/or robotic means;
harvesting the seedling leaves by mechanical and/or robotic means to provide harvested seedling leaves;
transferring the harvested seedling leaves to a holding container by mechanical and/or robotic means, wherein the holding container comprises a chopping means, a cutting means, and/or a blending means and chopping, cutting, and/or blending the harvested seedling leaves to provide leaf segments of an appropriate size for a bacterial infection;
providing to the holding container a bacterial infection solution;
infecting the leaf segments in the holding container for a sufficient amount of time to provide bacterially infected leaf segments;
removing by mechanical and/or robotic means the bacterial solution from the bacterially infected leaf segments; and
providing further sub-culturing, resting, selection, heat-treatment, regeneration, maturation, or rooting processes by automated, mechanical, and/or robotic means.

2. The method of claim 1, wherein the sterilizing comprises an ethanol treatment, a chlorine gas treatment, a dilute bleach solution treatment, and combinations of the foregoing.

3. The method of claim 1, wherein the germination medium is a solid, a semi-solid, or a hydroponic medium.

4. The method of claim 1, wherein harvesting comprises cutting and/or chopping the harvested seedling leaves with scissors, blades, wires, lasers, high-pressure water jet streams, high-pressure air, and combinations of the foregoing.

5. The method of claim 1, wherein the holding container further comprises a bacterial infection solution.

6. The method of claim 5, wherein the bacterial infection solution comprises Agrobacterium.

7. The method of claim 1, wherein the chopping, cutting and or blending of the seedling leaves is performed in a blender or a food processor.

8. The method of claim 1, wherein the leaf segments of an appropriate size comprise leaf segments of from about 0.1 mm to about 1 mm, from about 1 mm to about 2 mm, from about 2 mm to about 3 mm, or from about 3 mm to about 1 cm in size.

9. A high-throughput, automated method of producing a population of leaf explants comprising:
transporting each seedling from a population of seedlings to a first cutting device;
removing a top portion of the seedlings using the first cutting device;
transporting the seedlings to a second cutting device after removing the top portion of the seedlings;
removing a second portion of the seedlings using the second cutting device to provide leaf segments;
loading the leaf segments into a loading device configured to dispense a predetermined amount of leaf segments into a container each comprising means for chopping, a cutting, and/or a blending the leaf segments;
dispensing the predetermined amount of leaf segments in the container;
and
chopping, a cutting, and/or a blending the leaf segments to provide leaf fragments of a predetermined size.

10. The method of claim 9, wherein the transporting steps includes using a conveyor.

11. The method of claim 10, further comprising
measuring a height of each seedling included in the population of seedlings using an optical sensor;
determining if the measured height of the seedlings is within a predetermined height threshold; and
transporting each seedling to the first cutting device if the measured height of the seedlings is within the predetermined height threshold.

12. The method of claim 11, further comprising
measuring a length of each leaf segment using an optical sensor after removing the second portion of the seedlings;
determining if the measured length of each leaf segment is within a predetermined length threshold; and
loading the leaf segments into the loading device if the measured length of the leaf segment is within the predetermined length threshold.

13. The method of claim 9, wherein the first cutting device includes one of scissors, blades, wires, lasers, high-pressure water jet streams, and high-pressure air.

14. The method of claim 9, wherein the second cutting device includes one of scissors, blades, wires, lasers, high-pressure water jet streams, and high-pressure air.

15. The method of claim 9, further comprising:
injecting a predetermined amount of bacterial infection solution into the container and
attaching a cap to the container to close the container and prevent the leaf segments and bacterial infection solution from moving outside the container.


SEQUENCE LISTING (21 sequences)

SEQ ID NO: 1 - DNA, 6270 bp, synthetic construct
  misc_feature 1..6270: note = PHP5096
  source 1..6270: mol_type = other DNA
gacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctgagttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgcgatctggggaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaacaatgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctcggcgtgatcatccagtgcctcgtgaccgagaccaa

SEQ ID NO: 2 - DNA, 28390 bp, synthetic construct
  misc_feature 1..28390: note = PHP71539
  source 1..28390: mol_type = other DNA
gacccgcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatgcttaagagatcggggtcgctttctcttgccttgatggtctccttctgttcgtcgagccttgccacgccactctcatctgctgagtttgaccatgttgctcgcaagtgtgccccatcagttgcgacatctacgcttgcggcgatagctaaggtggagagtcgctttgatcctttagcgattcatgacaacacgaccggcgaaacgcttcactggcaagatcacagccaagcaacccaagtcgtcaggcaccgtctcgatgcacggcattcgctggatgttggcctcatgcaaataaactctcgaaatttttctatgctcggtctgacacctgacggtgcgctccaggcgtgcacatcattatctgccgctgcaaacatgctgaaaagtcgttatgcaggcggcgaaacgattgacgagaagcaatttgcgcttcgtcgggcgatctccgcttacaacaccggtaatttcatcggcggttttgcaaacggctacgtgcgaaaagttgaaacagctgctcaatcgctggtgcccgcgttaatcgagcctccaaaagacgatcacgaggcgctaaaatccgaagagacgtgggatgtttgggggtcatatcagcgccgctcgcaggaggatggcgctggcggtttaatcgctccgccaccgccacaccaggacaacggcaaatccgcagacgacaatcaagtcttattcgacttatactaaggaggtgcgcattgatgcgatgctttgagagataccgtttacatctaaatcgcctctcgctctcgaatgcgatgatgcgcgtgatatcgagctgcgccccaagcttgtgcggtgcaattgcatggagcatttcctcatccggacccgccgcagcgcaatctgcgggtggcggcactgaccccgccacaatggttaacaatatatgcacgtttatccttggtccgttcggccagtcactcgctgttctcggcattgtcgctatcgggatctcctggatgttcgggcgggcttcgcttgggctggttgccggcgtcgtcggcggcattgttatcatgtttggggcgagcttcctcggccaaacgctcactggcggtagttgatggctgatcgtttggaagaatcgaccctttacctcgcagccacacggcccgcattgtttcttggggtgccactgacattggcagggttattcatgatgttcgccggctttgtcatcgttatcgttcagaacccgctctacgaagtcgttctcgtgccgttatggtttgcagcccggctcatcgtggagcgagactacaatgcggcgagcgtcgtcctgctatttttgcggaccgcgggaagaagcattgatagtgcagtttgggggggcgctactgttagcccaaatccaattagggttcccccacgagggagaggaatggtgtgatgctcggcgcgagtggaacgaccgaaagatccggtgagatctatctcccttatattggccacctcagcgaccatatcgtccttcttgaagacggatcgatcatgaccattgcgagaattgatggcgttgcattcgagcttgaggaaactgaaatgcgcaatgcgcgttgtcgtgcgttcaacacgctgttgcgcaatatcgctgatgatcatgtgtcaatatatgctcacctcgtacgtcatgccgacgtgccatcatcggcgccgcgacacttccgtagtgttttcgccgctagcctgaacgaagcttttgaacagcgcgtgctctccggccaactcctccgcaatgaacacttccttacgttgattgtctacccacaggcggctttagggaaggtaaagaggaggttcaccaagctaagcggaaaaagggaaaacgatctcacgggccagatcaggaacatggaagatctttggcatgttgtcgctggctctcttaaagcgtatggcctgcatcgtcttggcatccgcgagaagcagggtgtgctcttcaccgaaattggcgaagcgctacggttgatcatgactggtcggttcacaccggttccggtcgtcagcggctcactcggcgcttcgatttataccgacagagtcatttgcggcaagcgaggactcgagatcagaacgccaaaagacagttacgttggatccatctattcgtttcgcgaataccctgcaaaaacacggccgggcatgctcaacgcgctgctatccctcgattttccacttgttctcacgcagagtttttcgttcctgactcgcccgcaagcgcacgcgaaacttagcctcaaatcgagccagatgctgagttccggcgataaagccgtgactcaaatcggcaaattatccgaggctgaggacgcacttgcgagcaacgaattcgttatgggctcacatcatttgagcctttgcgtctatgcagacgatctcaatagtcttggggacaggggcgcgcgggctcggacacgaatggcggatgcaggtgccgtggttgtccaagaaggtattggtatggaagcggcctattggtcccaattgccggggaattttaagtggcgcacacgccctggcgcaatcacttcacgcaatttcgcagggtttgtctctttcgaaaactttccagagggcgccagctcaggccactggggcaacgcgattgcccgatttcgtaccaatggcggaacgcctttcgactatatcccgcatgagcacgatgttggcatgacggcaatattcgggcctatcgggaggggtaagacgacgctcatgatgtttgttctagccatgctcgaacagagcatggtcgaccgtgcaggtacggtcgtgttctttgacaaggaccggggtggcgaattgctggttcgcgccacaggaggaacatatttggcacttcacagaggcacacccagcgggttggcgccgttgcgtggcctagaaaacacagcagcctcacacgattttctgcgcgaatggatcgtggctctcatcgagagtgatggtcggggtgggatttctccggaagagaaccgccgtctggtccgtggtatccatcgtcagctctcgtttgatccacaaatgcgttcaatcgcggggttacgtgaatttttgttgcatgggcccgccgaaggcgcaggagcgcggctccaacgctggtgccggggccatgcgcttggctgggcatttgacggcgaagttgacgaagtaaagttagatccgtcgattaccggcttcgacatgacgcatcttctcgaatacgaggaagtatgcgctcccgctgcagcatatctcctgcatcggattggagccatgatcgacggccgccgttttgtgatgagctgcgatgagtttcgcgcctatttgttaaaccctaaattttcgactgtcgtcgacaaattcctcctgaccgttcgaaaaaacaacgggatgctaatactggcaacgcagcaaccagagcatgttctggaatcgccgctaggagccagcttggttgcgcaatgtatgacgaagattttctatccatcaccaaccgcagatcgatcggcttatgtcgatggactgaaatgtaccgaaaaggaatttcaggcgatccgtgaagacatgacggtcggcagccgtaagtttcttcttaaacgagaaagtggaagcgtcatctgcgaatttgatctgcgggatatgcgtgaatatgtcgccgtgctttcggggcgtgccaacacggtgcgctttgcaactcgactacgcgaggcacaagaaggcaactcatctggctggctcagcgaattcatggcccgtcaccacgaggcagaagattgataaggtaggaaacgatgaagacgacgcaacttattgcaacagttttgacctgcagctttctatatattcagcccgcgcgggcgcagtttgttgttagcgacccggcaacggaggctgagacgctcgcgactgcgctcgcgactgcggagaatctcactcagactatagcgatggttacgatgttgacgtcggcctacggcgttactggactactgacttcgctcaaccagaaaaatcagtatccttcgacgaaggacctagacaatgaaatgttttcgccgcgaatgccaatgtcgaccacggcacgtgcgatcaccagcgatacagatcgtgcagtcgtgggtagtgatgctgaagcggacctgttgcgatcgcagatcaccggttccgcaaacagcgctggcattgcggctgacaatctggaaacgatggacaaacgcttgacggcgaatgctgatacgtctgctcagctttcccgatctcgcaatatcatgcaggcaaccgtgaccaatggtttgcttctcaagcagatccatgacgcaatgattcaaaatgtacaggcgacaagcctattaacgatgactaccgcgcaggccggccttcacgaggcggaagaggcggccgctcaacgcaaggagcatcaaaagaccgctgtcatctttggtgccctcccctaaggctgggcgatttgttcatccgcccgcatcctcgccgaatgcgagctcattttatccaacattatgcgacaaaccagtcaagttcaggtccaatcgatgaatttcacgattccggcgccgtttacggccattcatacgatcttcgatgtagccttcacgacaggcttggactcgatgcttgagactatccaggaggcggtgagtgcgccattgatcgcctgtgtcactctttggattattgttcagggtattttagtcatacgcggcgaagtcgatacccgtagcggtatcactcgggtgatcacggtcaccatcgttgttgctctaattgttgggcaggctaactaccaagactatgtggtttccatcttcgaaaagacggtcccaaactttgttcagcagtttagtgtaaccggcttgcctctgcagactgttccggcacagttggatacaatgttcgccgtgacccaggccgtttttcagaaaatcgcatccgaaatcggtccgatgaacgaccaggacatccttgctttccaaggggcacagtgggtcctttacggcacgctctggtctgccttcggagtttacgacgccgttggaattctcacgaaagtgcttctcgcgatcgggcctctgatcctcgtcggatatatttttgatcgcacgcgggacatcgcagctaagtggatcgggcaacttatcacctacggtctcttgcttctcctcttaaacctcgtggcaacgatcgtcatcctaaccgaagcgactgcgctcacccttatgcttggtgtaatcaccttcgccggtacgaccgcggccaagatcattggtctttacgaactcgatatgttttttctgacaggggatgcgctcattgtcgctttgccggcgatcgccggcaacattggaggcagttactggagcggcgcaacccaatctgccagcagcttgtaccgtcgcttcgctcaggttgagcgaggctaggtcgcgcaaaaattcgcctcaatggagaattctatgaagtattgcctgctgtgcctagttgtcgctttgagcggctgccagacaaacgacacattagcgagctgcaaaggcccgatcttcccgctgaatgtggggcgatggcagcctactccgtcagatcttcagctcggcaattcgggtggacgctatgacggggcctgaatatgccatgctagtggcgcgcgaaagccttgccgagcactataaggaagtagaagcctttcaaaccgcgcgagcgaaatcggcgcgacgtctctccaaactcattgcagctgtcgcagctatcgcgattttgggaaatgttgctcaagcgttcgctatagccacaatggtgccgttgagcaggcttgtgcccgtatatctatggatacggccggacggcaccgttgacagcgaggtgtctgtctcgcgattgcctgcaactcaagaggaggccgtcgttaacgcctcattgtgggagtacgttcgcctgcgcgagagttatgatgccgacaccgctcagtacgcctacgacctggtatcgaacttcagtgccccaacagtgcgccaggattaccagcaattcttcaactatcccaatcccagttcgcctcaagtcattcttggcaaacgcggcagggtggaggtcgagcacatcgcttcaaatgatgtaactccaagcacgcagcaaattcgctataaaaggaccctcgtcgttgacggcaaaatgcctgtggtgagtacgtggaccgcgacagttcgctacgaaaaggtgaccagcttgcccggcagattgagactaaccaacccggcaggtctggttgtcacctcctatcagacatcggaagataccgtttcaaacgtaggccacagcgaaccatgatcagaaaagcacttttcattttagcatgtttatttgccgctgcgactggtgcggaggctgaagacactccaatggcgggcaagctagatccgcgcatgcgttatttggcttacaatcccgatcaagtggtgcgcctctcgacggcggttggagctactttggtcgtaacattcgccacgaacgaaacggtgacagcggttgccgtttcaaatagcaaagatctagcagccctaccgcggggaaattatctatttttcaaggcaagccaggtcctcacgcctcagccagtaatcgtgctaaccgcaagcgactccgggatgcgccgttatgttttcagtataagttccaagactctgtcccacctcgataaagagcagcccgatctctattacagcgtccaattcgcctaccccgccgacgatgcggcggctcggcgaagggaggcacaacagaaggctgttgtggacagactacacgcggaagcacaatatcaacggaaagctgagaatttattggatcagcctgtcacagcccttggtgcggcggacagtaattggcactacgtcgcccaaggcgatcgttcgctgttgccactcgaagtcttcgacaatggatttacgacggtattccactttccgggcaatgtacgcataccctccatctacaccatcaatcctgatggcaaggaagctgttgccaactattcagttaaagggagcgatgtcgagatttcttcggtttcccgaggttggcgtctgagggatggccacacagtactatgtatctggaacaccgcttacgatcccgttggccaaaggccgcaaacgggcacggtgaggcccgatgtgaaacgcgtcctgaagggggcgaagggatgaataacgatagtcagcaagcggcacatgaggttgatgcatctggatccctggtctccgacaaacatcgccggcgtctttcggggtctcagaaattgatcgtcggaggtgtcgttctcgcgttatcattaagcctcatttggctaggtgggcgccaaaagaaggtgaatgagaacgcatcgccgtcaactttgatcgcaacaaacaccaagccatttcatccagctccgattgaggtgccgccggatcctccagcggttcaagaggctgttcagcctgctgctcctctaccgccgaggggcgaaccggagcggcatgagccacggccggaagaaacaccgatttttgcatatagcagcggcgatcaaggggtcagcaaacgcgccattcagggcgacacgggccgaagacaagaaggcaagcgtgacgacaactccttgccgaatggcgaagtgtccggcgagaacgatttgtcgatacgtatgaaacccaccgagctgcagcccagcagcgccacgctcttgccgcaccccgattttatggtaacgcaagggacaataattccgtgcatcttgcaaaccgcaatcgacacaaatttggcaggctatgtaaagtgtgtcttgcctcaggatattcgtggaacaacgaacaatatcgtgcttcttgatcgtggcaccaccgttgttggcgaaatacagcgtggcttgcaacagggagatgggcgcgtttttgtgttgtgggatcgcgccgagacacctgaccatgcgatgatctcgttaacatcgccaagcgcggacgaactcggtcgctcaggattgccgggctcggtcgacagccacttctggcagcgttttagcggagctatgctcttgagtgttgttcaaggcgccttccaggcagctagcacctacgccggcagctcgggtggcgggatgagcttcaacagctttcaaaataacggtgagcagacaactgagacagcccttaaggcaaccatcaacataccgccaaccctgaagaagaatcagggtgacaccgtttccattttcgtagcacgggacctcgatttctttggtgtttaccagctccgcctgactggcggcgccacgcgggggaggaaccgccgctcttaatgaattcaaatttccgcttagagataggatacattgtaaatggaagtggatccgcaactacgctttcttctgaagccgattttggaatggctcgatgacccgaagactgaagaaattgcgatcaatcgacctggagaggcatttgtgcgccaagccggcatttttaccaagatgcctttgcccgtctcttatgatgatcttgaagatatcgctattttagcgggcgcgctgagaaagcaggatgtcggaccacgtaaccccctctgcgccactgaacttcctggtggtgaacgactacaaatctgtctgccgccgaccgttccctcgggcaccgtcagcttgaccattcgacggccaagctcgcgtgtttctggtcttaaagaagtctcctcccgttatgatgcttcgaggtggaaccagtggcagacacgaaggaaacgccaaaatcaggatgatgaagctatccttcagcattttgacaacggggatttggaagcgtttctgcacgcatgcgtcgtcagccgactgacgatgttgctatgtggccctaccggaagcggcaagacaacaatgagcaagaccttgatcagcgccatccccccccaggaaaggctaatcaccatagaagatacgctcgaactcgtcattccacacgataatcatgttagactactctactccaagaacggtgctgggctgggtgctgtgagcgccgagcacttgctccaagcaagtctgcgtatgcggccggaccggatattgcttggcgagatgcgcgacgatgcagcatgggcttatctgagtgaagtcgtctcgggacatccgggatcgatttcaacaatacacggcgcgaatccaatccaaggattcaagaaactgttttcccttgtcaaaagtagcgcccaaggtgctagcttggaagatcgcacactgattgacatgctctctacggcgatcgatgtcatcattccattccgtgcctatgaggacgtttatgaagtaggcgagatctggctcgcggcggacgcacgacgccggggcgagaccataggcgatctccttaatcaatagtagctgtaacctcgaagcgtttcacttgtaacaacgattgagaacttttgtcataaaattgaaatacttggttcgcattttcgtcatccgcggtcagccgcaattctgacgaactgcccatttagctggagatgattgtacatccttcacgtgaaaatttctcaagcgctgtgaacaagggttcagattttagattgagaggtgagccgttgaaacacgttcttcttatcgatgacgatgtcgctatgcggcatcttattatcgaataccttacgatccacgccttcaaagtgaccgcggtagccgacagcacccagttcactagagtactctcttccgcgacggtcgatgtcgtggttgttgatctaaatttaggtcgtgaagatgggcttgagatcgttcgaaatctggcggcaaagtctgatattccaatcataattatcagtggcgaccgccttgaggagacggataaagttgttgcactcgagctaggagcaagtgattttatcgctaagccgtttagtacgagagagtttcttgcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttacgacaggagtatagatgttctcattttgcggctgcgccgcaaacttgaggcggatccgtcaagccctcaactgataaaaacagcaagaggtgccggttatttctttgacgcggacgtgcaggtttcgcacggggggacgatggcagcctgagccaattgcatttgattaatttaggtgactgaggacgcggccagcggcctcaaacctacactcaatatttggtgaggggttccgataggtccctcttcaccaattgctcgatggcttctctccagcaaagaatgacgcgagcgcggcggtagccagcttgtggccgaaagctcgagcggtctccaaccccaacggatcaaaatgacttcgagcgacctcgagcaacgcaaccgggaacatgcgtgaggtctgaacgagaacggatttttctgtagttgaagggatcggataacttttcggggccacgcgaaatgatccatctgccagcatgctttcgaaatcgtccaacgcgcgccttaaaatcatttgtagcgacttcgagggactgtattgccgaacgaggttgtcatatgttttcgacacttgaggcgcgggcggtcgcgctgaaagaaaaacctggagctttttcggggacggaggtggactaagggcatccacagttagcttaagttgtcgatcgggactgtaaatgtgatcggcgacgagaggctcacgttgctggtctttctcgtcggctttttcaggcaagtgctggaggtccagcttctggggaacaagtgtcgggttgggatggtggatctcgggtcgagcaccagcaagccgccgtgcttcgccgaccgacaatgcgggcttgcgaattgccatcttcaagcctccaagattttgctgatcagtttcgaaatgaccacgacttcctccatcgcaatccgaagattcctctctatgaggcgcatcgtcggatcagttcccgtgtttagtaatgtaagatgcaacatgccgcgttctttcatcgcggcaaatgcatctctttcatgcatgggagacggtacaactggaaggctctctagcgtctctgacatcctgcgttgcgatgttgtcaatcggccgaccgggacgcgttggcgcaaaacagctgtaggaattgccaaattttcactcaacagcagctcgatgacgtagcggtaggtagatagtgcctcatcgatgtcgagcggcgttagcatggtggggatcagaagcaggtttgagctagcgatgattgtgttgttgagctcgctcgagccgccacgcgtatcggccaacgcataatcaaatccttcgagctcggcattttcataggctgcttcaagaaggggcatttcgtcggcggaatagacttcacagcgaggatcccaggtactgctttgtaaggcgttttctctccatcgcgtcagaggccggttttcgtcggcatcaaagagggccactcgtttaccgtcatttgccaaagcagcgcaaaggcccatgagtgcggtggttttgccagcaccccctttgaaagaacaaaacgtcaaaagttgcatattctgatcccgcctatcctgtgaaaccggagtgcatttgtatttttgttcgtataaatgtttttgtgattatcgatgagtaaaagcgttgttacactatttttatttcacattcgttataagacaattgcaaatgtagcaagtatattcagtattgactgtaaatgtactgttgatttcatattgagcagggctagacttccatccgtctacccgggcacatttcgtgctggagtatccagaccttccgctttctttggaggaagctatgtcaaaacacaccagagccacgtcgagtgagactaccatcaaccagcatcgatccctgaaagttgaagggttcaaggtcgtgagtgcccgtctgcgatcggccgagtatgaaaccttttcctatcaagcgcgcctgctgggactttcggatagtatggcaattcgcgttgcggtgcgccgcatcgggggctttctcgaaatagatgcagacacacgagaaaagatggaagccatacttcagtccatcggaatactctcaagcaatgtatccatgcttctatctgcctacgccgaagaccctcgatcggatctggaggctgtgcgagatgaacgtattgcttttggcgaggctttcgccgccctcgatggactactgcgctccattttgtccgtatcccggcgacggatcgacggtcgctcgttactgaaaggtgccttgtagcacttgaccacgcacctgacgggagaaaattggatgcccgatcgcgctcaagtaatcattcgcattgtgccaggaggtggaaccaagacccttcagcagataatcaatcagctggagtacctgtcccgaaagggaaagctggaactgcagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtaagcttccccgcaggtaccgaccaaaccgcagcttatgaagcaagccgggaatgggcagccgagatgtttgggtcaggatacgggggtggccgctataactatctgacagcctaccacgtcgaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaatatcgacgccttgagcggatgcaggctcaaaagattcaattcgaagatacagattttgatgagacctcgcctgaggaagatcgtcgggacctcagtcaatcgttcgatccatttcgatcggacgcatctgccggcgaaccggaccgtgcaacccgacatgacaaacaaccgcttgaaccgcacgcccgtttccaggagcccgccggctccagcatcaaagccgacgcacggatccgcgtaccattggagagcgagcggggtgcccaaccatccgcgtccaaaatccctgtaactgggcatttcgggattgagacttcgtatgtcgctgaagccagcgtgcccaaacaaagcggcaattccgatacttctcgcccggtgactgacgttgccatgcacacagtcgagcgccagcagcgatcaaaacgacgtcatgacgaggaggcaggtccgagcggagcaaaccgtaaaagattgaaggccgcgcaagttgattccgaggcaaatgtcggtgagcccgacggtcgcgatgacagcaacaaggcggctgatccggtgtctgcttccatccgtaccgagcaaccggaagcttctccaacgtgtccgcgtgaccgtcacgatggagaattgggagaacgcaaacgtgcaagaggtaatcgtcgcgacgatgggcgcggggggacctagactagtagacaggaaggaccgaataatggcaaatggtcagttcacgatacgctctgctcgcccggcctccgtcggactgacaggcgaacggcgtggagccgcatccgcctctagctctgcactgtccaatgttcaaagagatgttagggataggctgattccaactagctcaccaagattaccaaatgcagccatattgcgtgattcctcgggaagagcgtcgactggtctgcggtacatggcggctactcttcattggtctgcgatcgcgccattatcgctaataaacagcaacgacctggctccggccgcttatgactttgagacgcgaaataacgcaagaaatgtgactgccaaagtcggcagggcagtccctgttcccaagcaaggcgggctcggcaaaacgctcgcacccgtaccccttagtacacgtatatcaagggtcaattccgaccgaagactgcccgctgacgcagaagaccgccctgaaacgcgcgacccccagaaaggacgtggcagtcatggtgcgacgccaaccttacatgaaaagattggaaccgcgtttgctcgaagattgcgaaagcatacgtactatattgtttgcagttgctgccagacccggagcgcgttgacgatgggtgcaaagatttcggtgaagtcatgaactccagcaagacttcgccccagcgtatgaccctgagcatcgtatgttcgctggcagccggtttttgtgcggccagctgctatgtaacgttccgccggggcttcaacggcgaagcgatgatgacgttcgacgttttcgctttttggtatgagaccccgctttacttgggttatgccagcaccgtcttctggcgtggtttatctgttgtcatctttacctcgctgatcgttctttcaagtcagctcatcatatcgctgcgcaatcagaagcatcatgggacagctcgttgggcagaaattggcgaaatgcggcatgctggttatctgcagcgttacagtcgcatcaaggggccgatctttggaaagacatgtggtcccctttggttcggcagttatttgaccaatggcgaacagccacacagtcttgtcgtcgcgccaacgcgtgctggcaaaggcgtcggcatcgtcattccaacgctgttgaccttcaagggctcggtaatcgcccttgacgtcaagggagaattgtttgaactgacgtccagagcacgcaaagcgagcggcgacgcagttttcaagttctcccccctagatcctgagcggaagactcattgttacaatccggtcctggatattgccgcacttccgcccgaacgccagttcactgaaacacgccgtctagctgcgaaccttattacggctaagggaaagggagcagaaggctttattgacggcgcacgtgacctgttcgtcgcgggaatccttacctgcattgagcgtggcacaccaacgattggcgcggtatatgacctatttgcgcagcctggcgaaaagtataagctttttgcgcaactcgcggaggaaagcctaaacaaagaggctcagcgtatcttcgataatatggcgggcaacgacacgaaaattctgacatcgtacacctctgtgctgggcgacggtggactgaacctgtgggctgatccgcttatcaaagcagcgacaagccggtcagacttttccgtttacgatctccggaggaagaagacctgcatttatctttgtgtcagtcccaacgatctggaggtcttggcaccacttatgcgcctgatgtttcagcagctcgtgtcaatcttgcagagatcgctgccaggtgaagacgagtgccatgaagttttatttctcctcgacgaattcaaacacctgggcaagcttgaggccatagagaccgcgatcacaaccatcgccggttacagaggccgctttatgtttattattcaaagtctttcggccttgtcgggcacatacgatgacgcaggaaaacaaaactttctgagcaatactggcgtacaagtatttatggccacggctgatgacgaaactccaacctacatctcaaaagctatcggcgaatatacgtttaaagcgcgttcgacctcttacagtcaagccagaatgttcgaccacaacatccagatttctgatcaaggtgcagcccttttgcgccccgaacaagtgcgcctgctagacgatcagagtgaaatcgttctcatcaaagggcgacctccactcaaattacgaaaggtgcagtattattccgatcgtacgctgaaaggccttttcgaacgccagatgggctctctgcctgagcccgcacccttgatgctttccgactatagcaacgatcaagttcaataccacttggctccgatagcaaattttaatgaggatgctgcaccgcaaaacagaactgtggccgaggaccatggaagtgttaaagtcggtgctgatatccctgaacgcgtgatgggaataaatggtgacgaggaacaagccgatgcgggcgagataccgccggaatcggttgtgcctccagaattgacgctcgctctgaccgctcaacagcaattgttggaccagattattgcacttcagcaaagatcgaggtccgcaccggcatagcctgcgaaatgatcttaatggtgcaattcgtttcaggcggcatgttgcggttcaacaaagtgtacgccagatcccaattggccctttacgaggtgtcggtatgacaggaaagtcgaaagttcacataagaggttcggctgacgcgcttcctgacgttcctggcggaagtactaccgccccttttttaaccgaaccttctcgggatcaggttgatgcctcgtttgaggtccaaaccgactacagccagtctacttccgtgtcgtttacctatgatggtgttggacttggtcctgccgagcgtgcggcttacgagaactggtgcgaaccgggccggcccacttggaaagatcttataatcaaggcacgtgtcgatccgattgacgatgtgacctggctccgagatttagaagaggacaccccctcaaccttcagatacgaagggatgcctctgggcatcggggaacgacaggcctacgaaaattggcaagaggacgctcagccgacatgggaagaccttgttgtcagcgcacgcttgacggaacttggccgtccacacgggattaccggcgagtatacatccctcgcaggatcgaagaatacaagttcaatttcattgaagcgaaagcggagcaacttaattgatgatgagaattcatccggatcgttttcatatgacgggatgaagctcggggaagccgagcgttctgcatatggtgactgggccgaggcggagccacccacgtggaaagatttggtattgagggcacgcgtttcctcgatcaatgactctgcttggctttttgattcacaaacatcttcatcatcatttgaatacaacggtgttcccttgggcgagccggaacggcaggctctcagacaatggcaaggagacgctcagcctacctgggaagatctcgttgttaacgcgcgtatggcagaactttgccatgctggttggattgaaggtcaaaaaggttgctttgaagagcgcggggaggctctgcccgcgtcggaacgcggttcgcaacgccccattggtcaacggacagattcctccgattcttttgtgtatgatggcacaaggctcggagcacctgagcgaactgcttatgaacgctggagtaagagggaacgcccgacttgggaagatctcatcttagatgcacaccaggccaggactgaaagtgacgctgttacgacccaagcgattggtcagtcgtcctcaccggttttcttatatgaaggaaagtcgctcggagacagggaacgaaaggcttacgaaaaatggcggcagccagcccaaccgcgatggcaaaatcttgtagtgaacgctcgtctggcagaaatcgatccctcagcctggattgccgatgagcgcgatccgcttgatgatagcgacgcgcttggtcgcccgtcgtacacaagcttgacggatagatcagacgtccctttagacgatcaatcaatctatcgtcgttccgacctagtaagggagcaggtgccagaatcgtctcaaaggcaattcgcagcatgttcagaatctgaaacgaggcctgtgcaatggtttactgcttctgggtcagatgcaaacaatacggaaaatatcaccgccagcgatcccgtcgatcgcacgggtggagttaagcggctaggctccaaaagcgacagaaccgttacagcttctatccatgacgtgaattccagcacaaggcgactgttgcttaacgaatttggatcggaggctccgcgcccttcgccagaaaagactgttcgcttaagaagcgacaatattggcacctatgggagccggaaaaatgaacgagcgcggctcgcgaccgaaaccggtgcgtatgagtcggagcatattttcgggttcaaggctgtccacgatactgcgagagcgacgaaagagggccggcgtctcgaaaggcccatgcccgcctaccttgaggataaggggcttcatcgccaacatattggcaccgggagaggacggaccaaacttgtcgggcgcggatggccggatgacacaagctatcgctcggatcaaagggcaactctgtcggaccccgttgcgcgctcggaaggcgcgacggcctcaaatgggtatcaattgaaccaattgggttacgcgcaccaactcgctagcgatggcctgcaaagtgaatcgcccgatggtgttgccttgccaattcaagtggcaacaacgagctacaactatacagtgagccgcgatcctgtccttgttccgccggataaaaacgaagcccctcaattgctgcatcttggtccccgtggtcaaaccgaagctgttcttgcccgcgaaacagcattgactggaaaatggccgactctcgagcgtgagcagcaagtgtatcgcgagtttttggccttatatgacgtaaaaaaagatcttgaggccaaatcagtcggcgtaagacggaaaaaaaaagaagttatttctgcgttagaccgaactgcgcgcttgataagcacgtcgccttcgaaagctcgatccaaagcagagactgaaaaagccattgatgagctcgatgatcgacgagtttatgatccgcgtgatcgagctcaagacaaagcgtttaaacgctgataagtcgccaatatagtgatcatttgcagtattcgcatcgatcgctggttgatattctgccgctggtcgaccggctgctcgtcgccaaaaatgctcacagggatacgatggcctcggtcaggccgcgtgcgtcctgtctttccagttcctccctttcagctcgattgtggcatcatttattgcctgctcattgcagttgaaacgcgatatccgtttcaagacccgggtatggatggtactttggagatatgagcacttgccagacttcccggtcggagacttaaaaattccgataagaggttacggacgacggaccttataagtggatcgtctagtggtggcgccgatcaaaacagttccgcccccggctttgctcaaagtagcaaagcagctttatctcgggttgcggaggattttctagaaaaccgcaattttgcaggagagaacatatggccatcatcaagccgcatgtgaacaaaaataggacaacctcgccgatagagagaccggagtctctcatagaggaaatgagcggcagtcatccgccgagtggttttaccaacctggatctcgctatgatcgagctggaggactttgtccatcggtgcccgctcccagaagacaatcttgctggtcagaaggagtgagacgatggatccgtctagcaatgagaatgtctatgtgggtcgcggtcacaacatcgaaaatgatgatgacactgaccccaggcgttggaagaaggcgaatatcagttccaacaccatctccgatattcagatgacgaatggcgaagacgtacaatcagggagccctacccgaacggaagttgtaagcccacgtctggattatggatcggtcgactcctcctccagcctttattctggcagcgagcacggaaatcaagctgagattcaaaaagagctgtccgtcttgttctcgaacatgtctttgccaggcaacgatcggcgcccggacgaatacattctcgtgcatcaaacgggacaagatgcttttactggtattgccaaaggcaacctcgaccaaatgcccaccaaggcggaatttaacgcgtgctgccgtctctacagggacggagccggtaattactacccgccacctctcgcattcgacaagattagcgttccagagcaactggaggaaaaatgggggatgatggaggcgaaggaacgtaacaaactgcggtttcagtacaagttggacgtatggaatcatgcgcacgctgatatggggatcacgggcacagagatcttttatcaaacagataagaacataaagctcgaccggaattataaactaagacctgaagaccgatacgtacaaacagaaaaatacgggcgccgggaaattcaaaagcgatatcaacacgaactccaggctggttcgctgctgcccgatattatgatcaaaactccccaaaatgacatccacttcgtgtacaggtttgccggcgacaattacgccaacaaacagttcagcgagtttgaacacaccgtcaagcgcaggtatggcgacgagactgagatcaaattgaagtcaaagtcaggcattatgcatgactcgaaatatctggaatcctgggaacggggcagtgcggatattcgcttcgcggaattcgttggggaaaatagagctcacaatcggcagtttccaactgcgacagtaaatatgggacagcagccagacgggcagggcggtttgacccgcgaccgtcatgtgagcgttgacttcctaatgcaaagcgcacccaattcgccttgggcgcaagctttgaaaaagggagaactgtgggatcgcgttcagttgcttgctcgcgacggcaaccgctatctgtcgccgcccagattggaatattctgaccctgcacatttcaccgagttgatgaaccgggttggtttacccgcatcgatgggtcggcaaagccatgcggctagtatcaaattcgaaaagtttgacgcgcaggcagcggttattgtcttaaatggcccagagttacgtgacattcatgacttgtctcctgaaaaactgcaaaatttgtccaccaaagatgtcatcgtcgccgatcgcaatgagaatggtcagagaactggcacgtacaccagcgtcgcggaatatgagcgcttgcagttaaggctgccacccgatgcagcgggggtgcttggtgaagcaactgacaaatattcacgtgatttcgttcggccagagccggcgtcgcgtccaatcagtgacagccgcaggatatacgaaagtcgaccgcgtagccaaagcgtcaacagcttttgacgttcctgctgccgcgtcaacgaggaagctcgtttgacccgggtttgccaatgaaagggctcaatcatggtgaacactacaaagaaaagttttgcgaagtcgcttacggcagatatgcgccgttctgctcagcgcgttgtcgagcaaatgcgaaaagcattgattaccgaagaagaggcgctcaagcggcaagccagactggagagtcccgataggaagcgaaagtatgctgctgatatggcgatagtcgacaaactcgacgtagggtttcgaggcgaaataggctataaaattcttggaaataaccggcttcgagtagacaaccataaagaattaacgcgtgagcacggtagacttcgcaaaaccaaaacggttctgaagcgtaacccggtgacgcaggaagtctatttgggtttatatgaaaggaagtcctggttaagtgtcagcagccatttgtatgctgcggacggcacactccgcatgaagcacgtgaaatacaaagacggacgttttgaggaaaaatgggagcgcgacgaaaatggcgacctgatccgcacaaggtacgccaaccgtggcaggctctttcaacctgtatccgagaaaatgggcgcgccgtatcggagcggccctgacgaccggctctatcgcgatctaacccgtcgaaacggtttcagacgggagacattcgaacgggacgatcacggaaacctcgagcgtatcggcagcaaccatgtcggcttttccaagatttcagtgaaggcacccaatcgtcaaacctcccagacgaagattcaaaaacttggtggcgctttcaacaaatcttttaggtcccttctggacaaggagggcaatgaaatgggccgcgatattttgagccatcgacggctctataacaagcggtctgctgtctacgatgaagctaccggacaattgaagagtgccaagcataccttcggcaagatctacaggagcgaaaccgaatatctcagcgcgggcctcaagaaggtttcaaaaaagatactcggggtgacggtctaccggaaatttgcggcgctcagcgagcgagaatccgaggctgagagactgcgtagttttgaatccggtgcgcatcgccagatctggcaggagcgggcagcgattcccggttcgcccctcccggagactgatgacattcatttcgcacagcagtcgcacctagccaaagccaaccctgatcacgtcgaagctgacgtcacgcgtgtgacagatcaacatgttgatgttgctggacaaacatcatcgtctccccaacggaacttggaaggatggttagattctcaatcacgatacaagccagcaaacatgctgttgtcaaatccagaccttcaagcgaacggacctcgcccatacgaagggttagctcatctcaccctccggcgcgataatgaatctgacgggcacaaggagaatgatcagcggctgcgacatttctcccagccagagccgttggtgttaccgcatcccgggtcgccggaaataactaaggtgtttggctcgcggggagagccggcacacccgagtggaaatctgcacacggcggttggagaaacggcttgcgaaggaccggtgatgtcttcatcctcggacaatcatcagccagctccaggacagcaagaacttttaagtttccttcataatgcgccagccccagtttctgtggcaatacatgatgatcaagagcgacttgcgggggaggcgcccggcggctctttcagaggtagctcagggcgaacgagttcaatgtcggagagtatcttcgacgaagatgtacaagggcatttggtacgggattattcgatcaatactactaacgggtttattgacccgcaatcgttgttcggtgaaccggacttatcgagaggtccaaaatcggggccagaaattccatcggaagattaccatttgtcagcttcggaacaggaaaatttgctgaatcaattgcttagtgtgccactgccggttccttcaccgaagcccgaatgcgcgaggtctatgattttcgaaggttcacgttcaagagagcgttccacctccagagggttctaaggtggaacgcgtctaggcttgcctatcatcaccacctcatgatggtcgcggagaccttcgaagagatatccgacatcattggagaagctgggccggaaactagtgtacctcgcgaatgcatctagatccaatccaatatgcatggcatgccgaatccatgtgggagtttattcttgacacagatatttatgatataataactgagtaagcttaacataaggaggaaaaacatatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaataatgtctaacaattcgttcaagccgacgccgcttcgcggcgcggcttaactcaagcgttagatgcactatacgtaccaatccaaatcggatccccctgcagggctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatctatcgatggtacctatctgcagacggaagggattcctgttaattaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctcgcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggcagcgcaggccgcccagcggcaacgcatcgaggcagaagcacgccccggtgaatcgtggcaagcagccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcaggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatgcatataattgtggtttcaaaatcggctccgtcgatactatgttatacgccaactctgaaaacaactttgaaaaagctgttttctggtattt

SEQ ID NO: 3 - DNA, 17189 bp, synthetic construct
  misc_feature 1..17189: note = PHP81858
  source 1..17189: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccacgcgtgaagagcggttaccagagctggtcacctttgtccaccaagatggaactgtcttcggccagaatggccatctggattcagcaggcctagaaggccatttatcgctatcaactttgtatagaaaagttgggccgaattcgagctcggtacggccagaatccggtaagtgactagggtcacgtgaccctagtcacttaaattcggccagaatggccatctggattcagcaggcgaggccggccacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccggcggatctcgactctagaggatcgctcaggaaggccgctgagatagagccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggttaccagagctggtcacctaagtgactagggtcacgtgaccctagtcacttattcccagcttcaagtttgtacaaaaaagcaggctccggccagaatggcccggaccgggttaccgaattcgagctcggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggcctccgggcacccagctttcttgtacaaagtggccgttaacggatcggccagatccctatagtattttaaaattgcattaacaaacatgtcctaattggtactcctgagatactataccctcctgttttaaaatagttggcattatcgaattatcattttactctttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaagcttggtcacccggtccgggcctagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatccagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaattccggtccgggcctagaaggccagcttcggccgccccgggcaactttattatacaaagttgatagataaatcagatatctggttcctaaggacccggcggaccgattaaactgattcggtccgacgcgttacctaataacttcgtatagcatacattatacgaagttattagctaactaactagtaagcttggtcacccggtccgtcaatattctgattccgatgacttcgtaggttcctagctcaagccgctcgtgtccaagcgtcacttacgattagctaatgattacggcatctaggaccgactagtggcgcgccgactagctagctaaggtacgacttagctatagttccggacttaccggcgccgctagcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatcaattcgctagcgaagttcctattccgaagttcctattctctagaaagtataggaacttcagatccaccgggatccaccatggttgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggatccaccatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccaccggtcgccaccatggccctgtccaacaagttcatcggcgacgacatgaagatgacctaccacatggacggctgcgtgaacggccactacttcaccgtgaagggcgagggcagcggcaagccctacgagggcacccagacctccaccttcaaggtgaccatggccaacggcggccccctggccttctccttcgacatcctgtccaccgtgttcatgtacggcaaccgctgcttcaccgcctaccccaccagcatgcccgactacttcaagcaggccttccccgacggcatgtcctacgagagaaccttcacctacgaggacggcggcgtggccaccgccagctgggagatcagcctgaagggcaactgcttcgagcacaagtccaccttccacggcgtgaacttccccgccgacggccccgtgatggccaagaagaccaccggctgggacccctccttcgagaagatgaccgtgtgcgacggcatcttgaagggcgacgtgaccgccttcctgatgctgcagggcggcggcaactacagatgccagttccacacctcctacaagaccaagaagcccgtgaccatgccccccaaccacgtggtggagcaccgcatcgccagaaccgacctggacaagggcggcaacagcgtgcagctgaccgagcacgccgtggcccacatcacctccgtggtgcccttctgaagcggccgcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggccgccaccgcggtggagctcgaatgcggccctagcgtatacgaagttcctattccgaagttcctattcttcaaaaagtataggaacttctgtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 4 - DNA, 7122 bp, synthetic construct
  misc_feature 1..7122: note = PHP89030
  source 1..7122: mol_type = other DNA
cccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccggccagaatggcccggaccgggttacgttaccgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcacccgtacgcgtgcgatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaaattccggactagtggcgcgcctcaacgagatcatgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgaccaccggtgcctaggcccgggcacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaagg

SEQ ID NO: 5 - DNA, 6805 bp, synthetic construct
  misc_feature 1..6805: note = PHP89179
  source 1..6805: mol_type = other DNA
cccagtcttccgactgagcctttcgttttatttgatgcctggcagttccctactctcgcgttcatggagctccaaataatgattttattttgactgatagtgacctgttcgttgcaacaaattgataagcaatgcttttttataatgccaactttgtatagaaaagttgggccgagctcggtacggccagaatggcccggaccgggttaccgaattcttaccctagctccctgcggctgttacgcggtcccccatcaatcttctgttcttgcggttgtagcctgtgtaacagtgctagagtatgtatgataaataggttttaagtctgcttacatgacattttttattgtggaagagacatataaaaattagagagagtggttctcatgcaacggcggacggcccggtgctaaaagagcttcaagacaaaataatgaaacaggaagagagtagatttatctaagagccaactttattatatgaatgtgtttattgttggctttagatgatatggtaaggagttagagctaataatagataggctctattattattattattaattaaactcgctctaaggaggaaagtgggaggaagggacgaggacgaagactactggaagcatcgtgcatggatgatggatgtggtgtctcttaatgtaggtggccggaggatgtacgtgttaattgcgcgataagcactcagatccaaccgcaaactacctccacactgacacactgatagagagaaagagagacctccgacgactgccgccgcagatgagccacgtacgtatacgacgtctgccggccggctcaggctgccgccatcaccctgctcgaaagtcgcgttaggcggcgccagctacataggagtatctagtctagccagttagtatactactactgcgctgatgatgaattaactctgcatagatactgtacttgcctccctccaacacccaaccacctcctgctcggctcttaataacttggacacggatcgatgccatccaaggaagaacacgacgacgacgacggaacatccaccatgcaagcttgcatccatacgccgatacgcgtgcatccatccatccaccattatttccattttccaccgatcacacgtacacaggcctatttaaggagcgacatcccactgcaactctcctcaccactcatcaccagctagctctagcaaagcacttgccatctaccgaccgccgcattccaaacagcccgacgagctagcagagcggcaggcacctccctcctcaaggaacccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcgggaatttaaatgctcggcgttaggcttttagtccgctaggtggctagaccgaggttaggacgctagttcactaggggcctagtggtgttttaagacgtcacggtcacctgaagaaactatgtgctgtagtatagccgctgcccgctggctagctagctagttgagtcatttagcggcgatgattgagtaataatgtgtcacgcatcaccatgcatgggtggcagtgtcagtgtgagcaatgacctgaatgaacaattgaaatgaaaagaaaaaagtattgttccaaattaaacgttttaaccttttaataggtttatacaataattgatatatgttttctgtatatgtctaatttgttatcatccatttagatatagacaaaaaaaatctaagaactaaaacaaatgctaatttgaaatgaagggagtatatattgggataatgtcgatgagatccctcgtaatatcaccgacatcacacgtgtccagttaatgtatcagtgatacgtgtattcacatttgttgcgcgtaggcgtacccaacaattttgatcgactatcagaaagtcaacggaagcgagtcgacctcgagggggggccccggccgaagcttcggccggggcccatcgataacgagtatcggtccccattgacacatctactcctaacgctaacaacattcttcacttctagtgtatggcatcgacaattggtactttaatcttgggtcttcacttttacaactaaacaacccaaatataaatattatttgagtgggtttggtgttcatcagaacattcccatctttggtaatgctacctagcataaaagtgagctatcacgttgggcagcccattccatgcaatttttaccttcctcaaaaaaaatctttgatatatttatggtataataatttaaacttcacattgatcataattttttcagcaaataagttccataatttggtgtttgcaatatcgaaattgttactcatagggtccagttgatttaatgacattagtgttgttaagttcacatgcttcaaagaattctacatgtctacaatgctgcatttgtctgtaaaatttgggttcttgtaaaaaatacatgtgttgaatatttaccaaaaaaatataatgcgatatgactttgtgtgagcatgtttggacacagttacaagtttacttctaaacttatattatgttttgcttcgtgcaagtacaagtgatttgaatatccaaggccaaagcatgtgtccatcactatatcaaatcaacataccatatatgtatatactcaacaaaaagcacaaataatcacatctatgttataaacacacccccacacaaccaaacattaacattcaataggttgtgctaggacatcattgggatgatgaagtaatattgtattatctatgtattgaccaagcatgagtttaaaattacgatttgatgttttcagttatttatatgatgaacatagttcagaaatatatcgagataaatctcaatcaatatattgattatttctaggaaacacatgccggtatgagggcaccattgtcgagtacatcttaagtgagttaacggatcctctagagtcgacctgcaggcatgcccgcggatatcgatgggccccggccgaagcttcaagtttgtacaaaaaagttgaacgagaaacgtaaaatgatataaatatcaatatattaaattagattttgcataaaaaacagactacataatactgtaaaacacaacatatgcagtcactatgaaccaactacttagatggtattagtgacctgtagaattcgagctctagagctgcagggcggccgcgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaagg

SEQ ID NO: 6 - DNA, 19770 bp, synthetic construct
  misc_feature 1..19770: note = PHP93739
  source 1..19770: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacctagctaataacttcgtatagcatacattatacgaagttatggttaccgaattcgagctcggtaccctggttcacaaactcaatacaacacacagtggagtagggtattacgctccggcggcccgaaccactctaaatccttgtgtcctcgtgtgctcttccaccattaatgactgtaattgcagggacacgaagagttgtagcgcttttgccaacaagatccgctatatgctagtagagtggaacctgcacagcaacaagcatccaataatctgagaataagaattatgtgtggtgatggttctcatctctgcaactcgccattttcgcgagagaaccaaatccaagttggtgaagatcatgatttgggctcatgggctggcatagtagctattacctttgcaattgtggttcgaatctatccgatattctccttttcagggagagctaatgcttatgcttattgggtattttggtttgacactgcttcacgcccaaaaaaaggcaactatgtctgagttggatatggcccaaaaaaaggcagctatgtctgagttggatatggaagtcttctttcgtttagggtgaagtaagaccaagctcatgagcttattatcttaggtcggaacaaattagttgataggcatggcacatttatcaaattataatgattttaacagaaaagcaacacctctttttcagcagcaccagctttgcaagctgcaattgacactccaagcatagtatttgctccaaactcagcctgcatttcgcacaatatcatatctatttccatttggataaaatatattttccagaaatatgggaacagaatcaagatacagaaaaaagttgtggtagtagtggtaagtggaaacacaagtacagtgaaaagatagcattatttctgaacagtcaaattaaccgaaataagtagtatcacatactgcccctaaattgactacacccaatgtaaagaattgcttgagcatattgagccatcagttgtttgtttgtgtaacggatttgacaatgcagagttcaggagcaaaaaaccgcaaaggagagaaagagggaaaggatctcctagtatcccaccttgtggcgcgccctgtcaagtgcatgatggcctggtcgatctgcgtctgctgctgcggttccatcccaacgagtgcctccgacaccttgtcgttgatcacccgcaccgcgtaggcgacccccctggcgaggaacttccgcctctcggaggccccggccgcgtcggcggcggcgccctcgagcgcgcccacgctagccgtagatgctcggtgcacggccttgttggtgtgcaactcgacctcaacggccggtgcgctacgcccgtcaaggatctggcgcgcccgcaccctcgtgatcacggtgggcgcctcccgcctcatgtggccgacgatgaaaagcgccgggttaggggccttggcgcgcaccgccgtattcagggcctcctcgctcttgcgcgagagcaggtgcttccccaggtactcctgcactgacaaggctgccacagcggtggaatttgtcctcacgacttcgccacgtcgtcctcacctccctggcttcccacgactccgccgtatcgaggtgctcggtgggtcgtcttaatccgaagtccgaactaggaagacgagacagagaggtctgaggaatgggcctcgtttgatgttgagctgaattatttttccagcataagcccaggtcttggtccatgaacaaaattactagaaacccagcccagtactacgatctaaaagagggactgccactggatagctctctctagcattctccacgctccaatacagcggcgtcgtgtctatccgggtctatccgcgaacacgtgagaactctccagaaactgctttctcctccacttcatctctctcgctttccctctataaaaagaccccttctaggaattgagggagacagcaagcaccaagcagcgatccgaagctcaatcaattcactcaaacctcttccccaaatcttcgattagatcttcgttgacaagaagactagaaccgaacctgacatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgatccgatcgaggccggccacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccggcggatctcgactctagaggatcgctcaggaaggccgctgagatagagccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcgggaatttaaatgctcggcgttaggcttttagtccgctaggtggctagaccgaggttaggacgctagttcactaggggcctagtggtgttttaagacgtcacggtcacccggtccgggcctagaaggccagcttcaagtttgtacaaaaaagcaggctccggccagaatggcccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagcactgccttcatacgctatttatttgcttggtactgtttcttttgtcgatgctcaccctgttgtttggtgttacttctgcaggtcgactttaacttagcctaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaatattggtaccgtcatgggtcgtttaagctgccgatgtgcctgcgtcgtctggtgccctctctccatatggaggttgtcaaagtatctgctgttcgtgtcatgagtcgtgtcagtgttggtttaataatggaccggttgtgttgtgtgtgcgtactacccagaactatgacaaatcatgaataagtttgatgtttgaaattaaagcctgtgctcattatgttctgtctttcagttgtctcctaatatttgcctgcaggtactggctatctaccgtttcttacttaggaggtgtttgaatgcactaaaactaatagttagtggctaaaattagttaaaacatccaaacaccatagctaatagttgaactattagctatttttggaaaattagttaatagtgaggtagttatttgttagctagctaattcaactaacaatttttagccaactaacaattagtttcagtgcattcaaacacccccttaatgttaaggtggttctatctaccgtctcctaatatatggttgattgttcggtttgttgctatgctattgggttctgattgctgctagttcttgctgaatccagaagttctcgtagtatagctcagattcatattatttatttgagtgataagtgatccaggttattactatgttagctaggttttttttacaaggataaattatctgtgatcataattcttatgaaagctttatgtttcctggaggcagtggcatgcaatgcatgacagcaacttgatcacaccagctgaggtagatacggtaacaaggttcttaaatctgttcaccaaatcattggagaacacacatacacattcttgccagtcttggttagagaaatttcatgacaaaatgccaaagctgtcttgactcttcacttttggccatgagtcgtgacgcgacttattgaactagtctagggcgcggctaacaacctagcttagattgccttgtctaaacccttaggctaacgcgccactctagaaggccgatctcccgggataacttcgtatagcatacattatacgaagttatgaattccggaccgggttaccgaattcctgcccttaaggccaattgttcaagattcattcaacaattgaaacatctcccatgattaaatcagtataaggttgctatggtcttgcttgacaaagtttttttttgagggaatttcaactaaatttttgagtgaaactatcaaatactgattttaaaaaatttttataaaaggaagcgcagagataaaaggccatctatgctacaaaagtacccaaaaatgtaatcctaaagtatgaattgcattttttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggatatgtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcaggtcgactttaacttagcctaggatccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagggtaccaatggccagttaacagatccagctgctgctgttctagggttcacaagtctgcctatttgtcttccccaatggagctatggttgtctggtctggtccttggtcgtgtcccgtttcattgtgtactatttacctgtaatgtgtatccttaagtctggtttgatggtgtctgaaacgttttgctgtggtagagcagcatggaagaactataatgaataagtgatccctaatcattgtgtccaaattttgcttctgctatacccttttgtgctgtttcttatgttttgcttaaaaatttgatctgacaaacaaatttgtctaaattatgctcttgttctgactgtgtactgtacacatttgtattgctaccggtttggcacatagttttctctttgattgcagatcaaacccttctgattgcagattgcttggctatggctgatgtgctcactgttcttctttagcttgtatattgctgatggtggcttggctacggctgaaaaagttgctgctgcgccgacttggcaactgttgctccagtaatgctttgtcctctcttcctagccgccgacttggcacttggcagctgttgctcctgtaatgcgttgttatctcttatctcttgagatgtactatgattttgaggctgtttgacttgtgccagattgccagtgctgatgctatattatctcaggtttttatgctagtatatgttttactgtgtgctgcctgtgtcgagttacgtaatctattgaaccgctatgcgtgttgatgccctgttgttgcttgccagattgatgcccctgttgcttgcagcttgcaagctgtagtgaaatcctaaaattcgaaaccaaatctattctgaacttttgaagatctgtgatgtccggaagaaaagattcaaacagataaacgggactctccagaatctataaaataaatgatcgacaaacgctttgctttggtaggcttcctagttagttagcggaccgggatccgacggcgtcttcaccgccccgccgccgcccgagccggacttgtcgctgccgccgccgctgctcatcttgcgcgcttgtgtccctccctcagtccctgcgttcgcggatcgcgggcgggtgcgacccgtttggactccggccggtagccttcgtgccgacctcgctcggtccaccggtttgtcggggacccacatgtcagtgaaggtacgcgtccacgtccacagaccctactaccccagacgagtccaaatcgctgttccctcacactctcgtgtaccagcgcgcggcggaaagcacctttccaacaaccgccgacacgtcggtcccacgtcggcatgctccaccggtcagtgtttggcgtgtacggctcccggcgacgaacgagtgtggcctaagctttcctactcccatcccatctgagccacacatctctctcacgccacgcgcacaaaacagccaggctcccacgcgcatcaaccggctctccctttccgtgccactcgcccccaagccccaaaccctcgccgccgcctccgagacagccgccgccacctaggccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgagccccgcaagggtgctgacatcctcgtcgagtccctcgagcgctgcggcgtccgcgacgtcttcgcctaccccggcggcgcgtcgatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtcccgatggtcgccatcacgggacaggtggcgcgacgcatgattggcaccgacgccttccaggagacgcccatcgtcgaggtcacgcgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagccgtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgacttcgaataggtacccggggatcctctagtgccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattcaatcactagagtcgacctgcaaagcttcggtccgggcctagaaggcccgcgccacttacgcttgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctgcgtttaaacgtgcaagcgctactagacaattcagtacattaaaaacgtccgcaatgtgttattaagttgtctaagcgtcaatttgtttacaccacaatatatcctgcca

SEQ ID NO: 7 - DNA, 20881 bp, synthetic construct
  misc_feature 1..20881: note = PHP96037
  source 1..20881: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacctagctaataacttcgtatagcatacattatacgaagttatggttaccgagctcccctggccgaagcttacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactcccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcgggaatttaaatgctcggcgttaggcttttagtccgctaggtggctagaccgaggttaggacgctagttcactaggggcctagtggtgttttaagacgtcacggtcaccgcggccgcatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagcaacgagatcatgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgaccgtcgacccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatcccattcgaaacgcgtggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcaccggcgcgccgcgacttattgaactagtctagggcgcggctaacaacctagcttagattgccttgtctaaacccttaggctaacgcgccactctagaaggccgatctccccgggttaccgagctccaccttgctagttggatattacctcttctcttcaaagtatccttgaacgctcaccggttatcaaatctctacactatagctctgtagtcttgctagatagttagttctttagctctctctagaggttcacaaactcaatacaacacacagtggagtagggtattacgctccggcggcccgaaccactctaaatccttgtgtcctcgtgtgctcttccaccattaatgactgtaattgcagggacacgaagagttgtagcgcttttgccaacaagatccgctatatgctagtagagtggaacctgcacagcaacaagcatccaataatctgagaataagaattatgtgtggtgatggttctcatctctgcaactcgccattttcgcgagagaaccaaatccaagttggtgaagatcatgatttgggctcatgggctggcatagtagctattacctttgcaattgtggttcgaatctatccgatattctccttttcagggagagctaatgcttatgcttattgggtattttggtttgacactgcttcacgcccaaaaaaaggcaactatgtctgagttggatatggcccaaaaaaaggcagctatgtctgagttggatatggaagtcttctttcgtttagggtgaagtaagaccaagctcatgagcttattatcttaggtcggaacaaattagttgataggcatggcacatttatcaaattataatgattttaacagaaaagcaacacctctttttcagcagcaccagctttgcaagctgcaattgacactccaagcatagtatttgctccaaactcagcctgcatttcgcacaatatcatatctatttccatttggataaaatatattttccagaaatatgggaacagaatcaagatacagaaaaaagttgtggtagtagtggtaagtggaaacacaagtacagtgaaaagatagcattatttctgaacagtcaaattaaccgaaataagtagtatcacatactgcccctaaattgactacacccaatgtaaagaattgcttgagcatattgagccatcagttgtttgtttgtgtaacggatttgacaatgcagagttcaggagcaaaaaaccgcaaaggagagaaagagggaaaggatctcctagtatcccaccttgtggcgcgccctgtcaagtgcatgatggcctggtcgatctgcgtctgctgctgcggttccatcccaacgagtgcctccgacaccttgtcgttgatcacccgcaccgcgtaggcgacccccctggcgaggaacttccgcctctcggaggccccggccgcgtcggcggcggcgccctcgagcgcgcccacgctagccgtagatgctcggtgcacggccttgttggtgtgcaactcgacctcaacggccggtgcgctacgcccgtcaaggatctggcgcgcccgcaccctcgtgatcacggtgggcgcctcccgcctcatgtggccgacgatgaaaagcgccgggttaggggccttggcgcgcaccgccgtattcagggcctcctcgctcttgcgcgagagcaggtgcttccccaggtactcctgcactgacaaggctgccacagcggtggaatttgtcctcacgacttcgccacgtcgtcctcacctccctggcttcccacgactccgccgtatcgaggtgctcggtgggtcgtcttaatccgaagtccgaactaggaagacgagacagagaggtctgaggaatgggcctcgtttgatgttgagctgaattatttttccagcataagcccaggtcttggtccatgaacaaaattactagaaacccagcccagtactacgatctaaaagagggactgccactggatagctctctctagcattctccacgctccaatacagcggcgtcgtgtctatccgggtctatccgcgaacacgtgagaactctccagaaactgctttctcctccacttcatctctctcgctttccctctataaaaagaccccttctaggaattgagggagacagcaagcaccaagcagcgatccgaagctcaatcaattcactcaaacctcttccccaaatcttcgattagatcttcgttgacaagaagactagaaccgaacctgacatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgatccgatatcgatcaccttgctagttggatattacctcttctcttcaaagtatccttgaacgctcaccggttatcaaatctctacactatagctctgtagtcttgctagatagttagttctttagctctcgggccctggccgaagcttcgtgcgcacgataacttcgtatagcatacattatacgaagttataacagcggaccgggttaccgaattcctgcccttaaggccaattgttcaagattcattcaacaattgaaacatctcccatgattaaatcagtataaggttgctatggtcttgcttgacaaagtttttttttgagggaatttcaactaaatttttgagtgaaactatcaaatactgattttaaaaaatttttataaaaggaagcgcagagataaaaggccatctatgctacaaaagtacccaaaaatgtaatcctaaagtatgaattgcattttttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggatatgtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcaggtcgactttaacttagcctaggatccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagggtaccaatggccagttaacagatccagctgctgctgttctagggttcacaagtctgcctatttgtcttccccaatggagctatggttgtctggtctggtccttggtcgtgtcccgtttcattgtgtactatttacctgtaatgtgtatccttaagtctggtttgatggtgtctgaaacgttttgctgtggtagagcagcatggaagaactataatgaataagtgatccctaatcattgtgtccaaattttgcttctgctatacccttttgtgctgtttcttatgttttgcttaaaaatttgatctgacaaacaaatttgtctaaattatgctcttgttctgactgtgtactgtacacatttgtattgctaccggtttggcacatagttttctctttgattgcagatcaaacccttctgattgcagattgcttggctatggctgatgtgctcactgttcttctttagcttgtatattgctgatggtggcttggctacggctgaaaaagttgctgctgcgccgacttggcaactgttgctccagtaatgctttgtcctctcttcctagccgccgacttggcacttggcagctgttgctcctgtaatgcgttgttatctcttatctcttgagatgtactatgattttgaggctgtttgacttgtgccagattgccagtgctgatgctatattatctcaggtttttatgctagtatatgttttactgtgtgctgcctgtgtcgagttacgtaatctattgaaccgctatgcgtgttgatgccctgttgttgcttgccagattgatgcccctgttgcttgcagcttgcaagctgtagtgaaatcctaaaattcgaaaccaaatctattctgaacttttgaagatctgtgatgtccggaagaaaagattcaaacagataaacgggactctccagaatctataaaataaatgatcgacaaacgctttgctttggtaggcttcctagttagttagcggaccgaagcttcggtccgggcctagaaggccagcttcggccgcccctcggtaccctgggatctgcccttactttaacgcctctaaccaacacccctttatctttataaggaacaataaacagaatttgccccactgttctaaatcacctaataatatccccagctaaaaacaataaaggtttcctagaattaagacaagcatgactgttcctccaggagggtttggaacattgttgcagtcttgcagatacgggcgaagggtgagaaacagagcggagggctggaggtgacctcggtagtcgacgccggagttgagcttgacaacgacggggcggcccctgatggacttgaggaagtccgatggcgtcttcaccgtcccgccggcgcccgaggcgggcctgtcgctgccgccgccgccgctgctcatcttgcgcgctgtgcccccggcggtgtccctgtgttgcggatcgcgggtgggccaggtggatgcgagggcgacccgtttggactccggccggagccgccggatccctggtcggtgtcagtgccgtttactctgggccccacgtgtcagtaccgtctgtagatgacaacaacccgtcgtccacagtcatgtccaaaatatcctttcttcttttttttcgattcggatatctatcttccttttttttttccaaaaatcttcttgacgcaccagcgcgcacgtttgtggtaaacgccgacacgtcggtcccacgtcgatagaccccacccaccagtgagtagcgtgtacgtattcgggggtgacggacgtgtcgccgtcgtcttgctagtcccattcccatctgagccacacatctctgaacaaaaaaaaggagggaggcctccacgcacatccccctccgtgccacccgccccaaaccctcgcgccgcctccgagacagccgccgcaaccatggccaccgccgccgccgcgtctaccgcgctcactggcgccactaccgctgcgcccaaggcgaggcgccgggcgcacctcctggccacccgccgcgccctcgccgcgcccatcaggtgctcagcggcgtcacccgccatgccgatggctcccccggccaccccgctccggccgtggggccccaccgagccccgcaagggtgctgacatcctcgtcgagtccctcgagcgctgcggcgtccgcgacgtcttcgcctaccccggcggcgcgtccatggagatccaccaggcactcacccgctcccccgtcatcgccaaccacctcttccgccacgagcaaggggaggcctttgccgcctccggctacgcgcgctcctcgggccgcgtcggcgtctgcatcgccacctccggccccggcgccaccaacctagtctccgcgctcgccgacgcgctgctcgattccgtccccatggtcgccatcacgggacaggtggcgcgacgcatgattggcaccgacgccttccaggagacgcccatcgtcgaggtcacccgctccatcaccaagcacaactacctggtcctcgacgtcgacgacatcccccgcgtcgtgcaggaggctttcttcctcgcctcctctggtcgaccagggccggtgcttgtcgacatccccaaggacatccagcagcagatggcggtgcctgtctgggacaagcccatgagtctgcctgggtacattgcgcgccttcccaagccccctgcgactgagttgcttgagcaggtgctgcgtcttgttggtgaatcgcggcgccctgttctttatgtgggcggtggctgcgcagcatctggtgaggagttgcgacgctttgtggagctgactggaatcccggtcacaactactcttatgggcctcggcaacttccccagcgacgacccactgtctctgcgcatgctaggtatgcatgggacggtgtatgcaaattatgcagtggataaggccgatctgttgcttgcacttggtgtgcggtttgatgatcgcgtgacagggaagattgaggcttttgcaagcagggctaagattgtgcacgttgatattgatccggctgagattggcaagaacaagcagccacatgtgtccatctgtgcagatgttaagcttgctttgcagggcatgaatgctcttcttgaaggaagcacatcaaagaagagctttgactttggctcatggaacgatgagttggatcagcagaagagggaattcccccttgggtataaaacatctaatgaggagatccagccacaatatgctattcaggttcttgatgagctgacgaaaggcgaggccatcatcggcacaggtgttgggcagcaccagatgtgggcggcacagtactacacttacaagcggccaaggcagtggttgtcttcagctggtcttggggctatgggatttggtttgccggctgctgctggtgcttctgtggcaaacccaggtgtcactgttgttgacatcgatggagatggtagctttctcatgaacgttcaggagctagctatgatccgaattgagaacctcccagtgaaggtctttgtgctaaacaaccagcacctggggatggtggtgcagttggaggacaggttctataaggccaacagagcgcacacatacttgggaaacccagagaatgaaagtgagatatatccagatttcgtgacgatcgccaaagggttcaacattccagccgtccgtgtgacaaagaagaacgaagtccgcgcagcgataaagaagatgctcgagactccagggccgtacctcttggatataatcgtcccacaccaggagcatgtgttgcctatgatccctagtggtggggctttcaaggatatgatcctggatggtgatggcaggactgtgtactgacttcgaataggtacccggggatcctctagtgccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattcaatcactagagtcgacctgcaaagcttcggtccgggcctagaaggcccgcgccacttacgcttgattccgatgacttcgtaggttcctagctcaagccgctcgtgtccaagcgtcacttacgattagctaatgattacggcatctaggaccgactagttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatctgcgtttaaacgtgcaagcgctactagacaattcagtacattaaaaacgtccgcaatgtgttattaagttgtctaagcgtcaatttgtttacaccacaatatatcctgcca

SEQ ID NO: 8 - DNA, 20950 bp, synthetic construct
  misc_feature 1..20950: note = PHP97334
  source 1..20950: mol_type = other DNA
gtttacccgccaatatatcctgtcaaacactgcggccgcacttgaccgcggatggagccggagctgtggtgaggccatcgacgggaggtgggggagaacacagtctcagtgttctttgtgagtccgcaaggtttttagcttttctgtattaattaaggttgctgttggctgtagaatacggtcgtggaagagctgtatcggtagctactacgtaagctattatatactggaagtctggagctcgtcgatctccggtcgtcttcctccatttctcctcgtcttgctttgactgctgctaccatttgcttctgttttctgatgaagcatgccatgtcggccaccagtgtttgtgtggtttttgcagggaatggctttcaagtgctgtgattgaatggccctcgtcgtcctcggctgagctcacatggacgcgtcatgtgagtgagatatatgatcatatgatgagtggggttcgggtgagtgcgtacttgaagtaggatcccgctttcactcattcattcattggcctttaaaccgggaaattttcgcgaggagatggagggaagcaagcagggtgacgagtgcagagtgccgcgagcgctttgtcaaaaggcaccgccgtaggcaaaaaaatcaatggccctgactgactcgatggtagtactactgtagagactagagagggttgctccccatatcttcgtgtgacttttactccagcattgcatttccgtcattgagacgttaattaatagcagctttcgggacagatgactgtggccatgtttcgtcacggccgcttcgtgccagctgggggtactatagtactgtgattttgttagtaaaaactttcattaggccaccattgacgaccgggccttcgcttttgctgcgctaattaaccatttcccggcgaccgaagggaacatgaacatgccccgacacatgagggcatgaggactagtgttctccctgtcgttcgttgacttgactagtgatagtttaaacgctcttcaactggaagagcggttactaccggttcactagctagctgctaatcgagctagttaccctatgaggtgacatgaagcgctcacggttactatgacggttagcttcacgactgttggtggcagtagcgtacgacttagctatagttccggacttacctagctaataacttcgtatagcatacattatacgaagttatggttaccgagctgttaccaagcggccgcacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactcccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagaacagatcattcgacaaagcagcattagtccgttgatcggtggaagaccactcgtcagtgttgagttgaatgtttgatcaataaaatacggcaatgctgtaagggttgttttttatgccattgataatacactgtactgttcagttgttgaactctatttcttagccatgccaagtgcttttcttattttgaataacattacagcaaaaagttgaaagacaaaaaaaaaaacccccgaacagagtgctttgggtcccaagcttctttagactgtgttcggcgttccccctaaatttctccccctatatctcactcacttgtcacatcagcgttctctttccccctatatctccacgctctacagcagttccacctatatcaaacctctataccccaccacaacaatattatatactttcatcttcaactaactcatgtaccttccaatttttttctactaataattatttacgtgcacagaaacttagcaaggagagagagagcgggaatttaaatgctcggcgttaggcttttagtccgctaggtggctagaccgaggttaggacgctagttcactaggggcctagtggtgttttaagacgtcacggtcactacgaatcgaccgaagcttgcatgggccgcatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagcaacgagatcatgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgaccgtcgacccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatcccagggccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacctagtggtacctgacatcttatagtctgcaacctctcgtgtctgaattcctatctttatcaagtgttattgcttccacgactataggacagctttcgtcgaaagcttttgctcatgtgatctcgaaggattcatctagtctgatttttcgtgacttgtatcggttttattggattcatccaacatatatcaataaaaaatgagttgtgtttcctttcttcctagttcagttaaaattatttccctcctgcgcttgtgctgtaattgtctgtgtacctgttgtttgtgactgtgttagttcccttggatatgatttcgtatttgatatgtacatggagatagcttagcttcattattggagtatgaagttagtatgacatagtcactctcctggaaaattgacactgcaaaccatatttttattctgaaccacaaatcctagtcagtccgctggcatatgccgtccgtttgctgaatccagaacgtgggtttggagatgtacggctgagatgcctctatgcgaaggggatttcgtggtgaaacgagatgggagtagagcaacgcccgtggaagatgcttcaaacttccacacttttgagcaacgatcggcagtagtaaggtagacgatttcaagatcaaagcatatgaagataaacaacatcaacaacaaaatttgttggggttctatagagagaaacagagctacatacatacactgttttgtatctaccatctgagatgatgaaaagatgaaaaactaaagaatgccccggcgccaacgccaggacacgccgcgcgcgcgtcacccgagccatctcttgacccagccggcgctgtatatttacacacgttgcagcatcgatcaccacctgttcgatcgcgtcgccgtcaccggcgcgccgcgacttattgaactagtctagggcgcggctaacaacctagcttagattgccttgtctaaacccttaggctaacgcgccactctagaaggccgatctcccccgaacgcgccagatccgatatcgatgggccctggccgaagcttggtcacccgcaccttgctagttggatattacctcttctcttcaaagtatccttgaacgctcaccggttatcaaatctctacactatagctctgtagtcttgctagatagttagttctttagctctctctagaggttcacaaactcaatacaacacacagtggagtagggtattacgctccggcggcccgaaccactctaaatccttgtgtcctcgtgtgctcttccaccattaatgactgtaattgcagggacacgaagagttgtagcgcttttgccaacaagatccgctatatgctagtagagtggaacctgcacagcaacaagcatccaataatctgagaataagaattatgtgtggtgatggttctcatctctgcaactcgccattttcgcgagagaaccaaatccaagttggtgaagatcatgatttgggctcatgggctggcatagtagctattacctttgcaattgtggttcgaatctatccgatattctccttttcagggagagctaatgcttatgcttattgggtattttggtttgacactgcttcacgcccaaaaaaaggcaactatgtctgagttggatatggcccaaaaaaaggcagctatgtctgagttggatatggaagtcttctttcgtttagggtgaagtaagaccaagctcatgagcttattatcttaggtcggaacaaattagttgataggcatggcacatttatcaaattataatgattttaacagaaaagcaacacctctttttcagcagcaccagctttgcaagctgcaattgacactccaagcatagtatttgctccaaactcagcctgcatttcgcacaatatcatatctatttccatttggataaaatatattttccagaaatatgggaacagaatcaagatacagaaaaaagttgtggtagtagtggtaagtggaaacacaagtacagtgaaaagatagcattatttctgaacagtcaaattaaccgaaataagtagtatcacatactgcccctaaattgactacacccaatgtaaagaattgcttgagcatattgagccatcagttgtttgtttgtgtaacggatttgacaatgcagagttcaggagcaaaaaaccgcaaaggagagaaagagggaaaggatctcctagtatcccaccttgtggcgcgccctgtcaagtgcatgatggcctggtcgatctgcgtctgctgctgcggttccatcccaacgagtgcctccgacaccttgtcgttgatcacccgcaccgcgtaggcgacccccctggcgaggaacttccgcctctcggaggccccggccgcgtcggcggcggcgccctcgagcgcgcccacgctagccgtagatgctcggtgcacggccttgttggtgtgcaactcgacctcaacggccggtgcgctacgcccgtcaaggatctggcgcgcccgcaccctcgtgatcacggtgggcgcctcccgcctcatgtggccgacgatgaaaagcgccgggttaggggccttggcgcgcaccgccgtattcagggcctcctcgctcttgcgcgagagcaggtgcttccccaggtactcctgcactgacaaggctgccacagcggtggaatttgtcctcacgacttcgccacgtcgtcctcacctccctggcttcccacgactccgccgtatcgaggtgctcggtgggtcgtcttaatccgaagtccgaactaggaagacgagacagagaggtctgaggaatgggcctcgtttgatgttgagctgaattatttttccagcataagcccaggtcttggtccatgaacaaaattactagaaacccagcccagtactacgatctaaaagagggactgccactggatagctctctctagcattctccacgctccaatacagcggcgtcgtgtctatccgggtctatccgcgaacacgtgagaactctccagaaactgctttctcctccacttcatctctctcgctttccctctataaaaagaccccttctaggaattgagggagacagcaagcaccaagcagcgatccgaagctcaatcaattcactcaaacctcttccccaaatcttcgattagatcttcgttgacaagaagactagaaccgaacctgacatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgaggtacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgatccgatatcgacgtctaaaaacggcccctaactaacgcttagttgcccgctagattagtcccccgttagtgcctagttagacacacgacgaactaggggctagtgccaccttgctagttggatattacctcttctcttcaaagtatccttgaacgctcaccggttatcaaatctctacactatagctctgtagtcttgctagatagttagttctttagctctcgcacgataacttcgtatagcatacattatacgaagttataacagcggaccgggttaccgaattcctgcccttaaggccaattgttcaagattcattcaacaattgaaacatctcccatgattaaatcagtataaggttgctatggtcttgcttgacaaagtttttttttgagggaatttcaactaaatttttgagtgaaactatcaaatactgattttaaaaaatttttataaaaggaagcgcagagataaaaggccatctatgctacaaaagtacccaaaaatgtaatcctaaagtatgaattgcattttttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggatatgtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcaggtcgactttaacttagcctaggatccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagggtaccaatggccagttaacagatccagctgctgctgttctagggttcacaagtctgcctatttgtcttccccaatggagctatggttgtctggtctggtccttggtcgtgtcccgtttcattgtgtactatttacctgtaatgtgtatccttaagtctggtttgatggtgtctgaaacgttttgctgtggtagagcagcatggaagaactataatgaataagtgatccctaatcattgtgtccaaattttgcttctgctatacccttttgtgctgtttcttatgttttgcttaaaaatttgatctgacaaacaaatttgtctaaattatgctcttgttctgactgtgtactgtacacatttgtattgctaccggtttggcacatagttttctctttgattgcagatcaaacccttctgattgcagattgcttggctatggctgatgtgctcactgttcttctttagcttgtatattgctgatggtggcttggctacggctgaaaaagttgctgctgcgccgacttggcaactgttgctccagtaatgctttgtcctctcttcctagccgccgacttggcacttggcagctgttgctcctgtaatgcgttgttatctcttatctcttgagatgtactatgattttgaggctgtttgacttgtgccagattgccagtgctgatgctatattatctcaggtttttatgctagtatatgttttactgtgtgctgcctgtgtcgagttacgtaatctattgaaccgctatgcgtgttgatgccctgttgttgcttgccagattgatgcccctgttgcttgcagcttgcaagctgtagtgaaatcctaaaattcgaaaccaaatctattctgaacttttgaagatctgtgatgtccggaagaaaagattcaaacagataaacgggactctccagaatctataaaataaatgatcgacaaacgctttgctttggtaggcttcctagttagttagcggaccgaagcttcggtccgggcctgcggccgcagatatcagaacctgcgtctggacgcacaagtcatagcattatcggctaaaatttcttaatttctaaattagtcatatcggctaagaaagtggggagcactatcatttcgtagaacaagaacaaggtatcatatatatatatatatataatatttaaactttgttaagtggaatcaaagtgctagtattaatggagtttcatgtgcattaaattttatgtcacatcagcaattttgttgacttggcaaggtcatttagggtgtgtttggaagacaggggctattaggagtattaaacatagtctaattacaaaactaattgcacaaccgctaagctgaatcgcgagatggatctattaagcttaattagtccatgatttgacaatgtggtgctacaataaccatttgctaatgatggattacttaggtttaatagattcgtctcgtgatttagcctatgggttctgctattaattttgtaattagctcatatttagttcttataattagtatccgaacatccaatgtgacatgctaaagtttaaccctggtatccaaatgaagtcttatgagagtttcatcactccggtggtatatgtacttaggctccgttttcttccaccgacttatttttagcacccgtcacattgaatgtttagatactaattagaagtattaaacgtagactatttacaaaatccattacataagacgaatctaaacggcgagacgaatctattaaacctaattagtccatgatttgacaatgtgttgctacagtaaacatttgctaatgatggattaattaggcttaatagattcgtctcgccgtttagcctccacttatgtaatgggttttctaaacaatctacgtttaatactcctaattagtatctaaatattcaatgtgacacgtgctaaaaataagtcagtggaaggaagagaacgtccccttagttttccatcttattaattgtacgatgaaactgtgcagccagatgattgacaatcgcaatacttcaactagtgggccatgcacatcagcgacgtgtaacgtcgtgagttgctgttcccgtagagaaatatcaactggtgggccacgcacatcagcgtcgtgtaacgtggacggaggagccccgtgacggcgtcgacatcgaacggccaccaaccacggaaccacccgtccccacctctcggaagctccgctccacggcgtcgacatctaacggctaccagcaggcgtacgggttggagtggactccttgcctctttgcgctggcggcttccggaaattgcgtggcggagacgaggcgggctcgtctcacacggcacggaagacgtcacgggttccttccccacctctcctcttccccaccgccataaatagccgaccccctcgcctttctccccaatctcatctcgtctcgtgttgttcggagcacaccacccgccccaaatcgttcttcccgcaagcctcggcgatccttcacccgcttcaaggtacggcgatcgtcttcctcctctagatcggcgtgatctgcaagtagttgatttggtagatggttaggatctgtgcactgaagaaatcatgttagatccgcgatgtttctgttcgtagatggctgggaggtggaatttttgtgtagatctgatatgttctcctgtttatcttgtcacgctcctgcgatttgtggggattttaggtcgttgatctgggaatcgtggggttgcttctaggctgttcgtagatgaggtcgttctcacggtttactggatcattgcctagtagatcagctcgggctttcgtctttgtatatggtgcccatacttgcatctatgatctggttccgtggtgttacctaggtttctgcgcctgattcgtccgatcgattttgttagcatgtggtaaacgtttggtcatggtctgatttagattagagtcgaataggatgatctcgatctagctcttgggattaatatgcatgtgtcaccaatctgttccgtggttaagatgatgaatctatgcttagttaatgggtgtagatatatatgctgctgttcctcaatgatgccgtagcttttacctgagcagcatggatcgtcctgttacttaggtagatgcacatgcttatagatcaagatatgtactgctactgttggaattctttagtatacctgatgatcatccatgctcttgttacttgttttggtatacttggatgatggcatgctgctgctttttgttgatttgagcccatccatatctgcatatgtcacatgattaagatgattacgctgtttctgtatgatgccatagcttttatgtgagcaacatgcatcctcctggttatatgcattaatagatggaagatatctattgctacaatttgatgattattttgtacatacgatgatcaagcatgctcttcatactttgttgatatacttggataatgaaatgctgctgcacgttcattctatagcactaatgatgtgatgaacacgcacgacctgtttgtggcatctgtttgaatgtgttgttgctgttcactagagactgttttattaacctactgctagatacttacccttctgtctgtttattcttttgcagccagaatagcggatccgcccatggtcgaacaggacggcctgcatgcaggatcaccagcggcctgggtcgaaagactcttcggctacgactgggcacaacagaccatcgggtgtagcgacgcagctgttttcaggctgtccgcacagggcagacccgttttgttcgtcaaaacagacctgtctggcgcgctcaacgaactgcaagacgaggccgcaagactgagctggcttgctaccacaggcgttccttgtgctgcagtcctcgacgtcgttactgaggctggaagagactggctgctcctgggtgaagtcccaggacaggaccttttgtccagccatctggcacctgccgaaaaggtcagcatcatggcagacgccatgagaaggctgcacacactcgaccctgcaacatgccccttcgatcaccaggcaaaacacaggatcgagcgcgcaagaacgcgcatggaggctggtttggtcgaccaagatgacctcgacgaggaacaccaaggactggctcctgctgaactcttcgccagactcaaggcgagaatgccagacggtgacgacctggtcgtcacacacggcgacgcttgtctcccaaacataatggtcgagaacggacgcttcagcggtttcatcgactgcggtagactcggagtcgctgacagataccaggacatcgccctggcaacacgcgacatcgcagaggaactcggaggcgaatgggctgacaggtttctcgtgctctacggtatcgctgctccagacagccagaggatcgcattctacaggctcctcgacgagttcttttagttcgaacggtacccggggatcctctagtgccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattcaatcactagagtcgacctgcatgattccgatgacttcgtaggttcctagctcaagccgctcgtgtccaagcgtcacttacgattagctaatgattacggcatctaggaccgactagctagctagctaagagctcggtaccgagctcgaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgcca

SEQ ID NO: 9 - DNA, 5488 bp, synthetic construct
  misc_feature 1..5488: note = PHP8418-0004
  source 1..5488: mol_type = other DNA
ggaagttcctattccgaagttcctattctctagaaagtataggaacttcagatccaccgggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggggtggaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcgaccttcttatgtgcttctagtctccaaatgtggttgatagttattttgctctaagatcaacagtaatgaagtataaatcatcgttgtggtgtgctactcggttaattgagcattaacacacacaaacatgacgaggatggtataatctccaaaaatgtgtactttgttaggtgggaccctatagccttgattaatgtgctatgttaggcatgcctggaaacgtgtgacgcatatgttttgtgaacctgttgatattatatgtgcttttatattaccatattttattaaaatactaatatttattactagtaagatataacattctatctagcttaaaaactaaccataaatattccataataactagatttaccaaactaatatactaaatatacataataaatacaaaattaacaagacaataatcaatatttatgagcttaatatatttagacattatggttggtcgacgataatcatgctaacttttcgtaattgcttgattgaaatatgcttagaataatgcctctttgttctacatggcaaatagggaccattatggtgtaacaccctgggaaccacaaacaccccgaaatgctactaaactacacaactaaccttcatatataaaatttcgacagcatctcctttgaaaatttgcatagacgtggaagcaacagagtataaacagatatcatgataagaaaacatactagacattaataatctgctagaaatgggaagaatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccaccggtcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatacgaagttcctattccgaagttcctattcttcaaaaagtataggaacttctgtacacca

SEQ ID NO: 10 - DNA, 18124 bp, synthetic construct
  misc_feature 1..18124: note = PHP54733
  source 1..18124: mol_type = other DNA
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggactaactaactagtcgagctagttaccctatgaggtgacatgaagcgctcacggttactatgacggttagcttcacgactgttggtggcagtagcgtacgacttagctatagttccggacttacccttaagataacttcgtatagcatacattatacgaagttatgggcccaccggttacgtaacgcgtctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaggcttgggcacccgctccgggcttagaaggccagcttcaagtttgtacaaaaaagcaggcttcgaaggagatagaaccgatccaccatgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagcgcgaggtttgtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaacatggtgatgcctaggtctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactgagctagacccagctttcttgtacaaagtggccgttaacggatgcagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaaggttgcgcccgccccagcttggtcagccgctccgggcttagaaggccgatctcgcgggcacccagctttcttctacaaagtggccgtttacggatcggctagaatgtcccgcaccggcttaccgatttgcacctcgctaccctgggttcgaccgaagctgaccgatgcttgcgcccgctcgcgaggccggccacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcccggcggatctcgactctagaggatcgctcaggaaggccgctgagatagagccatggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgctatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccaacagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtgagagcaacgcgagctgccactgctcttcactgatgtctctggaatggaaggaggaggaagtgagcatagcgttggtgcgttgctgtcaagggcgaattgtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgggtaccatttaaattgcgcccgccacggccgtggaggtcgtattccggtcagcttgcatccctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcttcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcgggcatggcggcgcgcagctcttcagtgtctggaacgacacttaagcgtacgtgccggcctggctctccgaaagggcgtattccagcacactggcggccgttactagacccaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggcctccggatacgtaataacttcgtatagcatacattatacgaagttatacctggagctcgtttaaacgctcttcaactggaagagcggttaccagagctggtcacctttgtccaccaagatggaactggcgcgcctcattaattaagtcagcggccgctctagttgaagacacgttcatgtcttcatcgtaagaagacactcagtagtcttcggccagaatggccatctggattcagcaggcctagaaggccatttaaatcctgaggatctggtcttcctaaggacccgggatatcgctatcaactttgtatagaaaagttgggccgaattcgagctcggtacggccagaatggcccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactctagaggatccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctccggactcagatctcgatagagtcaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggcaagcttgtttttctcagacagttttctaaaaaaagggcgtttctggggaagttcgagatggttcgtaaggtgttactggctcctgtgaaccaatacatgatactgccatgataagggttataattagtcaagcagagtaagaagaaacaacagtagcagtgactccgattcctgaagatgagtcatatttgtcttgtgctcctgctgtatgaaatggatcgcatgtgtatattcgtcgccgcgccgcactggtgtaacctgttgcctcagagtttgcttttagctggttctgttttaaaaataagtactgttttttggttggctgcaagccattctgaacttcagtttaccaattgtttttatgttgtggttgaatattttaattttttatttaatgtttggttctttttttatatatatttgcaaaaatgatacaagtggtcaagttttcatatagtatgggctctatttcctagagctctacctctaggaacgaattttgtggaggttttcttttggctagttaggcaaagtccccatatcttgcaggctaaatcaagaagaagctctgtcaaacagttttttttactgaaaagtgattaaagagtagtttctcctagatcacttcagagtttatcctagagaatcatgggaatcaaattcagttagaggatcatttcttacaaagaatcaactttcgtagagaatctaaagcagaaagagctttgacaaacttacccttagagcaattccaacattctcgcgtgagtttcttcgcgccgttgttttgcggtgacttcatctggacgtcccgcgacatagagacgcttgtattgatcatgagagcttgtgtggtcatacacaatataattgttaaagatgaaagagatgtggaccttaatgagcgattcgactttgatggtgaaaatgtgcaaccttctcatggtatttctactcgcacactagctgaatttattgaagctcataaaaagatccgagacaaagaaatacattttcaattgaaagaagacctaatcaagcacttatggaagcttggtcacccggtccgggcctagaaggccagcttcaagtttgtacaaaaaagcaggctccggccagaatggcccggaccgggttaccgaattcgagctcggtaccctgggatccgatatcgatgggccctggccgaagcttggtcacccggtccgggcctagaaggccgatctcccgggcacccagctttcttgtacaaagtggccgttaacggatcggccagaatggcccggaccgggttaccctgcccttaaggccaattgttcaagattcattcaacaattgaaacatctcccatgattaaatcagtataaggttgctatggtcttgcttgacaaagtttttttttgagggaatttcaactaaatttttgagtgaaactatcaaatactgattttaaaaaatttttataaaaggaagcgcagagataaaaggccatctatgctacaaaagtacccaaaaatgtaatcctaaagtatgaattgcattttttttttgtttggacgaaaggaaaggagtattaccacaagaatgatatcatcttcatatttagatcttttttgggtaaagcttgagattctctaaatatagagaaatcagaagaaaaaaaaaccgtgttttggtggttttgatttctagcctccacaataactttgacggcgtcgacaagtctaacggacaccaagcagcgaaccaccagcgccgagccaagcgaagcagacggccgagacgttgacaccttcggcgcggcatctctcgagagttccgctccggcgctccacctccaccgctggcggtttcttattccgttccgttccgcctcctgctctgctcctctccacaccacacggcacgaaaccgttacggcaccggcagcacccagcacgggagaggggattcctttcccaccgttccttccctttccgccccgccgctataaatagccagccccatccccagcttttttccccaatctcatctcctctctcctgttgttcggagcacacgcacaatccgatcgatccccaaatccccttcgtctctcctcgcgagcctcgtggatcccagcttcaaggtacggcgatcgatcatcccccctccttctctctaccttcttttctctagactacatcggatggcgatccatggttagggcctgctagtttcccttcctgttttgtcgatggctgcgaggcacaatagatctgatggcgttatgacggctaacttgtcatgttgttgcgatttatagtccctttaggagatcagtttaatttctcggatggttcgagatcggtggtccatggttagtaccctaagatccgcgctgttagggttcgtagatggaggcgacctgttctgattgttaacttgtcagtacctgggaaatcctgggatggttctagctcgtccgcagatgagatcgatttcatgatcctctgtatcttgtttcgttgcctaggttccgtctaatctatccgtggtatgatgtagatgttttgatcgtgctaactacgtcttgtaaagttaattgtcaggtcataatttttagcatgcctttttttttgtttggttttgtctaattgggctgtcgttctagatcagagtagaagactgttccaaactacctgctggatttattgaacttggatctgtatgtgtgtcacatatcttcataaattcatgattaagatggattgaaatatcttttatctttttggtatggatagttctatatgttggtgtggctttgttagatgtatacatgcttagatacatgaagcaacgtgctgctactgtttagtaattgctgttcatttgtctaataaacagataaggatatgtatttatgttgctgttggttttgctggtactttgttggatacaaatgcttcaatacagaaaacagcatgctgctacgatttaccatttatctaatcttatcatatgtctaatctaataaacaaacatgcttttaaattatcttcatatgcttggatgatggcatacacagcggctatgtgtggttttttaaatacccagcatcatgggcatgcatgacactgctttaatatgctttttatttgcttgagactgtttcttttgtttatactgaccctttagttcggtgactcttctgcaggtcgactttaacttagcctaggatccatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgcgtttacaacaagctgtaaatcactagcgaacgcgtaggtacccgggccgtgggtcgtttaagctgccgctgtacctgtgtcgtctggtgccttctggtgtacctgggaggttgtcgtctatcaagtatctgtggttggtgtcatgagtcagtgagtcccaatactgttcgtgtcctgtgtgcattatacccaaaactgttatgggcaaatcatgaataagcttgatgttcgaacttaaaagtctctgctcaatatggtattatggttgtttttgttcgtctcctaatatttgcctgggatcaaattttattggctggtgttcatttgacctccatgttcttgctaggctccattttttactctacagccataatatgtttgattgtttggtttgttctttgttgtacacctggttctgtcgagcttagttttcgacactggcttacagcttaacatgttgctattttattgggttctgattgctattttattgggttctgattgctagtttttgctgaatccaaaaaccatgttatttatttaagcgatccaggttattattatgatggtggctaagtttttttttttccaagggtaaattttctggattctccagtgtttctgtggccgaattgggcaactttattatacaaagttgatagatatcggaccgattaaactttaattcggtccggtggcgccgctagcgtatacgaagttcctattccgaagttcctattctccagaaagtataggaacttctgtacacctgagctgattccgatgacttcgtaggttcctagctcaagccgctcgtgtccaagcgtcacttacgattagctaatgattacggcatctaggaccgactagctaactaactagtacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 11 - DNA, 18676 bp, synthetic construct
  misc_feature 1..18676: note = PHP70298
  source 1..18676: mol_type = other DNA
atgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaataatgtctaacaattcgttcaagccgacgccgcttcgcggcgcggcttaactcaagcgttagatgcactatacgtaccaatccaaatcggatccccctgcagggctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatctatcgatggtacctatctgcagacggaagggattcctgttaattaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctcgcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggcagcgcaggccgcccagcggcaacgcatcgaggcagaagcacgccccggtgaatcgtggcaagcagccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcaggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatgcatataattgtggtttcaaaatcggctccgtcgatactatgttatacgccaactctgaaaacaactttgaaaaagctgttttctggtatttgacccgcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatgcttaagagatcggggtcgctttctcttgccttgatggtctccttctgttcgtcgagccttgccacgccactctcatctgctgagtttgaccatgttgctcgcaagtgtgccccatcagttgcgacatctacgcttgcggcgatagctaaggtggagagtcgctttgatcctttagcgattcatgacaacacgaccggcgaaacgcttcactggcaagatcacagccaagcaacccaagtcgtcaggcaccgtctcgatgcacggcattcgctggatgttggcctcatgcaaataaactctcgaaatttttctatgctcggtctgacacctgacggtgcgctccaggcgtgcacatcattatctgccgctgcaaacatgctgaaaagtcgttatgcaggcggcgaaacgattgacgagaagcaatttgcgcttcgtcgggcgatctccgcttacaacaccggtaatttcatcggcggttttgcaaacggctacgtgcgaaaagttgaaacagctgctcaatcgctggtgcccgcgttaatcgagcctccaaaagacgatcacgaggcgctaaaatccgaagagacgtgggatgtttgggggtcatatcagcgccgctcgcaggaggatggcgctggcggtttaatcgctccgccaccgccacaccaggacaacggcaaatccgcagacgacaatcaagtcttattcgacttatactaaggaggtgcgcattgatgcgatgctttgagagataccgtttacatctaaatcgcctctcgctctcgaatgcgatgatgcgcgtgatatcgagctgcgccccaagcttgtgcggtgcaattgcatggagcatttcctcatccggacccgccgcagcgcaatctgcgggtggcggcactgaccccgccacaatggttaacaatatatgcacgtttatccttggtccgttcggccagtcactcgctgttctcggcattgtcgctatcgggatctcctggatgttcgggcgggcttcgcttgggctggttgccggcgtcgtcggcggcattgttatcatgtttggggcgagcttcctcggccaaacgctcactggcggtagttgatggctgatcgtttggaagaatcgaccctttacctcgcagccacacggcccgcattgtttcttggggtgccactgacattggcagggttattcatgatgttcgccggctttgtcatcgttatcgttcagaacccgctctacgaagtcgttctcgtgccgttatggtttgcagcccggctcatcgtggagcgagactacaatgcggcgagcgtcgtcctgctatttttgcggaccgcgggaagaagcattgatagtgcagtttgggggggcgctactgttagcccaaatccaattagggttcccccacgagggagaggaatggtgtgatgctcggcgcgagtggaacgaccgaaagatccggtgagatctatctcccttatattggccacctcagcgaccatatcgtccttcttgaagacggatcgatcatgaccattgcgagaattgatggcgttgcattcgagcttgaggaaactgaaatgcgcaatgcgcgttgtcgtgcgttcaacacgctgttgcgcaatatcgctgatgatcatgtgtcaatatatgctcacctcgtacgtcatgccgacgtgccatcatcggcgccgcgacacttccgtagtgttttcgccgctagcctgaacgaagcttttgaacagcgcgtgctctccggccaactcctccgcaatgaacacttccttacgttgattgtctacccacaggcggctttagggaaggtaaagaggaggttcaccaagctaagcggaaaaagggaaaacgatctcacgggccagatcaggaacatggaagatctttggcatgttgtcgctggctctcttaaagcgtatggcctgcatcgtcttggcatccgcgagaagcagggtgtgctcttcaccgaaattggcgaagcgctacggttgatcatgactggtcggttcacaccggttccggtcgtcagcggctcactcggcgcttcgatttataccgacagagtcatttgcggcaagcgaggactcgagatcagaacgccaaaagacagttacgttggatccatctattcgtttcgcgaataccctgcaaaaacacggccgggcatgctcaacgcgctgctatccctcgattttccacttgttctcacgcagagtttttcgttcctgactcgcccgcaagcgcacgcgaaacttagcctcaaatcgagccagatgctgagttccggcgataaagccgtgactcaaatcggcaaattatccgaggctgaggacgcacttgcgagcaacgaattcgttatgggctcacatcatttgagcctttgcgtctatgcagacgatctcaatagtcttggggacaggggcgcgcgggctcggacacgaatggcggatgcaggtgccgtggttgtccaagaaggtattggtatggaagcggcctattggtcccaattgccggggaattttaagtggcgcacacgccctggcgcaatcacttcacgcaatttcgcagggtttgtctctttcgaaaactttccagagggcgccagctcaggccactggggcaacgcgattgcccgatttcgtaccaatggcggaacgcctttcgactatatcccgcatgagcacgatgttggcatgacggcaatattcgggcctatcgggaggggtaagacgacgctcatgatgtttgttctagccatgctcgaacagagcatggtcgaccgtgcaggtacggtcgtgttctttgacaaggaccggggtggcgaattgctggttcgcgccacaggaggaacatatttggcacttcacagaggcacacccagcgggttggcgccgttgcgtggcctagaaaacacagcagcctcacacgattttctgcgcgaatggatcgtggctctcatcgagagtgatggtcggggtgggatttctccggaagagaaccgccgtctggtccgtggtatccatcgtcagctctcgtttgatccacaaatgcgttcaatcgcggggttacgtgaatttttgttgcatgggcccgccgaaggcgcaggagcgcggctccaacgctggtgccggggccatgcgcttggctgggcatttgacggcgaagttgacgaagtaaagttagatccgtcgattaccggcttcgacatgacgcatcttctcgaatacgaggaagtatgcgctcccgctgcagcatatctcctgcatcggattggagccatgatcgacggccgccgttttgtgatgagctgcgatgagtttcgcgcctatttgttaaaccctaaattttcgactgtcgtcgacaaattcctcctgaccgttcgaaaaaacaacgggatgctaatactggcaacgcagcaaccagagcatgttctggaatcgccgctaggagccagcttggttgcgcaatgtatgacgaagattttctatccatcaccaaccgcagatcgatcggcttatgtcgatggactgaaatgtaccgaaaaggaatttcaggcgatccgtgaagacatgacggtcggcagccgtaagtttcttcttaaacgagaaagtggaagcgtcatctgcgaatttgatctgcgggatatgcgtgaatatgtcgccgtgctttcggggcgtgccaacacggtgcgctttgcaactcgactacgcgaggcacaagaaggcaactcatctggctggctcagcgaattcatggcccgtcaccacgaggcagaagattgataaggtaggaaacgatgaagacgacgcaacttattgcaacagttttgacctgcagctttctatatattcagcccgcgcgggcgcagtttgttgttagcgacccggcaacggaggctgagacgctcgcgactgcgctcgcgactgcggagaatctcactcagactatagcgatggttacgatgttgacgtcggcctacggcgttactggactactgacttcgctcaaccagaaaaatcagtatccttcgacgaaggacctagacaatgaaatgttttcgccgcgaatgccaatgtcgaccacggcacgtgcgatcaccagcgatacagatcgtgcagtcgtgggtagtgatgctgaagcggacctgttgcgatcgcagatcaccggttccgcaaacagcgctggcattgcggctgacaatctggaaacgatggacaaacgcttgacggcgaatgctgatacgtctgctcagctttcccgatctcgcaatatcatgcaggcaaccgtgaccaatggtttgcttctcaagcagatccatgacgcaatgattcaaaatgtacaggcgacaagcctattaacgatgactaccgcgcaggccggccttcacgaggcggaagaggcggccgctcaacgcaaggagcatcaaaagaccgctgtcatctttggtgccctcccctaaggctgggcgatttgttcatccgcccgcatcctcgccgaatgcgagctcattttatccaacattatgcgacaaaccagtcaagttcaggtccaatcgatgaatttcacgattccggcgccgtttacggccattcatacgatcttcgatgtagccttcacgacaggcttggactcgatgcttgagactatccaggaggcggtgagtgcgccattgatcgcctgtgtcactctttggattattgttcagggtattttagtcatacgcggcgaagtcgatacccgtagcggtatcactcgggtgatcacggtcaccatcgttgttgctctaattgttgggcaggctaactaccaagactatgtggtttccatcttcgaaaagacggtcccaaactttgttcagcagtttagtgtaaccggcttgcctctgcagactgttccggcacagttggatacaatgttcgccgtgacccaggccgtttttcagaaaatcgcatccgaaatcggtccgatgaacgaccaggacatccttgctttccaaggggcacagtgggtcctttacggcacgctctggtctgccttcggagtttacgacgccgttggaattctcacgaaagtgcttctcgcgatcgggcctctgatcctcgtcggatatatttttgatcgcacgcgggacatcgcagctaagtggatcgggcaacttatcacctacggtctcttgcttctcctcttaaacctcgtggcaacgatcgtcatcctaaccgaagcgactgcgctcacccttatgcttggtgtaatcaccttcgccggtacgaccgcggccaagatcattggtctttacgaactcgatatgttttttctgacaggggatgcgctcattgtcgctttgccggcgatcgccggcaacattggaggcagttactggagcggcgcaacccaatctgccagcagcttgtaccgtcgcttcgctcaggttgagcgaggctaggtcgcgcaaaaattcgcctcaatggagaattctatgaagtattgcctgctgtgcctagttgtcgctttgagcggctgccagacaaacgacacattagcgagctgcaaaggcccgatcttcccgctgaatgtggggcgatggcagcctactccgtcagatcttcagctcggcaattcgggtggacgctatgacggggcctgaatatgccatgctagtggcgcgcgaaagccttgccgagcactataaggaagtagaagcctttcaaaccgcgcgagcgaaatcggcgcgacgtctctccaaactcattgcagctgtcgcagctatcgcgattttgggaaatgttgctcaagcgttcgctatagccacaatggtgccgttgagcaggcttgtgcccgtatatctatggatacggccggacggcaccgttgacagcgaggtgtctgtctcgcgattgcctgcaactcaagaggaggccgtcgttaacgcctcattgtgggagtacgttcgcctgcgcgagagttatgatgccgacaccgctcagtacgcctacgacctggtatcgaacttcagtgccccaacagtgcgccaggattaccagcaattcttcaactatcccaatcccagttcgcctcaagtcattcttggcaaacgcggcagggtggaggtcgagcacatcgcttcaaatgatgtaactccaagcacgcagcaaattcgctataaaaggaccctcgtcgttgacggcaaaatgcctgtggtgagtacgtggaccgcgacagttcgctacgaaaaggtgaccagcttgcccggcagattgagactaaccaacccggcaggtctggttgtcacctcctatcagacatcggaagataccgtttcaaacgtaggccacagcgaaccatgatcagaaaagcacttttcattttagcatgtttatttgccgctgcgactggtgcggaggctgaagacactccaatggcgggcaagctagatccgcgcatgcgttatttggcttacaatcccgatcaagtggtgcgcctctcgacggcggttggagctactttggtcgtaacattcgccacgaacgaaacggtgacagcggttgccgtttcaaatagcaaagatctagcagccctaccgcggggaaattatctatttttcaaggcaagccaggtcctcacgcctcagccagtaatcgtgctaaccgcaagcgactccgggatgcgccgttatgttttcagtataagttccaagactctgtcccacctcgataaagagcagcccgatctctattacagcgtccaattcgcctaccccgccgacgatgcggcggctcggcgaagggaggcacaacagaaggctgttgtggacagactacacgcggaagcacaatatcaacggaaagctgagaatttattggatcagcctgtcacagcccttggtgcggcggacagtaattggcactacgtcgcccaaggcgatcgttcgctgttgccactcgaagtcttcgacaatggatttacgacggtattccactttccgggcaatgtacgcataccctccatctacaccatcaatcctgatggcaaggaagctgttgccaactattcagttaaagggagcgatgtcgagatttcttcggtttcccgaggttggcgtctgagggatggccacacagtactatgtatctggaacaccgcttacgatcccgttggccaaaggccgcaaacgggcacggtgaggcccgatgtgaaacgcgtcctgaagggggcgaagggatgaataacgatagtcagcaagcggcacatgaggttgatgcatctggatccctggtctccgacaaacatcgccggcgtctttcggggtctcagaaattgatcgtcggaggtgtcgttctcgcgttatcattaagcctcatttggctaggtgggcgccaaaagaaggtgaatgagaacgcatcgccgtcaactttgatcgcaacaaacaccaagccatttcatccagctccgattgaggtgccgccggatcctccagcggttcaagaggctgttcagcctgctgctcctctaccgccgaggggcgaaccggagcggcatgagccacggccggaagaaacaccgatttttgcatatagcagcggcgatcaaggggtcagcaaacgcgccattcagggcgacacgggccgaagacaagaaggcaagcgtgacgacaactccttgccgaatggcgaagtgtccggcgagaacgatttgtcgatacgtatgaaacccaccgagctgcagcccagcagcgccacgctcttgccgcaccccgattttatggtaacgcaagggacaataattccgtgcatcttgcaaaccgcaatcgacacaaatttggcaggctatgtaaagtgtgtcttgcctcaggatattcgtggaacaacgaacaatatcgtgcttcttgatcgtggcaccaccgttgttggcgaaatacagcgtggcttgcaacagggagatgggcgcgtttttgtgttgtgggatcgcgccgagacacctgaccatgcgatgatctcgttaacatcgccaagcgcggacgaactcggtcgctcaggattgccgggctcggtcgacagccacttctggcagcgttttagcggagctatgctcttgagtgttgttcaaggcgccttccaggcagctagcacctacgccggcagctcgggtggcgggatgagcttcaacagctttcaaaataacggtgagcagacaactgagacagcccttaaggcaaccatcaacataccgccaaccctgaagaagaatcagggtgacaccgtttccattttcgtagcacgggacctcgatttctttggtgtttaccagctccgcctgactggcggcgccacgcgggggaggaaccgccgctcttaatgaattcaaatttccgcttagagataggatacattgtaaatggaagtggatccgcaactacgctttcttctgaagccgattttggaatggctcgatgacccgaagactgaagaaattgcgatcaatcgacctggagaggcatttgtgcgccaagccggcatttttaccaagatgcctttgcccgtctcttatgatgatcttgaagatatcgctattttagcgggcgcgctgagaaagcaggatgtcggaccacgtaaccccctctgcgccactgaacttcctggtggtgaacgactacaaatctgtctgccgccgaccgttccctcgggcaccgtcagcttgaccattcgacggccaagctcgcgtgtttctggtcttaaagaagtctcctcccgttatgatgcttcgaggtggaaccagtggcagacacgaaggaaacgccaaaatcaggatgatgaagctatccttcagcattttgacaacggggatttggaagcgtttctgcacgcatgcgtcgtcagccgactgacgatgttgctatgtggccctaccggaagcggcaagacaacaatgagcaagaccttgatcagcgccatccccccccaggaaaggctaatcaccatagaagatacgctcgaactcgtcattccacacgataatcatgttagactactctactccaagaacggtgctgggctgggtgctgtgagcgccgagcacttgctccaagcaagtctgcgtatgcggccggaccggatattgcttggcgagatgcgcgacgatgcagcatgggcttatctgagtgaagtcgtctcgggacatccgggatcgatttcaacaatacacggcgcgaatccaatccaaggattcaagaaactgttttcccttgtcaaaagtagcgcccaaggtgctagcttggaagatcgcacactgattgacatgctctctacggcgatcgatgtcatcattccattccgtgcctatgaggacgtttatgaagtaggcgagatctggctcgcggcggacgcacgacgccggggcgagaccataggcgatctccttaatcaatagtagctgtaacctcgaagcgtttcacttgtaacaacgattgagaacttttgtcataaaattgaaatacttggttcgcattttcgtcatccgcggtcagccgcaattctgacgaactgcccatttagctggagatgattgtacatccttcacgtgaaaatttctcaagcgctgtgaacaagggttcagattttagattgagaggtgagccgttgaaacacgttcttcttatcgatgacgatgtcgctatgcggcatcttattatcgaataccttacgatccacgccttcaaagtgaccgcggtagccgacagcacccagttcactagagtactctcttccgcgacggtcgatgtcgtggttgttgatctaaatttaggtcgtgaagatgggcttgagatcgttcgaaatctggcggcaaagtctgatattccaatcataattatcagtggcgaccgccttgaggagacggataaagttgttgcactcgagctaggagcaagtgattttatcgctaagccgtttagtacgagagagtttcttgcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttacgacaggagtatagatgttctcattttgcggctgcgccgcaaacttgaggcggatccgtcaagccctcaactgataaaaacagcaagaggtgccggttatttctttgacgcggacgtgcaggtttcgcacggggggacgatggcagcctgagccaattgcatttgattaatttaggtgactgaggacgcggccagcggcctcaaacctacactcaatatttggtgaggggttccgataggtccctcttcaccaattgctcgatggcttctctccagcaaagaatgacgcgagcgcggcggtagccagcttgtggccgaaagctcgagcggtctccaaccccaacggatcaaaatgacttcgagcgacctcgagcaacgcaaccgggaacatgcgtgaggtctgaacgagaacggatttttctgtagttgaagggatcggataacttttcggggccacgcgaaatgatccatctgccagcatgctttcgaaatcgtccaacgcgcgccttaaaatcatttgtagcgacttcgagggactgtattgccgaacgaggttgtcatatgttttcgacacttgaggcgcgggcggtcgcgctgaaagaaaaacctggagctttttcggggacggaggtggactaagggcatccacagttagcttaagttgtcgatcgggactgtaaatgtgatcggcgacgagaggctcacgttgctggtctttctcgtcggctttttcaggcaagtgctggaggtccagcttctggggaacaagtgtcgggttgggatggtggatctcgggtcgagcaccagcaagccgccgtgcttcgccgaccgacaatgcgggcttgcgaattgccatcttcaagcctccaagattttgctgatcagtttcgaaatgaccacgacttcctccatcgcaatccgaagattcctctctatgaggcgcatcgtcggatcagttcccgtgtttagtaatgtaagatgcaacatgccgcgttctttcatcgcggcaaatgcatctctttcatgcatgggagacggtacaactggaaggctctctagcgtctctgacatcctgcgttgcgatgttgtcaatcggccgaccgggacgcgttggcgcaaaacagctgtaggaattgccaaattttcactcaacagcagctcgatgacgtagcggtaggtagatagtgcctcatcgatgtcgagcggcgttagcatggtggggatcagaagcaggtttgagctagcgatgattgtgttgttgagctcgctcgagccgccacgcgtatcggccaacgcataatcaaatccttcgagctcggcattttcataggctgcttcaagaaggggcatttcgtcggcggaatagacttcacagcgaggatcccaggtactgctttgtaaggcgttttctctccatcgcgtcagaggccggttttcgtcggcatcaaagagggccactcgtttaccgtcatttgccaaagcagcgcaaaggcccatgagtgcggtggttttgccagcaccccctttgaaagaacaaaacgtcaaaagttgcatattctgatcccgcctatcctgtgaaaccggagtgcatttgtatttttgttcgtataaatgtttttgtgattatcgatgagtaaaagcgttgttacactatttttatttcacattcgttataagacaattgcaaatgtagcaagtatattcagtattgactgtaaatgtactgttgatttcatattgagcagggctagacttccatccgtctacccgggcacatttcgtgctggagtatccagaccttccgctttctttggaggaagctatgtcaaaacacaccagagccacgtcgagtgagactaccatcaaccagcatcgatccctgaaagttgaagggttcaaggtcgtgagtgcccgtctgcgatcggccgagtatgaaaccttttcctatcaagcgcgcctgctgggactttcggatagtatggcaattcgcgttgcggtgcgccgcatcgggggctttctcgaaatagatgcagacacacgagaaaagatggaagccatacttcagtccatcggaatactctcaagcaatgtatccatgcttctatctgcctacgccgaagaccctcgatcggatctggaggctgtgcgagatgaacgtattgcttttggcgaggctttcgccgccctcgatggactactgcgctccattttgtccgtatcccggcgacggatcgacggtcgctcgttactgaaaggtgccttgtagcacttgaccacgcacctgacgggagaaaattggatgcccgatcgcgctcaagtaatcattcgcattgtgccaggaggtggaaccaagacccttcagcagataatcaatcagctggagtacctgtcccgaaagggaaagctggaactgcagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtaagcttccccgcaggtaccgaccaaaccgcagcttatgaagcaagccgggaatgggcagccgagatgtttgggtcaggatacgggggtggccgctataactatctgacagcctaccacgtcgaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaatatcgacgccttgagcggatgcaggctcaaaagattcaattcgaagatacagattttgatgagacctcgcctgaggaagatcgtcgggacctcagtcaatcgttcgatccatttcgatcggacgcatctgccggcgaaccggaccgtgcaacccgacatgacaaacaaccgcttgaaccgcacgcccgtttccaggagcccgccggctccagcatcaaagccgacgcacggatccgcgtaccattggagagcgagcggggtgcccaaccatccgcgtccaaaatccctgtaactgggcatttcgggattgagacttcgtatgtcgctgaagccagcgtgcccaaacaaagcggcaattccgatacttctcgcccggtgactgacgttgccatgcacacagtcgagcgccagcagcgatcaaaacgacgtcatgacgaggaggcaggtccgagcggagcaaaccgtaaaagattgaaggccgcgcaagttgattccgaggcaaatgtcggtgagcccgacggtcgcgatgacagcaacaaggcggctgatccggtgtctgcttccatccgtaccgagcaaccggaagcttctccaacgtgtccgcgtgaccgtcacgatggagaattgggagaacgcaaacgtgcaagaggtaatcgtcgcgacgatgggcgcggggggacctagactagtgtacctcgcgaatgcatctagatccaatccaatatgcatggcatgccgaatccatgtgggagtttattcttgacacagatatttatgatataataactgagtaagcttaacataaggaggaaaaacat

SEQ ID NO: 12 - DNA, 45770 bp, synthetic construct
  misc_feature 1..45770: note = RV005393
  source 1..45770: mol_type = other DNA
atggatggtactttggagatatgagcacttgccagacttcccggtcggagacttaaaaattccgataagaggttacggacgacggaccttataagtggatcgtctagtggtggcgccgatcaaaacagttccgcccccggctttgctcaaagtagcaaagcagctttatctcgggttgcggaggattttctagaaaaccgcaattttgcaggagagaacatatggccatcatcaagccgcatgtgaacaaaaataggacaacctcgccgatagagagaccggagtctctcatagaggaaatgagcggcagtcatccgccgagtggttttaccaacctggatctcgctatgatcgagctggaggactttgtccatcggtgcccgctcccagaagacaatcttgctggtcagaaggagtgagacgatggatccgtctagcaatgagaatgtctatgtgggtcgcggtcacaacatcgaaaatgatgatgacactgaccccaggcgttggaagaaggcgaatatcagttccaacaccatctccgatattcagatgacgaatggcgaagacgtacaatcagggagccctacccgaacggaagttgtaagcccacgtctggattatggatcggtcgactcctcctccagcctttattctggcagcgagcacggaaatcaagctgagattcaaaaagagctgtccgtcttgttctcgaacatgtctttgccaggcaacgatcggcgcccggacgaatacattctcgtgcatcaaacgggacaagatgcttttactggtattgccaaaggcaacctcgaccaaatgcccaccaaggcggaatttaacgcgtgctgccgtctctacagggacggagccggtaattactacccgccacctctcgcattcgacaagattagcgttccagagcaactggaggaaaaatgggggatgatggaggcgaaggaacgtaacaaactgcggtttcagtacaagttggacgtatggaatcatgcgcacgctgatatggggatcacgggcacagagatcttttatcaaacagataagaacataaagctcgaccggaattataaactaagacctgaagaccgatacgtacaaacagaaaaatacgggcgccgggaaattcaaaagcgatatcaacacgaactccaggctggttcgctgctgcccgatattatgatcaaaactccccaaaatgacatccacttcgtgtacaggtttgccggcgacaattacgccaacaaacagttcagcgagtttgaacacaccgtcaagcgcaggtatggcgacgagactgagatcaaattgaagtcaaagtcaggcattatgcatgactcgaaatatctggaatcctgggaacggggcagtgcggatattcgcttcgcggaattcgttggggaaaatagagctcacaatcggcagtttccaactgcgacagtaaatatgggacagcagccagacgggcagggcggtttgacccgcgaccgtcatgtgagcgttgacttcctaatgcaaagcgcacccaattcgccttgggcgcaagctttgaaaaagggagaactgtgggatcgcgttcagttgcttgctcgcgacggcaaccgctatctgtcgccgcccagattggaatattctgaccctgcacatttcaccgagttgatgaaccgggttggtttacccgcatcgatgggtcggcaaagccatgcggctagtatcaaattcgaaaagtttgacgcgcaggcagcggttattgtcttaaatggcccagagttacgtgacattcatgacttgtctcctgaaaaactgcaaaatttgtccaccaaagatgtcatcgtcgccgatcgcaatgagaatggtcagagaactggcacgtacaccagcgtcgcggaatatgagcgcttgcagttaaggctgccacccgatgcagcgggggtgcttggtgaagcaactgacaaatattcacgtgatttcgttcggccagagccggcgtcgcgtccaatcagtgacagccgcaggatatacgaaagtcgaccgcgtagccaaagcgtcaacagcttttgacgttcctgctgccgcgtcaacgaggaagctcgtttgacccgggtttgccaatgaaagggctcaatcatggtgaacactacaaagaaaagttttgcgaagtcgcttacggcagatatgcgccgttctgctcagcgcgttgtcgagcaaatgcgaaaagcattgattaccgaagaagaggcgctcaagcggcaagccagactggagagtcccgataggaagcgaaagtatgctgctgatatggcgatagtcgacaaactcgacgtagggtttcgaggcgaaataggctataaaattcttggaaataaccggcttcgagtagacaaccataaagaattaacgcgtgagcacggtagacttcgcaaaaccaaaacggttctgaagcgtaacccggtgacgcaggaagtctatttgggtttatatgaaaggaagtcctggttaagtgtcagcagccatttgtatgctgcggacggcacactccgcatgaagcacgtgaaatacaaagacggacgttttgaggaaaaatgggagcgcgacgaaaatggcgacctgatccgcacaaggtacgccaaccgtggcaggctctttcaacctgtatccgagaaaatgggcgcgccgtatcggagcggccctgacgaccggctctatcgcgatctaacccgtcgaaacggtttcagacgggagacattcgaacgggacgatcacggaaacctcgagcgtatcggcagcaaccatgtcggcttttccaagatttcagtgaaggcacccaatcgtcaaacctcccagacgaagattcaaaaacttggtggcgctttcaacaaatcttttaggtcccttctggacaaggagggcaatgaaatgggccgcgatattttgagccatcgacggctctataacaagcggtctgctgtctacgatgaagctaccggacaattgaagagtgccaagcataccttcggcaagatctacaggagcgaaaccgaatatctcagcgcgggcctcaagaaggtttcaaaaaagatactcggggtgacggtctaccggaaatttgcggcgctcagcgagcgagaatccgaggctgagagactgcgtagttttgaatccggtgcgcatcgccagatctggcaggagcgggcagcgattcccggttcgcccctcccggagactgatgacattcatttcgcacagcagtcgcacctagccaaagccaaccctgatcacgtcgaagctgacgtcacgcgtgtgacagatcaacatgttgatgttgctggacaaacatcatcgtctccccaacggaacttggaaggatggttagattctcaatcacgatacaagccagcaaacatgctgttgtcaaatccagaccttcaagcgaacggacctcgcccatacgaagggttagctcatctcaccctccggcgcgataatgaatctgacgggcacaaggagaatgatcagcggctgcgacatttctcccagccagagccgttggtgttaccgcatcccgggtcgccggaaataactaaggtgtttggctcgcggggagagccggcacacccgagtggaaatctgcacacggcggttggagaaacggcttgcgaaggaccggtgatgtcttcatcctcggacaatcatcagccagctccaggacagcaagaacttttaagtttccttcataatgcgccagccccagtttctgtggcaatacatgatgatcaagagcgacttgcgggggaggcgcccggcggctctttcagaggtagctcagggcgaacgagttcaatgtcggagagtatcttcgacgaagatgtacaagggcatttggtacgggattattcgatcaatactactaacgggtttattgacccgcaatcgttgttcggtgaaccggacttatcgagaggtccaaaatcggggccagaaattccatcggaagattaccatttgtcagcttcggaacaggaaaatttgctgaatcaattgcttagtgtgccactgccggttccttcaccgaagcccgaatgcgcgaggtctatgattttcgaaggttcacgttcaagagagcgttccacctccagagggttctaaggtggaacgcgtctaggcttgcctatcatcaccacctcatgatggtcgcggagaccttcgaagagatatccgacatcattggagaagctgggccggaaactagtgtacctcgcgaatgcatctagatccaatccaatatgcatggcatgccgaatccatgtgggagtttattcttgacacagatatttatgatataataactgagtaagcttaacataaggaggaaaaacatatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaataatgtctaacaattcgttcaagccgacgccgcttcgcggcgcggcttaactcaagcgttagatgcactatacgtaccaatccaaatcggatccccctgcatattttcctcctcgagttggatgaactcgccgagttcatcgtcaactgaaacagacacggccggattctgtgagacaggttgaaccgcagctctcttccattgataataggtctgaacggaaatacccacgatcttaacggcgtccttcaaggttgcgccgccagcgacctgagcttcgatttgaccgatcttctccagtttttctcggttgctgaggccgcgggttttcggcttcacggatttgaacgatcccgtgcgggctgtttcggctggtgctttctttgctcttctacctctaggagcagccggctcaacttcggcagcagcagtaccgtccggcggattctggatctcttcgtcagccattaatcgtcctctgtgtgggttattgctttgtctgccagctcgatccaagagtcaacgtttgtgcctagagtcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatctagcgtggactcaaggctctcgcgaatggctcgcgttggaaactttcattgacacttgaggggcaccgcagggaaattctcgtccttgcgagaaccggctatgtcgtgctgcgcatcgagcctgcgcccttggcttgtctcgcccctctccgcgtcgctacggggcttccagcgcctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcaattcagcgggttcgtgcgatccgtctgcatgatggtgatagccgtcagcatgattttcgtgtcgtcgaacttggtgaagggcattctcggcggcgatcacgacgccggccctgcggagccttcgccgcgtgcgcgattcatggcggccgtggaggccaaggatttcgcgcgagtgcaagagctgatcgaggcgcgtggagccaagtcggcggctgattatgtccttgcgcagctcgccgtggccgaaggtctggaccgcaagcctggtgcgcgcgtcgtggtcgggaaagcggcgggcagcatggcaatgccgcctgcggcgctgggttttacgccaaggggagaagcggcatacgccatcgagcggtcagcctatggtgagccgaggtccagcattgcgaagcagtaccagcaggaatggaaccggaaggcggcgacctggtgggcgatggccggtgtggccggcatcatcggcgcgatcctggcggcggcggcaaccggctttgttgggctggcagtgtcgatccgcaaccgagtgaagcgcgtgcgcgacctgttggtgatggagccgggtgcagagccataagcggcaagagacgaaagcccggtttccgggcttttgttttgttacgccaaggacgagttttagcggctaaaggtgttgacgtgcgagaaatgtttagctaaacttctctcatgtgctggcggctgtcaccgctatgttcaaccaaggcgcggagcaaattatgggtgttatccatgaagaaacggcttaccgaaagccagttccaggaggcgatccaggggctggaagtggggcagcagaccatcgagatagcgcggggcgtcttagtcgatgggaagccacaggcgacgttcgcaacgtcgctgggactgaccaggggcgcagtgtcgcaagcggtgcatcgcgtgtgggccgcgttcgaggacaagaacttgcccgaggggtacgcgcgggtaacggcggttctgccggaacatcaggcgtacatcgtccggaagtgggaagcggacgccaagaaaaaacaggaaaccaaacgatgaaaactttggtcacggccaaccagaaaggcggcgtcggcaagacttcgacccttgtgcatcttgccttcgactttttcgagcgcggcttgcgggttgccgtgatcgacctggacccccagggcaatgcgtcctacacgctcaaggactttgctaccggcctgcatgcaagcaagctgttcggcgctgtccctgccggcggctggaccgaaaccgcacccgcagccggcgacggccaggccgcgcgcctcgccctcatcgagtccaacccggtactggcgaacgccgaacggctgtcgctggacgacgcccgcgagctgttcggggcgaacatcaaggccctggcgaaccaaggcttcgacgtgtgcctgatcgacacggccccgacccttggcgtcggcctggcggccgccctcttcgcggccgactatgtgctgtcccccatcgagcttgaggcgtacagcatccagggcatcaagaagatggtcacgaccattgcgaacgtgcgccagaagaacgccaagctgcaattccttggcatggtgcccagcaaggtcgatgcgcggaatccgcgccacgcgcgccaccaagccgagctgctggccgcgtaccccaagatgatgattccggccaccgttggcctgcgcagcagcatcgccgatgccctcgcatccggtgtgccggtctggaagatcaagaaaacggccgcgcgcaaggcatcgaaagaggttcgcgccctggctgattacgtgttcacgaagatggagatttcccaatgactgcggctcaagccaagaccaccaagaaaaacaccgctgcggccgctcaggaagccgcaggcgcggcgcagccgtccggcctggggttggatagcatcggcgacctgtcgagcctcctggacgctcctgcggcgtctcagggcggttccggccctatcgagctggacctggacctgatcgacgaagatccgcatcagccgcggacggccgacaaccccggcttttccccggagagcatcgcggaaatcggtgccacgatcaaagagcgcggggtgaagtcacccatttcggtgcgcgagaaccaggagcagccgggccgctatatcatcaatcacggcgcccgccgctaccgtggctcgaagtgggccggcaagaagtccatcccggcgttcatcgacaacgactacaacgaagccgaccaggttatcgagaacctgcaacgcaacgagctgaccccgcgcgaaattgccgacttcattggccgcgagctggcgaagggcaagaagaaaggcgatatcgccaaggaaatcggcaagtcgccggcgttcatcacccagcacgtcacgctgctggacctgccggagaagatcgccgatgcgttcaacaccggccgcgtgcgcgacgtgaccgtggtgaacgagctggtgacggccttcaagaagcgcccggaggaagtcgaggcgtggcttgacgacgacacccaggaaatcacgcgcggcacggtcaagctgctgcgcgagttcctggacgagaagggccgcgatcccaacaccgtcgatgccttcaacggccagactgatgccgagcgtgacgcggaggccggcgacggccaggacggcgaggacggcgaccaggacggtaaggacgccaaggaaaagggcgcgaaggagccggacccggacaagctgaaaaaggccatcgtccaggtcgagcacgacgagcgccctgcccgccttatcctcaaccgtcggccgccggcggaaggctatgcctggttgaagtacgaggacgacggccaggagttcgaggcgaaccttgccgacgtgaaactggtcgcgctcatcgagggctgatccccaaagacagcggcgcgggccacccgcgccgcacagacaacggttccgctacaaggaggaccgaagaatgaatccgatgctgttctacatcgcgggaggcgtaggcgcggcgttgctgctggtttccgcgatcatgctgttcaagctgcgcgagccgaagaaggaacaccgaccgcagcgcaaggcggcggccccgacgccgcagccggtcgataacgagctgctgcgcacctttcgccggccgtcgcaggccagccaacccgagccatccacgacgccgccggcgcggcgcactgccccgccgagcagcgaacaatccagagggaatgacggcgatttcgtcacgtcggccctggtggccggggcgaccaacagcaccatgctgggctacctggccggcggctcgctgaccggggccatgctgggcgatgcgctgcggccggatacgccgtctgcggcgttcgcggacccctcgccgtgcttcggcagcagcgacacggacagcggctggaccgataccggcagctcttgcgatttcagttcgtccgacaccgatacgaacaactggtgagggcggaccatgactgacgaacaaaagaaacggctcctgatgttgcaagctatccggcgcagcatccaggcaatgcgcaggggtgtttcggggccggatagtttcaagggctggttgaccggcatcggcatcatcgcggcggggttcctggttggcgagctgctgccatcccctgcccccggcgtcggcctggttgtgccaggggcctgcattgccggcttcgtcgtcggctgctggctcaacaggaaaggccgctcatggccgacagccatttacgaccaattggcccgttaccagccgctcaacgagcgggcgtaccgcgacctgcaagagacggtgaaggaacgcggcctcgactgcgatgcggtcatggagtggacctacatcgagcaggccgagttcatgcccctggtgcccaccagggaggacctagcgcgcgacaagttcctgaccgcgacgcgggccgatagggccgaggacgatgcgcgtgcgcgcttcgtcgtgctccatcccaagaagcgcggccccgacgcatgaagaagcccgacgcccgctaatccccttcccgctgaccctttgcccccgccgaccgtgcggggctttttttgcctgcccttcagccgctaaaaaacctgccgttaagtcgagaactgttcagctaaactcttgcgctgataaggtaggtaagagtattattattcttaccaattaccggagccaacatggaactgaacaaagagacgcgcgaccgcatttttgcggctgcggacgagctgttcgagcaaggcgaccgggagaacttcccgaccgtggatgcagtgcgtaaggccgcccgcgtcaacatgaacgacgccagcgccggcatgcgcgaatggcgacgccagcgcacggcgcaggccgccccgctggcagtccaggtgccggacgcggtgcagcaggccggcaaccaggccgtggccgccctctggcaagccgcccaggcccttgccaatgaatcgttgcaggcagcgcaagccggctgggatcgtgagcgcaacgagttggaagccgtgcgcggggagctggccgacgccttcgaggcgcaggcccgcgagctggaagaagcccaggcgcgcgttacattgctagagcagcaggccgccgaggcggccgagctggccgcacgccagcggcaagccctggccgaagcacgcaccgccctggcaggagccgagcagcgggccgcgctggcgacgcagcgggccgacgaggtggagcgtcgagcgcaggagctgcgcgccgagctggactacgctcaccaggacgcccgcgcattcaaagaagagagccgcaagaccctggacgcggccaaccaggaaatgcaggcgcttcgcggcgagcgcgacgccaccaggcgcgaagcggagagtttgcgcacggagttggcagcggtgaaagcgaaggcgcaggccgacgccgaggcccaccaggagcaaaggaagttggcggcgcaagaggcggccaggcaggccgagcgctttacgacggtgcaagccgagcgtgacgaagcgcggcaagaggcaaaggcagcccaagaagaggccgctatgttgaggggtcggctagacgcggaaacgagtgggaggaaacacaaagcgggaggaaagtaactgataaatcgaacgcagttcactaaaatagatggaacaaccttgacgcgcccggctctagggaaagccggcctcgaactgccgaggtcggcttttttttagccggcaagcgaccaggacacgtttaacaccagaggcaaccgtcaggagtcacccaatgaaacaccgcctcctggtcatgaatgggcaaagaattgtccagaccgagaaccaaggcgcatggacgaaccagaaggtagataaagccggggccctgaaacctggcatttacaacatctatatggcccaaaaggccgataagtcgcagcgccacgacggcagcatcgtccacgcagatagcggcagcatttaccaacaggtggggaagaactttgtaatgcacgcccggtcagatttcgataaagtacctgaaatcgggagtgcgaaaagcatcacctatgacgcaagtggcaaggcacaggtaagtgccgaaagcgtcaagttgagtcgcgggcgcactcgttagagttttagcggctaaaaagggaggcgtcttgagcagctacagcagagccgagcgcgcaccgtggggagacttcccgaaggtggttcgcaacggcgaccttggttctttgaccaacgagcctgaataccaggctgcgaagcagggcgacgcggaagcggcgcttaatttggtcgagcgcctgatttcggacgacactgttgcccagctcaagacgctgatcggcgacgacaagccccgcatcgttccagtcctcgccgtcgaggcggcggggaacaacaagattccggccatgatggcggtcgttctggccgaccgccttggccttgaggtcgaaacggacattgtgcagcgggagaaggtcgcccgaaccggagccggttcagatcatcgcctggcgttcaacccgactttcgagggggaggtgatccccggccagaagtacatcgttgtcgatgacacgctgacgatgggcggcaccattgcctcattacgcggctacatcgagaacaacggcggcaaggtcatggcggcgtccgtcatgaccgcccatgaaggcgcgcttgacctggcggtcaagccgaagatgcttgcgggcatcaatgagaaacacggtccagcaatggacgcattttggaaggaaacctttggctatggcatcgaccgactcacccaaggagaagccggacacctccgcgctgccccgtccgttgacgcaatccgagatcgcatcgctgcggctcgaaatgaagcagtcaatcgagtgggggcaagccgaactgcgacgacggcgcgagcagggcaacagcagtccccggcagtaaagcagtccagcggcaacagcggcgaggatttgctacaggcggcccaggaggccgagacggaacagcaggccctcttagaatccgcgcccatcgagcagacctaccagcagacccttgcgctctacgtgcaggcaaagcatgaccaggtggagcgcatcgaggaccgcctagaacagctcgtagaccgtcagcaggcgcgtttgcagcagctacagtccaatgcccccggcttgctgtccctgccgcgtacaaaggccgcctggcagcagcagcaggcccaacagcaagcccgcttgcagaccctgcacacgcggctggacatggtgcgcgaaatcaaggaaggcatgggcattcacgcgcccaagatcgaggaactggcgacgcgcaagatgcgcgcggagcatcccgagctggccggcgactgggattcgatgcgtgaagcagcccgacggcaccaggccctgatgcgcaagcaggagcaggagaagaaacaggcccaggagcgcgagcgcgggcgcagtcagagcttgggcctgtcaaacaagcccagctaacgcaaaaacaaagcccggcaacgccgggctttttcatctgcgcctctgcgattcataacgaggcccacaccaccagggcggccgcgaacaacaccatgccgccggcgatcatgttcatcatcgccacgcgccgcgcgtccgcgaccttggccgcgagctggcggccaaggccgtcgtcgatttccctgcggatcgcttcggccgcctgcgcggcgctgtccttcattaccttcgccattgcgtccttgctggccgccagggccgcgttcagcatccgctccgctttggccttggcgtcctcgccccaacgatgggcgatcccttccagctcttccttgaacgcggcaaggatttcctcttgcttggccgcactgtcggccatgagccgggcgttgatggtatgcaggatcagcaccgggtcgtcgcggccgacggcgatgccgtgcttggccgcaatctcccggatcagctcttcaatctggtcgctcatagcacggccgccgcgtcgagctgttcaaacaggccgcgccgcacgatcttgaggcgttgccgcgtcatgatcgtgagcgattcatcggccagcgcctggtcgaacgtcagccgctcttgcagcatgtcgctgaaatcgcggccgtaggtttcttccttgagggccggaatctggatgatggacgacacgcgggccttgttggccgtgtacgccttcatctgctcaaagctcttgccctcatgctcgataggcccccaatacgggttcagccagaccacgaaaagcgcttcggccgggaactggctggcgagctgggcgaagccgctcaccgtgtccaggagagcctggccgccggtgacgacggtatggatgaccagctcatgccccatttcttgcagcagagccggcacctggttgctgatgaggtaatgcgacagaggcacgaacgagctggcaccgttgtcgatcaccacgtcatccttggtcggcgcaatcagctcgaccagggtgtcgaagttgcgcgagttaatttcgtcgccggccatgatgttcagccggcggacgttcagggccttgtagccctcgaacgtcgcgttcaccgggtcggtgtcgatgcacaagggtgtctgccccttgtccatcttgtactgcgcaatgatcgcggcgatggccgacttgccgaccccgcccttgccctgcaaaaccatgtgaattttcgccattacagtagatcctttttgtccggtgttgggttgaaggtgaagccggtcggggccgcagcgggggccggcttttcagccttgcccccctgcttcggccgccgtggctccggcgtcttgggtgccggcgcgggttccgcagccttggcctgcggtgcgggcacatcggcgggcttggccttgatgtgccgcctggcgtgcgagcggaacgtctcgtaggagaacttgaccttccccgtttcccgcatgtgctcccaaatggtgacgagcgcatagccggacgctaacgccgcctcgacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgctcttcttcaatcgctgccgtcagacggtcagggaacgccgacaggccccgctccgcgtagttcggagcaggacgccgcacggcgtccacatggatcatccacccacttcccaggcccgcgagggcctggttgatgcgggcggacactacttcgcgctgctggtcggtgctgcttgcgttgtcatcgcccttgtacagccaggcagccataaagctgccgttcttgcccacgattacgccgtcatcgacgacagcggcgtagttgagcagatcggccaggccggcgtccttggaacgatgctttttcagtttcagttcggcatcgaccgcgcggatgcgggcaaagaggatgaacaacagaagcgcgccgaggcccgcgattgcaatcgcaattgcttggatcatcggtattgcttcccttggctattggtgttctcgcggaacggggtcgagcgggccgggtaatacggcttgtaccggcggtgacgcaggtacacgaaccgcatcttcggatcggccttcgccatgattcggaacgcatagagcgccccgaaccacaggatcagaccgaccacggtggcccgcagctcttgggcgctgaaaatcagcgcaaacgccatcaggcccgagaacatcaccagttcacgatcaccacccatgaacaggttttctcggttgcctgcgcgacggatggggatcgtgcgcagagccatgattaggcgagccgtccagccgctaccgcacgcacggcatccgccgccgcgacttgcacctggtgcagcgccccgttgccgagggccgcgatttcggcaccacgaccgaagaaggtgctcatcacgttctgcgcgccgaccagcagcgccatcaccagaaccaggaagatcagggttcggaagaaggcgttgagttcgccgccgaagatcagcacgccgccggcgacgacgatgccgatgatggacagcgcgaaggccaccgggccggttacggagttgcgcaggttcgtcagccagctctcatatggcaagctgccgccggtgccttccgaggccatcgccggatgcgcggataacgcgagagcgagaacgaagaacacggcaaggtagaacaaaatgccgcgattcatggtcagacggaacggaacagccgttgtcattggaaatactccttacagggttttggtgatgtactggccgttctcgtaaccaagaacttcgagaatttcttgcactcgacggccgctaggggtcctggcgatatggacgaccacatgaaccgcctcgccaatcagcggctcaatgggtttcggtgaatccgggtgcatgctgataagcatggcgagccggctcaggcccgctttggggttgtttgcgtgcagggtggcggcacctccttcatgcccggtgttccaggccatcaacagatcaagggcttcggggccacgtacctcaccgaccaggatgcggtcggggcgcatacgcagcgttgtcttgagcagcagcgtcatcgagacgtcgatgctggtgtggtattggacggcgttctctgcggcgcactggatttcgccggtgtcctcgatgatgacgacgcgctcagacgggttgaaggcgaccatttcattgatgatcgcgttgacgagcgtggtcttgcccgagccagtaccgccaatgacgaggatgtttcgatgcgccgcgacggcgcttttaatgacctcgtattgctcgcgggtcatgatgcccgcctcgacgtactgttccagcgtgaagatggcgaccgcgcgcttgcggatcgcaaaggttggcgcggccacgaccggcggcaattggccggcaaagcggctgccatccaaggggaactcgccttccaggatgggcgaatgccgcgtgacctctttgccgtggaatccggccaccgtttctataatcgcctgcgactggctgggccgcatgtcgcagatgtaccgcatcggctcgccaaggcgttcgtgccacaccttgccgtccgcgttgagcatgacttcaacggttttcgggtcgttgagcgcggccaacaggtccgcacccatgtcgcgttccagcttgcgcttggcccgctccttgatggtctgaaactcttctttcccgctcacttttcagtaacccctacgcaacaaatcattagcaacattatcgcacaggcaaacgatataccgaaagtgtttagcctgcaagacaaacagctaatcgaaccgcacgacgtaggggttctgatagaaaacaactgtcaaagcgcacccgcccgatgccattcgcggcacggcttccgttgaggatgtcgatatgatgcgcgagccgacggcccgcagagaaggggccgttttagcggctaaagaaggaagtgcaagccctaacccttggcgtcagagccttccacgcagcttttttcgggtgtcgtcgccccatttctttacgataaacgccttatgtgacggcaaaaccacactgatgcgttcgtatccgggcggcacgctgctcttgaaaggatgacccgcaatctccgcgagtgcctcgcggtcaaggtcggtggactccaggagaagaggtaggggagtttccagggcgtcggcaatggcctccatcaccttcaacgaggggttggccttaccgttggttaagtctgataaaaacgaaattgaaacccctgccctctccgacagctcatgtttcgtcatgccccgctcatcgagcagacgaaggatgttggtgaaaaatatctggttgtacacagcggaagccgcccctcgcacctttggtcgcggcccgcaaaattttagccgctaaagttcttgacagcggaaccaatgtttagctaaactagagtctcctttctcaaggagactttcgatatgagccataatcagttccagtttatcggtaatcttacccgtgacaccgaggtacgtcatggcaattctaacaagccgcaagcaattttcgatatagcggttaatgaagagtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccggggaccgccgtgttgctaggatggttgttcttggatcagacgctgagtgcgcttcaaatcatcggcgtcctgctcgtgatcgggagtatctggctgggccaacgttccaaccgcactcctagattgcgcgtcccccttggtcaaattgggtatacccatttgggcctagtctagccggcatggcgcattacagcaatacgcaatttaaatgcgcctagcgcattttcccgaccttaatgcgcctcgcgctgtagcctcacgcccacatatgtgctaatgtggttacgtgtattttatggaggttatccaatgagccgcctgacaatcgacatgacggaccagcagcaccagagcctgaaagccctggccgccttgcagggcaagaccattaagcaatacgccctcgaacgtctgttccccggtgacgctgatgccgatcaggcatggcaggaactgaaaaccatgctggggaaccgcatcaacgatgggcttgccggcaaggtgtccaccaagagcgtcggcgaaattcttgatgaagaactcagcggggatcgcgcttgacggcctacatcctcacggctgaggccgaagccgatctacgcggcatcatccgctacacgcgccgggagtggggcgcggcgcaggtgcgccgctatatcgctaagctggaacagggcatagccaggcttgccgccggcgaaggcccgtttaaggacatgagcgaactctttcccgcgctgcggatggcccgctgcgaacaccactacgttttttgcctgccgcgtgcgggcgaacccgcgttggtcgtggcgatcctgcatgagcgcatggacctcatgacgcgacttgccgacaggctcaagggctgatttcagccgctaaaaatcgcgccactcacaacgtcctgatggcgtactaagctgggcctaggaaatatcagccgcttgtgtgttcaattcttctctgtttcacttgaaacaaattgaatagatattcccgctttcaaagccatttacaaatcctctcgtgcagcctaacgccattggcgtggaacaagcgttggcacgaggaagtaagtgcgatgaacggaaggtattcaccgacgcggcaggattttaagacaggcgcgaagccttggtctatattagcccttatcgttgccgcaatgattttcgcgttcatggcggttgcgtcctggcaggacaatgggactacccaggcaatccttagccaaatacgatcgattaacgctgacagcgcctcactgcagcgcgatgtactccgcgctcacacgggcaccgtggcgaactaccgccccattatctccaggctgggagctctgcggaagaatctggaagatttgaagcaattatttaggcaatctcatattgtaagtgagagcaatgctgctcaactgctacgccggctagaagtgtctctaaattcggctgacgcggcggtcgccgcctttggtgcgcaaaatgtccgcctgcaagattcgctggccagtttcactcgtgctttaagcaatcttccaggaaaggcctcaggcgatcagactttagaaaaaccaacagaattggctagcatgatgctccaatttcttcggcaaccaagcccggctatttcattcgagatcagccttgaactagagaggctccaaaaacaacgcggtcttgatgaagctcccgtgcgcatactttcgcgtgaaggtcccattatcttatcgcttttgccacaggtgaacgatctggtgaacatgattcagacgtctgacaccgcagaaattgcggaaatgttgcagcgtgagtgtttggaggtctatagcttgaaaaatgtggaggagcggagcgcacgtatctttcttgggtccgcttcagtgggtctttgcctgtacatcatcaccttagtctataggctacgcaaaaaaaccgattggttagcgcggcgtttagattacgaagagctaatcaaagagatcggagtatgttttgaaggtgaggcggccactacgtcgtccgcgcaagctgcacttggtattattcagcgcttctttgatgccgatacgtgcgcgttagctctagtggaccatgaccgtaggtgggctgtcgaaacattcggtgcgaagcacccaaaacccgtgtgggacgacagggtgctacgcgaaatagtctctcgtaccaaagcgaacgaacgggcgacggtattccgcatcgtatcgacgcaaaaaatcgtacatttgcctcccgaaattccaggtctttcgatactactggctcacaaatccacagataaactaattgcggtttgttcactgggttaccaaagctatcgccctcgaccttgccaaggcgaaattcagcttcttgaactcgccaccgcctgcctctgtcactatatcgatgttcggcgtaagcagaccgaatgcgacgttttggccagacgattggagcatgcgcaacgccttgaggcagttggtacacttgccggcggaatagcacatgaatttaataacattttggggtcaatcctcgggcacgcagaattggcccaaaactcggtgtctcgaacatctgtcacccgaagatacattgactatatcatttcgtcaggcgacagagccatgctcattatcgatcagatcttgacgctgagccgaaaacaagagcgcgtgatcaagccatttagtgtctcagagcttgtgaccgaaatcgctcccttgctacgtatggctcttcccccaaccatcgagcttagtttcagatttgatcaaatgcagagcgtgatcgaaggaagcccgcttgaacttcaacaggtactaattaacctctgcaagaatgcttcccaagccatgactgcaaatggtcaaatcgacatcttcgtcggccaagcttatttaccagctaagaaaattctggcgcatggtgttatgccacctggcgactatgttctactatctgtcagcgacaatggtggaggcatttcagaggctgtgctaccctacatttttgaacccttctttacgacacgtgctcgcaacggtggaacgggtctcggcctcgcttctgtgcatggtcatatcagcgcgtttgcaggttacatcgacgttagttcaactgttgggcatgggacgcgctttgacatttatctccctccgtcttctaaggagcccgtcaatcccgacagctttttcggccgcaataaggcaccgcgtggaaacggggagattgtggcacttgttgagcccgatgacctcctgcgggaggcgtatgaagacaagatcgccgctctgggatatgagccggtcggttttcgtacctttagtgaaattcgcgattggatttcaaaaggcaatgaagccgatctggtcatggtcgaccaagcgtctcttcctgaagatcaaagtcctaattccgtggatttagtgctcaagaccgcctccatcatcattggcggaaatgatctcaaaatgcccctttcaagggagaatgcgaccagggacctttatctgccgaagccgatatcgtccagaactatggcgcatgcaatcctaaccaaaatcaagacgtagagttgcgacgtatcaggactgggcaatcgcggccgccgggcttacgtgtcttttgtaacaaattgcgacgcagttgatatccacttgaaacattagtccgaaattatcgagatctccgctgataaggtatcgaaatggcgataaaattggtattgatactcgtattcacactctttcccgcggcagacgctgcatatgcgaatgaccgcgccaacggtttcatgtggtcaaacgggggcgaaactggagtgaggcttcctcttcgggtgttcaatgccaagccagccaagaacacggtggcgatcatttattccggagacgctggatggcaaaatatcgatgaggcgattggtacctatctgcagacggaagggattcctgtcattggcgtcagttcacttcggtatttctggtcggagcggtccccaagcgaaactgctaaggatcttggtcacataatcgatgtctacaccaagcatttcggtgtgcagaatgttttacttgtaggatattctttcggcgcggacgtcatgccggcaagcttcaataggcttacgcttgagcaaaaaaatcgggttaagcaaatctctctcttggcattgtcacatcaagtcgactatgtcgtctcatttaggggctggctccaactcgaaacggaaggtaagggcggcaatcctctggatgatctcagatccattgaccctgcaatcgtccaatgcatgtacgggcgcgaagaccgtaataatgcttgcccatctctccgacagaccggcgcagaggtgataggcttcagcggaggccatcactttgataatgatttcaaaaaactgtctacgcgcgtcgtctcaggcctcgtggcacgcctaagtcatcagtaatctttagttcctgcaccgcgggacccgcgaggtttctcgcttcaattgaaatcataaagaagcaattgaaaattttcgagtaaccgaccctcccgataatcttcaacataaaacaacgcacttcttccaacgggagaggcggtgttagttgcgagctaaggagataaggtatgcttaagagatcggggtcgctttctcttgccttgatggtctccttctgttcgtcgagccttgccacgccactctcatctgctgagtttgaccatgttgctcgcaagtgtgccccatcagttgcgacatctacgcttgcggcgatagctaaggtggagagtcgctttgatcctttagcgattcatgacaacacgaccggcgaaacgcttcactggcaagatcacagccaagcaacccaagtcgtcaggcaccgtctcgatgcacggcattcgctggatgttggcctcatgcaaataaactctcgaaatttttctatgctcggtctgacacctgacggtgcgctccaggcgtgcacatcattatctgccgctgcaaacatgctgaaaagtcgttatgcaggcggcgaaacgattgacgagaagcaatttgcgcttcgtcgggcgatctccgcttacaacaccggtaatttcatcggcggttttgcaaacggctacgtgcgaaaagttgaaacagctgctcaatcgctggtgcccgcgttaatcgagcctccaaaagacgatcacgaggcgctaaaatccgaagagacgtgggatgtttgggggtcatatcagcgccgctcgcaggaggatggcgctggcggtttaatcgctccgccaccgccacaccaggacaacggcaaatccgcagacgacaatcaagtcttattcgacttatactaaggaggtgcgcattgatgcgatgctttgagagataccgtttacatctaaatcgcctctcgctctcgaatgcgatgatgcgcgtgatatcgagctgcgccccaagcttgtgcggtgcaattgcatggagcatttcctcatccggacccgccgcagcgcaatctgcgggtggcggcactgaccccgccacaatggttaacaatatatgcacgtttatccttggtccgttcggccagtcactcgctgttctcggcattgtcgctatcgggatctcctggatgttcgggcgggcttcgcttgggctggttgccggcgtcgtcggcggcattgttatcatgtttggggcgagcttcctcggccaaacgctcactggcggtagttgatggctgatcgtttggaagaatcgaccctttacctcgcagccacacggcccgcattgtttcttggggtgccactgacattggcagggttattcatgatgttcgccggctttgtcatcgttatcgttcagaacccgctctacgaagtcgttctcgtgccgttatggtttgcagcccggctcatcgtggagcgagactacaatgcggcgagcgtcgtcctgctatttttgcggaccgcgggaagaagcattgatagtgcagtttgggggggcgctactgttagcccaaatccaattagggttcccccacgagggagaggaatggtgtgatgctcggcgcgagtggaacgaccgaaagatccggtgagatctatctcccttatattggccacctcagcgaccatatcgtccttcttgaagacggatcgatcatgaccattgcgagaattgatggcgttgcattcgagcttgaggaaactgaaatgcgcaatgcgcgttgtcgtgcgttcaacacgctgttgcgcaatatcgctgatgatcatgtgtcaatatatgctcacctcgtacgtcatgccgacgtgccatcatcggcgccgcgacacttccgtagtgttttcgccgctagcctgaacgaagcttttgaacagcgcgtgctctccggccaactcctccgcaatgaacacttccttacgttgattgtctacccacaggcggctttagggaaggtaaagaggaggttcaccaagctaagcggaaaaagggaaaacgatctcacgggccagatcaggaacatggaagatctttggcatgttgtcgctggctctcttaaagcgtatggcctgcatcgtcttggcatccgcgagaagcagggtgtgctcttcaccgaaattggcgaagcgctacggttgatcatgactggtcggttcacaccggttccggtcgtcagcggctcactcggcgcttcgatttataccgacagagtcatttgcggcaagcgaggactcgagatcagaacgccaaaagacagttacgttggatccatctattcgtttcgcgaataccctgcaaaaacacggccgggcatgctcaacgcgctgctatccctcgattttccacttgttctcacgcagagtttttcgttcctgactcgcccgcaagcgcacgcgaaacttagcctcaaatcgagccagatgctgagttccggcgataaagccgtgactcaaatcggcaaattatccgaggctgaggacgcacttgcgagcaacgaattcgttatgggctcacatcatttgagcctttgcgtctatgcagacgatctcaatagtcttggggacaggggcgcgcgggctcggacacgaatggcggatgcaggtgccgtggttgtccaagaaggtattggtatggaagcggcctattggtcccaattgccggggaattttaagtggcgcacacgccctggcgcaatcacttcacgcaatttcgcagggtttgtctctttcgaaaactttccagagggcgccagctcaggccactggggcaacgcgattgcccgatttcgtaccaatggcggaacgcctttcgactatatcccgcatgagcacgatgttggcatgacggcaatattcgggcctatcgggaggggtaagacgacgctcatgatgtttgttctagccatgctcgaacagagcatggtcgaccgtgcaggtacggtcgtgttctttgacaaggaccggggtggcgaattgctggttcgcgccacaggaggaacatatttggcacttcacagaggcacacccagcgggttggcgccgttgcgtggcctagaaaacacagcagcctcacacgattttctgcgcgaatggatcgtggctctcatcgagagtgatggtcggggtgggatttctccggaagagaaccgccgtctggtccgtggtatccatcgtcagctctcgtttgatccacaaatgcgttcaatcgcggggttacgtgaatttttgttgcatgggcccgccgaaggcgcaggagcgcggctccaacgctggtgccggggccatgcgcttggctgggcatttgacggcgaagttgacgaagtaaagttagatccgtcgattaccggcttcgacatgacgcatcttctcgaatacgaggaagtatgcgctcccgctgcagcatatctcctgcatcggattggagccatgatcgacggccgccgttttgtgatgagctgcgatgagtttcgcgcctatttgttaaaccctaaattttcgactgtcgtcgacaaattcctcctgaccgttcgaaaaaacaacgggatgctaatactggcaacgcagcaaccagagcatgttctggaatcgccgctaggagccagcttggttgcgcaatgtatgacgaagattttctatccatcaccaaccgcagatcgatcggcttatgtcgatggactgaaatgtaccgaaaaggaatttcaggcgatccgtgaagacatgacggtcggcagccgtaagtttcttcttaaacgagaaagtggaagcgtcatctgcgaatttgatctgcgggatatgcgtgaatatgtcgccgtgctttcggggcgtgccaacacggtgcgctttgcaactcgactacgcgaggcacaagaaggcaactcatctggctggctcagcgaattcatggcccgtcaccacgaggcagaagattgataaggtaggaaacgatgaagacgacgcaacttattgcaacagttttgacctgcagctttctatatattcagcccgcgcgggcgcagtttgttgttagcgacccggcaacggaggctgagacgctcgcgactgcgctcgcgactgcggagaatctcactcagactatagcgatggttacgatgttgacgtcggcctacggcgttactggactactgacttcgctcaaccagaaaaatcagtatccttcgacgaaggacctagacaatgaaatgttttcgccgcgaatgccaatgtcgaccacggcacgtgcgatcaccagcgatacagatcgtgcagtcgtgggtagtgatgctgaagcggacctgttgcgatcgcagatcaccggttccgcaaacagcgctggcattgcggctgacaatctggaaacgatggacaaacgcttgacggcgaatgctgatacgtctgctcagctttcccgatctcgcaatatcatgcaggcaaccgtgaccaatggtttgcttctcaagcagatccatgacgcaatgattcaaaatgtacaggcgacaagcctattaacgatgactaccgcgcaggccggccttcacgaggcggaagaggcggccgctcaacgcaaggagcatcaaaagaccgctgtcatctttggtgccctcccctaaggctgggcgatttgttcatccgcccgcatcctcgccgaatgcgagctcattttatccaacattatgcgacaaaccagtcaagttcaggtccaatcgatgaatttcacgattccggcgccgtttacggccattcatacgatcttcgatgtagccttcacgacaggcttggactcgatgcttgagactatccaggaggcggtgagtgcgccattgatcgcctgtgtcactctttggattattgttcagggtattttagtcatacgcggcgaagtcgatacccgtagcggtatcactcgggtgatcacggtcaccatcgttgttgctctaattgttgggcaggctaactaccaagactatgtggtttccatcttcgaaaagacggtcccaaactttgttcagcagtttagtgtaaccggcttgcctctgcagactgttccggcacagttggatacaatgttcgccgtgacccaggccgtttttcagaaaatcgcatccgaaatcggtccgatgaacgaccaggacatccttgctttccaaggggcacagtgggtcctttacggcacgctctggtctgccttcggagtttacgacgccgttggaattctcacgaaagtgcttctcgcgatcgggcctctgatcctcgtcggatatatttttgatcgcacgcgggacatcgcagctaagtggatcgggcaacttatcacctacggtctcttgcttctcctcttaaacctcgtggcaacgatcgtcatcctaaccgaagcgactgcgctcacccttatgcttggtgtaatcaccttcgccggtacgaccgcggccaagatcattggtctttacgaactcgatatgttttttctgacaggggatgcgctcattgtcgctttgccggcgatcgccggcaacattggaggcagttactggagcggcgcaacccaatctgccagcagcttgtaccgtcgcttcgctcaggttgagcgaggctaggtcgcgcaaaaattcgcctcaatggagaattctatgaagtattgcctgctgtgcctagttgtcgctttgagcggctgccagacaaacgacacattagcgagctgcaaaggcccgatcttcccgctgaatgtggggcgatggcagcctactccgtcagatcttcagctcggcaattcgggtggacgctatgacggggcctgaatatgccatgctagtggcgcgcgaaagccttgccgagcactataaggaagtagaagcctttcaaaccgcgcgagcgaaatcggcgcgacgtctctccaaactcattgcagctgtcgcagctatcgcgattttgggaaatgttgctcaagcgttcgctatagccacaatggtgccgttgagcaggcttgtgcccgtatatctatggatacggccggacggcaccgttgacagcgaggtgtctgtctcgcgattgcctgcaactcaagaggaggccgtcgttaacgcctcattgtgggagtacgttcgcctgcgcgagagttatgatgccgacaccgctcagtacgcctacgacctggtatcgaacttcagtgccccaacagtgcgccaggattaccagcaattcttcaactatcccaatcccagttcgcctcaagtcattcttggcaaacgcggcagggtggaggtcgagcacatcgcttcaaatgatgtaactccaagcacgcagcaaattcgctataaaaggaccctcgtcgttgacggcaaaatgcctgtggtgagtacgtggaccgcgacagttcgctacgaaaaggtgaccagcttgcccggcagattgagactaaccaacccggcaggtctggttgtcacctcctatcagacatcggaagataccgtttcaaacgtaggccacagcgaaccatgatcagaaaagcacttttcattttagcatgtttatttgccgctgcgactggtgcggaggctgaagacactccaatggcgggcaagctagatccgcgcatgcgttatttggcttacaatcccgatcaagtggtgcgcctctcgacggcggttggagctactttggtcgtaacattcgccacgaacgaaacggtgacagcggttgccgtttcaaatagcaaagatctagcagccctaccgcggggaaattatctatttttcaaggcaagccaggtcctcacgcctcagccagtaatcgtgctaaccgcaagcgactccgggatgcgccgttatgttttcagtataagttccaagactctgtcccacctcgataaagagcagcccgatctctattacagcgtccaattcgcctaccccgccgacgatgcggcggctcggcgaagggaggcacaacagaaggctgttgtggacagactacacgcggaagcacaatatcaacggaaagctgagaatttattggatcagcctgtcacagcccttggtgcggcggacagtaattggcactacgtcgcccaaggcgatcgttcgctgttgccactcgaagtcttcgacaatggatttacgacggtattccactttccgggcaatgtacgcataccctccatctacaccatcaatcctgatggcaaggaagctgttgccaactattcagttaaagggagcgatgtcgagatttcttcggtttcccgaggttggcgtctgagggatggccacacagtactatgtatctggaacaccgcttacgatcccgttggccaaaggccgcaaacgggcacggtgaggcccgatgtgaaacgcgtcctgaagggggcgaagggatgaataacgatagtcagcaagcggcacatgaggttgatgcatctggatccctggtctccgacaaacatcgccggcgtctttcggggtctcagaaattgatcgtcggaggtgtcgttctcgcgttatcattaagcctcatttggctaggtgggcgccaaaagaaggtgaatgagaacgcatcgccgtcaactttgatcgcaacaaacaccaagccatttcatccagctccgattgaggtgccgccggatcctccagcggttcaagaggctgttcagcctgctgctcctctaccgccgaggggcgaaccggagcggcatgagccacggccggaagaaacaccgatttttgcatatagcagcggcgatcaaggggtcagcaaacgcgccattcagggcgacacgggccgaagacaagaaggcaagcgtgacgacaactccttgccgaatggcgaagtgtccggcgagaacgatttgtcgatacgtatgaaacccaccgagctgcagcccagcagcgccacgctcttgccgcaccccgattttatggtaacgcaagggacaataattccgtgcatcttgcaaaccgcaatcgacacaaatttggcaggctatgtaaagtgtgtcttgcctcaggatattcgtggaacaacgaacaatatcgtgcttcttgatcgtggcaccaccgttgttggcgaaatacagcgtggcttgcaacagggagatgggcgcgtttttgtgttgtgggatcgcgccgagacacctgaccatgcgatgatctcgttaacatcgccaagcgcggacgaactcggtcgctcaggattgccgggctcggtcgacagccacttctggcagcgttttagcggagctatgctcttgagtgttgttcaaggcgccttccaggcagctagcacctacgccggcagctcgggtggcgggatgagcttcaacagctttcaaaataacggtgagcagacaactgagacagcccttaaggcaaccatcaacataccgccaaccctgaagaagaatcagggtgacaccgtttccattttcgtagcacgggacctcgatttctttggtgtttaccagctccgcctgactggcggcgccacgcgggggaggaaccgccgctcttaatgaattcaaatttccgcttagagataggatacattgtaaatggaagtggatccgcaactacgctttcttctgaagccgattttggaatggctcgatgacccgaagactgaagaaattgcgatcaatcgacctggagaggcatttgtgcgccaagccggcatttttaccaagatgcctttgcccgtctcttatgatgatcttgaagatatcgctattttagcgggcgcgctgagaaagcaggatgtcggaccacgtaaccccctctgcgccactgaacttcctggtggtgaacgactacaaatctgtctgccgccgaccgttccctcgggcaccgtcagcttgaccattcgacggccaagctcgcgtgtttctggtcttaaagaagtctcctcccgttatgatgcttcgaggtggaaccagtggcagacacgaaggaaacgccaaaatcaggatgatgaagctatccttcagcattttgacaacggggatttggaagcgtttctgcacgcatgcgtcgtcagccgactgacgatgttgctatgtggccctaccggaagcggcaagacaacaatgagcaagaccttgatcagcgccatccccccccaggaaaggctaatcaccatagaagatacgctcgaactcgtcattccacacgataatcatgttagactactctactccaagaacggtgctgggctgggtgctgtgagcgccgagcacttgctccaagcaagtctgcgtatgcggccggaccggatattgcttggcgagatgcgcgacgatgcagcatgggcttatctgagtgaagtcgtctcgggacatccgggatcgatttcaacaatacacggcgcgaatccaatccaaggattcaagaaactgttttcccttgtcaaaagtagcgcccaaggtgctagcttggaagatcgcacactgattgacatgctctctacggcgatcgatgtcatcattccattccgtgcctatgaggacgtttatgaagtaggcgagatctggctcgcggcggacgcacgacgccggggcgagaccataggcgatctccttaatcaatagtagctgtaacctcgaagcgtttcacttgtaacaacgattgagaacttttgtcataaaattgaaatacttggttcgcattttcgtcatccgcggtcagccgcaattctgacgaactgcccatttagctggagatgattgtacatccttcacgtgaaaatttctcaagcgctgtgaacaagggttcagattttagattgagaggtgagccgttgaaacacgttcttcttatcgatgacgatgtcgctatgcggcatcttattatcgaataccttacgatccacgccttcaaagtgaccgcggtagccgacagcacccagttcactagagtactctcttccgcgacggtcgatgtcgtggttgttgatctaaatttaggtcgtgaagatgggcttgagatcgttcgaaatctggcggcaaagtctgatattccaatcataattatcagtggcgaccgccttgaggagacggataaagttgttgcactcgagctaggagcaagtgattttatcgctaagccgtttagtacgagagagtttcttgcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttacgacaggagtatagatgttctcattttgcggctgcgccgcaaacttgaggcggatccgtcaagccctcaactgataaaaacagcaagaggtgccggttatttctttgacgcggacgtgcaggtttcgcacggggggacgatggcagcctgagccaattgcatttgattaatttaggtgactgaggacgcggccagcggcctcaaacctacactcaatatttggtgaggggttccgataggtccctcttcaccaattgctcgatggcttctctccagcaaagaatgacgcgagcgcggcggtagccagcttgtggccgaaagctcgagcggtctccaaccccaacggatcaaaatgacttcgagcgacctcgagcaacgcaaccgggaacatgcgtgaggtctgaacgagaacggatttttctgtagttgaagggatcggataacttttcggggccacgcgaaatgatccatctgccagcatgctttcgaaatcgtccaacgcgcgccttaaaatcatttgtagcgacttcgagggactgtattgccgaacgaggttgtcatatgttttcgacacttgaggcgcgggcggtcgcgctgaaagaaaaacctggagctttttcggggacggaggtggactaagggcatccacagttagcttaagttgtcgatcgggactgtaaatgtgatcggcgacgagaggctcacgttgctggtctttctcgtcggctttttcaggcaagtgctggaggtccagcttctggggaacaagtgtcgggttgggatggtggatctcgggtcgagcaccagcaagccgccgtgcttcgccgaccgacaatgcgggcttgcgaattgccatcttcaagcctccaagattttgctgatcagtttcgaaatgaccacgacttcctccatcgcaatccgaagattcctctctatgaggcgcatcgtcggatcagttcccgtgtttagtaatgtaagatgcaacatgccgcgttctttcatcgcggcaaatgcatctctttcatgcatgggagacggtacaactggaaggctctctagcgtctctgacatcctgcgttgcgatgttgtcaatcggccgaccgggacgcgttggcgcaaaacagctgtaggaattgccaaattttcactcaacagcagctcgatgacgtagcggtaggtagatagtgcctcatcgatgtcgagcggcgttagcatggtggggatcagaagcaggtttgagctagcgatgattgtgttgttgagctcgctcgagccgccacgcgtatcggccaacgcataatcaaatccttcgagctcggcattttcataggctgcttcaagaaggggcatttcgtcggcggaatagacttcacagcgaggatcccaggtactgctttgtaaggcgttttctctccatcgcgtcagaggccggttttcgtcggcatcaaagagggccactcgtttaccgtcatttgccaaagcagcgcaaaggcccatgagtgcggtggttttgccagcaccccctttgaaagaacaaaacgtcaaaagttgcatattctgatcccgcctatcctgtgaaaccggagtgcatttgtatttttgttcgtataaatgtttttgtgattatcgatgagtaaaagcgttgttacactatttttatttcacattcgttataagacaattgcaaatgtagcaagtatattcagtattgactgtaaatgtactgttgatttcatattgagcagggctagacttccatccgtctacccgggcacatttcgtgctggagtatccagaccttccgctttctttggaggaagctatgtcaaaacacaccagagccacgtcgagtgagactaccatcaaccagcatcgatccctgaaagttgaagggttcaaggtcgtgagtgcccgtctgcgatcggccgagtatgaaaccttttcctatcaagcgcgcctgctgggactttcggatagtatggcaattcgcgttgcggtgcgccgcatcgggggctttctcgaaatagatgcagacacacgagaaaagatggaagccatacttcagtccatcggaatactctcaagcaatgtatccatgcttctatctgcctacgccgaagaccctcgatcggatctggaggctgtgcgagatgaacgtattgcttttggcgaggctttcgccgccctcgatggactactgcgctccattttgtccgtatcccggcgacggatcgacggtcgctcgttactgaaaggtgccttgtagcacttgaccacgcacctgacgggagaaaattggatgcccgatcgcgctcaagtaatcattcgcattgtgccaggaggtggaaccaagacccttcagcagataatcaatcagctggagtacctgtcccgaaagggaaagctggaactgcagcgttcagcccggcatctcgatattcccgttccgccggatcaaatccgtgagcttgcccaaagctgggttacggaggccgggatttatgacgaaagtcagtcagacgatgacaggcaacaagacttaacaacacacattattgtaagcttccccgcaggtaccgaccaaaccgcagcttatgaagcaagccgggaatgggcagccgagatgtttgggtcaggatacgggggtggccgctataactatctgacagcctaccacgtcgaccgcgatcatccacatttacatgtcgtggtcaatcgtcgggaacttctggggcaggggtggctgaaaatatccaggcgccatccccagctgaattatgacggcttacggaaaaagatggcagagatttcacttcgtcacggcatagtcctggatgcgacttcgcgagcagaaaggggaatagcagagcgaccaatcacatatgctgaatatcgacgccttgagcggatgcaggctcaaaagattcaattcgaagatacagattttgatgagacctcgcctgaggaagatcgtcgggacctcagtcaatcgttcgatccatttcgatcggacgcatctgccggcgaaccggaccgtgcaacccgacatgacaaacaaccgcttgaaccgcacgcccgtttccaggagcccgccggctccagcatcaaagccgacgcacggatccgcgtaccattggagagcgagcggggtgcccaaccatccgcgtccaaaatccctgtaactgggcatttcgggattgagacttcgtatgtcgctgaagccagcgtgcccaaacaaagcggcaattccgatacttctcgcccggtgactgacgttgccatgcacacagtcgagcgccagcagcgatcaaaacgacgtcatgacgaggaggcaggtccgagcggagcaaaccgtaaaagattgaaggccgcgcaagttgattccgaggcaaatgtcggtgagcccgacggtcgcgatgacagcaacaaggcggctgatccggtgtctgcttccatccgtaccgagcaaccggaagcttctccaacgtgtccgcgtgaccgtcacgatggagaattgggagaacgcaaacgtgcaagaggtaatcgtcgcgacgatgggcgcggggggacctagactagtagacaggaaggaccgaataatggcaaatggtcagttcacgatacgctctgctcgcccggcctccgtcggactgacaggcgaacggcgtggagccgcatccgcctctagctctgcactgtccaatgttcaaagagatgttagggataggctgattccaactagctcaccaagattaccaaatgcagccatattgcgtgattcctcgggaagagcgtcgactggtctgcggtacatggcggctactcttcattggtctgcgatcgcgccattatcgctaataaacagcaacgacctggctccggccgcttatgactttgagacgcgaaataacgcaagaaatgtgactgccaaagtcggcagggcagtccctgttcccaagcaaggcgggctcggcaaaacgctcgcacccgtaccccttagtacacgtatatcaagggtcaattccgaccgaagactgcccgctgacgcagaagaccgccctgaaacgcgcgacccccagaaaggacgtggcagtcatggtgcgacgccaaccttacatgaaaagattggaaccgcgtttgctcgaagattgcgaaagcatacgtactatattgtttgcagttgctgccagacccggagcgcgttgacgatgggtgcaaagatttcggtgaagtcatgaactccagcaagacttcgccccagcgtatgaccctgagcatcgtatgttcgctggcagccggtttttgtgcggccagctgctatgtaacgttccgccggggcttcaacggcgaagcgatgatgacgttcgacgttttcgctttttggtatgagaccccgctttacttgggttatgccagcaccgtcttctggcgtggtttatctgttgtcatctttacctcgctgatcgttctttcaagtcagctcatcatatcgctgcgcaatcagaagcatcatgggacagctcgttgggcagaaattggcgaaatgcggcatgctggttatctgcagcgttacagtcgcatcaaggggccgatctttggaaagacatgtggtcccctttggttcggcagttatttgaccaatggcgaacagccacacagtcttgtcgtcgcgccaacgcgtgctggcaaaggcgtcggcatcgtcattccaacgctgttgaccttcaagggctcggtaatcgcccttgacgtcaagggagaattgtttgaactgacgtccagagcacgcaaagcgagcggcgacgcagttttcaagttctcccccctagatcctgagcggaagactcattgttacaatccggtcctggatattgccgcacttccgcccgaacgccagttcactgaaacacgccgtctagctgcgaaccttattacggctaagggaaagggagcagaaggctttattgacggcgcacgtgacctgttcgtcgcgggaatccttacctgcattgagcgtggcacaccaacgattggcgcggtatatgacctatttgcgcagcctggcgaaaagtataagctttttgcgcaactcgcggaggaaagcctaaacaaagaggctcagcgtatcttcgataatatggcgggcaacgacacgaaaattctgacatcgtacacctctgtgctgggcgacggtggactgaacctgtgggctgatccgcttatcaaagcagcgacaagccggtcagacttttccgtttacgatctccggaggaagaagacctgcatttatctttgtgtcagtcccaacgatctggaggtcttggcaccacttatgcgcctgatgtttcagcagctcgtgtcaatcttgcagagatcgctgccaggtgaagacgagtgccatgaagttttatttctcctcgacgaattcaaacacctgggcaagcttgaggccatagagaccgcgatcacaaccatcgccggttacagaggccgctttatgtttattattcaaagtctttcggccttgtcgggcacatacgatgacgcaggaaaacaaaactttctgagcaatactggcgtacaagtatttatggccacggctgatgacgaaactccaacctacatctcaaaagctatcggcgaatatacgtttaaagcgcgttcgacctcttacagtcaagccagaatgttcgaccacaacatccagatttctgatcaaggtgcagcccttttgcgccccgaacaagtgcgcctgctagacgatcagagtgaaatcgttctcatcaaagggcgacctccactcaaattacgaaaggtgcagtattattccgatcgtacgctgaaaggccttttcgaacgccagatgggctctctgcctgagcccgcacccttgatgctttccgactatagcaacgatcaagttcaataccacttggctccgatagcaaattttaatgaggatgctgcaccgcaaaacagaactgtggccgaggaccatggaagtgttaaagtcggtgctgatatccctgaacgcgtgatgggaataaatggtgacgaggaacaagccgatgcgggcgagataccgccggaatcggttgtgcctccagaattgacgctcgctctgaccgctcaacagcaattgttggaccagattattgcacttcagcaaagatcgaggtccgcaccggcatagcctgcgaaatgatcttaatggtgcaattcgtttcaggcggcatgttgcggttcaacaaagtgtacgccagatcccaattggccctttacgaggtgtcggtatgacaggaaagtcgaaagttcacataagaggttcggctgacgcgcttcctgacgttcctggcggaagtactaccgccccttttttaaccgaaccttctcgggatcaggttgatgcctcgtttgaggtccaaaccgactacagccagtctacttccgtgtcgtttacctatgatggtgttggacttggtcctgccgagcgtgcggcttacgagaactggtgcgaaccgggccggcccacttggaaagatcttataatcaaggcacgtgtcgatccgattgacgatgtgacctggctccgagatttagaagaggacaccccctcaaccttcagatacgaagggatgcctctgggcatcggggaacgacaggcctacgaaaattggcaagaggacgctcagccgacatgggaagaccttgttgtcagcgcacgcttgacggaacttggccgtccacacgggattaccggcgagtatacatccctcgcaggatcgaagaatacaagttcaatttcattgaagcgaaagcggagcaacttaattgatgatgagaattcatccggatcgttttcatatgacgggatgaagctcggggaagccgagcgttctgcatatggtgactgggccgaggcggagccacccacgtggaaagatttggtattgagggcacgcgtttcctcgatcaatgactctgcttggctttttgattcacaaacatcttcatcatcatttgaatacaacggtgttcccttgggcgagccggaacggcaggctctcagacaatggcaaggagacgctcagcctacctgggaagatctcgttgttaacgcgcgtatggcagaactttgccatgctggttggattgaaggtcaaaaaggttgctttgaagagcgcggggaggctctgcccgcgtcggaacgcggttcgcaacgccccattggtcaacggacagattcctccgattcttttgtgtatgatggcacaaggctcggagcacctgagcgaactgcttatgaacgctggagtaagagggaacgcccgacttgggaagatctcatcttagatgcacaccaggccaggactgaaagtgacgctgttacgacccaagcgattggtcagtcgtcctcaccggttttcttatatgaaggaaagtcgctcggagacagggaacgaaaggcttacgaaaaatggcggcagccagcccaaccgcgatggcaaaatcttgtagtgaacgctcgtctggcagaaatcgatccctcagcctggattgccgatgagcgcgatccgcttgatgatagcgacgcgcttggtcgcccgtcgtacacaagcttgacggatagatcagacgtccctttagacgatcaatcaatctatcgtcgttccgacctagtaagggagcaggtgccagaatcgtctcaaaggcaattcgcagcatgttcagaatctgaaacgaggcctgtgcaatggtttactgcttctgggtcagatgcaaacaatacggaaaatatcaccgccagcgatcccgtcgatcgcacgggtggagttaagcggctaggctccaaaagcgacagaaccgttacagcttctatccatgacgtgaattccagcacaaggcgactgttgcttaacgaatttggatcggaggctccgcgcccttcgccagaaaagactgttcgcttaagaagcgacaatattggcacctatgggagccggaaaaatgaacgagcgcggctcgcgaccgaaaccggtgcgtatgagtcggagcatattttcgggttcaaggctgtccacgatactgcgagagcgacgaaagagggccggcgtctcgaaaggcccatgcccgcctaccttgaggataaggggcttcatcgccaacatattggcaccgggagaggacggaccaaacttgtcgggcgcggatggccggatgacacaagctatcgctcggatcaaagggcaactctgtcggaccccgttgcgcgctcggaaggcgcgacggcctcaaatgggtatcaattgaaccaattgggttacgcgcaccaactcgctagcgatggcctgcaaagtgaatcgcccgatggtgttgccttgccaattcaagtggcaacaacgagctacaactatacagtgagccgcgatcctgtccttgttccgccggataaaaacgaagcccctcaattgctgcatcttggtccccgtggtcaaaccgaagctgttcttgcccgcgaaacagcattgactggaaaatggccgactctcgagcgtgagcagcaagtgtatcgcgagtttttggccttatatgacgtaaaaaaagatcttgaggccaaatcagtcggcgtaagacggaaaaaaaaagaagttatttctgcgttagaccgaactgcgcgcttgataagcacgtcgccttcgaaagctcgatccaaagcagagactgaaaaagccattgatgagctcgatgatcgacgagtttatgatccgcgtgatcgagctcaagacaaagcgtttaaacgctgataagtcgccaatatagtgatcatttgcagtattcgcatcgatcgctggttgatattctgccgctggtcgaccggctgctcgtcgccaaaaatgctcacagggatacgatggcctcggtcaggccgcgtgcgtcctgtctttccagttcctccctttcagctcgattgtggcatcatttattgcctgctcattgcagttgaaacgcgatatccgtttcaagacccgggt

SEQ ID NO: 13 - DNA, 38871 bp, synthetic construct
  misc_feature 1..38871: note = RV007497
  source 1..38871: mol_type = other DNA
cgcgccagcgagttgctttctctcgttggctgatgggaagaaagcgcataggagtcgccgccaaattcttgtcggaccagcttggtttgttcggtcggtgtttttcaccgtggatgcggagtaatgagaaactgggtcagaccactgggctccgttgattccgcgaaatctgcttagttggattggctgtcagaaggagaggtgaccgcatgatttgggatcatttatcaccttcccgaagggttctgcttgatatcaaatcgtggtctgtattggtactggtaattgcctcgatatcttttgcggttctggccataggatcttggcaagacaacgaaagcaacgaagcgatcctgacagagctgcaatcgatcgacgtggattgcgcgatgctgcagcggaatgtgttgcgtgctcacgccggccttcttcggaattaccatcctcttatcgtccctctggggcgactgcgcacgagcgtcgcagatttacagcagctttttaggcgggcgggcctcgacggggccggtgaattttctgaattgctggcgcgggtgaagagctcagtagatgcgaccgacgcagccgttgcagcgttcggtacgcaaaatgtaatgttccaggattcgttggcgacctttaaccagtcgatagcttcgcttccgaggtctttggatacgagggatttgaactcgctagaagtcgctgagttgggctatctgatgctccagttttcgtctcagccaaatttcgagtttgcacgggaaatcaaccaacgtctcgaccggcttcaggtttcggcgaacggggataaggcaggcgtcaaggagatcgttcgcagtgggaggattatcctgactctactgccgcgattgaaagacaccgttggagtgatccaaacttctgaaaccatcgacaacacgaagaagttgcagcgagcataccttgaagcttatagcttggccagcgcgggtgaacagcgggtacgggtcttcctcggcgcagtttcggttttcttttgcttttgcataatttccctagtccataggttacatctgcgaaccaaggtgttgacgagacgactggacttcgaagaagtgatcaaaaagattgggctttgcttcgaagatgcttcagaagcaaaaccgtcgttggaatcctcaacgaatgccgcgttgggaattatccaattattctttgaagctcatcaatgcgcgctgggactggtcaacatcaatgagaatgacgttggcgtcaccttttttggaagcactcctgcgccagagtggaacgaacaacgggtgcgcgaaattgtttccacggtaggggccgaggacggcgggcggattttccgagcctatcctcaacgaaaagcgagctgcttcggtgaggactctccatttctgtgggtgctgcttgcgttcaaggtatccgatcgaatagtcgctgtctttggcctgggatttgatcgagagcatctacagacgccgaccgcacgtgaaatgcagctgatggaactagcggctgcatgtgttagccactatgcggtcattcggcgcaagcagtcccagcgagatattctggaacgtcgtttgaaacatgcggaacggcttgaagcagttggtacgcttgccggcggcattgcgcatgaatttaacaacattttgggtgcagttctgggttacgcggaaatggcacacaacgttctgcgccgtcgtactcatgctcgagactacatcgatcacatcatttctgaaggcaatcgagcgcggttgattgtcaaccagatccttgcccttagtcgaaagcgggatcgaacaacaaaaccattcgatctttcggagttagtgaccgtgatcgcgccgtcgttacgtgttgcgttgcctccgggcgtcgagctcgatttcaaatatcagagcgctccaatggttatcgaaggaaactcacttgaggtcgagcaaatactgatgaatttgtgcaaaaactctgctgaagcatgccgagagttagggcgtgtggaggtcagcgttaggaggtctgtcgtacggaaacacaaggttcttacaaacggtactattcccaccggcgagtatatccttctttcagttgaggataacggcgcaggcataacagaggcggtattatcgcacattttcgagcctttttttactacccgcgcccgcagcggtggcacgggcctgggattatccacggttcacggtcacgttagcgcgatggctggttacatcgacgtggtatcaactgtcggcagaggtacgcgcttcgacgtctacttgccaccttcgtcgaagcagccggtaaattcagagagtttttttgaccctggaagaatcccgcttgggaacggcgagattgtcgcggtggtcgaacccgacttagccacgctggaaatgtacgaggaaaagatcgcggcgctgggctatgagccagtcggctttaacacgttggatggtttaatcgactgggtgttggaaggaaaagaacctgatcttgttctaattgatcaaccgtctttgctggatggagacggtgcagggtccttaatctcaaagctggcaaaagcgcccatcatcattattggcgaaaaccagaaaaatgttcccctttctgcggatcgcgagggatctgcccgttttttggaaaagccgatttcagcaaagactctcgcctatgtcgttcgtgcgaatatcagaactgaacgaatggtcggctcgaatagcagcggaatcgtgattgcgtcataaaccgatacgccctctgaacagaagccggccgcacaacgcgattgcttattaatttaatccccagtcgcggaatcagccggtcagtcccgacatccgcaacaacgcaactccggttgcggctgcaatcaccgccagtgatatacaggcaaaaaaccggtaaacctgctcttttgatgttaagaggagatggatcgaagcgatcatcagaaggaaaatcgtcgcaagtgcgccatacgcccaatactcgttcgaataaagccatgttcccaaagccaacagaccgcaaacaaaaaaaacgagttcgagcttttgcaagacaacacctcaaaaagatctcgttgtgatcctaggacatcggtctcgcaggtcaatactgcgatcgcgtttcatgcaacggacttgatgtatctgccgcgcggcactaggagacgtgaggaacggccccctgtctagggtgcagtcggtgccgatctccgcttcgggggcgctttgcgaaatctcctgcggctgcaccatcctccgcgccagcctgcgaaaattttcgaaaaccgccgctatggtgaattcgctctcatggcctagggatcaccgcttgggccgccgattccctcgattcaagaaatcaaacggcttcgcttcgatgcttcaaatgaaaccgagaggtaagataataaaatcagaaagtaacagacacctcccatagtcgcgactgttagcgatcgagtcatttcatgctgaccaagatcattgcgatacagtggggaataaacaatgcggaagacggcaggatctttcgggatcatttttctgatggtccagccaagcaaagctgcgccgctctcgttcgctgaatttaaccaactcgctcgcgaatgcgcaccttcagtcgctccatcgacgcttggagcgattgcgaaggttgaaagtcagtttgatccgctcgtgctgcatgacaataccaccggcgaaactttacatggcaagaatccggcggatgctacgcaaagcgtaaacaatcgcgtcgcagcagggcattcagtcgatgtaggcctaatgcaagtgaactcgaaaaatttcgcgaacttgggcttgactgccggcaatgcaatcaacccttgcgtgtccctgtcggcggcggcggacctgctcgtacggcattatagtggcggcgacacggtagagtcagaacaacttgccatccggcgagcaatttcggcttacaataccggcaacccgacacgcggcttcgcgaacggctatgtgcggagggtcgaagtggcggctcagctactcgtccccccgctggctcagtcggggaaaaaaggcgaccgtgatgagcggagtccagaagaaccctggaacgtctggggttcatacgaccgctcccactcagcagtcggttcgtccgcgccgccagaggcgcagcagccgaatgagcgcaaatcccccgaacaagatcaagttttcgaaccgaatgatggagatgcaccataatggcatggcttgaaggataccgcgcaccgtcgaaattccgtggcctctggcacagagctgtgcgcctgctggctccgcacgtgccgagcgtcaccggcgcgatcggctggagcttgtttttctgcgagccagctgccgcgcaggccgctgggggcactgaccccgccaccatggtcaacaatatctgcacgtttattctcggtccgtttggtcaatcgctcgccgttcttggtatcgtagccatcggggtatcgtggatgttcggccgagcttcccttggtctggtagcaggtgttgttggcgggatcgttatcatgtttggagccagctttctcggccaaacactgacagggggcgggggatgaatgccgatcttgaagaagccacactctacttggcggcaacgagacctgcattgttctttggggtaccgctgacgcttgctggagttttcatgatgttggcggggtttctcatcgttgttgtgcagaaccccctctatgaaattgttctcttgccgctgtggcttggagcgcggctcatcgtagagcgggactataacgcggcaagcgtggtcttgctgtttctccagacggcagggagaagcgttgacggccatgcgtggggcggcgcgagcgtcagtcccaaccccatcagagtggcgtcgcgaggaagaggaatgacgtaatgttcggagcaagcggacggaccgagagatctggggaaatctacctgccttacgtagggcacctcagcgaccatgtcgttcttctagaggacggttccatcttgaccatggcgcatatcagtggccttcccttcgaactggaggaagtagaggtccgaaatgcgcgctgccgtgcctttaacacgctctttcgcaatattgccgatgataacgtctcagtatatgcccatctcgtccgtcacaacgatgtgccagtaccgccggtacgccattttcgcagcaccttcgcgtcaagcctaagcgaaacgttcgagcggcgtgttctgtctggtaaactgtttcgaaacgaccacttcctcacgctcattgtctctcctcgcaccgctctcggtaaagtgggaagcagattcaccaggcgctatgggaagaacccaggcgatcttgcgcatcagatccgacatctggaggatctttggaacgttgtcgctggcgcgcttgatgggtatggacttcgtcgacttggcatccgagaaaaaaatcaggtgcttttcacggaaattggtgaagcccttcgcctgataatgacttgtcgttttttaccggtgccggtcgttagcggatccctcggcgcctcagtttataccgaccgggtcatctgcggcaagcgtgccctcgagattagagcgccgaaagatcgttacgttggatctatcttttcattccgcgagtacccggcaaagacgcggcccggaatgcttaacacactcctgtcatcaagttttccgctcgttttgagccagagtttctctttcctcacacgtgcacaggctcacgccaagctcagcctcaaatcgaaccagatgactagctccggtgacaaggcggtcacccagatcggtgaactcgcccaagcagaagattcattggccagtagcgaattcgtcatgggttcgcatcatctcagcctttgtgtctatggcgatgatctcgatacccttgccgactatggtgcgcgagcccgcacgagcttgtcggacgcgggcgcggttatcgtgcaggaaagcattggaatggaggctgcatactggtctcagcttccaggaaaccacagatggcgtacccgtcccggagcgatcacttcacgcaactttgccggcttggtttcattcgagaacttccctatcggaaggaagtcgggccattggggaggtgcggttgctcgtttccgtacaaacggcgggacgcccttcgattatattccgcacgaaaacgacgtgggcatgaccgcgatcttcgggccgattggcaggggaaaaacgacacttatgaccttcatcctggcgatgcttgagcagagcgtggttgaccgcagcgggacgatcgtttttttcgataaggatcgtggcggtgaactgcttgtgcgcgccaccgggggcacgtacctcacgttacgacgaggtgtcccaagcggccttgcacctttgcgcggcctagaggatacggcagcggcgcgggactttctccgcgagtggatcgtggcgctgattgaaagcgacggcagggggggaatttcgccggaggaaaatcgccgcttagagcgcggtatccaacggcaactttcgtttgagcctaacatgagatcccttgcgggcttaagggagttcttattgcacgggccctccgaaggtgcgggagctagattgcagcggtggtgccgaggcaacgctcttggctgggcgtttgacggcgaatcggacgaagtgaagttagatccttcgattacgggcttcgatatgacccatctcctcgaatatgaggaggtatgcgcgccggccgcagcataccttttgcatcgaataggagcgatggtagatggccgacgcttcgtcatgagctgcgatgagtttcgttcctacctgcttaatcagaaattcgcagcagtcgtcgacaagttcctgctgaccgtccggaagaacaacggaatgttagttcttgcgacgcagcaacccgagcacgtacttgattcaccattgggcgcaagcctcgtagcacaatgtatgaccaaaatcttctacccatcacccactgcagaccgctctgcttacatagacgggttgaagtgcaccgagcgagaatttcaggctattcgcgaggagatggcgattggcagccgcaaatttctgctgaagcgcgagagcggaagtgttgtgtgcgaattcgatctgcgcgacatgccggaatacgtcgccgtactatccgggcgtgccaatacggtgcgttttgcccagcagttgcgcgagacgcatggagaggaaccctctgcctggctggaaaaattcatgacgcgctaccatgaggcacaggattgatctggagttcgaagtcatgaatattacgaagctcgtaataaacgcacttttcatatgccttgttctgtcggggactgccaaggcgcagtttgttgtcagcgaccctgcaacggaggccgaaacgctgacgacagcgatcaatactgcggcgaacctcgagcaattgatcacgatggtgacaatgttgacctcgccctttggcgtcaccggcatgttatcagcgatcgaccagaaaaaccaatatccctctgccggccaactcgacaaggaaatgttttcgccgcagatgcctgcttcgacaactgcgcgtgcaattaccttagatgccgatcgcgcagtcgtgggcgacgacgctgaaggaaatcttttgcgacagcagattgcaggcgccgcaaatgctgccggtgtcgcggctgacaatctggatgcaatggacaagcgccttgcggcaaattctgagacatcgggccagctctcccgctcacgcaatatcatgcaggccaccgtcaccaacggtttgcttctcaaacagatccatgacgcaattattcaaaacattcaggcgaccagcctgttgacgatgaccaccgcacaggcggggctgcacgaggcggaggaggcggcgacccaacgaaaggaacatcaggcaactgcgcttatatttggcgccgctcaactacactgatccgctactgttcattcagggccctctttctccgtagaaaatcgaagctaaatccaggtaagggcaatgaattttagcattcctgcgccttttacggcgatacatacgatattcgacctcgccttcaccgtcgggctggacacgttgcttggggacattcaacgggcagtcagcgctcctctcgtggcctgcgtgacgctatggatcattgtccagggcatcctcgtaatgcgtggggaaatggacgcacggggcggaatcacccgggtaatcatggtatcggtagttgttgctcttatcgttgagcaggcggaatatcatgattacgttgtctcggttttcgaggatacgattccgaatttcattcaacagttcggtatcagtgggcttcctttgcagacgattcctgctcagctcgacacgatgttttcactcacgcaagtagcctttcagaagattgcttctgaaattgggccgatgaacgatcaagacatcctcgcctttcaaggcgcacaatggattttctatggaacgctttggactgcgtttggaatctacgatgccgtcggaatcctcaccaaggtgctattggcgatcggtccattgatgctcgttggctatctcttcgatcgcactagagacatggccgcgaaatggatcggccagctcgtcacttatggcatacttctgcttcttctaaacatagtggcaactatcgtggttctgacggaagcgacggcactcgtgctaatgctcggggttatcacttctgctggcacaacggcggccaagataataggcctctatgaactcgacatgttctttctgaccggcgacgccttgatcgtcgctcttccggcaatcgccgggaatattggagggagctattggagcggtgcgacgcaaacggccggtagcttaaatcgccatttcgcccggacaatccgccgttagtcccagcttcacaaatggaggatttccttgaagtactttctattgtttttgatcatcggcttggcgtcttgtcagacaagcgatcagttggcgacttgtaaggggcctatttttccgctgaacgtcgggcgatggcagcctgctcagtcggaccttcagcctaccaacgcaggagaggctaatgaacggctctgaatacgcgctgttagtagagcgggaagcattggcggaccactataaggaagtagaagcatttcagtctgcacgtgctagatcagctcggcgaatctctagagccttggctgctttggctgtcattgcagtcgcaggaaacgtggcgcaggccttcgctatcgccgtcatgcttccgctgaacaaacttgttccagtatatctgtgggtgcgcccagacggtacagttgacagtgaggtatctatttcgcggttgccggcgacgcaggagcaagcggtcgtgaatgcgtctctatgggagtatgttcgtctgcgggagagctactcggcggatacagcccaatacgcctatgatctggtctcgagcttcagtgccccaacggtgcgtcaagattatcagcagttcttcaattaccccagtcctagctccccccaaaccatcattggtaagcgcggaaagctggaagccgagcacatcggctcaaacgaacttatgactggcgtccagcagatccgctacaaacgcactctcatcatggaagggcaagctccaatagtaaccacctggaccgcaacggtacattatgaaacggtaactaacttgcccggccgattgaggctgacaaatccaggcggcttaatcgttacctcctatcaaacttcggaagataccgtttcgaacacgacgcggagccagcaatgatcaagaatttgtttctgggcctggtttgtatcctctttgtaaccagcggcgcgaacgcggaagacacgccagcggcaggcaaactggatccacgtatgcgctatctcgcctacaatcccgatgaggttgtgcacctttcaaccgctgttggagccacattggtcgtaacatttgggtccaatgaagcggtgacagccgtcgccgtttccaacagcaaagatctcgctgcgctcccacgcggcaattatctgtttttcaaggccagcaaagtgctgcagccgcagcctgtgatcgtccttaccgcaagcgacgcaggaatgcggcgctatgttttcagcctcgcgaccagaacgatgtctcggctcgataaagagcagcctgacctctactacagcgtacagttcacttatcctgccgacgttgccgctgctcgccgaaaagaagcggagcagagagatcttgcggatcggatgcgggcgcaagcacaatatcagcgtcgagccgaggacttgctcgagcgtccgccagcaggtggcagcacagatgcgaaaaattggagctatgtcgcgcaaggagaccgctcgctattaccgctcgaggttttcgacaacgggtattccacgacatttcgctttcccggaaacgtgcgtgtaccttcaatctacgtgattaacccagatggcaaggaagccacggccaactattcagttaagggagattacgttgaagtagcatcagtttccagggaatggcgtctgcgggacggccatacagtactttgcatctggaataaggcatatgacgccgtcggacggaagccgggcactggcacagtcagacccgatgtagtgcgcgtgctgaaggagacgaggtgatggaggacgtgaatgcccaatccagagaaggtatcgacgcgcccggatccctcgtcaccgatcctcatggccggcgcctctcggggtcgcaaaagctccttgttgcgggtttagtcctggtactgtcgttgagcctcatttggctcggggcgcgttcaaagaaaaggaccgagccgtctccaccaaacacaatgatcgatgccaacacgaagccctttcggccggccccgattgatattcccgccaaaccggcaatgacaccgccagctgcggaagcaacggttttgccgtcggatcaacatcagcgagaacacaacgaactgaggcccgaagaaacaccgatctttgcctacagcggaggtgatcagaatggtgtcaaatccgccccacacgctgatattcagaacggtggccaagacaatagaaacgccaactccctagccgcgtcggaggattctgctgagaacgatctctctgtgcgcttgaaaccgacagtgttgcagcctagcctcgctatacttttgccgcacccagatttcactgttacgcaaggaacgatcattccctgcatactgcaaaccgccattgatacaaatctggcagggtatgtgaaatgtgtacttcctcaagacatacgcggagcaaccggaaatgttgtgcttctggatcgcggaacgacagttgtcggcgaaatccagcgtggactgcagcagggagacgcccgcgttttcgttctctggaatcgcgcggagacgccctcacacgccgtcgtctctctttcctccccgggggccgacgaactcggccgttccggactgccgggtacggtcgacaatcatttctggaaacgctttagcggggccatgctcctgagtgtcgtccagggcgcagtccaggcggcaagtagctacgcagggaattcgaccggcgggaccagcttcaatagtttccaaaacaatggcgaacaagcggccgatacggccctaagggcggccataaacattcccccaaccttgaaaaaaaatcagggtgacacggtctcgatcttcgtcgcgcgggatttggatttctcaggaatctaccagcttcatatgactggtggatcggtgaaacgtcggcatctgcgctaatgcacagatagccaaagctgcgagatgcatccaatggaagtaaatccgcaattgcgcgcccttcttaacccagtcttgcaatggctcgatgacccgaggactgaagaagttgccataaacggaccgggagaagcctttgtacgccaaagtggcgtcttcacgaggttcgccgcaccgttctcttatgatgatctcgaagacatcgccattctagcaggggcattgcgaaaacaagatgtcggcccccgcaaccctctttgcgctaccgagctcccaggtggcgagcggatgcaaatctgcttgccgccaacggtaccctctggcaccgtgagcttgacaattcgacgaccgagcaatcgtgtctctgaattgggggaagtctcggctcgctacgatgttcgtcgatggaatcagtggcaaatccggacgcagcgacgagatcaactagacgaagcgattctgcgcgactacgacaatggcgatctggaatcatttttacgtgcatgtgttatcggtaagcggacgatgttgctttgtgggccgaccggaagcggcaagaccacgatgagcaagaccttgatcagtgctattccgcgggaagaaaggctgataaccattgaagatacgctcgaactcgtcattccacatgagaaccacgtcagactgctctactccaagagtggggcagggctaggtgcagtgactgcggaacagctgctccaggcaagccttcggatgcggcctgaccgaatattgctcggtgagatgcgcgacgacgcggcgtgggcgtacctgagtgaggtggtctcaggccaccccggatcgatttcaaccatacatggggcgaatcccgtccaaggcttcaaaaagctgttctcacttgtgaagagcagtgtgcagggggcatgcttggaagatcgcacactgattgacatgttggcaacggcaatcgatgtcattgttcccttccgtgcttacggcgatgtttacgaagtgggcgaagtatggctcgccgccgatgcccgccgacgcggtgagacgataggggatcttctaaaccagcaatagagccgcacggaggtctgctcgcgcagattttaaagcgttacaagtgtagcgcgctgcagcaactcctgtcatcaatctgaaaccttccgttagcatttttgtcatgcacggcttggccgaaattttgccgaactaccctcatagtgagagaaaatgatcaggttgaggctgagacgtacatcctctgcttgcacgaatctatcgattctgcagaatcccatacggaggcgaactggtgaaacatgttcttgtcatcgatgacgatgccgcgatgcgccatcttattaccgagtatttaacgattcatgccttaagggtcaccgcggtatcagatagccagcaattcaaccgggtacttgcatcagagacggtcgatgtcgtcgtcgtggatctcaatttagggcgtgaagacggacttgagatcgttcgcagtctcgccacaaaatccgatgttccaattataatcatcagcggtgatcgccttgaggaggcggataaagtggtggcgctcgagctgggagcaacggattttatcgcgaaacctttcggaacgcgcgagttcctagcacgcatccgtgtcgcattacgcgtgcgaccgaatgtcatgcgaaccaaagaccggcgttcattttgtttcgccggctggacacttagtctcagacagcggcgattggtatccgcacaaaatggcgaggtgaagcttacggcaggggaattcaatctgctcgtcgctttcctggaaaagccacgcgacgtgctttcccgagaacagctcctcatcgcgagccgagtgcgtgaggaggaagtatatgacagaagcattgatgttctcattttgaggctccgccgaaagcttgagcaggatgcagcgagcccaaacttgatcaaaactgccaggggtgccggctatttcttcgacgctgatgtggatgtttcctatggaggtctgatggcggcctgaagcagcgacgcatatgcctttttcgatctgatcctaggacgcggactcgttaattgaagcgatccagattctcgtcgcggcaagtttgagcccggcaaggtagttgaacaatctcctttccaggaaattgaatcacacaacgtcatcaacatgaaccccgttatggggacacagcctagagtgagctggctgtgttgtagctgggtaggggctgcctcattgcctgctgtcgaatacaataccgccgtcaagtgaccggcctcatcatattagcgttcttttcttatcttctcttcggcaaagaaagatgcaagtgcggccgtagccagtttgtggccaaaagccctggcggtctccaatctcaatggatcaaagtgattgcgtgcgacttctaacagcgataccgggaacatgcgagaagtcaggacaacgataggtttttcgactacttttgagatcgggtagctttgaggccccgtgctaaatgatccgtccgccaacatgttctcaaaatcggcgagcgcacggcgcaagatcatctgcagcgccttggtagcgctgtactgcagaatcaagttgtcatatatcttcgatacctcaggagcgggggggcgtgccgaaagaaagacctggactttttccggcggcgcagtcgaactcaaggcgtccgccgttagcattgattgccgatcgaagttgtcacgacgtttggcggcgataggaggcgctggtcgtttttcctttccggctctttcaggtaattgcgaaggcgccaagttttgcgaggaaagagctgggattgggtgaacgatcggtcgcgcggcagcaagccgtcttgcttcaccgacagacaaagcaggtttgcggatccccatcttcaattccccaaggtatcgccaatcaattctgaaattgttacgagttcctccatcgcaattctgaggttccgctcaaggaggcgcatcgtcggatcatttgtcgtgttcagcaatgtgagatgcaacatgccacgctctttcattgcagcgaacgcgtctcgttcatgcattggagattgtacgacaggcagattcaccagcatgtctgacatcgcgcgttgtgacgtggttaatcggccaaccgggactcgctggcgtaacacggcggtcggaatcactaagttctcgctcagcagcagctcgacaacataacgatatgtcgaaagtgcctcatcgatatccagcggcgttaacattgtcgggatcagaagcaggtttgagctagcgatgattgtgttattgagctcgctcgaaccaccatgcgtatcggccagcgcataatcaaattcctgaagctcggcgtcctcataagccgcttcaagaagcgacatttcttctgcagcgtacacctcgcaggatgtatcccaggtgttgctacgcatcgcgttttctttccatcgcgttaacggccggttctcgtccgcatcgaagagagccactcttttgccgtctcttgcaaacgcagcgcaaaggcccatgagcgccgtggttttgccggcgcctcctttaaatgagcaaaatgtcagaagtttcataagtttatcctggcgacgttgtgaacgaaagtgcgtctgtatcattgtttgtatgtgtgactgtctgtatagcgaaattatcaggagtatatttcaattgcattgaaggatagttgcaaatgtaactatatagatccgaatatgaacagtatattcttgaatttattctgttggcggctaggctccctcagtctcaactggagaatttcactccggaacaaatccagatcattccgttttctatggagcaagccatgtcgcaaagcgaaaaatccacttcaagtgacattgtcgaccggcgtgaaggcccgaagattgaaggtttcaaggtcgttagcactcgtttgcgatcgtcggaatatgagagcttctctcgccaagcccgtctgctggggctctccgacagcatggccataagagttgcggttcgccgcattgccggctttctcgaaatcgatgcagagactcgtcagatgatggaagccattcttcattcaataggagcactctcaaacaacattgccgcgctgctgtgtgcctatgctgaaaatccgacaacggatttgggcgctctgcaagctgaacgaaacgctttcggtaaatcgttcgctgatctcgacggtttgctccgttccattttgtccgtatcacggcgacggatcgacggttgctccatgctggcggacgccttgcagcattaacgtagcacttgggggaatctgttggatgcccgatcggtctcaagtcatcatccgcattgtgccgggaggtgggacgaagaccctccaacagattatcaaccagttggagtatctatctcggaagggcaagttggagctgcagcgttcggcccgacatctcgatactttcgtaccaccggatgaaatccgcgaactcgccctaagctgggttcaagaaaccgggacttatcacgaaagtcggccagacgaggaaaggcaacaggagttgacgacccacattattgtaagcttccctgccggtacaagtcaggtagcagcttatgcagcgagccgggagtgggcagccgagatgtttgggtcaggcgcagggggaggccgatacaactatcttacagcctttcacatcgatcgcgatcacccacatctgcatgtggtcgttaatcgtcgcgaacttttggggcacggctggctgaaaatatcccggcgccacccccaactgaattatgacgccttgcgcattacaatggccgagatttcacttcgtcatggcgtcgtcctcgaggcgacgagccgagctgagcgcggcatctcggagcggccgatgacttttgcccagtatcgacgcctggagcggcagcaggccaatcaaattcgtttcgaggatattgatttcgaagaattctcgcctgggggaaacgacggagaaccggatcaatcttttaattcttcgtacggttcgctgcctcaaaacgtgtcagaaaacttgcgacgaaacggcggtctgcaatcggtgtctgaagtacgttcccgggcactgatcggatcgaacggcaacggtgtcacgcccgatcgtgtatcatctggaaacgattcccgttctcaatccggcagcgataaagcctttgtggacgatggcaacttgaggaacggccctctcaccatcgccggagacggccaggatcttgaaggccgttctggcatgcatcgtttggcaaccgaacccgtcacgcacacaacaagcgaagatgatgttcggcaacggcctcatcgaaaacggcctcgcggtgatgaggaagagcagagcggcgcaaaactgaccagggtagacggaattcggacgggagtgacaatcagcgccgtaccggttgcccaggacgatccgattacatcgccgatccagccccctggatcaaatccgttggatgacccggggcagaccaacatcgctacggacgcattgcctccgacagccgatcgccggcagcagggagagccaaattctaagcgtcctcgtgatgatgacgccgagccgagcattcgtaagcggtcaagggacgggcgcagccaaggggatgagggaaacagaaggtagaaaaggatcgatcatggcagatgaagaatttcgtcgagacttcagccgctccgctacgttgaactcgaataacgaaggcgctacaggcgcaccaatcccggatacatcggtgtcgactgccttccgacaaaaccgcagagaacacttaacgaccagttccttacggccattaattactggtggggccggcgaccttccaggcacttcccccgaaaatcaacggccgacaacccatgccccgaatacagcagaaatactcccggtgagatcagccgaagctccggctggccaatcagcggtctttccgaggagcaaggggtattttcgtacggccatgcaatacctgcgggagattgaaatgcagtccactgccagggcggaccacgaggcgacgtcgtcgtcacgttttggcaagggggaaagaaagcagctgcgcgaacacgagcaaaccggtgatcaaagtgaatcaatggcgaagcggagtaccagctcgggaatgcaggtgggaactattgccgcgtctttggctatgaaagatagctcgaccgcgacgccggcttcgcaagggccgcaccggtctgggatggagctggataatatcgaagggactggagagagccgtacgcaatctcagcacgaaattgcgacacaacctattgccacaagcgcttttggttcaggtcatgatgtgatcattcctggagagggccagcctgaagaaacattcgacggcgcaccatattggatggtagaaagcggggatagcgagaccaatagccttccgtcgatccaacaaggcttatcttggccagggttagatgaccttgatttgctcgccgacagcagttcaccgctcagtactcaggttgttcctcctgacgtagccgctacgaatttaagcactgaggcagggtccgccagcgtcgaaagcacccaaatcctcgccgacagtgcctattccgacgacgcgaccgaaaccggtggccaaacagcttcaggatcgatccatgaaggttcagttcgttcgcttggctcaccggaggaacgcttcgacgcctttcccgagttgaccgacgaggacttggcgcagattgacgcattctcgcgagcccgctctccttcgaggcgagatgctgcgcacgctattcatgcggtcagcttggatgatgccttctcacgcaaggatcttgccgcgggccacgacggccggcttacgaatcctgcgttccgtcaggaggcttctctgcaggaatcgagcccatcagaaacgaagtccgacgacttcccggaattaaccgacgaggacctggcacggatcgacgcactctccgaagcccgctcaacttcgaggctggatgttgcacgcactattcatgcggtcaggttggaggcctctcggcaggacccgagcccatcagaaatgaagtccgacgacttccccgaattgaccaaagaggacttagcccggatcgacgcactctctggaccccgcccgactacggggcgggaaggtgcgaacactattcagccagtcaggttggacgatgctttctcagccgaagattataccgtgggccgcggcggccggcatacgaatcctgcgttccatgaggaagcttctcggcaggacccgagcccatcagagatgaagttcgatggcttgccggagttgaccgacgaggatctggtaaggattgacgcaatctctgaaccgcgctcgaagagatctcgctccgagcaggttgtcgggggaaaatctcataggctcaaactgaacgaggctatcgaggaggcgggctcgcccactaccgtttcgggaacaagcgccgccagggcggcacctatcacgctcgaaagcggagtacctaggttgccgcaggctacccatacggaacttcctggctcggacgttgcgggtccactgcggatcgggaaggcgtcgtgtgccgatatacggcgagatgtttcggataaagggaaacgaaaagtctcgtcagattatcagattcagcaaggtcctggcgcgtcctatgcttaccagccgcgacgcatgccgacggaagcgattttcgatgctgaacggcttggtgacgaagtgctcatcgggaatgagccagttccgcggtggccttacccctgggagcacgttgttcgttttgaaacaggcaagcagctcttcgaaagattgcagaagtggccgactgcggaggagttggaaaacagtgtgatcctcgtcaaagcggatagcgcgcaacgatcctatatgccccgaaatcagctgctcttgcatctcggatccgaaaagtattacgaacgaatgaaagcgctcggtctatccgataataccttcagcggattgaagtccgatagcaaatatttttatgatacacctgctggaccacgggtaagtgaacatgattatgagttcccgcggcaggcaggcgtgcataatccagaggtgatggttcagcatcatagcggaaccttcttcgcgttttggccaaaacttctcaggctggaaaagcttgcgacagtaacgaatacgtatggcgcggaaaatgtttggctcaaatcgccagccaatgcttacatgacgccggaaacctataattccaaggttgcgaacggcgccgaaaaccttttcatcgagttcctacgtcccgctcctgtccacgaccacctgcgggaacctggaacttcaaacgcaaaaactgttgacggacgaaacgaagatcgcgcgcaacaggccgaccggttagcagatcagtcgatgggccggaacggggacggccgtgacgcgcagggtggtaagagaaccttggaaccgcgcgtgcgcgaaaggtatgggctgtagatccagctgcccgtaaagcaaacagccgtccaataggataaacggagcgtggtcacgtgatgaattcaggcaagtacacgccaataggcctggctgcgagcatagcatgttcattggccgtggggttttgtgcggccagtctctatgtcacatttcgccatggctttacgggcgaaacgatgatgactttcaacgtttttgcgttcctgtacgagacaccgccttatttgggatacgcaagtcctacgttctatcgcggactagccatcatcgttgcgacgtcaacgctcgtgttgctatgccaactactgttatcgatgcgcgagcgcgaacatcacggcactgctcgctgggccggctccggcgagatgcggcacgccaggtacctccggcgctacagtcacgtcacgggtcccatcttcggcaagacatgtggaccacgttggtttggcagctacctaagtaacggagaacaacctcacagccttgttgtcgcgccaactcgcgctggtaagggtgtcggcgtcgttatcccgacgttgctaaccttcaagggctcagtaattgccttggatgtgaaaggcgagctatttgaactcacatcgagggcgcgtaaatcaagtggcgacgcggttttcaagttctcgcccttagatccagagcggcgaacgcattgttacaatcccgtgctggatattgccgcgttgccgcccgaacggcgctttacagaaacgcgccgcctcgctgcgaatctcatcacagccaaaggcaagggagccgaaggtttcatcgacggggcacgggatcttttcgtagcaggcatattgagttgcattgagcgcggcacaccaacgatcggtgccgtttacgatctgtttgctcagccgggtgaaaagtacaaactatttgcgcaactcgcggaggaaacacaaaacaaagaggctcagcgtatcttcgacaatatggcggggaatgacaccaaaatcctgacatcctacacatcggtgctcggcgacggcggactcaatctttgggccgatccactcgttaaagcggctacaagcacctcggatttttccgtctacgatctgcggcggaagaggacttgtatttatctttgtgtcagcccgaacgatctcgaagtcatagcgcccttgatgcgtcttctcttccagcaggttgtatcaatcctgcagcgatcgctgccacttggagatgaacggcacgaagttctgtttctcctcgatgagttcaagcacttgggcaagctggaagctgtggagacggcaattacgacgatcgccggctacaagggccgcttcatgtttattattcagagtctttcggctttgacaggaacttatgatgaagctggtaagcaaaattttctcagcaatacgggtgtgcaggtattcatggcgactgctgatgacgagacgcccaactacatttccaaagctatcggcgactacacattccaggctcgctcaacgtcgtacagccaagcgcgcatgtttgatcataacatccaaatctccgaccaaggagcaccgcttttgcgcgccgaacaagtccgcctactcgacgacgattatgaaatagtccttatcaaaggccaacctcccctcaaactgagaaaagtgcgatattattcggatttcatattgaagcgaattttcgaaagccaacacggttcccttcccgagcccgcatctttgatgttgccgggagacacaaacctcgttgaaggcaagctcgaccagggaacagccgatacaacttcgcaaaaaccggtgcagattgaagagaacgaccatggggaagtcgtttccccccaaaacagaactgtggctgatggagtaatgccaatagaagttcgcccccgttcaaacgaagttgatgacgagcgggaataccaaggcgcggacgccgcaacctctgagaagcttccggaaattgctccggctcttttggcgcagcgcgaactcctcgaccagatcatttcgctccagctacgaggtagaacagcctccggcacttgaagctcgatgtgataccgcttcggatcgagtcttgttagcgccgtcagatgtcccgtttcgggaatgcattaccaacgttgaaggttgccaattgatgagacatcgccatgaaaccgtcgggaagctcaaaaactggatacgccgggtcggctagatcctcgccccaggttcgcgcaggggttacgcctgtccttcatcccaaagagccttggaaccgttttgcttgctccccatctgacggccaaatggatcaccgggaaaatttatctgcgcaattcgcttacgatggaatgagactgggcgcagccgagcggtccgcgtacgagacatgggatagagcggaccggccgagttggaaagacctgatactgagcgctcgcctgaacgcgattgacagtttcgcttggaatgtcgatgtcggagaaagtacctcttcaacttttgtttatgatggtgttcctctggcggaaggggaacggcatgcctacgaggaatgggtggagccgggacagcccagttggcaacaactcgttgtgaacgcgcgtattgaagagctaaacacctctgctgcaattccgaatgagtgtagtccccttcaagaggaattccgatcggaggcgcctaagcgtaagcggacaagctccattggtcaaaagaacggtttccctgaaccattcgaatttgatgggatgagactcggctcgcctgagcgcgaagcatatgagaattggagtaaaccgcaaccgccatcctggaaagacctgatagttgacgcccgtcttgacgcaatcgacacctccacctggctcaacgaatcaaatgacacctcggtcttcgagtacgaaggcgtcccgcttggggagggggaacgcctagcatatgaaaaatggctcgagcccgcgcaaccaggatgggaagacctggttgtggacgcacgcgttgcggaattgcaccagtctgctccgacgtggtgcgaacaccaaccaggcaacgacgtttcttccaacgagtccgggcgcatctcgggcgtcccaattaacgccgagcaggtgtcgcgtgcgttcttcgtgtacgatggagtggcgctcggagcagccgagcgcgctgcgcatgatcgttggagcaggccggaccggcccacctgggaagatcttatcatagtcgcgcgccaagctgctatcgaaggcggtgccgtttcgaatgggatgatcgggaagacatcttcctcagtctttttatacgagggaatgttgcttggggatgcggagcgtcaggcgtacggacggtggaggcagctagcccagccgcggtggcaaaatctggtggtgaacgcgcgcctggcagagctcgacccggcggcctggattcccgatgaacatgatccgtctgaggatggcggggcgactggtcttctgtcgcaagcaagcacgaccaataagtcccgcctcagtttaggtgatcaaccggaagcgcgctcgcctagtctcgcacgtgagccagcacaacagccgactcacgtgcaaaacctgacgtgcgcacaattggaagcaagacgtgctctatatttcgggccctctgggagggatgcagaccaaaccgccagcatcgccgacagtaatcgcctcgacgaggtaagcaaagttaaacggctgggtgccaagagccgtcgaggcgctaaagcaacggcctatgacgtaatttcaagtgcggaaagactgtcgtctcacgagggttgtttgacggttcagtccactcagccagaaaaagccgcttgctcgagaagcgacaatatcggcacttatggaagtcgaaaaaacgaacgagctcggcttgcgaccgagaccgggaaatacgagtcggagcatattttcgggttcaaggttgtccacgataccctgcgctcgaccaaagagggccggcggctggaaaggcctatgcccgcctatctcgaatgcaaggaacttcatcgacaacatgttggtacgggaagggggcggacccgactggtggggcgcggctggccagatgacgcaagttaccgctcggatcaaagggcaactctatcggaccctgttgcgtccacggaaggtgcaacggcgtcaaacgggtatcaattaaaccagctaggctacgcacaccagctcgctaacgacgggctgcagagcgaaacgccagatggggttatcatgccgcttcaggtcgcgactactagctacaactatacggtaagccgcgatccggtactttctccgcccagtaaagagcaagctccggaattgttgcaccttgggccacgtggccagacagaagctgtactcgctcgcgaaacagcattgacaggaagatggccaacacgcgagcgtgagcagcaagtctatcgagagtttttagccctttatgacgtcaagaaggacctggaggccaagacacttgggttgcggaagaaaaaagctgcgcttgtttccgcgttgaaccggactgccgcctcaataggcacttcacccttgaaagcccaatcgtcgagcgcagaagttgaaaaagcaactcacgagtttgatgaacgacgggtttatgatccgcgcgatcgcggtcgggacaaagcattacaacggtgacaaagctggagatgaccatcgttagcggactttgtatcgatgcttaaaaatgatggttcagcatttgaaccgcaggctgtactgctactctttgaaatcccccgcaaaaaacgcgccgtagtgcaatcgatcgccagggcctgaatttcagttgaaacacgatgtccattcaacgccttaagcgcaggtgttagacttcgcatatccttgatccgcgttggagtgtgagttatgctcgaagacggccagaaaatgcaggaacgcgagctttcggtcctcggtggtgataagaccgaaagtgcagccaccttaccgaatgacattttggtggaagttgccaaacatctgccgaccgacgatccagtcgaaacggcagccaacctcacaagctttaaacttgcgagcccgtcggttcgggcatcggtcgatcagagcgatgtcggaacgttccaccgaagcgtaaatcgactgggtgcctcaagcaaggcattgtatgacctggctgtaccccggaacggctttgccgagtatccgaactatccggaaccggaccctgctggcgagtttgcgctggcgagccagcgcatcaggacaattggtccaactttgaagttccaatcgcccgccaggaaaacagcgatcgtcaatcacattctcaatatgtcggaaggcggtgagcaggcggaggcgattatgtcgatgacgtcccatttgagtgacctcgagagggccgacaagagacgccttatcgacagagcaattgaatacttcaaggtagacgggcctatcaactacgacagtcgccattatgcagcctacgcgattgcagcggcgcacaaccagttggaactcgaacacaagtcacaaatcttcgatgcaatggcgaagagaccccagcttgcgagactctacgccgacgaacgcatccatgcgcaggagcatttggggcttgcgtcgacatccgaaggtccgcacgagcggagcaataaacaactggccacggacattcttcagatcgaacaaaaaatccgcactgagctccttcccgaaactctcagtgctcacgaccagatggtaaaagcggaaggaatcgctggatcgattgagaaggcttaccgcgatgctcgcgcgaacttaacccgggcaccgcgagggaggtcaaattctgatctcagccggtagaacttggtctgaatattcgattatttctccgacgaaagcacttggttgacgagcgaatttttaaacgagcatcgataaagcgtgcacagggaaggcccacctaatggtcagtataaataaaaaagccccgcagaaatcttctacagcgaccacgcgcaattctgatgaacgcctggttgatcagttagggcaggcgtaccttgtggaacgcgaggcgcaggaaaggcgcgcgcgacttgaagcgccgccggcgttgaaaaaatatgcctcgaaactgcaactcctcaataagctcgatgcagatttccgtggtgtgatcgcccacaagccgtttcgcagcgagcaactgcgggtcgatagcaatggagaattgacccatagtcggggtctcataaaaaagaagaagaaggttttcattcgagatcaaacaactggctcccttcggcttattcactacgagaggtctgactggaatagtgtccgtcgctatgactacgaccagaacggcgtgttgagcgagaaacatatcaaaagcaaagccggtgccttcgaagaaaaatgggagcgggacgaaaacggtgaactgatgcgcacccgatacatcgatcggcgcagacttactggacgggcgttgcatcccatttccgaggaaatcggacgtccttatgagagtggcctggagaagaggctttatcgcgttttaacgcgtcgggagggctcccaacaaaagactttcgaacgcgatgacaagggtaatctggagctcattgcaagcaaacggatgggctattcgatgtcttcacaaaaggcaccggatcgacaaacatctagaacaagtatccgcaagcttggcggagctttcagtgaatcatatagatcccggctggataaggacggccacgagttaggtcgggatgtctccgcacatcgaagcctcctcaacaagcgttcagccgtctacgatgacgctacaggggagctaaagagcagcaagcacacgtttgggaagatatacaaagcggaagcgacatacctgaacgccgaaatcaaagaggtctccaaaaaaatcctcggagttacggtcggtaaagaattgaagacattaagtggacgcgaactcgaggctcaagtattacgtgccgcggaacgtgccctccataaacaggcctggcagcacccttcagctaacccctcgcggtcgcagcgggaaaataccaacagtcatcttggaaacgaacttgatgggccgctcgaatcggatatgaccgtcgtccatcaagccggatccgggttcgtcggcgacgatcgccacgttcatgatggcggtggatccaggttgaatgaattgcacgaatcgcgatcgtcgaatgatagggaaaagcttagttcgccgagagccgaaatttgcgaccgctcgatgaatcgggatcgcaccggctcaggtactttgtcgcgatgatagctaaactaggggggcacacaccgagttgtcaagtcgccccccggcaaatgagctcatccctgctgccatattcccaaactaccaacgtttcgcgtcaaactaggttcgcctaaaggcatgaaagtcgcgtttcaacgaacaggctcgatcgaaccagcgacccaacgttaatgattacaaactgcaaaccgtaacccttaaaggtaaacatttccaaagtttcatttgtagcgattttgaatttttcatgtgtagaagcgttcctgatatcaagttatagtcctcgcaatcgatccaccatagaaagattgctcatctgagcttaaggaattcaattcttcctcagatatacttggagccacgcgggccatctaactgacttctaggttttgatgaacaccatgtcccctcaatggctttgagaccgcaccggatgctcattgagttttgaaatcagcaccgttaccgaactttccatcctggctggctgatcacacacgattggaaagcgaggggaataggagtggcaagcatgccaaccgacgacattgtaatgtccgatcccggaatggctgctgttgacacgtctgtccctacgcgcttccagacagatcttcgccagttcagtaacctgctggacgagagcaatattgtggaatgggtcgtaaaccacgccgcaaatcgcaatgcgtgcttcggacaagaccaacttaagatttcggcggctcgggcactcacgcactacaaacggccggtacaagaaatcgacctgctattccaacaggtcgtcgcaagcgacaaactgacccggcttgattctccagatggccaacctctctacacctctacagagctgagagctgcggaacagaatactgtacgtcatgtcaccaatctttccgcagaagaaagcttcggcgttccgggaaccattgttgatatggtcgccgatagaggcattttcacaccggagctcagaggagcattacattatctgtctaggcgaaatcgagcttccactatggtgggcgtagcgggttccgcaaaaacaagtgtacttggagccctgaacgaagcggtggacaggttcaacgaggaagcgctcccgaccgagaaaataaagttgattgccttcgtacccaccaaccgggcggcccaggagcttcgtgagaaaggtcttcgcgacgtcgcgacgactttcaaggcgaaagaccgcaccatcggaaaaaataccatcgtcgtgatcgacgaaatgtcgatggccaagacgcaggagatcgcgcaactaatcgagacagttgcaaatgccacttcctatcacccaaccgaaaggccgaaactgatatgtgtgggagacgatcggcaactgccccctgttggacctggtgatctgcttccgattattatggagcaagccggctgctatgaactcgtccagccgcttcgacagctcgacgcgcggtcgcgaatagaaacagaaaagctcggacgtgacatacgacgcgatgccaagagcgccgtcgggcactatctcacagcgctcgaacaaatgggcctcgtccatttcgtatatgctcctcctagcggctcaaacgtcagggcgtcagaccagatcactgaccgcatgctggaggagatgaatagagtttgcgaccgatatccggtgtatgatcgtcttgtgatggcacactccaacaggagcgttaatcgcgccaatatcgctcttcatcagaggtttgttgacaaatccggattgcaagaccagcaaatgagtgtgtggtcaagcgtaatagatagtggaagttcggaatctggaatagatgagggttcgtcgagacgcgttcagcaaaggattaatctcgtcttgggagacaggattatattcagcgaaaaccgtctgcaagcggacatccgcaatggtacttttgcaactgtagtcggacttaatagcacagaagcttcccgagctgagaatggcacatctgctggggttcgcattactgcaaggttagacggatctggacgaaccgtcacttggaccgatagcgagtttaagggttttacctacggctatgctgctacaatttataagagtcagggggccacagttgatcacgccattctcctctgtgatggtgctctttcagataagctgacctatgtcggcctgacgaggcatcgcagtcgtctcgacgtcattgcttctcccctcgtggcaaacaatgttgaggctctcagtgcaaagctcactcagcaaagcgcgccaaataattccattcaattcccagcgctcgaacctgctgccttccaagagcgctcgtcggtttcgaacgcgcacttgctaaggccgctcgaccagatggaaatctctccaacgacgccgctttccttccaaggcagacaagaaagctccttcggggagaaccagctgcacgaaccatccacatcggatgtggaaatgccggaaagcgcggacctttacccgagcgccattgacttcgaagttttccagtcttctgtcatcacgactgaagagcaaatgcaaccggaattctgcgggtcggaatcgacggctaacacctcagcccacgaaagccaccaagtcacgatcgcaacggtaacaaacctggggatagcgatcgagaaacggaaacgagcggcggcaaaggaagaaatcgattctcgcaaaaaaatggcccgtcactcagttagttctgccgaggaagttgaagatatcacttccctgttcgaacgattgaaactggcaacctcggtcaccaacacagttcaaccgacgtatgcggacgtcgatcatatcatcggtactgatggccgagtgggtccaataccgcaccgggaagatgatccgggatcaaattgggaattgtcgtcaacgaacgccccctcaaacgtctcagagaaacgatcgaccaaagactcaggagaaaaaaatatggcttcgaatgaccagaaaatgcaaggcgctgactctgcaggcagcgagcgtcatttttcgcagacttccattgtggaagccctgcaggctggcgatcacaagaagctcgtggaaacgctcgccgcaaccggctcccttgtcgagaaggcggacgccgggtcggcgctgaaagcaatggcggatgcttacgaaagggatatcaaggcagatcccgaaaaggtccgcatgctctatgccaatcggcgcgttgatgtagatgcgctggccaaggagatcgaaaaacagggccttgcatccggtcgcctgagtggcgaccctgtcagcttcaaggcgttcaacaacaaggatatccgtttcagggtcggcgacaggctcagcgtcaccagcagctacaggaatctcattgccggtgaagccggccggatcgaaaagatcgaaggcaaatccgttaccttcaaacccgatggcagcagtcagagcaagaccttcgaggccagcaccggcgagccgggtgacaggggcgtcaagggctacaaaatcgaaggcccgatgacggtcatcggctccgcctatgaacttacgcgacatgatgacggcaaacgtgttgaccaaatctatctcttgaacgacgctgcccatggcatcaacgccaatagcgctttgctgagcaaggccgacgagcggacgcagcttttcacttcgctcaaggacaccgccgacagggacatcttggccaggcaactgggaaagccgggcagcgagctctaccaggacaaattcccagccttgccggactacacagccgccatcgatcggattgcaaaacgccaggaagttgtcgcggcacgtggcgaggagaaaacttccgccccgatagacctggcgccccaaacgccgcccgacgctgccgatgcggcagtcaataaccagaaaagaatcgtcgaggccttgcaggcaggcgaccacaagaagcttgtggaaacgctagccgcaaccggctcccttgttgagaacacaaatctcgtatcaacgttgaaagcgatagcgcagacttatgaaagagacattagtgcggaccccgagaaagtccgcgctctctacgccaacaaacgtgatcatgtagatttgcttgccgaagagatcgaaaagcagggccttgcatccggtcgcctgagtggcgaccctgtcagcttcaaggcgtttaacaacaaggatatccgtttcagggtcggtgacaggctcagcgtcactagcagctataggaatctcattgccggtgaagccggccggatcgaaaagatcgaaggcaaatccgttaccttcaaacccgatggcagcagtcagagcaagaccttcgaggccagcaccggcgagccgggtgacaggggtgtcaagggctataagattgaggggcccacgacaatcatcggctccgcctacgaaatcagaaatcacgacgatggcaggcgcgtcgatcaggtttatgtcttgcacagtgccattttcggctcaaatgccgcgagcgccctgttgagcaaggccgacgaacgaacccaggttttcaccgtgcaaagcgacacggctgacagacacatcttggccagccaactcagcaagcctggcagcgagctctatcaggacaaattcccagtctcgccggactacgcagccgcgatcgaccggatcgcgaaacgcgaggaacttagggccggacgtggcgaggaaagtcctactgttcaaatggagccggtgaaccaaacgccctacgaggcgggacattggacgcctgacagtcaaaaatctgtggtggaagccctgcaggctggcgatcacaagaagctcgtggaaacgctcgccgcaaccggctcccttgtcgagaaagcggacgccgggtcggcgctgaaagcaatggcggatgcttacgaaagggatatcaaggcagatcccgaaaaggtccgcatgctctatgccaatcggcgcgttgatgtagatgcgctggccaaggagatcgaaaaacagggccttgcatccggtcgcctgagtggcgaccctgtcagcttcaaggcgttcaacaacaaggatatccgtttcagggtcggcgacaggctcagcgtcaccagcagctacaggaatctcattgccggtgaagccggccggatcgaaaagatcgaaggcaaatccgttaccttcaaacccgatggcagcagtcagagcaagaccttcgaggccagcaccggcgagccgggtgacaggggcgtcaagggctacaaaatcgaaggcccgatgacggtcatcggctccgcctatgaacttacgcgacatgatgacggcaaacgtgttgaccaaatctatctcttgaacgacgctgcccatggcatcaacgccaatagcgctttgctgagcaaggccgacgagcggacgcagcttttcacttcgctcaaggacaccgccgacagggacatcttggccaggcaactgggaaagccgggcagcgagctctaccaggacaaattcccagccttgccggactacacagccgccatcgatcggattgcaaaacgccaggaagttgtcgcggcacgtggcgaggtcaagccgattgagcgcaccgaagcgcccgaaataccccaggttcaagctactatcgttggcgagacctcgatcattcggttgcccgatcggataaaaaccaatcgcgagaagccccaagaacttgatgtagtctcgaaagaagggcacgaaactacaggtagtctgaggcagagtgtccaagagagtaatgcagcaccaaaacagacttcgccgaaggcggcaaatgacgtggatcggctgacacgggattttgacgagcgcatccgcgtccgcggggatggacgtggactctaagtaaaagagggtgcgtaacgcgcaccctcttccactcgctgtccctgtatccgttctctgtcgcgagaacgtcgatcaccttttcggaactctttccagatctgcttgaggttccgttgccggagaaagcattggcgaggattgctgggatccggcggacggcgatgcgatgtgccggttcggtgaccaattccatcagagaccggccgacgtatggcctgccgaagtggaaacggctgatcagcttgcgaggccgagctacttccccggcaagcgtcgggttcggggcattgttttcctcgaccatggcggtattttgctgagttgcccggctaaactaggctttggcagcctgttctttgaactatgtacctcgcgaatgcatctagatccaatccaatatgcatggcatgccgaatccatgtgggagtttattcttgacacagatatttatgatataataactgagtaagcttaacataaggaggaaaaacatatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaataatgtctaacaattcgttcaagccgacgccgcttcgcggcgcggcttaactcaagcgttagatgcactatacgtaccaatccaaatcggatccccctgcagggctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatctatcgatggtacctatctgcagacggaagggattcctgttaattaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctcgcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggcagcgcaggccgcccagcggcaacgcatcgaggcagaagcacgccccggtgaatcgtggcaagcagccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggcaggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatgcatataattgtggtttcaaaatcggctccgtcgatactatgttatacgccaactctgaaaacaactttgaaaaagctgttttctggtatttaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcagg

SEQ ID NO: 14 - DNA, 896 bp, Zea mays
  source 1..896: mol_type = genomic DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctt

SEQ ID NO: 15 - DNA, 82 bp, Zea mays
  source 1..82: mol_type = genomic DNA
tccccaacctcgtgttgttcggagcgcacacacacacaaccagatctcccccaaatccacccgtcggcacctccgcttcaag

SEQ ID NO: 16 - DNA, 1010 bp, Zea mays
  source 1..1010: mol_type = genomic DNA
gtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 17 - DNA, 202 bp, Figwort mosaic virus
  source 1..202: mol_type = genomic DNA
atcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaa

SEQ ID NO: 18 - DNA, 184 bp, Peanut Chlorotic Streak Caulimovirus
  source 1..184: mol_type = genomic DNA
caacgagatcatgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgacc

SEQ ID NO: 19 - DNA, 187 bp, Mirabilis Mosaic Virus
  source 1..187: mol_type = genomic DNA
ccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatccca

SEQ ID NO: 20 - DNA, 1988 bp, Zea mays
  source 1..1988: mol_type = genomic DNA
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 21 - DNA, 573 bp, synthetic construct
  misc_feature 1..573: note = 3xENH (FMV ENH, PSCV ENH, MMV ENH)
  source 1..573: mol_type = other DNA
atcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaacaacgagatcatgagccaatcaaagaggagtgatgtagacctaaagcaataatggagccatgacgtaagggcttacgcccatacgaaataattaaaggctgatgtgacctgtcggtctctcagaacctttactttttatgtttggcgtgtatttttaaatttccacggcaatgacgatgtgaccccactaaaacattgctttgtcaaaagctaaaaaagatgatgcccgacagccacttgtgtgaagcatgagaagccggtccctccactaagaaaattagtgaagcatcttccagtggtccctccactcacagctcaatcagtgagcaacaggacgaaggaaatgacgtaagccatgacgtctaatccca